(19) United States
(12) Patent Application Publication
(10) Pub. No.: US 2025/0179586 A1
(43) Pub. Date: Jun. 5, 2025

(54) SYSTEMS AND METHODS FOR DETECTING SOMATIC VARIANTS DERIVED FROM CIRCULATING TUMOR NUCLEIC ACIDS

(71) Applicant: Tempus AI, Inc., Chicago, IL (US)

(72) Inventors: Kyle Ashley Beauchamp, San Mateo, CA (US); Maysun Mazhr Hasan, Bothell, WA (US); Sandra C. Hui, Oakland, CA (US); Kaveh Karbasi, Redwood City, CA (US); Christine Y. Lee, Menlo Park, CA (US); Anne Franklin Sonnenschein, Chicago, IL (US); Jason Yamada-Hanff, Los Angeles, CA (US); Wei Zhu, Naperville, IL (US)

(21) Appl. No.: 18/969,159

(22) Filed: Dec. 4, 2024

Related U.S. Application Data
(63) Continuation of application No. PCT/US24/58529, filed on Dec. 4, 2024.
(60) Provisional application No. 63/574,751, filed on Apr. 4, 2024.
(60) Provisional application No. 63/606,068, filed on Dec. 4, 2023.

(51) Int. Cl. C12Q 1/6886; G16B 20/00; G16B 40/20

(52) U.S. Cl. CPC C12Q 1/6886; G16B 20/00; G16B 40/20

(57) ABSTRACT

A method of identifying a variant as a somatic variant derived from cell free DNA (cfDNA) identifies the variant at a locus based on differences between the nucleic acid sequence for a cfDNA fragment in a plurality of cfDNA fragments and a nucleic acid sequence for the locus in a reference sequence, where the cfDNA fragments are from a liquid biopsy sample from a subject. A set of cfDNA fragments comprising the variant, in the plurality of cfDNA fragments, determines fragment length metrics. A variant allele fraction (VAF) is determined based on comparison of the number of cfDNA fragments having the variant and the total number of cfDNA fragments mapping to the locus. Clonal hematopoiesis prevalence metrics for the variant are obtained. The fragment length metrics, VAF, and hematopoiesis metrics are inputted into a model thereby obtaining, as model output, whether the variant is a somatic variant derived from cfDNA.

### CROSS-REFERENCE TO RELATED PATENT APPLICATIONS

This application is a continuation of International Application No. PCT/US24/58529, filed Dec. 4, 2024, which claims priority to U.S. Provisional Patent Application No. 63/574,751, entitled “Methods and Systems for Filtering Clonal Hematopoiesis Variants in a Liquid Biopsy Assay,” filed Apr. 4, 2024, which is hereby incorporated by reference. This application also claims priority to U.S. Provisional Patent Application No. 63/606,068, entitled “Methods and Systems for Filtering Clonal Hematopoiesis Variants in a Liquid Biopsy Assay,” filed Dec. 4, 2023, which is hereby incorporated by reference.

### TECHNICAL FIELD

The present disclosure relates generally to the use of cell-free DNA sequencing data to provide clinical support for personalized treatment of cancer.

### BACKGROUND

Precision oncology is the practice of tailoring cancer therapy to the unique genomic, epigenetic, and/or transcriptomic profile of an individual's cancer. Personalized cancer treatment builds upon conventional therapeutic regimens used to treat cancer based only on the gross classification of the cancer, e.g., treating all breast cancer patients with a first therapy and all lung cancer patients with a second therapy. This field was borne out of many observations that different patients diagnosed with the same type of cancer, e.g., breast cancer, responded very differently to common treatment regimens. Over time, researchers have identified genomic, epigenetic, and transcriptomic markers that improve predictions as to how an individual cancer will respond to a particular treatment modality.

There is growing evidence that cancer patients who receive therapy guided by their genetics have better outcomes. For example, studies have shown that targeted therapies result in significantly improved progression-free cancer survival. See, e.g., Radovich et al., Oncotarget, 7 (35): 56491-500 (2016). Similarly, reports from the IMPACT trial—a large (n=1307) retrospective analysis of consecutive, prospectively molecularly profiled patients with advanced cancer who participated in a large, personalized medicine trial-indicate that patients receiving targeted therapies matched to their tumor biology had a response rate of 16.2%, as opposed to a response rate of 5.2% for patients receiving non-matched therapy. Tsimberidou et al., ASCO 2018, Abstract LBA2553 (2018).

In fact, therapy targeted to specific genomic alterations is already the standard of care in several tumor types, e.g., as suggested in the National Comprehensive Cancer Network (NCCN) guidelines for melanoma, colorectal cancer, and non-small cell lung cancer. In practice, implementation of these targeted therapies requires determining the status of the diagnostic marker in each eligible cancer patient. While this can be accomplished for the few, well-known mutations associated with treatment recommendations in the NCCN guidelines using individual assays or small next generation sequencing (NGS) panels, the growing number of actionable genomic alterations and increasing complexity of diagnostic classifiers necessitates a more comprehensive evaluation of each patient's cancer genome, epigenome, and/or transcriptome.

For instance, some evidence suggests that use of combination therapies where each component is matched to an actionable genomic alteration holds the greatest potential for treating individual cancers. To this point, a retroactive study of cancer patients treated with one or more therapeutic regimens revealed that patients who received therapies matched to a higher percentage of their genomic alterations experienced a greater frequency of stable disease (e.g., a longer time to recurrence), longer time to treatment failure, and greater overall survival. Wheeler et al., Cancer Res., 76:3690-701 (2016). Thus, comprehensive evaluation of each cancer patient's genome, epigenome, and/or transcriptome should maximize the benefits provided by precision oncology, by facilitating more fine-tuned combination therapies, use of novel off-label drug indications, and/or tissue agnostic immunotherapy. See, for example, Schwaederle et al., J Clin Oncol., 33 (32): 3817-25 (2015); Schwaederle et al., JAMA Oncol., 2 (11): 1452-59 (2016); and Wheler et al., Cancer Res., 76 (13): 3690-701 (2016). Further, the use of comprehensive next generation sequencing analysis of cancer genomes facilitates better access and a larger patient pool for clinical trial enrollment. Coyne et al., Curr. Probl. Cancer, 41 (3): 182-93 (2017); and Markman, Oncology, 31 (3): 158, 168.

The use of large NGS genomic analysis is growing in order to address the need for more comprehensive characterization of an individual's cancer genome. See, for example, Fernandes et al., Clinics, 72 (10): 588-94. Recent studies indicate that of the patients for which large NGS genomic analysis is performed, 30-40% then receive clinical care based on the assay results, which is limited by at least the identification of actionable genomic alterations, the availability of medication for treatment of identified actionable genomic alterations, and the clinical condition of the subject. See, Ross et al., JAMA Oncol., 1 (1): 40-49 (2015); Ross et al., Arch. Pathol. Lab Med., 139:642-49 (2015); Hirshfield et al., Oncologist, 21 (11): 1315-25 (2016); and Groisberg et al., Oncotarget, 8:39254-67 (2017).

However, these large NGS genomic analyses are conventionally performed on solid tumor samples. For instance, each of the studies referenced in the paragraph above performed NGS analysis of FFPE tumor blocks from patients. Solid tissue biopsies remain the gold standard for diagnosis and identification of predictive biomarkers because they represent well-known and validated methodologies that provide a high degree of accuracy. Nevertheless, there are significant limitations to the use of solid tissue material for large NGS genomic analyses of cancers. For example, tumor biopsies are subject to sampling bias caused by spatial and/or temporal genetic heterogeneity, e.g., between two regions of a single tumor and/or between different cancerous tissues (such as between primary and metastatic tumor sites or between two different primary tumor sites). Such intertumor or intratumor heterogeneity can cause sub-clonal or emerging mutations to be overlooked when using localized tissue biopsies, with the potential for sampling bias to be exacerbated over time as sub-clonal populations further evolve and/or shift in predominance.

Additionally, the acquisition of solid tissue biopsies often requires invasive surgical procedures, e.g., when the primary tumor site is located at an internal organ. These procedures can be expensive, time consuming, and carry a significant risk to the patient, e.g., when the patient's health is poor and may not be able to tolerate invasive medical procedures and/or the tumor is located in a particularly sensitive or inoperable location, such as in the brain or heart. Further, the amount of tissue, if any, that can be procured depends on multiple factors, including the location of the tumor, the size of the tumor, the fragility of the patient, and the risk of comorbidities related to biopsies, such as bleeding and infections. For instance, recent studies report that tissue samples in a majority of advanced non-small cell lung cancer patients are limited to small biopsies and cannot be obtained at all in up to 31% of patients. Ilie and Hofman, Transl. Lung Cancer Res., 5 (4): 420-23 (2016). Even when a tissue biopsy is obtained, the sample may be too scant for comprehensive testing.

Further, the method of tissue collection, preservation (e.g., formalin fixation), and/or storage of tissue biopsies can result in sample degradation and variable quality DNA. This, in turn, leads to inaccuracies in downstream assays and analysis, including next-generation sequencing (NGS) for the identification of biomarkers. Ilie and Hofman, Transl Lung Cancer Res., 5 (4): 420-23 (2016).

In addition, the invasive nature of the biopsy procedure, the time and cost associated with obtaining the sample, and the compromised state of cancer patients receiving therapy render repeat testing of cancerous tissues impracticable, if not impossible. As a result, solid tissue biopsy analysis is not amenable to many monitoring schemes that would benefit cancer patients, such as disease progression analysis, treatment efficacy evaluation, disease recurrence monitoring, and other techniques that require data from several time points.

Cell-free DNA (cfDNA) has been identified in various bodily fluids, e.g., blood serum, plasma, urine, etc. Chan et al., Ann. Clin. Biochem., 40 (Pt 2): 122-30 (2003). This cfDNA originates from necrotic or apoptotic cells of all types, including germline cells, hematopoietic cells, and diseased (e.g., cancerous) cells. Advantageously, genomic alterations in cancerous tissues can be identified from cfDNA isolated from cancer patients. See, e.g., Stroun et al., Oncology, 46 (5): 318-22 (1989); Goessl et al., Cancer Res., 60 (21): 5941-45 (2000); and Frenel et al., Clin. Cancer Res. 21 (20): 4586-96 (2015). Thus, one approach to overcoming the problems presented by the use of solid tissue biopsies described above is to analyze cell-free nucleic acids (e.g., cfDNA) and/or nucleic acids in circulating tumor cells present in biological fluids, e.g., via a liquid biopsy.

Specifically, liquid biopsies offer several advantages over conventional solid tissue biopsy analysis. For instance, because bodily fluids can be collected in a minimally invasive or non-invasive fashion, sample collection is simpler, faster, safer, and less expensive than solid tumor biopsies. Such methods require only small amounts of sample (e.g., 10 mL or less of whole blood per biopsy) and reduce the discomfort and risk of complications experienced by patients during conventional tissue biopsies. In fact, liquid biopsy samples can be collected with limited or no assistance from medical professionals and can be performed at almost any location. Further, liquid biopsy samples can be collected from any patient, regardless of the location of their cancer, their overall health, and any previous biopsy collection. This allows for analysis of the cancer genome of patients from which a solid tumor sample cannot be easily and/or safely obtained. In addition, because cell-free DNA in the bodily fluids arise from many different types of tissues in the patient, the genomic alterations present in the pool of cell-free DNA are representative of various different clonal sub-populations of the cancerous tissue of the subject, facilitating a more comprehensive analysis of the cancerous genome of the subject than is possible from one or more sections of a single solid tumor sample.

Liquid biopsies also enable serial genetic testing prior to cancer detection, during the early stages of cancer progression, throughout the course of treatment, and during remission, e.g., to monitor for disease recurrence. The ability to conduct serial testing via non-invasive liquid biopsies throughout the course of disease could prove beneficial for many patients, e.g., through monitoring patient response to therapies, the emergence of new actionable genomic alterations, and/or drug-resistance alterations. These types of information allow medical professionals to more quickly tailor and update therapeutic regimens, e.g., facilitating more timely intervention in the case of disease progression. Sec, e.g., Ilie and Hofman, 2016, Transl. Lung Cancer Res. 5 (4): 420-23.

Nevertheless, while liquid biopsies are promising tools for improving outcomes using precision oncology, there are significant challenges specific to the use of cell-free DNA for evaluation of a subject's cancer genome. For instance, Clonal Hematopoiesis (CH) is a well-established confounder in next-generation sequencing (NGS)-based liquid biopsy cancer diagnostics. CH is a premalignant condition that is characterized by presence of genetic mutation in hematopoietic stem cells. Misclassification of CH variants as tumor variants can lead to false positive actionable variant detection, potentially resulting in incorrect interpretation of results and therapy selection. Moreover, CH variants may also interfere with quantitative variant monitoring, leading to inaccurate assessment of treatment response.

The information disclosed in this Background section is only for enhancement of understanding of the general background of the invention and should not be taken as an acknowledgement or any form of suggestion that this information forms the prior art already known to a person skilled in the art.

### SUMMARY

Given the above background, there is a need in the art for improved methods and systems for supporting clinical decisions in precision oncology using liquid biopsy assays. In particular, there is a need in the art for improved methods and systems for detecting clonal hematopoiesis variants in a liquid biopsy assay. The present disclosure solves this and other needs in the art. For example, models are described herein that use feature set derived from a nucleic acid sequencing reaction of cell-free DNA (cfDNA) fragments in a liquid biopsy sample to distinguish whether a candidate variant is of hematopoietic lineage or of solid tumor lineage. In some embodiments, these features are based on at least one or more fragment length metrics for cfDNA fragments encompassing the candidate variant, a variant allele fraction for the candidate variant, and one or more clonal hematopoiesis prevalence metrics for the candidate variant.

While filtering of CH variants is possible via matched sequencing of white blood cell and plasma DNA, emerging algorithmic approaches may enable a more resource-effective, time-sensitive approach with high precision. Advantageously, the models described herein accurately classify candidate variants as either of hematopoietic lineage or somatic lineage based on a single nucleic acid sequencing reaction of cfDNA fragments from the liquid biopsy sample and do not require additional nucleic acid sequencing of DNA from matched tumor tissue or buffy coat preparations.

For example, in one aspect, the present disclosure provides methods, systems programmed to execute such methods, and computer readable medium storing instructions for performing such methods, for detecting clonal hematopoiesis variants and solid tumor variants in a liquid biopsy assay.

The method includes obtaining a corresponding nucleic acid sequence of each cell-free DNA (cfDNA) fragment in a plurality of cfDNA fragments, from a plurality of sequence reads of a sequencing reaction of the plurality of cfDNA fragments from a liquid biopsy sample of the subject. The method then includes identifying a candidate somatic variant at a first nucleotide position based on at least a difference between a respective nucleic acid sequence for a respective cfDNA fragment in the plurality of cfDNA fragments and a corresponding nucleic acid sequence for a locus in a reference sequence to which the respective nucleic acid sequence maps.

The method includes using an identity of a first set of cfDNA fragments in the plurality of cfDNA fragments comprising the candidate somatic variant to determine one or more fragment length metrics for the candidate somatic variant. The method also includes determining a variant allele fraction for the candidate somatic variant in the plurality of cfDNA fragments based on (i) the number of times the candidate somatic variant is observed across the corresponding nucleic acid sequences for each cfDNA fragment in the plurality of cfDNA fragments and (ii) the number of times the first nucleotide position is observed across the corresponding nucleic acid sequences for each cfDNA fragment in the plurality of cfDNA fragments. The method also includes obtaining an estimated circulating tumor fraction (ctFE) for the subject. The method also includes obtaining one or more clonal hematopoiesis prevalence metrics for the first nucleotide position.

The method then includes inputting information into a model comprising a plurality of parameters thereby obtaining as output from the model an indication of whether the candidate somatic variant is (a) somatic or (b) other than somatic. The information inputted into the model comprises (i) the one or more fragment length metrics, (ii) the variant allele fraction for the candidate somatic variant or one or more features determined from the variant allele fraction for the candidate somatic variant, and (iii) the one or more clonal hematopoiesis metrics for the first nucleotide position, and/or an arithmetic combination of (i), (ii), and (iii).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

FIGS. 1A, 1B, 1C, and 1D collectively illustrate a block diagram of an example computing device for detecting clonal hematopoiesis variants and/or solid tumor variants in a liquid biopsy assay, in accordance with some embodiments of the present disclosure.

FIG. 2A illustrates an example workflow for generating a clinical report based on information generated from analysis of one or more patient specimens, in accordance with some embodiments of the present disclosure.

FIG. 2B illustrates an example of a distributed diagnostic environment for collecting and evaluating patient data for the purpose of precision oncology, in accordance with some embodiments of the present disclosure.

FIG. 3 provides an example flow chart of processes and features for liquid biopsy sample collection and analysis for use in precision oncology, in accordance with some embodiments of the present disclosure.

FIGS. 4A, 4B, 4C, 4D, and 4E collectively illustrate an example bioinformatics pipeline for precision oncology. FIG. 4A provides an overview flow chart of processes and features in a bioinformatics pipeline, in accordance with some embodiments of the present disclosure. FIG. 4B provides an overview of a bioinformatics pipeline executed with either a liquid biopsy sample alone or a liquid biopsy sample and a matched normal sample. FIG. 4C illustrates that paired end reads from tumor and normal isolates are zipped and stored separately under the same order identifier, in accordance with some embodiments of the present disclosure. FIG. 4D illustrates quality correction for FASTQ files, in accordance with some embodiments of the present disclosure. FIG. 4E illustrates processes for obtaining tumor and normal BAM alignment files, in accordance with some embodiments of the present disclosure.

FIGS. 5A, 5B, 5C, 5D, 5E, 5F, and 5G collectively provide a flow chart of processes and features validating a somatic sequence variant of a test subject, in which dashed boxes represent optional portions of the method, in accordance with some embodiments of the present disclosure.

FIG. 6 illustrates a model trained for the relationship between ctFE and variant allele fraction of somatic variants detected in liquid biopsy samples. Variant allele fractions for germline and CHIP variants do not correlate with the ctFE in liquid biopsy samples.

FIG. 7 illustrates the occurrences of likely CHIP variants in genes in testing and training data, as described in Example 3.

FIGS. 8A, 8B, 8C, and 8D collectively show performance of a random forest classifier, as described in Example 3.

FIG. 9 illustrates the GINI importance of each feature used in the random forest classifier, as described in Example 3.

FIG. 10 illustrates a data flow for random forest model training and validation of a CHIP classification model, as described in Example 3.

FIG. 11A illustrates that a circulating tumor fraction estimate (ctFE) for liquid biopsy assay has a strong correlation with the variant allele fraction of variants derived from ctDNA (blue circles), but not with CHIP variants (orange circles).

FIG. 11B illustrates that cell-free DNA (cfDNA) fragments from liquid biopsy assays containing circulating tumor variants have a distinct distribution relative to cfDNA fragments derived from non-tumor cells.

FIG. 11C illustrates the prevalence of CHIP variants in solid tumor sequencing data by gene.

FIG. 11D illustrates the relative frequency of common CHIP mutations occurring as tumor derived or CH derived within a solid tumor sequencing assay.

FIGS. 12A and 12B collectively illustrate performance metrics for a random forest model trained for classifying CHIP mutations, as described in Example 3. ROC curve and confusion matrix for test data. Performance calculated at variant level. True Positive (TP), False Positive (FP), False Negative (FN), True Negative (TN).

FIG. 13 illustrates performance of the random forest model described in Example 3 on the test set by gene. Although gene was the highest ranked feature in the model, both CH and tumor variants were seen and identified with high accuracy in many genes.

FIG. 14A illustrates the distribution of allele fractions for CH and tumor variants, as called in the model described in Example 3.

FIG. 14B illustrates the distribution of the number of CH variants identified per patient, using the model described in Example 3.

FIG. 15 illustrates a pipeline for whether a variant is a CH variant in accordance with an embodiment of the present disclosure.

FIG. 16 illustrates features used in a model for validating a somatic sequence variant in a tissue of a test subject in accordance with an embodiment of the present disclosure.

FIG. 17 illustrates a method for training a model for validating a somatic sequence variant in a tissue of a test subject in accordance with an embodiment of the present disclosure.

FIG. 18A illustrates data used for training a model for validating a somatic sequence variant in a tissue of a test subject in accordance with an embodiment of the present disclosure.

FIG. 18B illustrates Chip status distribution in data used for training a model for validating a somatic sequence variant in a tissue of a test subject in accordance with an embodiment of the present disclosure.

FIG. 18C illustrates number of variants in data used for training a model for validating a somatic sequence variant in a tissue of a test subject in accordance with an embodiment of the present disclosure.

FIG. 19 illustrates relative importance of various features used in a model for validating a somatic sequence variant in a tissue of a test subject in accordance with an embodiment of the present disclosure.

FIG. 20 illustrates truth table of a model for validating a somatic sequence variant in a tissue of a test subject, with respect to a training dataset, in accordance with an embodiment of the present disclosure.

Like reference numerals refer to corresponding parts throughout the several views of the drawings.

### DETAILED DESCRIPTION

### Introduction

As described above, conventional liquid biopsy assays do accurately distinguish between somatic and hematopoietic lineages for certain nucleotide variants without data from a second sequencing reaction of DNA isolated from a matched tumor or buffy coat preparation. In certain circumstances, obtaining a matched tumor sample is impracticable, such as for brain cancers. Moreover, performing additional sequencing reactions doubles the preparation time and cost. Advantageously, methods and systems are provided herein for detecting clonal hematopoiesis variants and/or solid tumor variants in a liquid biopsy assay using data from a single sequencing reaction of cfDNA fragments from a liquid biopsy assay.

The accurate identification of nucleotide variant lineage is critical because precision oncology therapies are tailored for each individual cancer based on, among other factors, the nucleotide variants in the genome of the cancer. Misidentification of nucleotide variants of hematopoietic lineage as somatic variants may result in the selection of an ineffective treatment of the cancer because the cancer being treated does not actually harbor that variant. Where the targeted therapy derives its beneficial effects from the presence of a particular nucleotide variant, treatment of cancers without the particular nucleotide variant may lack effectiveness.

The identification of actionable genomic alterations in a patient's cancer genome is a difficult and computationally demanding problem. For instance, the determination of various prognostic metrics useful for precision oncology, such as variant allelic ratio, copy number variation, tumor mutational burden, microsatellite instability status, etc., requires analysis of hundreds of millions to billions, of sequenced nucleic acid bases. An example of a typical bioinformatics pipeline established for this purpose includes at least five stages of analysis: assessment of the quality of raw next generation sequencing data, generation of collapsed nucleic acid fragment sequences and alignment of such sequences to a reference genome, detection of structural variants in the aligned sequence data, annotation of identified variants, and visualization of the data. See, Wadapurkar and Vyas, Informatics in Medicine Unlocked, 11:75-82 (2018), the content of which is hereby incorporated by reference, in its entirety, for all purposes. Each one of these procedures is computationally taxing in its own right.

For instance, the overall temporal and spatial computation complexity of simple global and local pairwise sequence alignment algorithms are quadratic in nature (e.g., second order problems), that increase rapidly as a function of the size of the nucleic acid sequences (n and m) being compared. Specifically, the temporal and spatial complexities of these sequence alignment algorithms can be estimated as O (mn), where O is the upper bound on the asymptotic growth rate of the algorithm, n is the number of bases in the first nucleic acid sequence, and m is the number of bases in the second nucleic acid sequence. See, Baichoo and Ouzounis, BioSystems, 156-157:72-85 (2017), the content of which is hereby incorporated by reference, in its entirety, for all purposes. Given that the human genome contains more than 3 billion bases, these alignment algorithms are extremely computationally taxing, especially when used to analyze next generation sequencing (NGS) data, which can generate more than 3 billion sequence reads per reaction.

This is particularly true when performed in the context of a liquid biopsy assay, because liquid biopsy samples contain a complex mixture of short DNA fragments originating from many different germline (e.g., healthy) and diseased (e.g., cancerous) tissues. Thus, the cellular origins of the sequence reads are unknown, and the sequence signals originating from cancerous cells, which may constitute multiple sub-clonal populations, must be computationally deconvoluted from signals originating from germline and hematopoietic origins, in order to provide relevant information about the subject's cancer. Thus, in addition to the computationally taxing processes required to align sequence reads to a human genome, there is a computation problem of determining whether a particular abnormal signal, e.g., one or more sequence reads corresponding to a genomic alteration, (i) is not an artifact, and (ii) originated from a cancerous source in the subject. This is increasingly difficult during the early stages of cancer-when treatment is presumably most effective-when only small amounts of ctDNA are diluted by germline and hematopoietic DNA.

Advantageously, the present disclosure provides various systems and methods that improve the computational elucidation of actionable genomic alterations from a liquid biopsy sample of a cancer patient. Specifically, the present disclosure improves upon the accuracy of distinguishing between (a) a somatic variant derived from cell free DNA (b) other than a somatic variant derived from cell free DNA. Moreover, because the methods described herein eliminate the need to process data from two different sequencing reactions, the disclosure lowers the computational budget for identifying actionable variants. As described above, the disclosed methods and systems are necessarily computer-implemented due to their complexity and heavy computational requirements, and thus solve a problem in the computing arts.

Advantageously, the methods and systems described herein provide an improvement to the abovementioned technical problem (e.g., performing complex computer-implemented methods for distinguishing between (a) a somatic variant derived from cell free DNA (b) other than a somatic variant derived from cell free DNA. The methods described herein therefore solve a problem in the computing arts by improving upon conventional methods for identifying such variants in a liquid biopsy assay.

The methods and systems described herein also improve precision oncology methods for assigning and/or administering treatment because of the improved accuracy of variant classification provided. Nucleotide variants can be reported as biomarkers and/or used in downstream analysis for identification of therapeutically actionable variants to be included in a clinical report for patient and/or clinician review. Additionally, therapeutically actionable somatic variants identified can be matched with appropriate therapies and/or clinical trials, allowing for more accurate assignment of treatments. The improved accuracy of biomarker detection increases the chance of efficacy and reduces the risk of patients undergoing unnecessary or potentially harmful regimens due to misdiagnoses.

### Definitions

As used herein, the term “subject” refers to any living or non-living organism including, but not limited to, a human (e.g., a male human, female human, fetus, pregnant female, child, or the like), a non-human mammal, or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a woman, or a child).

As used herein, the terms “control,” “control sample,” “reference,” “reference sample,” “normal,” and “normal sample” describe a sample from a non-diseased tissue. In some embodiments, such a sample is from a subject that does not have a particular condition (e.g., cancer). In other embodiments, such a sample is an internal control from a subject, e.g., who may or may not have the particular disease (e.g., cancer), but is from a healthy tissue of the subject. For example, where a liquid or solid tumor sample is obtained from a subject with cancer, an internal control sample may be obtained from a healthy tissue of the subject, e.g., a white blood cell sample from a subject without a blood cancer or a solid germline tissue sample from the subject. Accordingly, a reference sample can be obtained from the subject or from a database, e.g., from a second subject who does not have the particular disease (e.g., cancer).

As used herein the term “cancer,” “cancerous tissue,” or “tumor” refers to an abnormal mass of tissue in which the growth of the mass surpasses, and is not coordinated with, the growth of normal tissue, including both solid masses (e.g., as in a solid tumor) or fluid masses (e.g., as in a hematological cancer). A cancer or tumor can be defined as “benign” or “malignant” depending on the following characteristics: degree of cellular differentiation including morphology and functionality, rate of growth, local invasion and metastasis. A “benign” tumor can be well differentiated, have characteristically slower growth than a malignant tumor and remain localized to the site of origin. In addition, in some cases a benign tumor does not have the capacity to infiltrate, invade or metastasize to distant sites. A “malignant” tumor can be a poorly differentiated (anaplasia), have characteristically rapid growth accompanied by progressive infiltration, invasion, and destruction of the surrounding tissue. Furthermore, a malignant tumor can have the capacity to metastasize to distant sites. Accordingly, a cancer cell is a cell found within the abnormal mass of tissue whose growth is not coordinated with the growth of normal tissue. Accordingly, a “tumor sample” refers to a biological sample obtained or derived from a tumor of a subject, as described herein.

Non-limiting examples of cancer types include ovarian cancer, cervical cancer, uveal melanoma, colorectal cancer, chromophobe renal cell carcinoma, liver cancer, endocrine tumor, oropharyngeal cancer, retinoblastoma, biliary cancer, adrenal cancer, neural cancer, neuroblastoma, basal cell carcinoma, brain cancer, breast cancer, non-clear cell renal cell carcinoma, glioblastoma, glioma, kidney cancer, gastrointestinal stromal tumor, medulloblastoma, bladder cancer, gastric cancer, bone cancer, non-small cell lung cancer, thymoma, prostate cancer, clear cell renal cell carcinoma, skin cancer, thyroid cancer, sarcoma, testicular cancer, head and neck cancer (e.g., head and neck squamous cell carcinoma), meningioma, peritoneal cancer, endometrial cancer, pancreatic cancer, mesothelioma, esophageal cancer, small cell lung cancer, Her2 negative breast cancer, ovarian serous carcinoma, HR+ breast cancer, uterine serous carcinoma, uterine corpus endometrial carcinoma, gastroesophageal junction adenocarcinoma, gallbladder cancer, chordoma, and papillary renal cell carcinoma.

As used herein, the terms “cancer state” or “cancer condition” refer to a characteristic of a cancer patient's condition, e.g., a diagnostic status, a type of cancer, a location of cancer, a primary origin of a cancer, a cancer stage, a cancer prognosis, and/or one or more additional characteristics of a cancer (e.g., tumor characteristics such as morphology, heterogeneity, size, etc.). In some embodiments, one or more additional personal characteristics of the subject are used further describe the cancer state or cancer condition of the subject, e.g., age, gender, weight, race, personal habits (e.g., smoking, drinking, diet), other pertinent medical conditions (e.g., high blood pressure, dry skin, other diseases), current medications, allergies, pertinent medical history, current side effects of cancer treatments and other medications, etc.

As used herein, the term “liquid biopsy” sample refers to a liquid sample obtained from a subject that includes cell-free DNA. Examples of liquid biopsy samples include, but are not limited to, blood, whole blood, plasma, serum, urine, cerebrospinal fluid, fecal material, saliva, sweat, tears, pleural fluid, pericardial fluid, or peritoneal fluid of the subject. In some embodiments, a liquid biopsy sample is a cell-free sample, e.g., a cell free blood sample. In some embodiments, a liquid biopsy sample is obtained from a subject with cancer. In some embodiments, a liquid biopsy sample is collected from a subject with an unknown cancer status, e.g., for use in determining a cancer status of the subject. Likewise, in some embodiments, a liquid biopsy is collected from a subject with a non-cancerous disorder, e.g., a cardiovascular disease. In some embodiments, a liquid biopsy is collected from a subject with an unknown status for a non-cancerous disorder, e.g., for use in determining a non-cancerous disorder status of the subject.

As used herein, the term “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal material, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotide within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (CNV), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject. For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘mutations.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference sequence construct (e.g., a reference genome or set of reference genomes) for the species. In some instances, sequence isoforms found within the population of a species that do not affect a change in a protein encoded by the genome, or that result in an amino acid substitution that does not substantially affect the function of an encoded protein, are not variant alleles.

As used herein, the term “variant allele fraction,” “VAF,” “allelic fraction,” or “AF” refers to the number of times a variant or mutant allele was observed (e.g., a number of reads supporting a candidate variant allele) divided by the total number of times the position was sequenced (e.g., a total number of reads covering a candidate locus).

As used herein, the term “germline variants” refers to genetic variants inherited from maternal and paternal DNA. Germline variants may be determined through a matched tumor-normal calling pipeline.

As used herein, the term “somatic variants” refers to variants arising as a result of dysregulated cellular processes associated with neoplastic cells, e.g., a mutation. Somatic variants may be detected via subtraction from a matched normal sample.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

As used herein, the term “insertions and deletions” or “indels” refers to a variant resulting from the gain or loss of DNA base pairs within an analyzed region.

As used herein, the term “copy number variation” or “CNV” refers to the process by which large structural changes in a genome associated with tumor aneuploidy and other dysregulated repair systems are detected. These processes are used to detect large scale insertions or deletions of entire genomic regions. CNV is defined as structural insertions or deletions greater than a certain base pair (“bp”) in size, such as 500 bp.

As used herein, the term “gene fusion” refers to the product of large-scale chromosomal aberrations resulting in the creation of a chimeric protein. These expressed products can be non-functional, or they can be highly over or underactive. This can cause deleterious effects in cancer such as hyper-proliferative or anti-apoptotic phenotypes.

As used herein, the term “loss of heterozygosity” refers to the loss of one copy of a segment (e.g., including part or all of one or more genes) of the genome of a diploid subject (e.g., a human) or loss of one copy of a sequence encoding a functional gene product in the genome of the diploid subject, in a tissue, e.g., a cancerous tissue, of the subject. As used herein, when referring to a metric representing loss of heterozygosity across the entire genome of the subject, loss of heterozygosity is caused by the loss of one copy of various segments in the genome of the subject. Loss of heterozygosity across the entire genome may be estimated without sequencing the entire genome of a subject, and such methods for such estimations based on gene panel targeting-based sequencing methodologies are described in the art. Accordingly, in some embodiments, a metric representing loss of heterozygosity across the entire genome of a tissue of a subject is represented as a single value, e.g., a percentage or fraction of the genome. In some cases, a tumor is composed of various sub-clonal populations, each of which may have a different degree of loss of heterozygosity across their respective genomes. Accordingly, in some embodiments, loss of heterozygosity across the entire genome of a cancerous tissue refers to an average loss of heterozygosity across a heterogeneous tumor population. As used herein, when referring to a metric for loss of heterozygosity in a particular gene, e.g., a DNA repair protein such as a protein involved in the homologous DNA recombination pathway (e.g., BRCA1 or BRCA2), loss of heterozygosity refers to complete or partial loss of one copy of the gene encoding the protein in the genome of the tissue and/or a mutation in one copy of the gene that prevents translation of a full-length gene product, e.g., a frameshift or truncating (creating a premature stop codon in the gene) mutation in the gene of interest. In some cases, a tumor is composed of various sub-clonal populations, each of which may have a different mutational status in a gene of interest. Accordingly, in some embodiments, loss of heterozygosity for a particular gene of interest is represented by an average value for loss of heterozygosity for the gene across all sequenced sub-clonal populations of the cancerous tissue. In other embodiments, loss of heterozygosity for a particular gene of interest is represented by a count of the number of unique incidences of loss of heterozygosity in the gene of interest across all sequenced sub-clonal populations of the cancerous tissue (e.g., the number of unique frame-shift and/or truncating mutations in the gene identified in the sequencing data).

As used herein, the term “gene product” refers to an RNA (e.g., mRNA or miRNA) or protein molecule transcribed or translated from a particular genomic locus, e.g., a particular gene. The genomic locus can be identified using a gene name, a chromosomal location, or any other genetic mapping metric.

As used herein, the terms “expression level,” “abundance level,” or simply “abundance” refers to an amount of a gene product, (an RNA species, e.g., mRNA or miRNA, or protein molecule) transcribed or translated by a cell, or an average amount of a gene product transcribed or translated across multiple cells. When referring to mRNA or protein expression, the term generally refers to the amount of any RNA or protein species corresponding to a particular genomic locus, e.g., a particular gene. However, in some embodiments, an expression level can refer to the amount of a particular isoform of an mRNA or protein corresponding to a particular gene that gives rise to multiple mRNA or protein isoforms. The genomic locus can be identified using a gene name, a chromosomal location, or any other genetic mapping metric.

As used herein, the term “ratio” refers to any comparison of a first metric X, or a first mathematical transformation thereof X′ (e.g., measurement of a number of units of a genomic sequence in a first one or more biological samples or a first mathematical transformation thereof) to another metric Y or a second mathematical transformation thereof Y′ (e.g., the number of units of a respective genomic sequence in a second one or more biological samples or a second mathematical transformation thereof) expressed as X/Y, Y/X, log_{N}(X/Y), log_{N}(Y/X), X′/Y, Y/X′, log_{N}(X′/Y), or log_{N}(Y/X′), X/Y′, Y′/X, log_{N} (X/Y′), log_{N}(Y′/X), X′/Y′, Y′/X′, log_{N}(X′/Y′), or logs (Y′/X′), where N is any real number greater than 1 and where example mathematical transformations of X and Y include, but are not limited to. raising X or Y to a power Z, multiplying X or Y by a constant Q, where Z and Q are any real numbers, and/or taking an M based logarithm of X and/or Y, where M is a real number greater than 1. In one non-limiting example, X is transformed to X′ prior to ratio calculation by raising X by the power of two (X²) and Y is transformed to Y′ prior to ratio calculation by raising Y by the power of 3.2 (Y^{3.2}) and the ratio of X and Y is computed as log₂(X′/Y′).

As used herein, the term “relative abundance” refers to a ratio of a first amount of a compound measured in a sample, e.g., a gene product (an RNA species, e.g., mRNA or miRNA, or protein molecule) or nucleic acid fragments having a particular characteristic (e.g., aligning to a particular locus or encompassing a particular allele), to a second amount of a compound measured in a second sample. In some embodiments, relative abundance refers to a ratio of an amount of species of a compound to a total amount of the compound in the same sample. For instance, a ratio of the amount of mRNA transcripts encoding a particular gene in a sample (e.g., aligning to a particular region of the exome) to the total amount of mRNA transcripts in the sample. In other embodiments, relative abundance refers to a ratio of an amount of a compound or species of a compound in a first sample to an amount of the compound of the species of the compound in a second sample. For instance, a ratio of a normalized amount of mRNA transcripts encoding a particular gene in a first sample to a normalized amount of mRNA transcripts encoding the particular gene in a second and/or reference sample.

As used herein, the terms “sequencing,” “sequence determination,” and the like refer to any biochemical processes that may be used to determine the order of biological macromolecules such as nucleic acids or proteins. For example, sequencing data can include all or a portion of the nucleotide bases in a nucleic acid molecule such as an mRNA transcript or a genomic locus.

As used herein, the term “genetic sequence” refers to a recordation of a series of nucleotides present in a subject's RNA or DNA as determined by sequencing of nucleic acids from the subject.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing, for example, can provide sequence reads that can vary in size from tens to hundreds to thousands of base pairs. Illumina® parallel sequencing, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “read segment” refers to any form of nucleotide sequence read including the raw sequence reads obtained directly from a nucleic acid sequencing technique or from a sequence derived therefrom, e.g., an aligned sequence read, a collapsed sequence read, or a stitched sequence read.

As used herein, the term “read count” refers to the total number of nucleic acid reads generated, which may or may not be equivalent to the number of nucleic acid molecules generated, during a nucleic acid sequencing reaction.

As used herein, the term “read-depth,” “sequencing depth,” or “depth” can refer to a total number of unique nucleic acid fragments encompassing a particular locus or region of the genome of a subject that are sequenced in a particular sequencing reaction. Sequencing depth can be expressed as “Yx”, e.g., 50×, 100×, etc., where “Y” refers to the number of unique nucleic acid fragments encompassing a particular locus that are sequenced in a sequencing reaction. In such a case, Y is necessarily an integer, because it represents the actual sequencing depth for a particular locus. Alternatively, read-depth, sequencing depth, or depth can refer to a measure of central tendency (e.g., a mean or mode) of the number of unique nucleic acid fragments that encompass one of a plurality of loci or regions of the genome of a subject that are sequenced in a particular sequencing reaction. For example, in some embodiments, sequencing depth refers to the average depth of every locus across an arm of a chromosome, a targeted sequencing panel, an exome, or an entire genome. In such case, Y may be expressed as a fraction or a decimal, because it refers to an average coverage across a plurality of loci. When a mean depth is recited, the actual depth for any particular locus may be different than the overall recited depth. Metrics can be determined that provide a range of sequencing depths in which a defined percentage of the total number of loci fall. For instance, a range of sequencing depths within which 90% or 95%, or 99% of the loci fall. As understood by the skilled artisan, different sequencing technologies provide different sequencing depths. For instance, low-pass whole genome sequencing can refer to technologies that provide a sequencing depth of less than 5×, less than 4×, less than 3×, or less than 2×, e.g., from about 0.5× to about 3×.

As used herein, the term “sequencing breadth” refers to what fraction of a particular reference exome (e.g., human reference exome), a particular reference genome (e.g., human reference genome), or part of the exome or genome has been analyzed. Sequencing breadth can be expressed as a fraction, a decimal, or a percentage, and is generally calculated as (the number of loci analyzed/the total number of loci in a reference exome or reference genome). The denominator of the fraction can be a repeat-masked genome, and thus 100% can correspond to all of the reference genome minus the masked parts. A repeat-masked exome or genome can refer to an exome or genome in which sequence repeats are masked (e.g., sequence reads align to unmasked portions of the exome or genome). In some embodiments, any part of an exome or genome can be masked and, thus, sequencing breadth can be evaluated for any desired portion of a reference exome or genome. In some embodiments, “broad sequencing” refers to sequencing/analysis of at least 0.1% of an exome or genome.

As used herein, the term “sequencing probe” refers to a molecule that binds to a nucleic acid with affinity that is based on the expected nucleotide sequence of the RNA or DNA present at that locus.

As used herein, the term “targeted panel” or “targeted gene panel” refers to a combination of probes for sequencing (e.g., by next-generation sequencing) nucleic acids present in a biological sample from a subject (e.g., a tumor sample, liquid biopsy sample, germline tissue sample, white blood cell sample, or tumor or tissue organoid sample), selected to map to one or more loci of interest on one or more chromosomes. An example set of loci/genes useful for precision oncology, e.g., via solid or liquid biopsy assay, that can be analyzed using a targeted panel is described in Table 1. Another example set of loci/genes useful for precision oncology, e.g., via solid or liquid biopsy assay, that can be analyzed using a targeted panel is described in Table 2. In some embodiments, in addition to loci that are informative for precision oncology, a targeted panel includes one or more probes for sequencing one or more of a loci associated with a different medical condition, a loci used for internal control purposes, or a loci from a pathogenic organism (e.g., an oncogenic pathogen).

As used herein, the term, “reference exome” refers to any sequenced or otherwise characterized exome, whether partial or complete, of any tissue from any organism or pathogen that may be used to reference identified sequences from a subject. Typically, a reference exome will be derived from a subject of the same species as the subject whose sequences are being evaluated. Example reference exomes used for human subjects as well as many other organisms are provided in the on-line genome browser hosted by the National Center for Biotechnology Information (“NCBI”). An “exome” refers to the complete transcriptional profile of an organism or pathogen, expressed in nucleic acid sequences. As used herein, a reference sequence or reference exome often is an assembled or partially assembled exomic sequence from an individual or multiple individuals. In some embodiments, a reference exome is an assembled or partially assembled exomic sequence from one or more human individuals. The reference exome can be viewed as a representative example of a species' set of expressed genes. In some embodiments, a reference exome comprises sequences assigned to chromosomes.

As used herein, the term “reference genome” refers to any sequenced or otherwise characterized genome, whether partial or complete, of any organism or pathogen that may be used to reference identified sequences from a subject. Typically, a reference genome will be derived from a subject of the same species as the subject whose sequences are being evaluated. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or pathogen, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38). For a haploid genome, there can be only one nucleotide at each locus. For a diploid genome, heterozygous loci can be identified; each heterozygous locus can have two alleles, where either allele can allow a match for alignment to the locus.

As used herein, the term “bioinformatics pipeline” refers to a series of processing stages used to determine characteristics of a subject's genome or exome based on sequencing data of the subject's genome or exome. A bioinformatics pipeline may be used to determine characteristics of a germline genome or exome of a subject and/or a cancer genome or exome of a subject. In some embodiments, the pipeline extracts information related to genomic alterations in the cancer genome of a subject, which is useful for guiding clinical decisions for precision oncology, from sequencing results of a biological sample, e.g., a tumor sample, liquid biopsy sample, reference normal sample, etc., from the subject. Certain processing stages in a bioinformatics may be ‘connected,’ meaning that the results of a first respective processing stage are informative and/or essential for execution of a second, downstream processing stage. For instance, in some embodiments, a bioinformatics pipeline includes a first respective processing stage for identifying genomic alterations that are unique to the cancer genome of a subject and a second respective processing stage that uses the quantity and/or identity of the identified genomic alterations to determine a metric that is informative for precision oncology, e.g., a tumor mutational burden. In some embodiments, the bioinformatics pipeline includes a reporting stage that generates a report of relevant and/or actionable information identified by upstream stages of the pipeline, which may or may not further include recommendations for aiding clinical therapy decisions.

As used herein, the term “limit of detection” or “LOD” refers to the minimal quantity of a feature that can be identified with a particular level of confidence. Accordingly, level of detection can be used to describe an amount of a substance that must be present in order for a particular assay to reliably detect the substance. A level of detection can also be used to describe a level of support needed for an algorithm to reliably identify a genomic alteration based on sequencing data. For example, a minimal number of unique sequence reads to support identification of a sequence variant such as a SNV.

As used herein, the term “BAM File” or “Binary file containing Alignment Maps” refers to a file storing sequencing data aligned to a reference sequence (e.g., a reference genome or exome). In some embodiments, a BAM file is a compressed binary version of a SAM (Sequence Alignment Map) file that includes, for each of a plurality of unique sequence reads, an identifier for the sequence read, information about the nucleotide sequence, information about the alignment of the sequence to a reference sequence, and optionally metrics relating to the quality of the sequence read and/or the quality of the sequence alignment. While BAM files generally relate to files having a particular format, for simplicity they are used herein to simply refer to a file, of any format, containing information about a sequence alignment, unless specifically stated otherwise.

As used herein, the term “measure of central tendency” refers to a central or representative value for a distribution of values. Non-limiting examples of measures of central tendency include an arithmetic mean, weighted mean, midrange, midhinge, trimean, geometric mean, geometric median, Winsorized mean, median, and mode of the distribution of values.

As used herein, the term “Positive Predictive Value” or “PPV” means the likelihood that a variant is properly called given that a variant has been called by an assay. PPV can be expressed as (number of true positives)/(number of false positives+number of true positives).

As used herein, the term “assay” refers to a technique for determining a property of a substance, e.g., a nucleic acid, a protein, a cell, a tissue, or an organ. An assay (e.g., a first assay or a second assay) can comprise a technique for determining the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art can be used to detect any of the properties of nucleic acids mentioned herein. Properties of a nucleic acids can include a sequence, genomic identity, copy number, methylation state at one or more nucleotide positions, size of the nucleic acid, presence or absence of a mutation in the nucleic acid at one or more nucleotide positions, and pattern of fragmentation of a nucleic acid (e.g., the nucleotide position(s) at which a nucleic acid fragments). An assay or method can have a particular sensitivity and/or specificity, and their relative usefulness as a diagnostic tool can be measured using ROC-AUC statistics.

As used herein, the term “classification” can refer to any number(s) or other characters(s) that are associated with a particular property of a sample. For example, in some embodiments, the term “classification” can refer to a type of cancer in a subject, a stage of cancer in a subject, a prognosis for a cancer in a subject, a tumor load, a presence of tumor metastasis in a subject, and the like. The classification can be binary (e.g., positive or negative) or have more levels of classification (e.g., a scale from 1 to 10 or 0 to 1). The terms “cutoff” and “threshold” can refer to predetermined numbers used in an operation. For example, a cutoff size can refer to a size above which fragments are excluded. A threshold value can be a value above or below which a particular classification applies. Either of these terms can be used in either of these contexts.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, an “actionable genomic alteration” or “actionable variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to be associated with a therapeutic course of action that is more likely to produce a positive effect in a cancer patient that has the actionable variant than in a similarly situated cancer patient that does not have the actionable variant. For instance, administration of EGFR inhibitors (e.g., afatinib, erlotinib, gefitinib) is more effective for treating non-small cell lung cancer in patients with an EGFR mutation in exons 19/21 than for treating non-small cell lung cancer in patients that do not have an EGFR mutations in exons 19/21. Accordingly, an EGFR mutation in exon 19/21 is an actionable variant. In some instances, an actionable variant is only associated with an improved treatment outcome in one or a group of specific cancer types. In other instances, an actionable variant is associated with an improved treatment outcome in substantially all cancer types.

As used herein, a “variant of uncertain significance” or “VUS” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), whose impact on disease development/progression is unknown.

As used herein, a “benign variant” or “likely benign variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to not contribute to disease development/progression.

As used herein, a “pathogenic variant” or “likely pathogenic variant” refers to a genomic alteration (e.g., a SNV, MNV, indel, rearrangement, copy number variation, or ploidy variation), or value of another cancer metric derived from nucleic acid sequencing data (e.g., a tumor mutational burden, MSI status, or tumor fraction), that is known or believed to contribute to disease development/progression.

As used herein, an “effective amount” or “therapeutically effective amount” is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the therapeutic agent being administered.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

Reference will now be made in detail to embodiments, examples of which are illustrated in the accompanying drawings. In the following detailed description, numerous specific details are set forth in order to provide a thorough understanding of the present disclosure, including example systems, methods, techniques, instruction sequences, and computing machine program products that embody illustrative implementations. However, the illustrative discussions below are not intended to be exhaustive or to limit the implementations to the precise forms disclosed. Many modifications and variations are possible in view of the above teachings. The features described herein are not limited by the illustrated ordering of acts or events, as some acts can occur in different orders and/or concurrently with other acts or events.

The implementations provided herein are chosen and described in order to best explain the principles and their practical applications, to thereby enable others skilled in the art to best utilize the various embodiments with various modifications as are suited to the particular use contemplated. In some instances, well-known methods, procedures, components, circuits, and networks have not been described in detail so as not to unnecessarily obscure aspects of the embodiments. In other instances, it will be apparent to one of ordinary skill in the art that the present disclosure may be practiced without one or more of the specific details.

It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that though such a design effort might be complex and time-consuming, it will nevertheless be a routine undertaking of engineering for those of ordering skill in the art having the benefit of the present disclosure.

### Example System Embodiments

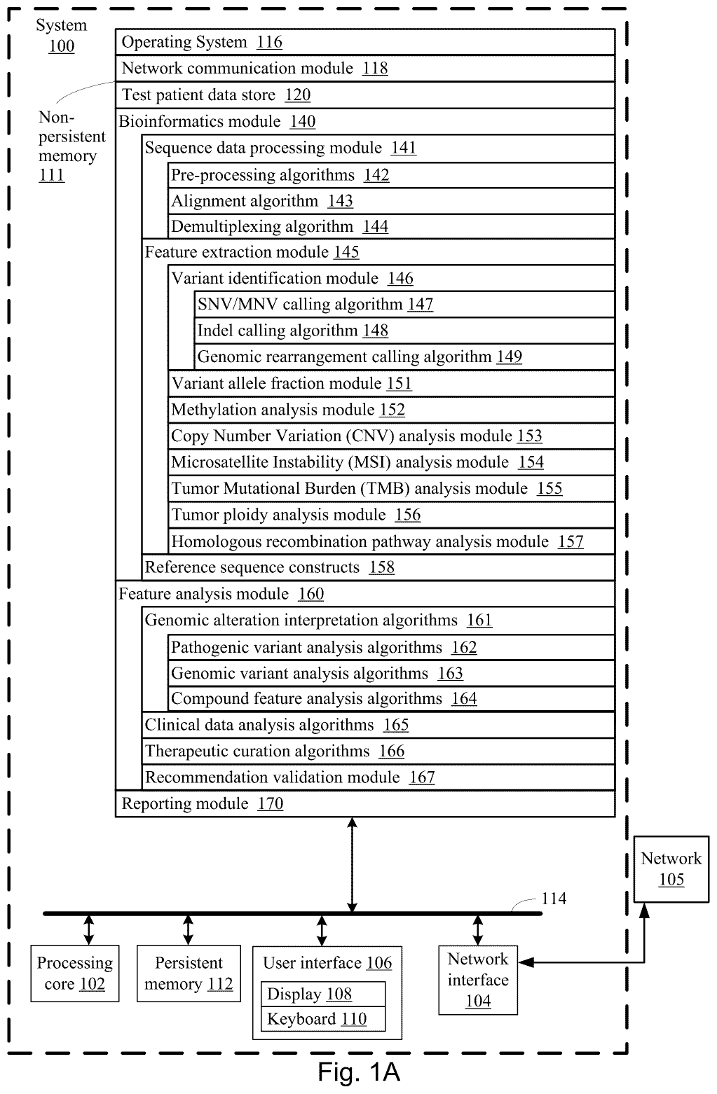

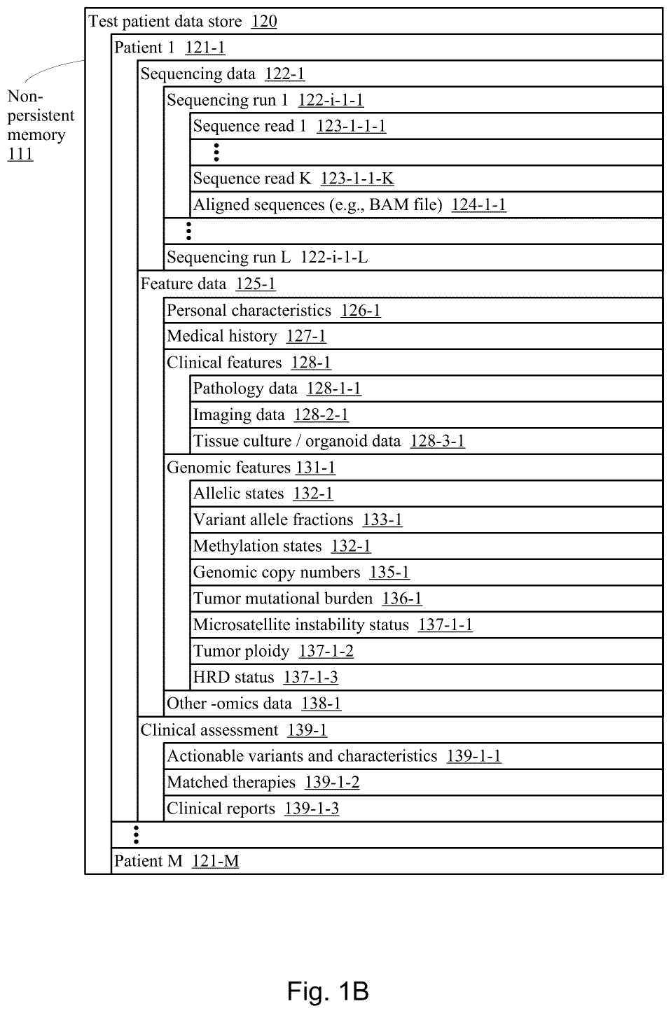

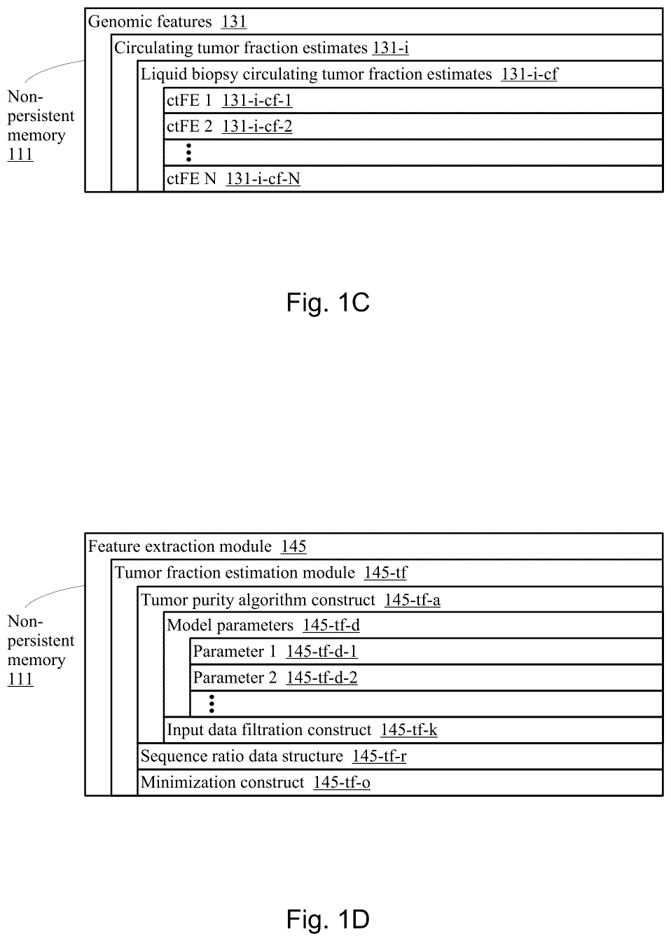

Now that an overview of some aspects of the present disclosure and some definitions used in the present disclosure have been provided, details of an exemplary system for detecting whether a variant in a biopsy from a subject is (a) a somatic variant derived from cell free DNA or (b) other than a somatic variant derived from cell free DNA are now described in conjunction with FIGS. 1A-1D. FIGS. 1A-1D collectively illustrate the topology of an example system for detecting whether a variant in a biopsy from a subject is (a) a somatic variant derived from cell free DNA or (b) other than a somatic variant derived from cell free DNA, in accordance with some embodiments of the present disclosure. Advantageously, the example system illustrated in FIGS. 1A-1D improves upon conventional methods for providing clinical support for personalized cancer therapy by detecting whether a variant in a biopsy from a subject is (a) a somatic variant derived from cell free DNA or (b) other than a somatic variant derived from cell free DNA.

FIG. 1A is a block diagram illustrating a system in accordance with some implementations. The device 100 in some implementations includes one or more processing units CPU(s) 102 (also referred to as processors), one or more network interfaces 104, a user interface 106, e.g., including a display 108 and/or an input 110 (e.g., a mouse, touchpad, keyboard, etc.), a non-persistent memory 111, a persistent memory 112, and one or more communication buses 114 for interconnecting these components. The one or more communication buses 114 optionally include circuitry (sometimes called a chipset) that interconnects and controls communications between system components. The non-persistent memory 111 typically includes high-speed random access memory, such as DRAM, SRAM, DDR RAM, ROM, EEPROM, flash memory, whereas the persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid state storage devices. The persistent memory 112 optionally includes one or more storage devices remotely located from the CPU(s) 102. The persistent memory 112, and the non-volatile memory device(s) within the non-persistent memory 112, comprise non-transitory computer readable storage medium. In some implementations, the non-persistent memory 111 or alternatively the non-transitory computer readable storage medium stores the following programs, modules and data structures, or a subset thereof, sometimes in conjunction with the persistent memory 112:
an operating system 116, which includes procedures for handling various basic system services and for performing hardware dependent tasks;
a network communication module (or instructions) 118 for connecting the system 100 with other devices and/or a communication network 105;
a test patient data store 120 for storing one or more collections of features from patients (e.g., subjects);
a bioinformatics module 140 for processing sequencing data and extracting features from sequencing data, e.g., from liquid biopsy sequencing assays;
a feature analysis module 160 for evaluating patient features, e.g., genomic alterations, compound genomic features, and clinical features; and
a reporting module 180 for generating and transmitting reports that provide clinical support for personalized cancer therapy.

Although FIGS. 1A-1D depict a “system 100,” the figures are intended more as a functional description of the various features that may be present in computer systems than as a structural schematic of the implementations described herein. In practice, and as recognized by those of ordinary skill in the art, items shown separately could be combined and some items could be separated. Moreover, although FIG. 1 depicts certain data and modules in non-persistent memory 111, some or all of these data and modules may be in persistent memory 112. For example, in various implementations, one or more of the above identified elements are stored in one or more of the previously mentioned memory devices and correspond to a set of instructions for performing a function described above. The above identified modules, data, or programs (e.g., sets of instructions) need not be implemented as separate software programs, procedures, datasets, or modules, and thus various subsets of these modules and data may be combined or otherwise re-arranged in various implementations.

In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of system 100, that is addressable by system 100 so that system 100 may retrieve all or a portion of such data when needed.

For purposes of illustration in FIG. 1A, system 100 is represented as a single computer that includes all of the functionality for providing clinical support for personalized cancer therapy. However, while a single machine is illustrated, the term “system” shall also be taken to include any collection of machines that individually or jointly execute a set (or multiple sets) of instructions to perform any one or more of the methodologies discussed herein.

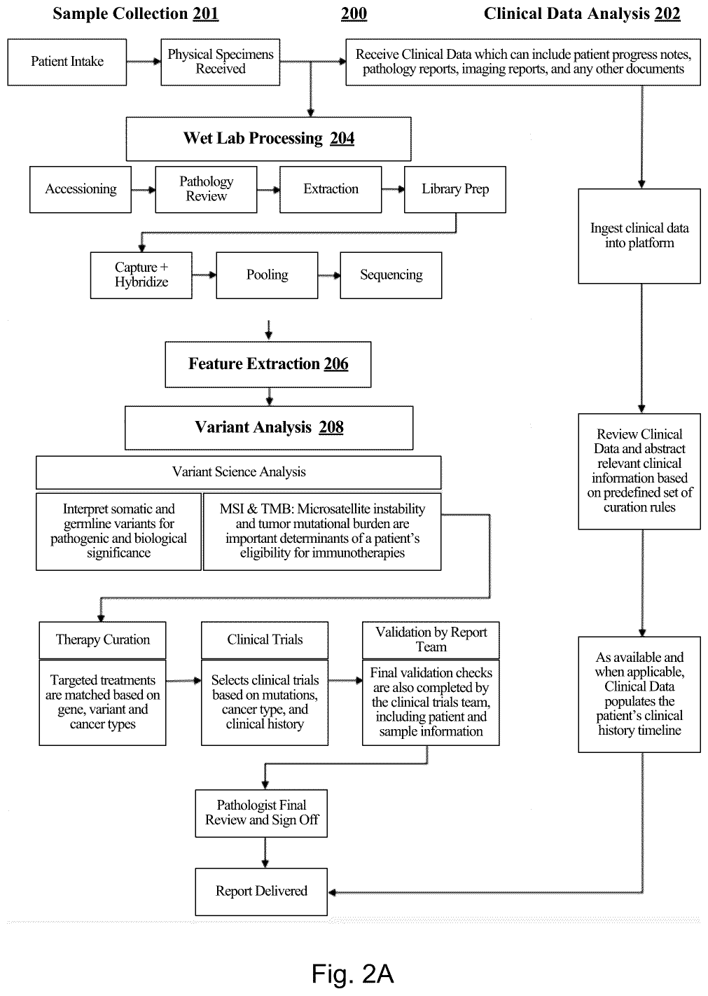

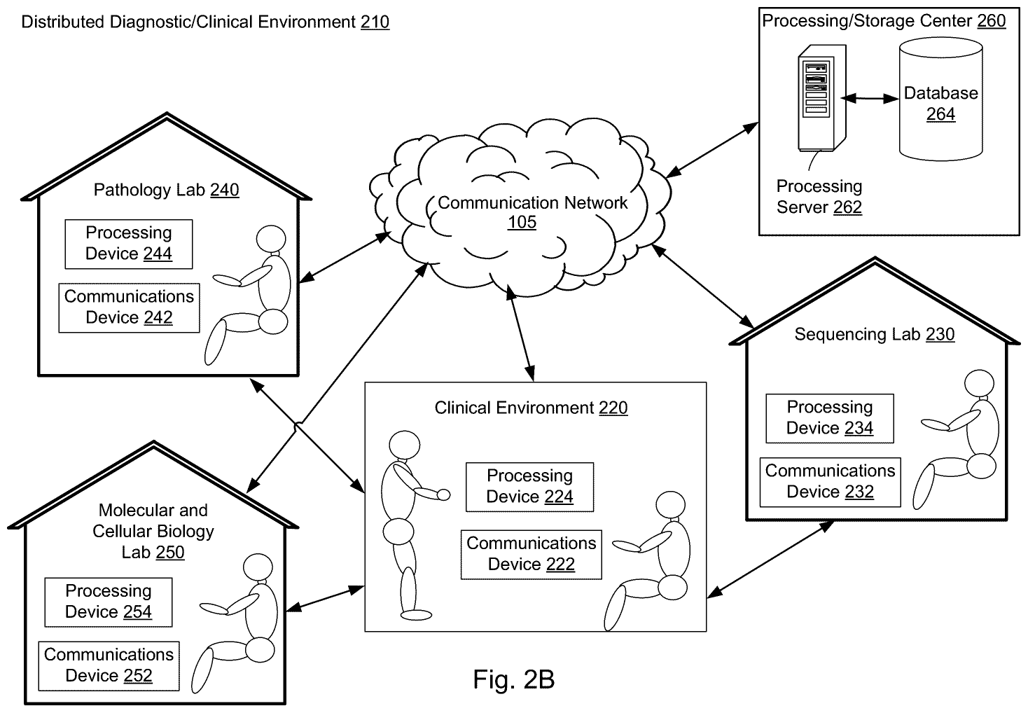

For example, in some embodiments, system 100 includes one or more computers. In some embodiments, the functionality for providing clinical support for personalized cancer therapy is spread across any number of networked computers and/or resides on each of several networked computers and/or is hosted on one or more virtual machines at a remote location accessible across the communications network 105. For example, different portions of the various modules and data stores illustrated in FIGS. 1A-1D can be stored and/or executed on the various instances of a processing device and/or processing server/database in the distributed diagnostic environment 210 illustrated in FIG. 2B (e.g., processing devices 224, 234, 244, and 254, processing server 262, and database 264).

The system may operate in the capacity of a server or a client machine in client-server network environment, as a peer machine in a peer-to-peer (or distributed) network environment, or as a server or a client machine in a cloud computing infrastructure or environment. The system may be a personal computer (PC), a tablet PC, a set-top box (STB), a Personal Digital Assistant (PDA), a cellular telephone, a web appliance, a server, a network router, a switch or bridge, or any machine capable of executing a set of instructions (sequential or otherwise) that specify actions to be taken by that machine.

In another implementation, the system comprises a virtual machine that includes a module for executing instructions for performing any one or more of the methodologies disclosed herein. In computing, a virtual machine (VM) is an emulation of a computer system that is based on computer architectures and provides functionality of a physical computer. Some such implementations may involve specialized hardware, software, or a combination of hardware and software.

One of skill in the art will appreciate that any of a wide array of different computer topologies are used for the application and all such topologies are within the scope of the present disclosure.

### Test Patient Data Store (120).

Referring to FIG. 1B, in some embodiments, the system (e.g., system 100) includes a patient data store 120 that stores data for patients 121-1 to 121-M (e.g., cancer patients or patients being tested for cancer) including one or more sequencing data 122, feature data 125, and clinical assessments 139. These data are used and/or generated by the various processes stored in the bioinformatics module 140 and feature analysis module 160 of system 100, to ultimately generate a report providing clinical support for personalized cancer therapy of a patient. While the feature scope of patient data 121 across all patients may be informationally dense, an individual patient's feature set may be sparsely populated across the entirety of the collective feature scope of all features across all patients. That is to say, the data stored for one patient may include a different set of features that the data stored for another patient. Further, while illustrated as a single data construct in FIG. 1B, different sets of patient data may be stored in different databases or modules spread across one or more system memories.

In some embodiments, sequencing data 122 from one or more sequencing reactions 122-i, including a plurality of sequence reads 123-i-1 to 123-i-K, is stored in the test patient data store 120. The data store may include different sets of sequencing data from a single subject, corresponding to different samples from the patient, e.g., a tumor sample, liquid biopsy sample, tumor organoid derived from a patient tumor, and/or a normal sample, and/or to samples acquired at different times, e.g., while monitoring the progression, regression, remission, and/or recurrence of a cancer in a subject. The sequence reads may be in any suitable file format, e.g., BCL, FASTA, FASTQ, etc. In some embodiments, sequencing data 122 is accessed by a sequencing data processing module 141, which performs various pre-processing, genome alignment, and demultiplexing operations, as described in detail below with reference to bioinformatics module 140. In some embodiments, sequence data that has been aligned to a reference construct, e.g., BAM file 124, is stored in test patient data store 120.

In some embodiments, the test patient data store 120 includes feature data 125, e.g., that is useful for identifying clinical support for personalized cancer therapy. In some embodiments, the feature data 125 includes personal characteristics 126 of the patient, such as patient name, date of birth, gender, ethnicity, physical address, smoking status, alcohol consumption characteristic, anthropomorphic data, etc.

In some embodiments, the feature data 125 includes medical history data 127 for the patient, such as cancer diagnosis information (e.g., date of initial diagnosis, date of metastatic diagnosis, cancer staging, tumor characterization, tissue of origin, previous treatments and outcomes, adverse effects of therapy, therapy group history, clinical trial history, previous and current medications, surgical history, etc.), previous or current symptoms, previous or current therapies, previous treatment outcomes, previous disease diagnoses, diabetes status, diagnoses of depression, diagnoses of other physical or mental maladies, and family medical history. In some embodiments, the feature data 125 includes clinical features 128, such as pathology data 128-1, medical imaging data 128-2, and tissue culture and/or tissue organoid culture data 128-3.

In some embodiments, yet other clinical features, such as previous laboratory testing results, are stored in the test patient data store 120. Medical history data 127 and clinical features may be collected from various sources, including at intake directly from the patient, from an electronic medical record (EMR) or electronic health record (EHR) for the patient, or curated from other sources, such as fields from various testing records (e.g., genetic sequencing reports).

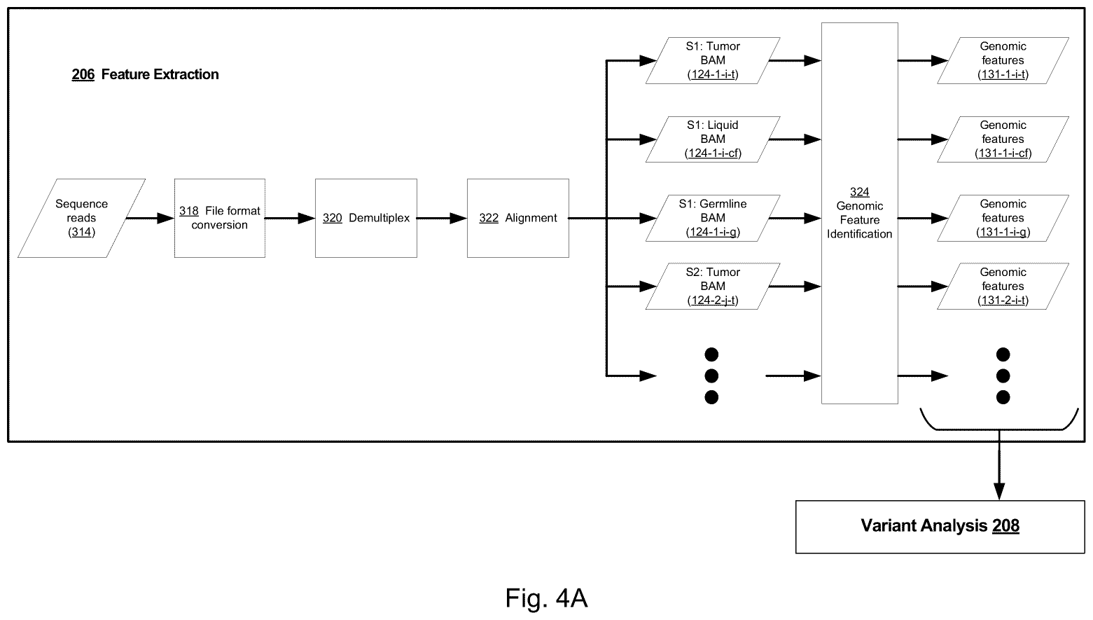

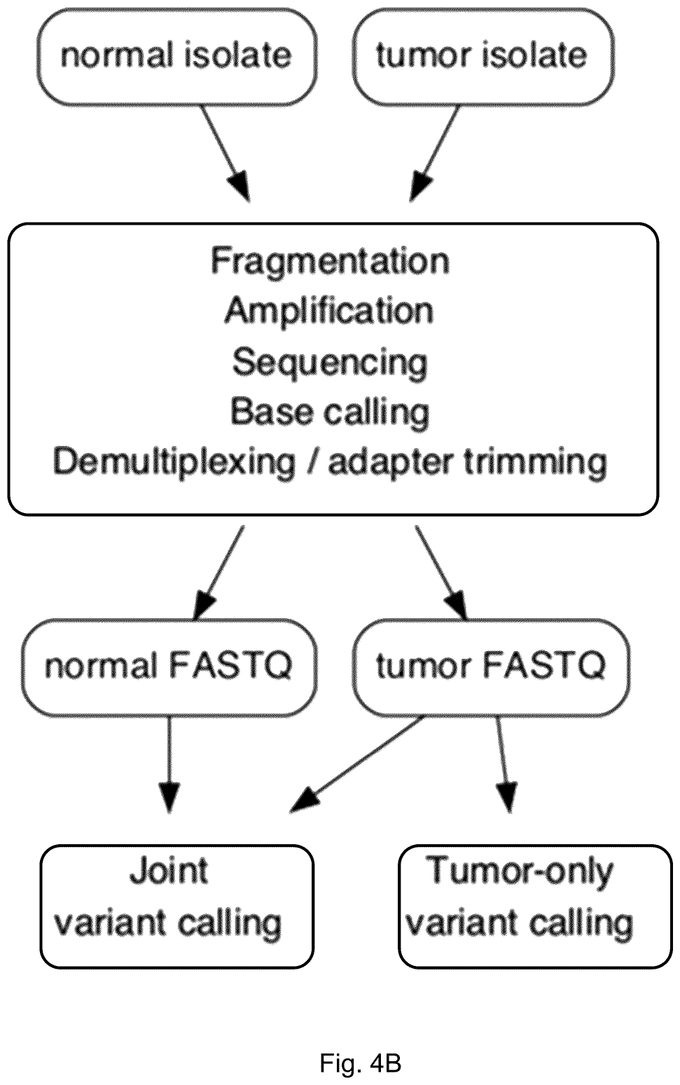

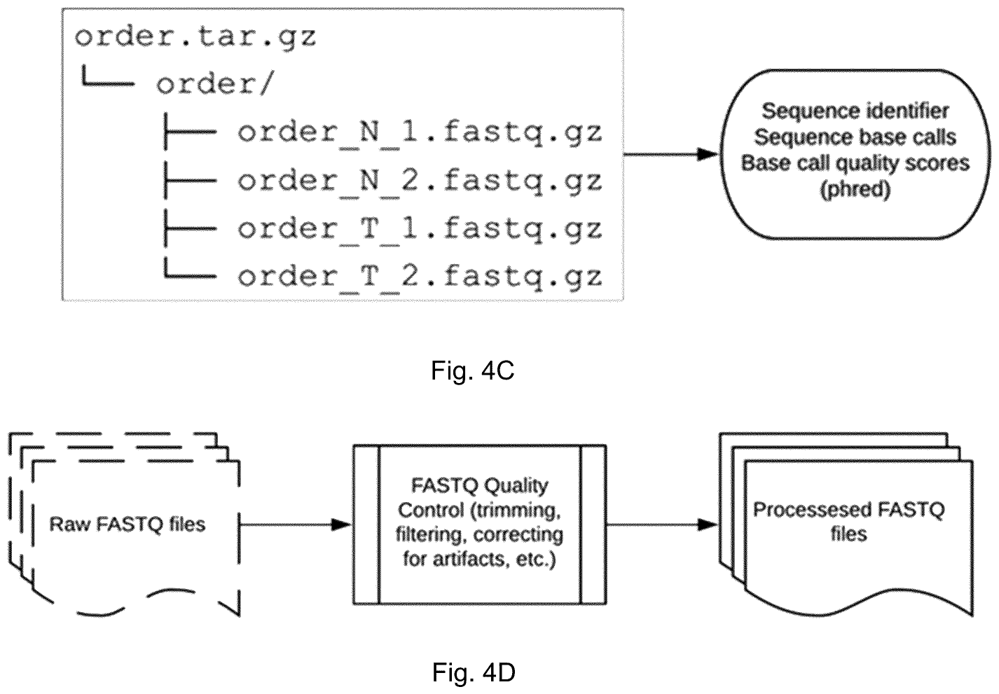

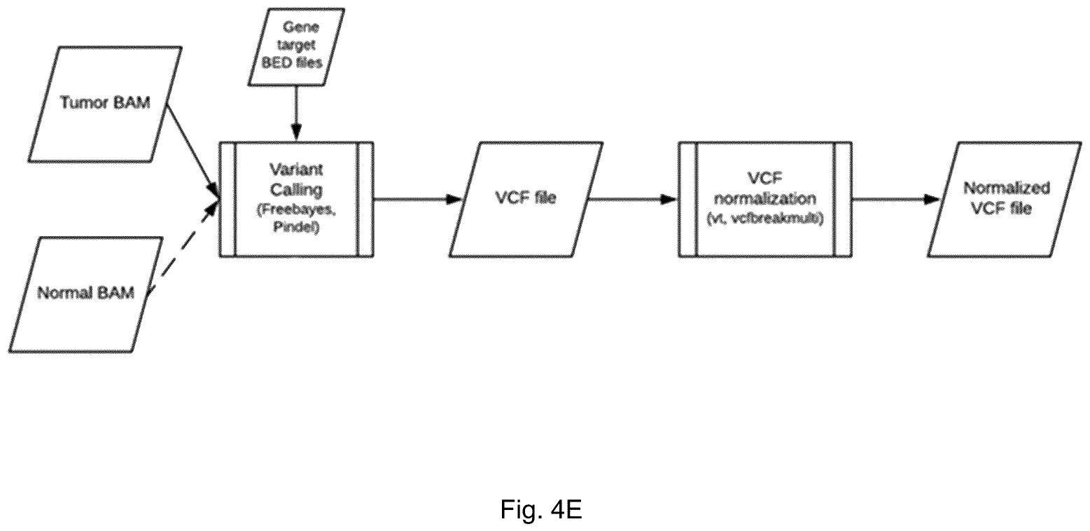

In some embodiments, the feature data 125 includes genomic features 131 for the patient. Non-limiting examples of genomic features include allelic states 132 (e.g., the identity of alleles at one or more loci, support for wild type or variant alleles at one or more loci, support for SNVs/MNVs at one or more loci, support for indels at one or more loci, and/or support for gene rearrangements at one or more loci), allelic fractions 133 (e.g., ratios of variant to reference alleles (or vice versa), methylation states 132 (e.g., a distribution of methylation patterns at one or more loci and/or support for aberrant methylation patterns at one or more loci), genomic copy numbers 135 (e.g., a copy number value at one or more loci and/or support for an aberrant (increased or decreased) copy number at one or more loci), tumor mutational burden 136 (e.g., a measure of the number of mutations in the cancer genome of the subject), and microsatellite instability status 137 (e.g., a measure of the repeated unit length at one or more microsatellite loci and/or a classification of the MSI status for the patient's cancer). In some embodiments, one or more of the genomic features 131 are determined by a nucleic acid bioinformatics pipeline, e.g., as described in detail below with reference to FIGS. 4A-4F. In particular, in some embodiments, the feature data 125 include circulating tumor fraction estimates 131-i, as determined using the improved methods for determining circulating tumor fraction estimates, as described in further detail below with reference to FIGS. 1C, 1D, and 4F. In some embodiments, one or more of the genomic features 131 are obtained from an external testing source, e.g., not connected to the bioinformatics pipeline as described below.

In some embodiments, the feature data 125 further includes data 138 from other-omics fields of study. Non-limiting examples of -omics fields of study that may yield feature data useful for providing clinical support for personalized cancer therapy include transcriptomics, epigenomics, proteomics, metabolomics, metabonomics, microbiomics, lipidomics, glycomics, cellomics, and organoidomics.

In some embodiments, yet other features may include features derived from machine learning approaches, e.g., based at least in part on evaluation of any relevant molecular or clinical features, considered alone or in combination, not limited to those listed above. For instance, in some embodiments, one or more latent features from evaluation of cancer patient training datasets improve the diagnostic and prognostic power of the various analysis algorithms in the feature analysis module 160.

The skilled artisan will know of other types of features useful for providing clinical support for personalized cancer therapy. The listing of features above is merely representative and should not be construed to be limiting.

In some embodiments, a test patient data store 120 includes clinical assessment data 139 for patients, e.g., based on the feature data 125 collected for the subject. In some embodiments, the clinical assessment data 139 includes a catalogue of actionable variants and characteristics 139-1 (e.g., genomic alterations and compound metrics based on genomic features known or believed to be targetable by one or more specific cancer therapies), matched therapies 139-2 (e.g., the therapies known or believed to be particularly beneficial for treatment of subjects having actionable variants), and/or clinical reports 139-3 generated for the subject, e.g., based on identified actionable variants and characteristics 139-1 and/or matched therapies 139-2.

In some embodiments, clinical assessment data 139 is generated by analysis of feature data 125 using the various algorithms of feature analysis module 160, as described in further detail below. In some embodiments, clinical assessment data 139 is generated, modified, and/or validated by evaluation of feature data 125 by a clinician, e.g., an oncologist. For instance, in some embodiments, a clinician (e.g., at clinical environment 220) uses feature analysis module 160, or accesses test patient data store 120 directly, to evaluate feature data 125 to make recommendations for personalized cancer treatment of a patient. Similarly, in some embodiments, a clinician (e.g., at clinical environment 220) reviews recommendations determined using feature analysis module 160 and approves, rejects, or modifies the recommendations, e.g., prior to the recommendations being sent to a medical professional treating the cancer patient.

### Bioinformatics Module (140)

Referring again to FIG. 1A, the system (e.g., system 100) includes a bioinformatics module 140 that includes a feature extraction module 145 and optional ancillary data processing constructs, such as a sequence data processing module 141 and/or one or more reference sequence constructs 158 (e.g., a reference genome, exome, or targeted-panel construct that includes reference sequences for a plurality of loci targeted by a sequencing panel).

In some embodiments, bioinformatics module 140 includes a sequence data processing module 141 that includes instructions for processing sequence reads, e.g., raw sequence reads 123 from one or more sequencing reactions 122, prior to analysis by the various feature extraction algorithms, as described in detail below. In some embodiments, sequence data processing module 141 includes one or more pre-processing algorithms 142 that prepare the data for analysis. In some embodiments, the pre-processing algorithms 142 include instructions for converting the file format of the sequence reads from the output of the sequencer (e.g., a BCL file format) into a file format compatible with downstream analysis of the sequences (e.g., a FASTQ or FASTA file format). In some embodiments, the pre-processing algorithms 142 include instructions for evaluating the quality of the sequence reads (e.g., by interrogating quality metrics like Phred score, base-calling error probabilities, Quality (Q) scores, and the like) and/or removing sequence reads that do not satisfy a threshold quality (e.g., an inferred base call accuracy of at least 80%, at least 90%, at least 95%, at least 99%, at least 99.5%, at least 99.9%, or higher). In some embodiments, the pre-processing algorithms 142 include instructions for filtering the sequence reads for one or more properties, e.g., removing sequences failing to satisfy a lower or upper size threshold or removing duplicate sequence reads.

In some embodiments, sequence data processing module 141 includes one or more alignment algorithms 143, for aligning pre-processed sequence reads 123 to a reference sequence construct 158, e.g., a reference genome, exome, or targeted-panel construct. Many algorithms for aligning sequencing data to a reference construct are known in the art, for example, BWA, Blat, SHRIMP, LastZ, and MAQ. One example of a sequence read alignment package is the Burrows-Wheeler Alignment tool (BWA), which uses a Burrows-Wheeler Transform (BWT) to align short sequence reads against a large reference construct, allowing for mismatches and gaps. Li and Durbin, Bioinformatics, 25 (14): 1754-60 (2009), the content of which is incorporated herein by reference, in its entirety, for all purposes. Sequence read alignment packages import raw or pre-processed sequence reads 122, e.g., in BCL, FASTA, or FASTQ file formats, and output aligned sequence reads 124, e.g., in SAM or BAM file formats.

In some embodiments, sequence data processing module 141 includes one or more demultiplexing algorithms 144, for dividing sequence read or sequence alignment files generated from sequencing reactions of pooled nucleic acids into separate sequence read or sequence alignment files, each of which corresponds to a different source of nucleic acids in the nucleic acid sequencing pool. For instance, because of the cost of sequencing reactions, it is common practice to pool nucleic acids from a plurality of samples into a single sequencing reaction. The nucleic acids from each sample are tagged with a sample-specific and/or molecule-specific sequence tag (e.g., a UMI), which is sequenced along with the molecule. In some embodiments, demultiplexing algorithms 144 sort these sequence tags in the sequence read or sequence alignment files to demultiplex the sequencing data into separate files for each of the samples included in the sequencing reaction.

Bioinformatics module 140 includes a feature extraction module 145, which includes instructions for identifying diagnostic features, e.g., genomic features 131, from sequencing data 122 of biological samples from a subject, e.g., one or more of a solid tumor sample, a liquid biopsy sample, or a normal tissue (e.g., control) sample. For instance, in some embodiments, a feature extraction algorithm compares the identity of one or more nucleotides at a locus from the sequencing data 122 to the identity of the nucleotides at that locus in a reference sequence construct (e.g., a reference genome, exome, or targeted-panel construct) to determine whether the subject has a variant at that locus. In some embodiments, a feature extraction algorithm evaluates data other than the raw sequence, to identify a genomic alteration in the subject, e.g., an allelic ratio, a relative copy number, a repeat unit distribution, etc.

For instance, in some embodiments, feature extraction module 145 includes one or more variant identification modules that include instructions for various variant calling processes. In some embodiments, variants in the germline of the subject are identified, e.g., using a germline variant identification module 146. In some embodiments, variants in the cancer genome, e.g., somatic variants, are identified, e.g., using a somatic variant identification module 150. While separate germline and somatic variant identification modules are illustrated in FIG. 1A, in some embodiments they are integrated into a single module. In some embodiments, the variant identification module includes instructions for identifying one or more of nucleotide variants (e.g., single nucleotide variants (SNV) and multi-nucleotide variants (MNV)) using one or more SNV/MNV calling algorithms (e.g., algorithms 147 and/or 151), indels (e.g., insertions or deletions of nucleotides) using one or more indel calling algorithms (e.g., algorithms 148 and/or 152), and genomic rearrangements (e.g., inversions, translocation, and fusions of nucleotide sequences) using one or more genomic rearrangement calling algorithms (e.g., algorithms 149 and/or 153).

A SNV/MNV algorithm 147 may identify a substitution of a single nucleotide that occurs at a specific position in the genome. For example, at a specific base position, or locus, in the human genome, the C nucleotide may appear in most individuals, but in a minority of individuals, the position is occupied by an A. This means that there is a SNP at this specific position and the two possible nucleotide variations, C or A, are said to be alleles for this position. SNPs underlie differences in human susceptibility to a wide range of diseases (e.g.—sickle-cell anemia, β-thalassemia and cystic fibrosis result from SNPs). The severity of illness and the way the body responds to treatments are also manifestations of genetic variations. For example, a single-base mutation in the APOE (apolipoprotein E) gene is associated with a lower risk for Alzheimer's disease. A single-nucleotide variant (SNV) is a variation in a single nucleotide without any limitations of frequency and may arise in somatic cells. A somatic single-nucleotide variation (e.g., caused by cancer) may also be called a single-nucleotide alteration. An MNP (Multiple-nucleotide polymorphisms) module may identify the substitution of consecutive nucleotides at a specific position in the genome.

An indel calling algorithm 148 may identify an insertion or deletion of bases in the genome of an organism classified among small genetic variations. While indels usually measure from 1 to 10 000 base pairs in length, a microindel is defined as an indel that results in a net change of 1 to 50 nucleotides. Indels can be contrasted with a SNP or point mutation. An indel inserts and/or deletes nucleotides from a sequence, while a point mutation is a form of substitution that replaces one of the nucleotides without changing the overall number in the DNA. Indels, being insertions and/or deletions, can be used as genetic markers in natural populations, especially in phylogenetic studies. Indel frequency tends to be markedly lower than that of single nucleotide polymorphisms (SNP), except near highly repetitive regions, including homopolymers and microsatellites.

A genomic rearrangement algorithm 149 may identify hybrid genes formed from two previously separate genes. It can occur as a result of translocation, interstitial deletion, or chromosomal inversion. Gene fusion can play an important role in tumorigenesis. Fusion genes can contribute to tumor formation because fusion genes can produce much more active abnormal protein than non-fusion genes. Often, fusion genes are oncogenes that cause cancer; these include BCR-ABL, TEL-AMLI (ALL with t(12; 21)), AMLI-ETO (M2 AML with t(8; 21)), and TMPRSS2-ERG with an interstitial deletion on chromosome 21, often occurring in prostate cancer. In the case of TMPRSS2-ERG, by disrupting androgen receptor (AR) signaling and inhibiting AR expression by oncogenic ETS transcription factor, the fusion product regulates prostate cancer. Most fusion genes are found from hematological cancers, sarcomas, and prostate cancer. BCAM-AKT2 is a fusion gene that is specific and unique to high-grade serous ovarian cancer. Oncogenic fusion genes may lead to a gene product with a new or different function from the two fusion partners. Alternatively, a proto-oncogene is fused to a strong promoter, and thereby the oncogenic function is set to function by an upregulation caused by the strong promoter of the upstream fusion partner. The latter is common in lymphomas, where oncogenes are juxtaposed to the promoters of the immunoglobulin genes. Oncogenic fusion transcripts may also be caused by trans-splicing or read-through events. Since chromosomal translocations play such a significant role in neoplasia, a specialized database of chromosomal aberrations and gene fusions in cancer has been created. This database is called Mitelman Database of Chromosome Aberrations and Gene Fusions in Cancer.

In some embodiments, feature extraction module 145 includes instructions for identifying one or more complex genomic alterations (e.g., features that incorporate more than a change in the primary sequence of the genome) in the cancer genome of the subject. For instance, in some embodiments, feature extraction module 145 includes modules for identifying one or more of copy number variation (e.g., copy number variation analysis module 153), microsatellite instability status (e.g., microsatellite instability analysis module 154), tumor mutational burden (e.g., tumor mutational burden analysis module 155), tumor ploidy (e.g., tumor ploidy analysis module 156), and homologous recombination pathway deficiencies (e.g., homologous recombination pathway analysis module 157).

For example, referring to FIG. 1D, in some embodiments, feature extraction module 145 comprises a tumor fraction estimation module 145-tf. In some embodiments, the tumor fraction estimation module 145-tf comprises a sequence ratio data structure 145-tf-r including a plurality of sequence ratios (e.g., coverage ratios) obtained from a sequencing of a test liquid biopsy sample of a subject. In some embodiments, the sequence ratio data structure 145-tf-r includes the sequence ratios that are used as input to determine tumor fraction estimates for the test liquid biopsy sample. In some embodiments, the tumor fraction estimation module 145-tf also comprises a tumor purity algorithm construct 145-tf-a that executes, for example, a maximum likelihood estimation (e.g., an expectation-maximization algorithm) to calculate an estimate of the circulating tumor fraction. The tumor plurality algorithm construct 145-tf-a comprises an optional input data filtration construct 145-tf-k (e.g., for removing one or more inputs passed from the sequence ratio data structure based on a minimum probe threshold or a position on a sex chromosome) and a plurality of model parameters 145-tf-d (e.g., 145-tf-d-1, 145-tf-d-2, . . . ) used for executing the algorithm. In some embodiments, model parameters include expected sequence ratios for a set of copy states at a given tumor purity; a distance (e.g., an error) from a test sequence ratio to the closest expected sequence ratio at the given tumor purity; a minimum distance (e.g., a minimum error) from a test sequence ratio to the closest expected sequence ratio at the given tumor purity (e.g., an assigned test copy state selected from a minimal distance expected copy state); and/or a tumor purity score (e.g., a sum of weighted errors).

In some embodiments, referring to FIG. 1C, the tumor fraction estimation module 145-tf is used to obtain one or more circulating tumor fraction estimates 131-i that are included as feature data 125 in a test patient data store 120. For example, in some embodiments, a plurality of circulating tumor fraction estimates is obtained from a test liquid biopsy sample of a subject 131-i-cf (e.g., 131-i-cf-1, 131-i-cf-2 . . . , 131-i-cf-N). In some embodiments, the plurality of circulating tumor fraction estimates is obtained from a single patient at different collection times.

### Feature Analysis Module (160)

Referring again to FIG. 1A, the system (e.g., system 100) includes a feature analysis module 160 that includes one or more genomic alteration interpretation algorithms 161, one or more optional clinical data analysis algorithms 165, an optional therapeutic curation algorithm 165, and an optional recommendation validation module 167. In some embodiments, feature analysis module 160 identifies actionable variants and characteristics 139-1 and corresponding matched therapies 139-2 and/or clinical trials using one or more analysis algorithms (e.g., algorithms 162, 163, 164, and 165) to evaluate feature data 125. The identified actionable variants and characteristics 139-1 and corresponding matched therapies 139-2, which are optionally stored in test patient data store 120, are then curated by feature analysis module 160 to generate a clinical report 139-3, which is optionally validated by a user, e.g., a clinician, before being transmitted to a medical professional, e.g., an oncologist, treating the patient.

In some embodiments, the genomic alteration interpretation algorithms 161 include instructions for evaluating the effect that one or more genomic features 131 of the subject, e.g., as identified by feature extraction module 145, have on the characteristics of the patient's cancer and/or whether one or more targeted cancer therapies may improve the clinical outcome for the patient. For example, in some embodiments, one or more genomic variant analysis algorithms 163 evaluate various genomic features 131 by querying a database, e.g., a look-up-table (“LUT”) of actionable genomic alterations, targeted therapies associated with the actionable genomic alterations, and any other conditions that should be met before administering the targeted therapy to a subject having the actionable genomic alteration. For instance, evidence suggests that depatuxizumab mafodotin (an anti-EGFR mAb conjugated to monomethyl auristatin F) has improved efficacy for the treatment of recurrent glioblastomas having EGFR focal amplifications. van den Bent et al., 2017, Cancer Chemother Pharmacol., 80 (6): 1209-17. Accordingly, the actionable genomic alteration LUT would have an entry for the focal amplification of the EGFR gene indicating that depatuxizumab mafodotin is a targeted therapy for glioblastomas (e.g., recurrent glioblastomas) having a focal gene amplification. In some instances, the LUT may also include counter indications for the associated targeted therapy, e.g., adverse drug interactions or personal characteristics that are counter-indicated for administration of the particular targeted therapy.

In some embodiments, a genomic alteration interpretation algorithm 161 determines whether a particular genomic feature 131 should be reported to a medical professional treating the cancer patient. In some embodiments, genomic features 131 (e.g., genomic alterations and compound features) are reported when there is clinical evidence that the feature significantly impacts the biology of the cancer, impacts the prognosis for the cancer, and/or impacts pharmacogenomics, e.g., by indicating or counter-indicating particular therapeutic approaches. For instance, a genomic alteration interpretation algorithm 161 may classify a particular CNV feature 135 as “Reportable,” e.g., meaning that the CNV has been identified as influencing the character of the cancer, the overall disease state, and/or pharmacogenomics, as “Not Reportable,” e.g., meaning that the CNV has not been identified as influencing the character of the cancer, the overall disease state, and/or pharmacogenomics, as “No Evidence,” e.g., meaning that no evidence exists supporting that the CNV is “Reportable” or “Not Reportable,” or as “Conflicting Evidence,” e.g., meaning that evidence exists supporting both that the CNV is “Reportable” and that the CNV is “Not Reportable.”

In some embodiments, the genomic alteration interpretation algorithms 161 include one or more pathogenic variant analysis algorithms 162, which evaluate various genomic features to identify the presence of an oncogenic pathogen associated with the patient's cancer and/or targeted therapies associated with an oncogenic pathogen infection in the cancer. For instance, RNA expression patterns of some cancers are associated with the presence of an oncogenic pathogen that is helping to drive the cancer. See, for example, U.S. patent application Ser. No. 16/802,126, filed Feb. 26, 2020, the content of which is hereby incorporated by reference, in its entirety, for all purposes. In some instances, the recommended therapy for the cancer is different when the cancer is associated with the oncogenic pathogen infection than when it is not. Accordingly, in some embodiments, e.g., where feature data 125 includes RNA abundance data for the cancer of the patient, one or more pathogenic variant analysis algorithms 162 evaluate the RNA abundance data for the patient's cancer to determine whether a signature exists in the data that indicates the presence of the oncogenic pathogen in the cancer. Similarly, in some embodiments, bioinformatics module 140 includes an algorithm that searches for the presence of pathogenic nucleic acid sequences in sequencing data 122. See, for example, U.S. Provisional Patent Application Ser. No. 62/978,067, filed Feb. 18, 2020, the content of which is hereby incorporated by reference, in its entirety, for all purposes. Accordingly, in some embodiments, one or more pathogenic variant analysis algorithms 162 evaluates whether the presence of an oncogenic pathogen in a subject is associated with an actionable therapy for the infection. In some embodiments, system 100 queries a database, e.g., a look-up-table (“LUT”), of actionable oncogenic pathogen infections, targeted therapies associated with the actionable infections, and any other conditions that should be met before administering the targeted therapy to a subject that is infected with the oncogenic pathogen. In some instances, the LUT may also include counter indications for the associated targeted therapy, e.g., adverse drug interactions or personal characteristics that are counter-indicated for administration of the particular targeted therapy.

In some embodiments, the genomic alteration interpretation algorithms 161 include one or more multi-feature analysis algorithms 164 that evaluate a plurality of features to classify a cancer with respect to the effects of one or more targeted therapies. For instance, in some embodiments, feature analysis module 160 includes one or more classifiers trained against feature data, one or more clinical therapies, and their associated clinical outcomes for a plurality of training subjects to classify cancers based on their predicted clinical outcomes following one or more therapies.

In some embodiments, the classifier is implemented as an artificial intelligence engine and may include gradient boosting models, random forest models, neural networks (NN), regression models, Naive Bayes models, and/or machine learning algorithms (MLA). An MLA or a NN may be trained from a training data set that includes one or more features 125, including personal characteristics 126, medical history 127, clinical features 128, genomic features 131, and/or other -omic features 138. MLAs include supervised algorithms (such as algorithms where the features/classifications in the data set are annotated) using linear regression, logistic regression, decision trees, classification and regression trees, naïve Bayes, nearest neighbor clustering; unsupervised algorithms (such as algorithms where no features/classification in the data set are annotated) using Apriori, means clustering, principal component analysis, random forest, adaptive boosting; and semi-supervised algorithms (such as algorithms where an incomplete number of features/classifications in the data set are annotated) using generative approach (such as a mixture of Gaussian distributions, mixture of multinomial distributions, hidden Markov models), low density separation, graph-based approaches (such as mincut, harmonic function, manifold regularization), heuristic approaches, or support vector machines.

NNs include conditional random fields, convolutional neural networks, attention based neural networks, deep learning, long short term memory networks, or other neural models where the training data set includes a plurality of tumor samples, RNA expression data for each sample, and pathology reports covering imaging data for each sample.

While MLA and neural networks identify distinct approaches to machine learning, the terms may be used interchangeably herein. Thus, a mention of MLA may include a corresponding NN or a mention of NN may include a corresponding MLA unless explicitly stated otherwise. Training may include providing optimized datasets, labeling these traits as they occur in patient records, and training the MLA to predict or classify based on new inputs. Artificial NNs are efficient computing models which have shown their strengths in solving hard problems in artificial intelligence. They have also been shown to be universal approximators, that is, they can represent a wide variety of functions when given appropriate parameters.

In some embodiments, system 100 includes a classifier training module that includes instructions for training one or more untrained or partially trained classifiers based on feature data from a training dataset. In some embodiments, system 100 also includes a database of training data for use in training the one or more classifiers. In other embodiments, the classifier training module accesses a remote storage device hosting training data. In some embodiments, the training data includes a set of training features, including but not limited to, various types of the feature data 125 illustrated in FIG. 1B. In some embodiments, the classifier training module uses patient data 121, e.g., when test patient data store 120 also stores a record of treatments administered to the patient and patient outcomes following therapy.

In some embodiments, feature analysis module 160 includes one or more clinical data analysis algorithms 165, which evaluate clinical features 128 of a cancer to identify targeted therapies which may benefit the subject. For example, in some embodiments, e.g., where feature data 125 includes pathology data 128-1, one or more clinical data analysis algorithms 165 evaluate the data to determine whether an actionable therapy is indicated based on the histopathology of a tumor biopsy from the subject, e.g., which is indicative of a particular cancer type and/or stage of cancer. In some embodiments, system 100 queries a database, e.g., a look-up-table (“LUT”), of actionable clinical features (e.g., pathology features), targeted therapies associated with the actionable features, and any other conditions that should be met before administering the targeted therapy to a subject associated with the actionable clinical features 128 (e.g., pathology features 128-1). In some embodiments, system 100 evaluates the clinical features 128 (e.g., pathology features 128-1) directly to determine whether the patient's cancer is sensitive to a particular therapeutic agent. Further details on example methods, systems, and algorithms for classifying cancer and identifying targeted therapies based on clinical data, such as pathology data 128-1, imaging data 138-2, and/or tissue culture/organoid data 128-3 are discussed, for example, in U.S. patent application Ser. No. 16/830,186, filed on Mar. 25, 2020, U.S. patent application Ser. No. 16/789,363, filed on Feb. 12, 2020, and U.S. Provisional Application No. 63/007,874, filed on Apr. 9, 2020, the contents of which are hereby incorporated by reference, in their entireties, for all purposes.

In some embodiments, feature analysis module 160 includes a clinical trials module that evaluates test patient data 121 to determine whether the patient is eligible for inclusion in a clinical trial for a cancer therapy, e.g., a clinical trial that is currently recruiting patients, a clinical trial that has not yet begun recruiting patients, and/or an ongoing clinical trial that may recruit additional patients in the future. In some embodiments, a clinical trial module evaluates test patient data 121 to determine whether the results of a clinical trial are relevant for the patient, e.g., the results of an ongoing clinical trial and/or the results of a completed clinical trial. For instance, in some embodiments, system 100 queries a database, e.g., a look-up-table (“LUT”) of clinical trials, e.g., active and/or completed clinical trials, and compares patient data 121 with inclusion criteria for the clinical trials, stored in the database, to identify clinical trials with inclusion criteria that closely match and/or exactly match the patient's data 121. In some embodiments, a record of matching clinical trials, e.g., those clinical trials that the patient may be eligible for and/or that may inform personalized treatment decisions for the patient, are stored in clinical assessment database 139.

In some embodiments, feature analysis module 160 includes a therapeutic curation algorithm 166 that assembles actionable variants and characteristics 139-1, matched therapies 139-2, and/or relevant clinical trials identified for the patient, as described above. In some embodiments, a therapeutic curation algorithm 166 evaluates certain criteria related to which actionable variants and characteristics 139-1, matched therapies 139-2, and/or relevant clinical trials should be reported and/or whether certain matched therapies, considered alone or in combination, may be counter-indicated for the patient, e.g., based on personal characteristics 126 of the patient and/or known drug-drug interactions. In some embodiments, the therapeutic curation algorithm then generates one or more clinical reports 139-3 for the patient. In some embodiments, the therapeutic curation algorithm generates a first clinical report 139-3-1 that is to be reported to a medical professional treating the patient and a second clinical report 139-3-2 that will not be communicated to the medical professional, but may be used to improve various algorithms within the system.

In some embodiments, feature analysis module 160 includes a recommendation validation module 167 that includes an interface allowing a clinician to review, modify, and approve a clinical report 139-3 prior to the report being sent to a medical professional, e.g., an oncologist, treating the patient.

In some embodiments, each of the one or more feature collections, sequencing modules, bioinformatics modules (including, e.g., alteration module(s), structural variant calling and data processing modules), classification modules and outcome modules are communicatively coupled to a data bus to transfer data between each module for processing and/or storage. In some alternative embodiments, each of the feature collection, alteration module(s), structural variant and feature store are communicatively coupled to each other for independent communication without sharing the data bus.

Further details on systems and exemplary embodiments of modules and feature collections are discussed in PCT Publication Number WO2020/142551, which is hereby incorporated herein by reference in its entirety.

### Example Methods

Now that details of a system 100 for detecting whether a variant in a biopsy from a subject is (a) a somatic variant derived from cell free DNA or (b) other than a somatic variant derived from cell free DNA have been disclosed, details regarding processes and features of the system, in accordance with various embodiments of the present disclosure, are disclosed below. Specifically, example processes are described below with reference to FIGS. 2A, 3, 4A-4E, and 5A-5G. In some embodiments, such processes and features of the system are carried out by modules 118, 120, 140, 160, and/or 170, as illustrated in FIG. 1A. Referring to these methods, the systems described herein (e.g., system 100) include instructions for detecting whether a variant in a biopsy from a subject is (a) a somatic variant derived from cell free DNA or (b) other than a somatic variant derived from cell free DNA that are improved compared to conventional methods.

### FIG. 2B: Distributed Diagnostic and Clinical Environment

In some aspects, the methods described herein for providing clinical support for personalized cancer therapy are performed across a distributed diagnostic/clinical environment, e.g., as illustrated in FIG. 2B. However, in some embodiments, the improved methods described herein for detecting clonal hematopoiesis variants and/or solid tumor variants in a liquid biopsy assay, are performed at a single location, e.g., at a single computing system or environment, although ancillary procedures supporting the methods described herein, and/or procedures that make further use of the results of the methods described herein, may be performed across a distributed diagnostic/clinical environment.

FIG. 2B illustrates an example of a distributed diagnostic/clinical environment 210. In some embodiments, the distributed diagnostic/clinical environment is connected via communication network 105. In some embodiments, one or more biological samples, e.g., one or more liquid biopsy samples, solid tumor biopsy, normal tissue samples, and/or control samples, are collected from a subject in clinical environment 220, e.g., a doctor's office, hospital, or medical clinic, or at a home health care environment (not depicted). Advantageously, while solid tumor samples should be collected within a clinical setting, liquid biopsy samples can be acquired in a less invasive fashion and are more easily collected outside of a traditional clinical setting. In some embodiments, one or more biological samples, or portions thereof, are processed within the clinical environment 220 where collection occurred, using a processing device 224, e.g., a nucleic acid sequencer for obtaining sequencing data, a microscope for obtaining pathology data, a mass spectrometer for obtaining proteomic data, etc. In some embodiments, one or more biological samples, or portions thereof are sent to one or more external environments, e.g., sequencing lab 230, pathology lab 240, and/or molecular biology lab 250, each of which includes a processing device 234, 244, and 254, respectively, to generate biological data 121 for the subject. Each environment includes a communications device 222, 232, 242, and 252, respectively, for communicating biological data 121 about the subject to a processing server 262 and/or database 264, which may be located in yet another environment, e.g., processing/storage center 260. Thus, in some embodiments, different portions of the systems and methods described herein are fulfilled by different processing devices located in different physical environments.

Accordingly, in some embodiments, a method for providing clinical support for personalized cancer therapy, e.g., with improved detection of detecting whether a variant in a biopsy from a subject is (a) a somatic variant derived from cell free DNA or (b) other than a somatic variant derived from cell free DNA, is performed across one or more environments, as illustrated in FIG. 2B. For instance, in some such embodiments, a liquid biopsy sample is collected at clinical environment 220 or in a home healthcare environment. The sample, or a portion thereof, is sent to sequencing lab 230 where raw sequence reads 123 of nucleic acids in the sample are generated by sequencer 234. The raw sequencing data 123 is communicated, e.g., from communications device 232, to database 264 at processing/storage center 260, where processing server 262 extracts features from the sequence reads by executing one or more of the processes in bioinformatics module 140, thereby generating genomic features 131 for the sample. Processing server 262 may then analyze the identified features by executing one or more of the processes in feature analysis module 160, thereby generating clinical assessment 139, including a clinical report 139-3. A clinician may access clinical report 139-3, e.g., at processing/storage center 260 or through communications network 105, via recommendation validation module 167. After final approval, clinical report 139-3 is transmitted to a medical professional, e.g., an oncologist, at clinical environment 220, who uses the report to support clinical decision making for personalized treatment of the patient's cancer.

### FIG. 2A: Example Workflow for Precision Oncology

FIG. 2A is a flowchart of an example workflow 200 for collecting and analyzing data in order to generate a clinical report 139 to support clinical decision making in precision oncology. Advantageously, the methods described herein improve this process, for example, by improving various stages within feature extraction 206, including detecting clonal hematopoiesis variants and/or solid tumor variants in a liquid biopsy assay.

Briefly, the workflow begins with patient intake and sample collection 201, where one or more liquid biopsy samples, one or more tumor biopsy, and one or more normal and/or control tissue samples are collected from the patient (e.g., at a clinical environment 220 or home healthcare environment, as illustrated in FIG. 2B). In some embodiments, personal data 126 corresponding to the patient and a record of the one or more biological samples obtained (e.g., patient identifiers, patient clinical data, sample type, sample identifiers, cancer conditions, etc.) are entered into a data analysis platform, e.g., test patient data store 120. Accordingly, in some embodiments, the methods disclosed herein include obtaining one or more biological samples from one or more subjects, e.g., cancer patients. In some embodiments, the subject is a human, e.g., a human cancer patient.

Sequence reads are then generated (312) from the sequencing library or pool of sequencing libraries. Sequencing data may be acquired by any methodology known in the art. For example, next generation sequencing (NGS) techniques such as sequencing-by-synthesis technology (Illumina), pyrosequencing (454 Life Sciences), ion semiconductor technology (Ion Torrent sequencing), single-molecule real-time sequencing (Pacific Biosciences), sequencing by ligation (SOLID sequencing), nanopore sequencing (Oxford Nanopore Technologies), Ultima Sequencing (Ultima Genomics, Fremont California), or paired-end sequencing. In some embodiments, massively parallel sequencing is performed using sequencing-by-synthesis with reversible dye terminators. In some embodiments, sequencing is performed using next generation sequencing technologies, such as short-read technologies. In other embodiments, long-read sequencing or another sequencing method known in the art is used.

Referring again to FIG. 2A, nucleic acid sequencing data 122 generated from the one or more patient samples is then evaluated (e.g., via variant analysis 206) in a bioinformatics pipeline, e.g., using bioinformatics module 140 of system 100, to identify genomic alterations and other metrics in the cancer genome of the patient. An example overview for a bioinformatics pipeline is described below with respect to FIGS. 4A-4E. Advantageously, in some embodiments, the present disclosure improves bioinformatics pipelines, like pipeline 206, by improving circulating tumor fraction estimates.

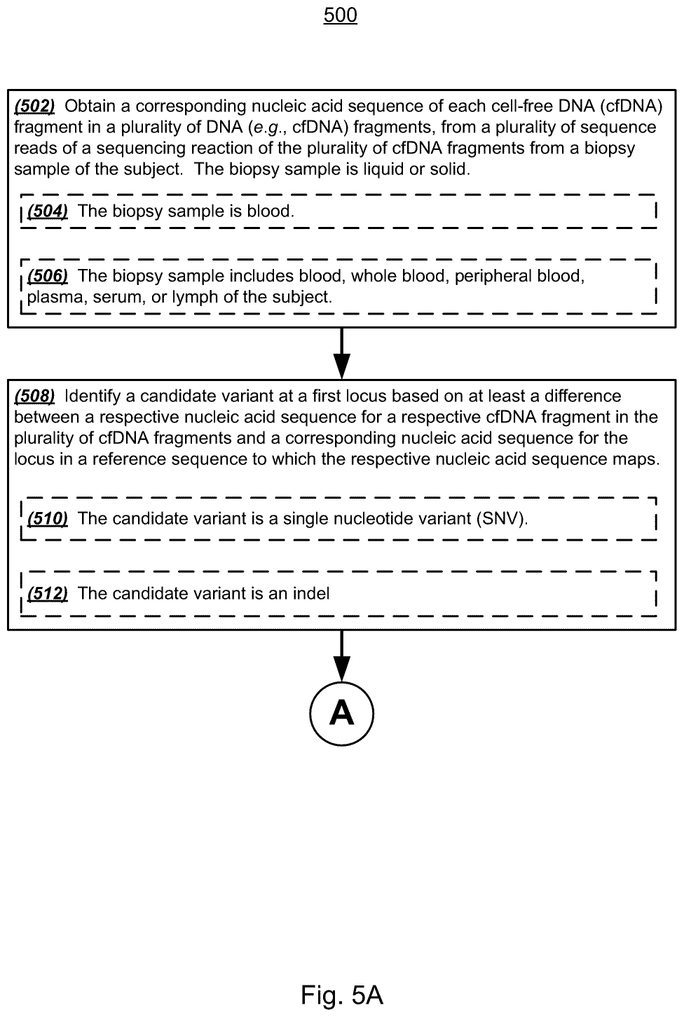

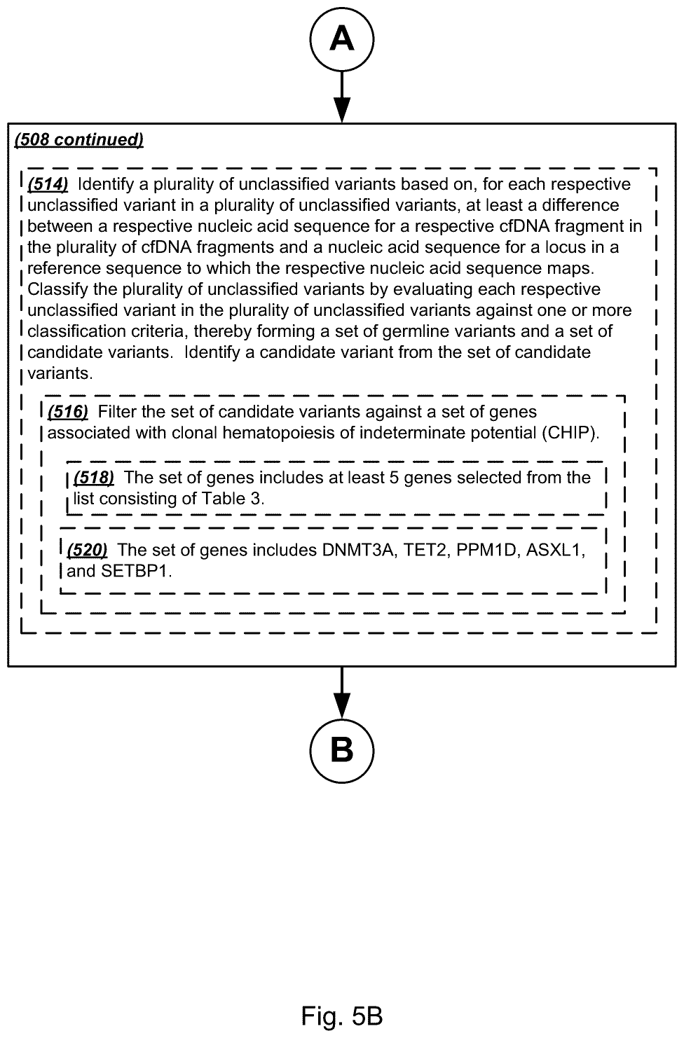

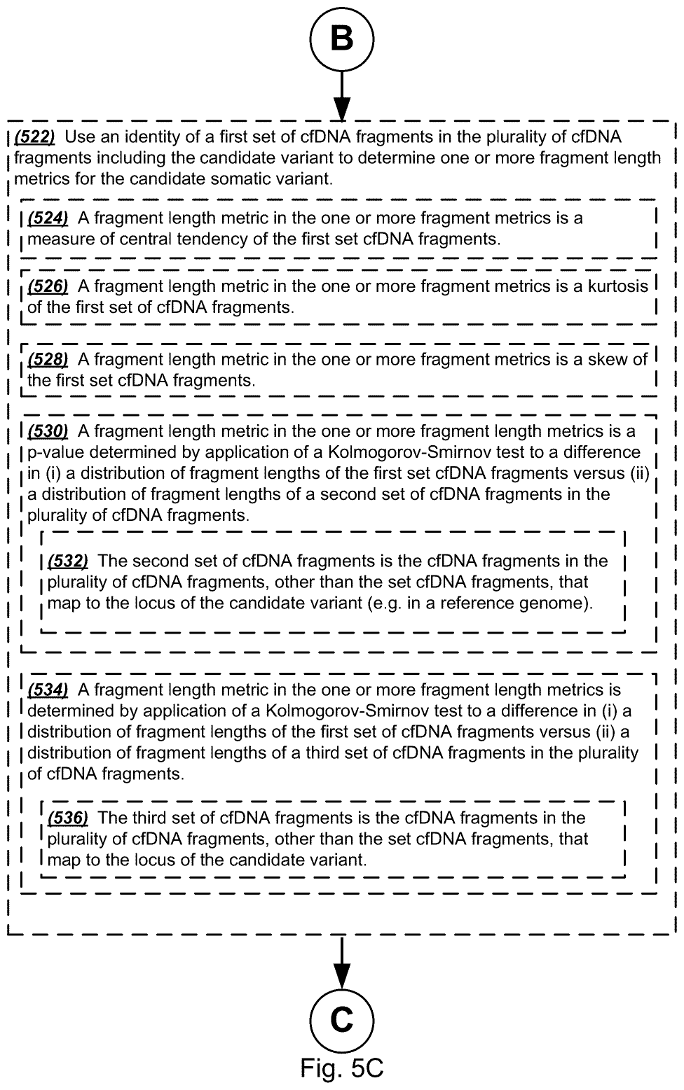

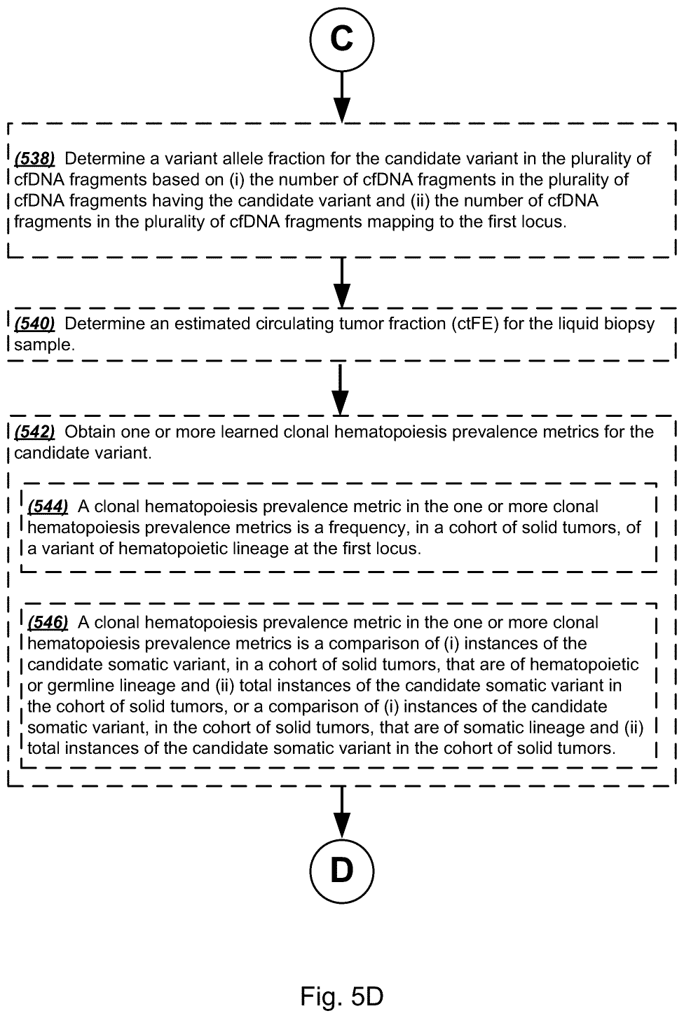

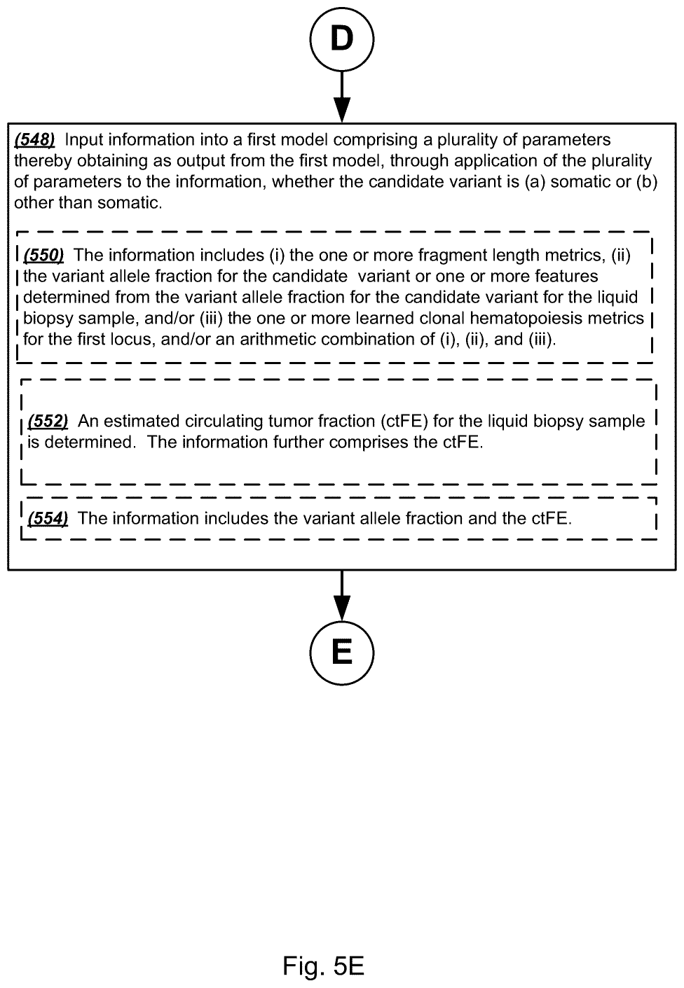

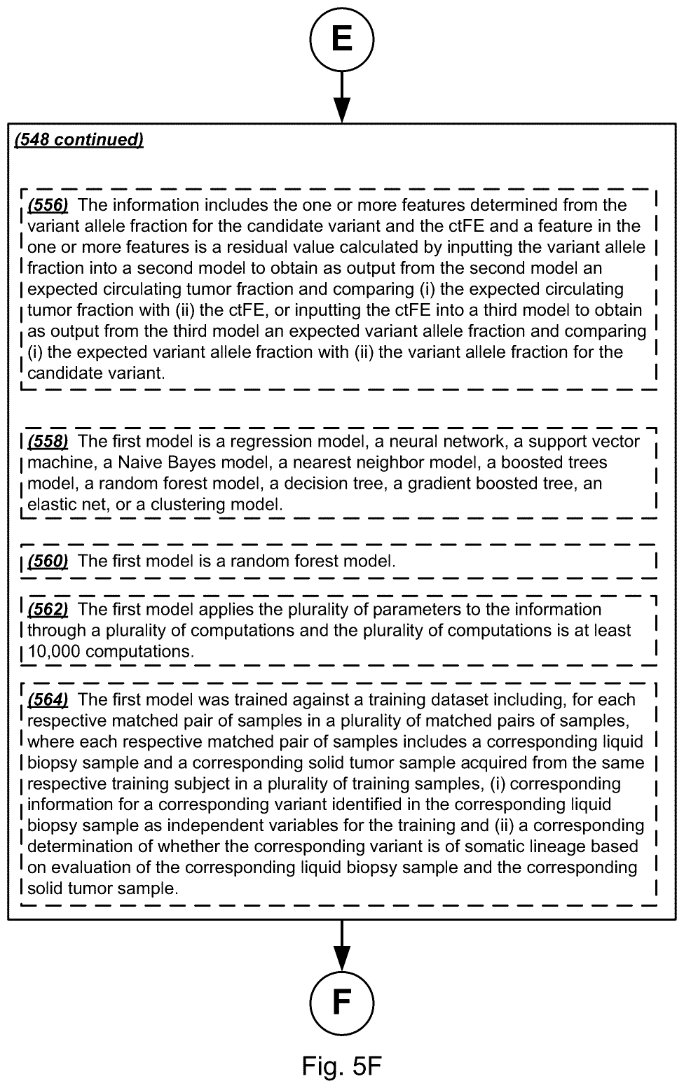

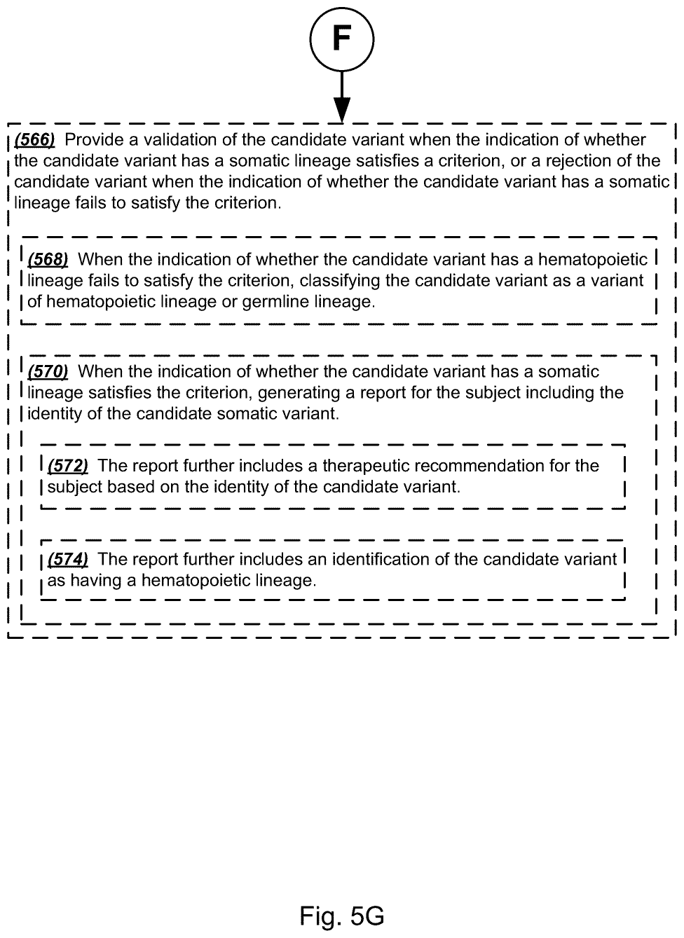

Further details of such sequencing are provided below in conjunction with FIG. 5.

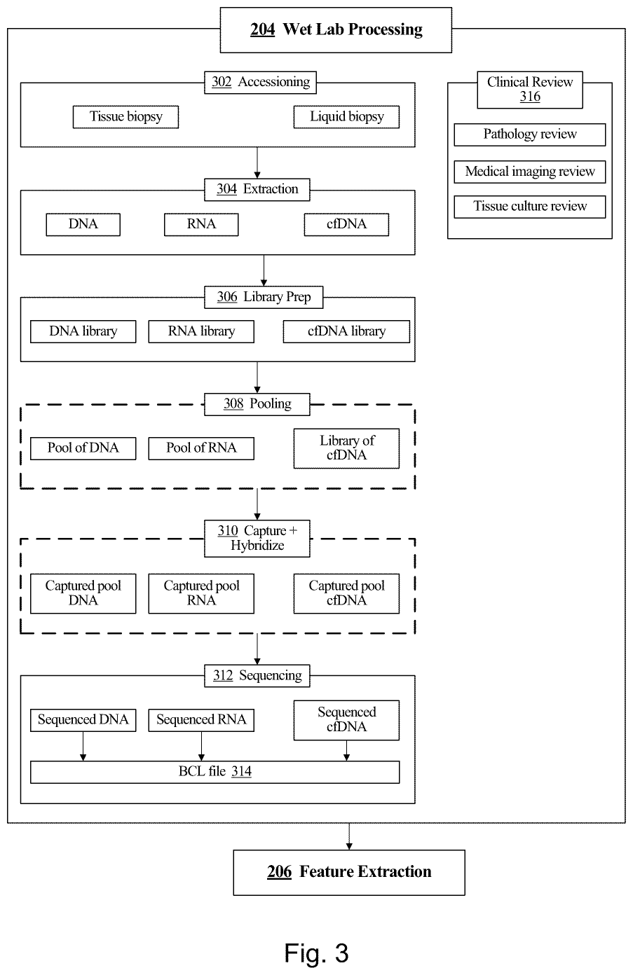

FIG. 4A illustrates an example bioinformatics pipeline 206 (e.g., as used for feature extraction in the workflows illustrated in FIGS. 2A and 3) for providing clinical support for precision oncology. As shown in FIG. 4A, sequencing data 122 obtained from the wet lab processing 204 (e.g., sequence reads 314) is input into the pipeline.

In various embodiments, the bioinformatics pipeline includes a circulating tumor DNA (ctDNA) pipeline for analyzing liquid biopsy samples. The pipeline may detect SNVs, INDELs, copy number amplifications/deletions and genomic rearrangements (for example, fusions). The pipeline may employ unique molecular index (UMI)-based consensus base calling as a method of error suppression as well as a Bayesian tri-nucleotide context-based position level error suppression. In various embodiments, it is able to detect variants having a 0.1%, 0.15%, 0.2%, 0.25%, 0.3%, 0.4%, or 0.5% variant allele fraction.

### Homologous Recombination Status (HRD):

In some embodiments, analysis of aligned sequence reads, e.g., in SAM or BAM format, includes analysis of whether the cancer is homologous recombination deficient (HRD status 137-3), using a homologous recombination pathway analysis module 157.

Homologous recombination (HR) is a normal, highly conserved DNA repair process that enables the exchange of genetic information between identical or closely related DNA molecules. It is most widely used by cells to accurately repair harmful breaks (e.g. damage) that occur on both strands of DNA. DNA damage may occur from exogenous (external) sources like UV light, radiation, or chemical damage; or from endogenous (internal) sources like errors in DNA replication or other cellular processes that create DNA damage. Double strand breaks are a type of DNA damage. Using poly (ADP-ribose) polymerase (PARP) inhibitors in patients with HRD compromises two pathways of DNA repair, resulting in cell death (apoptosis). The efficacy of PARP inhibitors is improved not only in ovarian cancers displaying germline or somatic BRCA mutations, but also in cancers in which HRD is caused by other underlying etiologies.

In some embodiments, HRD status can be determined by inputting features correlated with HRD status into a classifier trained to distinguish between cancers with homologous recombination pathway deficiencies and cancers without homologous recombination pathway deficiencies. For example, in some embodiments, the features include one or more of (i) a heterozygosity status for a first plurality of DNA damage repair genes in the genome of the cancerous tissue of the subject, (ii) a measure of the loss of heterozygosity across the genome of the cancerous tissue of the subject, (iii) a measure of variant alleles detected in a second plurality of DNA damage repair genes in the genome of the cancerous tissue of the subject, and (iv) a measure of variant alleles detected in the second plurality of DNA damage repair genes in the genome of the non-cancerous tissue of the subject. In some embodiments, all four of the features described above are used as features in an HRD classifier. More details about HRD classifiers using these and other features are described in U.S. Patent Application Publication No. 2020/0255909, the content of which is hereby incorporated by reference, in its entirety, for all purposes.

### Concurrent Testing

Unless stated otherwise, as used herein, the term “concurrent” as it relates to assays refers to a period of time between zero and ninety days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 90 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 60 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 30 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 21 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 14 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 7 days. In some embodiments, concurrent tests using different biological samples from the same subject (e.g., two or more of a liquid biopsy sample, cancerous tissue—such as a solid tumor sample or blood sample for a blood-based cancer—and a non-cancerous sample) are performed within a period of time (e.g., the biological samples are collected within the period of time) of from 0 days to 3 days.

In some embodiments, a liquid biopsy assay may be used concurrently with a solid tumor assay to return more comprehensive information about a patient's variants. For example, a blood specimen and a solid tumor specimen may be sent to a laboratory for evaluation. The solid tumor specimen may be analyzed using a bioinformatics pipeline to produce a solid tumor result. A solid tumor assay is described, for instance, in U.S. patent application Ser. No. 16/657,804, the content of which is hereby incorporated by reference, in its entirety, for all purposes. The cancer type of the solid tumor may include, for example, non small cell lung cancer, colorectal cancer, or breast cancer. Alterations identified in the tumor/matched normal result may include, for example, EGFR+ for non small cell lung cancer; HER2+ for breast cancer; or KRAS G12C for several cancers.

In some embodiments, a blood specimen may be divided into a first portion and a second portion. The first portion of the blood specimen and the solid tumor specimen may be analyzed using a bioinformatics pipeline to produce a tumor/matched normal result. The second portion of the blood specimen may be analyzed using a bioinformatics pipeline to produce a liquid biopsy result. For example, the blood specimen may be analyzed using at least an improvement in somatic variant identification, e.g., as described herein in the section entitled “Variant Identification.” For example, the blood specimen may be analyzed using an improvement in focal copy number identification, e.g., as described herein in the section entitled “Copy Number Variation.” For example, the blood specimen may be analyzed using an improvement in circulating tumor fraction determination, e.g., as described above in the section entitled “Systems and Methods for Improved Circulating Tumor Fraction Estimates” and/or “Systems and Methods for Improved Validation of Somatic Sequence Variants.”

Therapies may be identified for further consideration in response to receiving the tumor or tumor/matched normal result along with the liquid biopsy result. For example, if the results overall indicate that the patient has HER2+ breast cancer, neratinib may be identified along with the test results for further consideration by the ordering clinician.

The solid tumor or tumor/matched normal assay may be ordered concurrently; their results may be delivered concurrently; and they may be analyzed concurrently.

Systems and methods for detecting whether a variant in a biopsy from a subject is (a) a somatic variant derived from cell free DNA or (b) other than a somatic variant derived from cell free DNA.

An overview of methods for providing clinical support for personalized cancer therapy is described above with reference to FIGS. 2-4E above. Below, systems and methods for improved detection of clonal hematopoiesis variants and/or solid tumor variants, e.g., within the context of the methods and systems described above, are described with reference to FIGS. 5A-5G.

Many of the embodiments described below, in conjunction with FIGS. 5A-5G, relate to analyses performed using sequencing data for cfDNA obtained from a liquid biopsy sample of a subject, e.g., a cancer patient. Generally, these embodiments are independent and, thus, not reliant upon any particular DNA sequencing methods. However, in some embodiments, the methods described below include generating the sequencing data.

As described herein, in some embodiments, the methods described herein (e.g., method 500 as illustrated in FIGS. 5A-5G) include one or more data collection steps, in addition to data analysis and downstream steps. For example, as described herein, e.g., with reference to FIGS. 2 and 3, in some embodiments, the methods include collection of a liquid biopsy sample and, optionally, one or more matching biological samples from the subject (e.g., a matched cancerous and/or matched non-cancerous sample from the subject). Likewise, as described herein, e.g., with reference to FIGS. 2 and 3, in some embodiments, the methods include extraction of DNA from the liquid biopsy sample (cfDNA) and, optionally, one or more matching biological samples from the subject (e.g., a matched cancerous and/or matched non-cancerous sample from the subject). Similarly, as herein, e.g., with reference to FIGS. 2 and 3, in some embodiments, the methods include nucleic acid sequencing of DNA from the liquid biopsy (cfDNA) sample and, optionally, one or more matching biological samples from the subject (e.g., a matched cancerous and/or matched non-cancerous sample from the subject). Advantageously, the methods and systems described herein allow for accurate classification of variant lineage as either somatic or hematopoietic based on sequencing data from only cfDNA fragments. Accordingly, in some embodiments, a matched cancerous and/or matched non-cancerous sample from the subject is not used in the methods described herein.

However, in other embodiments, the methods described herein begin with obtaining nucleic acid sequencing results, e.g., raw or collapsed sequence reads of DNA from a liquid biopsy sample (cfDNA) and, optionally, one or more matching biological samples from the subject (e.g., a matched cancerous and/or matched non-cancerous sample from the subject), from which the genomic features needed for detecting clonal hematopoiesis variants and/or solid tumor variants can be determined. For example, in some embodiments, sequencing data 122 for a patient 121 is accessed and/or downloaded over network 105 by system 100.

In some embodiments, the method further comprises isolating the plurality of cell-free nucleic acids from the liquid biopsy sample of the test subject prior to the sequencing. In some embodiments, the sequencing is multiplexed sequencing. In some embodiments, the sequencing is short-read sequencing or long-read sequencing.

Similarly, in some embodiments, the methods described herein begin with obtaining the genomic features needed for filtering of clonal hematopoiesis variants from a sequencing of a liquid biopsy sample and, optionally, one or more matching biological samples from the subject (e.g., a matched cancerous and/or matched non-cancerous sample from the subject). For example, in some embodiments, (i) one or more fragment length metrics, (ii) a variant allele fraction for the candidate somatic variant and a ctFE for the liquid biopsy sample or one or more features determined from the variant allele fraction for the candidate somatic variant and the ctFE for the liquid biopsy sample, and (iii) one or more metrics of clonal hematopoiesis prevalence for the first nucleotide position, is accessed and/or downloaded over network 105 by system 100.

FIGS. 5A-5G collectively provide a flow chart of processes and features for validating a sequence variant of a test subject as a somatic variant derived from cell free DNA using a liquid biopsy assay, in accordance with some embodiments of the present disclosure.

Block 502. Referring to block 502, in some embodiments, the method includes obtaining a corresponding nucleic acid sequence of each cell-free DNA (cfDNA) fragment in a plurality of DNA fragments (e.g., cfDNA fragments), from a plurality of sequence reads of a sequencing reaction of the plurality of DNA fragments from one or more biological samples from a subject.

In some embodiments, the plurality of sequence reads is from a panel-enriched sequencing reaction that includes a first subset of sequence reads corresponding to cfDNA fragments targeted by one or more probes in a targeted enrichment panel. In some embodiments, the sequencing reaction is a total cfDNA fragment sequencing reaction. That is, in some embodiments, cfDNA fragments are not enriched using probes in a targeted enrichment panel prior to sequencing.

With reference to FIG. 2B, nucleic acid sequencing of one or more samples collected from the subject is performed, e.g., at sequencing lab 230, during wet lab processing 204. An example workflow for nucleic acid sequencing is illustrated in FIG. 3. In some embodiments, the one or more biological samples obtained at the sequencing lab 230 are accessioned (302), to track the sample and data through the sequencing process.

Next, nucleic acids, e.g., RNA and/or DNA are extracted (304) from the one or more biological samples. Methods for isolating nucleic acids from biological samples are known in the art, and are dependent upon the type of nucleic acid being isolated (e.g., cfDNA, DNA, and/or RNA) and the type of sample from which the nucleic acids are being isolated (e.g., liquid biopsy samples, white blood cell buffy coat preparations, formalin-fixed paraffin-embedded (FFPE) solid tissue samples, and fresh frozen solid tissue samples). The selection of any particular nucleic acid isolation technique for use in conjunction with the embodiments described herein is well within the skill of the person having ordinary skill in the art, who will consider the sample type, the state of the sample, the type of nucleic acid being sequenced and the sequencing technology being used.

For instance, many techniques for DNA isolation, e.g., genomic DNA isolation, from a tissue sample are known in the art, such as organic extraction, silica adsorption, and anion exchange chromatography. Likewise, many techniques for RNA isolation, e.g., mRNA isolation, from a tissue sample are known in the art. For example, acid guanidinium thiocyanate-phenol-chloroform extraction (see, for example, Chomczynski and Sacchi, 2006, Nat Protoc, 1 (2): 581-85, which is hereby incorporated by reference herein), and silica bead/glass fiber adsorption (see, for example, Poeckh et al., 2008, Anal Biochem., 373 (2): 253-62, which is hereby incorporated by reference herein). The selection of any particular DNA or RNA isolation technique for use in conjunction with the embodiments described herein is well within the skill of the person having ordinary skill in the art, who will consider the tissue type, the state of the tissue, e.g., fresh, frozen, formalin-fixed, paraffin-embedded (FFPE), and the type of nucleic acid analysis that is to be performed.

In some embodiments where the biological sample is a liquid biopsy sample, e.g., a blood or blood plasma sample, cfDNA is isolated from blood samples using commercially available reagents, including proteinase K, to generate a liquid solution of cfDNA.

In some embodiments, isolated DNA molecules are mechanically sheared to an average length using an ultrasonicator (for example, a Covaris ultrasonicator). In some embodiments, isolated nucleic acid molecules are analyzed to determine their fragment size, e.g., through gel electrophoresis techniques and/or the use of a device such as a LabChip GX Touch. The skilled artisan will know of an appropriate range of fragment sizes, based on the sequencing technique being employed, as different sequencing techniques have differing fragment size requirements for robust sequencing. In some embodiments, quality control testing is performed on the extracted nucleic acids (e.g., DNA and/or RNA), e.g., to assess the nucleic acid concentration and/or fragment size. For example, sizing of DNA fragments provides valuable information used for downstream processing, such as determining whether DNA fragments require additional shearing prior to sequencing.

Wet lab processing 204 then includes preparing a nucleic acid library from the isolated nucleic acids (e.g., cfDNA, DNA, and/or RNA). For example, in some embodiments, DNA libraries (e.g., gDNA and/or cfDNA libraries) are prepared from isolated DNA from the one or more biological samples. In some embodiments, the DNA libraries are prepared using a commercial library preparation kit, e.g., the KAPA Hyper Prep Kit, a New England Biolabs (NEB) kit, or a similar kit.

In some embodiments, during library preparation, adapters (e.g., UDI adapters, such as Roche SeqCap dual end adapters, or UMI adapters such as full length or stubby Y adapters) are ligated onto the nucleic acid molecules. In some embodiments, the adapters include unique molecular identifiers (UMIs), which are short nucleic acid sequences (e.g., 3-10 base pairs) that are added to ends of DNA fragments during adapter ligation. In some embodiments, UMIs are degenerate base pairs that serve as a unique tag that can be used to identify sequence reads originating from a specific DNA fragment. In some embodiments, e.g., when multiplex sequencing will be used to sequence DNA from a plurality of samples (e.g., from the same or different subjects) in a single sequencing reaction, a patient-specific index is also added to the nucleic acid molecules. In some embodiments, the patient specific index is a short nucleic acid sequence (e.g., 3-20 nucleotides) that are added to ends of DNA fragments during library construction, that serve as a unique tag that can be used to identify sequence reads originating from a specific patient sample. Examples of identifier sequences are described, for example, in Kivioja et al., 2011, Nat. Methods 9 (1): 72-74 and Islam et al., 2014, Nat. Methods 11 (2): 163-66, the contents of which are hereby incorporated by reference, in their entireties, for all purposes.

In some embodiments, an adapter includes a PCR primer landing site, designed for efficient binding of a PCR or second-strand synthesis primer used during the sequencing reaction. In some embodiments, an adapter includes an anchor binding site, to facilitate binding of the DNA molecule to anchor oligonucleotide molecules on a sequencer flow cell, serving as a seed for the sequencing process by providing a starting point for the sequencing reaction. During PCR amplification following adapter ligation, the UMIs, patient indexes, and binding sites are replicated along with the attached DNA fragment. This provides a way to identify sequence reads that came from the same original fragment in downstream analysis.

In some embodiments, DNA libraries are amplified and purified using commercial reagents, (e.g., Axygen MAG PCR clean up beads). In some such embodiments, the concentration and/or quantity of the DNA molecules are then quantified using a fluorescent dye and a fluorescence microplate reader, standard spectrofluorometer, or filter fluorometer. In some embodiments, library amplification is performed on a device (e.g., an Illumina C-Bot2) and the resulting flow cell containing amplified target-captured DNA libraries is sequenced on a next generation sequencer (e.g., an Illumina HiSeq 4000 or an Illumina NovaSeq 6000) to a unique on-target depth selected by the user. In some embodiments, DNA library preparation is performed with an automated system, using a liquid handling robot (e.g., a SciClone NGSx).

In some embodiments, where feature data 125 includes methylation states 132 for one or more genomic locations, nucleic acids isolated from the biological sample (e.g., cfDNA) are treated to convert unmethylated cytosines to uracils, e.g., prior to generating the sequencing library. Accordingly, when the nucleic acids are sequenced, all cytosines called in the sequencing reaction were necessarily methylated, since the unmethylated cytosines were converted to uracils and accordingly would have been called as thymidines, rather than cytosines, in the sequencing reaction. Commercial kits are available for bisulfite-mediated conversion of methylated cytosines to uracils, for instance, the EZ DNA Methylation™-Gold, EZ DNA Methylation™-Direct, and EZ DNA Methylation™-Lightning kit (available from Zymo Research Corp (Irvine, CA)). Commercial kits are also available for enzymatic conversion of methylated cytosines to uracils, for example, the APOBEC-Seq kit (available from NEBiolabs, Ipswich, MA).

In some embodiments, wet lab processing 204 includes pooling (308) DNA molecules from a plurality of libraries, corresponding to different samples from the same and/or different patients, to forming a sequencing pool of DNA libraries. When the pool of DNA libraries is sequenced, the resulting sequence reads correspond to nucleic acids isolated from multiple samples. The sequence reads can be separated into different sequence read files, corresponding to the various samples represented in the sequencing read based on the unique identifiers present in the added nucleic acid fragments. In this fashion, a single sequencing reaction can generate sequence reads from multiple samples. Advantageously, this allows for the processing of more samples per sequencing reaction.

In some embodiments, wet lab processing 204 includes enriching (310) a sequencing library, or pool of sequencing libraries, for target nucleic acids, e.g., nucleic acids encompassing loci that are informative for precision oncology and/or used as internal controls for the sequencing or bioinformatics processes. In some embodiments, enrichment is achieved by hybridizing target nucleic acids in the sequencing library to probes that hybridize to the target sequences, and then isolating the captured nucleic acids away from off-target nucleic acids that are not bound by the capture probes. Of course, some off-target nucleic acids will remain in the final sequencing pool.

In some embodiments, the plurality of sequence reads that is obtained from the above described sequencing includes at least 10,000 sequence reads, at least 50,000 sequence reads, at least 100,000 sequence reads, at least 500,000 sequence reads, at least 1 million sequence reads, at least 5 million sequence reads, at least 10 million sequence reads, or more. In some embodiments, the plurality of sequence reads includes no more than 1 billion sequence reads, no more than 500 million sequence reads, no more than 100 million sequence reads, no more than 50 million sequence reads, no more than 10 million sequence reads, no more than 5 million sequence reads, no more than 1 million sequence reads, or less. In some embodiments, the plurality of sequence reads is from 10,000 sequence reads to 1 billion sequence reads, from 10,000 sequence reads to 500 million sequence reads, from 10,000 sequence reads to 100 million sequence reads, from 10,000 sequence reads to 50 million sequence reads, from 10,000 sequence reads to 10 million sequence reads, from 10,000 sequence reads to 5 million sequence reads, or from 10,000 sequence reads to 1 million sequence reads. In some embodiments, the plurality of sequence reads is from 100,000 sequence reads to 1 billion sequence reads, from 100,000 sequence reads to 500 million sequence reads, from 100,000 sequence reads to 100 million sequence reads, from 100,000 sequence reads to 50 million sequence reads, from 100,000 sequence reads to 10 million sequence reads, from 100,000 sequence reads to 5 million sequence reads, or from 100,000 sequence reads to 1 million sequence reads. In some embodiments, the plurality of sequence reads is from 500,000 sequence reads to 1 billion sequence reads, from 500,000 sequence reads to 500 million sequence reads, from 500,000 sequence reads to 100 million sequence reads, from 500,000 sequence reads to 50 million sequence reads, from 500,000 sequence reads to 10 million sequence reads, from 500,000 sequence reads to 5 million sequence reads, or from 500,000 sequence reads to 1 million sequence reads. In some embodiments, the plurality of sequence reads is from 1 million sequence reads to 1 billion sequence reads, from 1 million sequence reads to 500 million sequence reads, from 1 million sequence reads to 100 million sequence reads, from 1 million sequence reads to 50 million sequence reads, from 1 million sequence reads to 10 million sequence reads, or from 1 million sequence reads to 5 million sequence reads.

In some embodiments, the plurality of DNA (e.g., cfDNA) fragments includes at least 1000 DNA (e.g., cfDNA) fragments, at least 5000 DNA (e.g., cfDNA) fragments, at least 10,000 DNA (e.g., cfDNA) fragments, at least 50,000 DNA (e.g., cfDNA) fragments, at least 100,000 DNA (e.g., cfDNA) fragments, at least 500,000 DNA (e.g., cfDNA) fragments, at least 1 million DNA (e.g., cfDNA) fragments, at least 5 million DNA (e.g., cfDNA) fragments, or more. In some embodiments, the plurality of DNA (e.g., cfDNA) fragments includes no more than no more than 100 million DNA (e.g., cfDNA) fragments, no more than 50 million DNA (e.g., cfDNA) fragments, no more than 10 million DNA (e.g., cfDNA) fragments, no more than 5 million DNA (e.g., cfDNA) fragments, no more than 1 million DNA (e.g., cfDNA) fragments, no more than 500,000 DNA (e.g., cfDNA) fragments, no more than 100,000 DNA (e.g., cfDNA) fragments or less. In some embodiments, the plurality of DNA (e.g., cfDNA) fragments is from 1000 DNA (e.g., cfDNA) fragments to 500 million DNA (e.g., cfDNA) fragments, from 1000 DNA (e.g., cfDNA) fragments to 100 million DNA (e.g., cfDNA) fragments, from 1000 DNA (e.g., cfDNA) fragments to 50 million DNA (e.g., cfDNA) fragments, from 1000 DNA (e.g., cfDNA) fragments to 10 million DNA (e.g., cfDNA) fragments, from 1000 DNA (e.g., cfDNA) fragments to 5 million DNA (e.g., cfDNA) fragments, from 1000 DNA (e.g., cfDNA) fragments to 1 million DNA (e.g., cfDNA) fragments, from 1000 DNA (e.g., cfDNA) fragments to 500,000 DNA (e.g., cfDNA) fragments, from 1000 DNA (e.g., cfDNA) fragments to 250,000 DNA (e.g., cfDNA) fragments, or from 1000 DNA (e.g., cfDNA) fragments to 100,000 DNA (e.g., cfDNA) fragments. In some embodiments, the plurality of DNA (e.g., cfDNA) fragments is from 5000 DNA (e.g., cfDNA) fragments to 500 million DNA (e.g., cfDNA) fragments, from 5000 DNA (e.g., cfDNA) fragments to 100 million DNA (e.g., cfDNA) fragments, from 5000 DNA (e.g., cfDNA) fragments to 50 million DNA (e.g., cfDNA) fragments, from 5000 DNA (e.g., cfDNA) fragments to 10 million DNA (e.g., cfDNA) fragments, from 5000 DNA (e.g., cfDNA) fragments to 5 million DNA (e.g., cfDNA) fragments, from 5000 DNA (e.g., cfDNA) fragments to 1 million DNA (e.g., cfDNA) fragments, from 5000 DNA (e.g., cfDNA) fragments to 500,000 DNA (e.g., cfDNA) fragments, from 5000 DNA (e.g., cfDNA) fragments to 250,000 DNA (e.g., cfDNA) fragments, or from 5000 DNA (e.g., cfDNA) fragments to 100,000 DNA (e.g., cfDNA) fragments. In some embodiments, the plurality of DNA (e.g., cfDNA) fragments is from 10,000 DNA (e.g., cfDNA) fragments to 500 million DNA (e.g., cfDNA) fragments, from 10,000 DNA (e.g., cfDNA) fragments to 100 million DNA (e.g., cfDNA) fragments, from 10,000 DNA (e.g., cfDNA) fragments to 50 million DNA (e.g., cfDNA) fragments, from 10,000 DNA (e.g., cfDNA) fragments to 10 million DNA (e.g., cfDNA) fragments, from 10,000 DNA (e.g., cfDNA) fragments to 5 million DNA (e.g., cfDNA) fragments, from 10,000 DNA (e.g., cfDNA) fragments to 1 million DNA (e.g., cfDNA) fragments, from 10,000 DNA (e.g., cfDNA) fragments to 500,000 DNA (e.g., cfDNA) fragments, from 10,000 DNA (e.g., cfDNA) fragments to 250,000 DNA (e.g., cfDNA) fragments, or from 10,000 DNA (e.g., cfDNA) fragments to 100,000 DNA (e.g., cfDNA) fragments. In some embodiments, the plurality of DNA (e.g., cfDNA) fragments is from 25,000 DNA (e.g., cfDNA) fragments to 500 million DNA (e.g., cfDNA) fragments, from 25,000 DNA (e.g., cfDNA) fragments to 100 million DNA (e.g., cfDNA) fragments, from 25,000 DNA (e.g., cfDNA) fragments to 50 million DNA (e.g., cfDNA) fragments, from 25,000 DNA (e.g., cfDNA) fragments to 10 million DNA (e.g., cfDNA) fragments, from 25,000 DNA (e.g., cfDNA) fragments to 5 million DNA (e.g., cfDNA) fragments, from 25,000 DNA (e.g., cfDNA) fragments to 1 million DNA (e.g., cfDNA) fragments, from 25,000 DNA (e.g., cfDNA) fragments to 500,000 DNA (e.g., cfDNA) fragments, from 25,000 DNA (e.g., cfDNA) fragments to 250,000 DNA (e.g., cfDNA) fragments, or from 25,000 DNA (e.g., cfDNA) fragments to 100,000 DNA (e.g., cfDNA) fragments.

In some embodiments, the obtaining, accessioning, storing, preparing, processing and/or analyzing the biopsy sample from the test subject comprises any of the methods and/or embodiments described above in the present disclosure. In some embodiments, the sequencing reaction comprises any of the methods and/or embodiments described above in the present disclosure.

In some embodiments, all, or nearly all, of the aligned sequence reads are evaluated to identify candidate sequence variants (e.g., candidate somatic sequence variants and/or candidate germline sequence variants). In other embodiments, a subset of the aligned sequence reads is evaluated to identify candidate sequence variants. For example, in one embodiment, targeted-panel sequencing reaction is used to generate sequencing data 122 and only sequence reads corresponding to the target panel (on-target reads) are evaluated to identify candidate sequence variants. In some embodiments, targeted-panel sequencing reaction is used to generate sequencing data 122 and a subset of sequence reads corresponding to a subset of the target panel are evaluated to identify candidate sequence variants. In some embodiments, a subset of the sequence reads corresponding to a subset of genes, regardless of whether the sequencing reaction is a targeted-panel sequencing reaction, a whole exome sequencing reaction, or a whole genome sequencing reaction, are evaluated to identify candidate sequence variants. In some embodiments, a subset of sequence reads corresponding to a defined set of regions within the genome, e.g., one or more genes, one or more introns, one or more exons, one or more subregion of an intron and/or exon associated with cancer etiology, etc., are evaluated to identify candidate sequence variants.

Alternatively, in some embodiments, regardless of what subset of aligned sequence reads are evaluated to identify candidate sequence variants, only a subset of candidate sequence variants is further validated. For example, in some embodiments, only candidate sequence variants corresponding to the target panel (on-target reads) are validated. Similarly, in some embodiments, only candidate sequence variants corresponding to a subset of the target panel are validated. Likewise, in some embodiments, only candidate sequence variants corresponding to a subset of genes, regardless of whether the sequencing reaction is a targeted-panel sequencing reaction, a whole exome sequencing reaction, or a whole genome sequencing reaction, are validated. Similarly, in some embodiments, only candidate variants corresponding to a defined set of regions within the genome, e.g., one or more genes, one or more introns, one or more exons, one or more subregion of an intron and/or exon associated with cancer etiology, etc., are validated.

Advantageously, enriching for target sequences prior to sequencing nucleic acids significantly reduces the costs and time associated with sequencing, facilitates multiplex sequencing by allowing multiple samples to be mixed together for a single sequencing reaction, and significantly reduces the computation burden of aligning the resulting sequence reads, as a result of significantly reducing the total amount of nucleic acids analyzed from each sample. Accordingly, in some embodiments, a panel-enriched sequencing reaction is performed at a read depth of at least 1,000×. In some embodiments, a panel-enriched sequencing reaction is performed at a read depth of at least 100×, at least 500×, at least 1000×, at least 5000×, at least 10,000×, at least 50,000×, or greater. In some embodiments, a panel-enriched sequencing reaction is performed at a read depth of no more than 100,000×, no more than 50,000×, no more than 10,000×, no more than 5000×, or less. In some embodiments, a panel-enriched sequencing reaction is performed at a read depth of from 100× to 50,000×, from 100× to 10,000×, from 100× to 5000×, from 100× to 1000×, or from 100× to 500×. In some embodiments, a panel-enriched sequencing reaction is performed at a read depth of from 500× to 50,000×, from 500× to 10,000×, from 500× to 5000×, or from 500× to 1000×. In some embodiments, a panel-enriched sequencing reaction is performed at a read depth of from 1000× to 50,000×, from 1000× to 10,000×, or from 1000× to 5000×.

In some embodiments, the enrichment is performed prior to pooling multiple nucleic acid sequencing libraries. However, in other embodiments, the enrichment is performed after pooling nucleic acid sequencing libraries, which has the advantage of reducing the number of enrichment assays that have to be performed.

In some embodiments, the enrichment is performed prior to generating a nucleic acid sequencing library. This has the advantage that fewer reagents are needed to perform both the enrichment (because there are fewer target sequences at this point, prior to library amplification) and the library production (because there are fewer nucleic acid molecules to tag and amplify after the enrichment). However, this raises the possibility of pull-down bias and/or that small variations in the enrichment protocol will result in less consistent results.

In some embodiments, nucleic acid libraries are pooled (two or more DNA libraries may be mixed to create a pool) and treated with reagents to reduce off-target capture, for example Human COT-1 and/or IDT xGen Universal Blockers. Pools may be dried in a vacufuge and resuspended. DNA libraries or pools may be hybridized to a probe set (for example, a probe set specific to a panel that includes loci from at least 100, 600, 1,000, 10,000, etc. of the 19,000 known human genes) and amplified with commercially available reagents (for example, the KAPA HiFi HotStart ReadyMix). For example, in some embodiments, a pool is incubated in an incubator, PCR machine, water bath, or other temperature-modulating device to allow probes to hybridize. Pools may then be mixed with Streptavidin-coated beads or another means for capturing hybridized DNA-probe molecules, such as DNA molecules representing exons of the human genome and/or genes selected for a genetic panel.

Pools may be amplified and purified more than once using commercially available reagents, for example, the KAPA HiFi Library Amplification kit and Axygen MAG PCR clean up beads, respectively. The pools or DNA libraries may be analyzed to determine the concentration or quantity of DNA molecules, for example by using a fluorescent dye (for example, PicoGreen pool quantification) and a fluorescence microplate reader, standard spectrofluorometer, or filter fluorometer. In one example, the DNA library preparation and/or capture is performed with an automated system, using a liquid handling robot (for example, a SciClone NGSx).

In some embodiments, e.g., where a whole genome sequencing method is used, nucleic acid sequencing libraries are not target-enriched prior to sequencing, in order to obtain sequencing data on substantially all of the competent nucleic acids in the sequencing library. Similarly, in some embodiments, e.g., where a whole genome sequencing method will be used, nucleic acid sequencing libraries are not mixed, because of bandwidth limitations related to obtaining significant sequencing depth across an entire genome. However, in other embodiments, e.g., where a low pass whole genome sequencing (LPWGS) methodology will be used, nucleic acid sequencing libraries can still be pooled, because very low average sequencing coverage is achieved across a respective genome, e.g., between about 0.5× and about 5×.

In some embodiments, a plurality of nucleic acid probes (e.g., a probe set) is used to enrich one or more target sequences in a nucleic acid sample (e.g., an isolated nucleic acid sample or a nucleic acid sequencing library), e.g., where one or more target sequences is informative for precision oncology. For instance, in some embodiments, one or more of the target sequences encompasses a locus that is associated with an actionable allele. That is, variations of the target sequence are associated with targeted therapeutic approaches. In some embodiments, one or more of the target sequences and/or a property of one or more of the target sequences is used in a classifier trained to distinguish two or more cancer states.

In some embodiments, the probe set includes probes targeting one or more gene loci, e.g., exon or intron loci. In some embodiments, the probe set includes probes targeting one or more loci not encoding a protein, e.g., regulatory loci, miRNA loci, and other non-coding loci, e.g., that have been found to be associated with cancer. In some embodiments, the plurality of loci includes at least 25, 50, 100, 150, 200, 250, 300, 350, 400, 500, 750, 1000, 2500, 5000, or more human genomic loci.

In some embodiments, a panel-enriched sequencing reaction uses a sequencing panel that enriches for at least 50 genes. In some embodiments, a panel-enriched sequencing reaction uses a sequencing panel that enriches for at least 25 genes, at least 50 genes, at least 100 genes, at least 250 genes, at least 500 genes, at least 1000 genes, at least 2500 genes, at least 5000 genes, or more. In some embodiments, a panel-enriched sequencing reaction uses a sequencing panel that enriches for no more than 40,000 genes, no more than 20,000 genes, no more than 10,000 genes, no more than 5000 genes, no more than 2500 genes, no more than 1000 genes, or less. In some embodiments, a panel-enriched sequencing reaction uses a sequencing panel that enriches for from 25 genes to 10,000 genes, from 25 genes to 5000 genes, from 25 genes to 2500 genes, from 25 genes to 1000 genes, from 25 genes to 500 genes, or from 25 genes to 250 genes. In some embodiments, a panel-enriched sequencing reaction uses a sequencing panel that enriches for from 50 genes to 10,000 genes, from 50 genes to 5000 genes, from 50 genes to 2500 genes, from 50 genes to 1000 genes, from 50 genes to 500 genes, or from 50 genes to 250 genes. In some embodiments, a panel-enriched sequencing reaction uses a sequencing panel that enriches for from 100 genes to 10,000 genes, from 100 genes to 5000 genes, from 100 genes to 2500 genes, from 100 genes to 1000 genes, from 100 genes to 500 genes, or from 100 genes to 250 genes.

In some embodiments, the plurality of probe sequences used to enrich cell-free DNA fragments in the liquid biopsy sample in a panel-enriched sequencing reaction collectively map to at least 25 different genes in a human reference genome. In some embodiments, a panel-enriched sequencing reaction uses a sequencing panel that enriches for at least 25 human genes, at least 50 human genes, at least 100 human genes, at least 250 human genes, at least 500 human genes, at least 1000 human genes, at least 2500 human genes, at least 5000 human genes, or more. In some embodiments, a panel-enriched sequencing reaction uses a sequencing panel that enriches for no more than 40,000 human genes, no more than 20,000 human genes, no more than 10,000 human genes, no more than 5000 human genes, no more than 2500 human genes, no more than 1000 human genes, or less. In some embodiments, a panel-enriched sequencing reaction uses a sequencing panel that enriches for from 25 human genes to 10,000 human genes, from 25 human genes to 5000 human genes, from 25 human genes to 2500 human genes, from 25 human genes to 1000 human genes, from 25 human genes to 500 human genes, or from 25 human genes to 250 human genes. In some embodiments, a panel-enriched sequencing reaction uses a sequencing panel that enriches for from 50 human genes to 10,000 human genes, from 50 human genes to 5000 human genes, from 50 human genes to 2500 human genes, from 50 human genes to 1000 human genes, from 50 human genes to 500 human genes, or from 50 human genes to 250 human genes. In some embodiments, a panel-enriched sequencing reaction uses a sequencing panel that enriches for from 100 human genes to 10,000 human genes, from 100 human genes to 5000 human genes, from 100 human genes to 2500 human genes, from 100 human genes to 1000 human genes, from 100 human genes to 500 human genes, or from 100 human genes to 250 human genes.

In some embodiments, the plurality of probe sequences used to enrich cell-free DNA fragments in the liquid biopsy sample in a first panel-enriched sequencing reaction collectively map to at least 25 different genes in a human reference genome. In some embodiments, the plurality of probe sequences collectively maps to at least 50, at least 100, at least 250, at least 500, or at least 1000 different genes in the human reference genome. In some embodiments, the plurality of probe sequences collectively maps to at least 10 of the genes listed in Table 1. In some embodiments, the plurality of probe sequences collectively maps to at least 20, 25, 30, 40, 50, 60, 75, 100, or all 105 of the genes listed in Table 1.

In some embodiments, the plurality of probe sequences collectively maps to at least 10 of the genes listed in Table 2. In some embodiments, the plurality of probe sequences collectively maps to at least 20, 25, 30, 40, 50, 60, 75, 100, or all 105 of the genes listed in Table 2.

For example, in some embodiments, a targeted enrichment panel comprises any of the embodiments described above in the present disclosure. In some embodiments, the targeted enrichment panel includes probes targeting one or more gene loci, e.g., exon or intron loci. In some embodiments, the targeted enrichment panel includes probes targeting one or more loci not encoding a protein, e.g., regulatory loci, miRNA loci, and other non-coding loci, e.g., that have been found to be associated with cancer. In some embodiments, the plurality of loci includes at least 25, 50, 100, 150, 200, 250, 300, 350, 400, 500, 750, 1000, 2500, 5000, or more human genomic loci.

In some embodiments, the targeted enrichment panel includes probes targeting one or more of the genes listed in Table 1. In some embodiments, the targeted enrichment panel includes probes targeting at least 5 of the genes listed in Table 1. In some embodiments, the targeted enrichment panel includes probes targeting at least 10 of the genes listed in Table 1. In some embodiments, the targeted enrichment panel includes probes targeting at least 25 of the genes listed in Table 1. In some embodiments, the targeted enrichment panel includes probes targeting at least 50 of the genes listed in Table 1. In some embodiments, the targeted enrichment panel includes probes targeting at least 75 of the genes listed in Table 1. In some embodiments, the targeted enrichment panel includes probes targeting at least 100 of the genes listed in Table 1. In some embodiments, the targeted enrichment panel includes probes targeting all of the genes listed in Table 1.

In some embodiments, the targeted enrichment panel includes probes targeting one or more of the genes listed in Table 2. In some embodiments, the targeted enrichment panel includes probes targeting at least 5 of the genes listed in Table 2. In some embodiments, the targeted enrichment panel includes probes targeting at least 10 of the genes listed in Table 2. In some embodiments, the targeted enrichment panel includes probes targeting at least 25 of the genes listed in Table 2. In some embodiments, the targeted enrichment panel includes probes targeting at least 50 of the genes listed in Table 2. In some embodiments, the targeted enrichment panel includes probes targeting at least 75 of the genes listed in Table 2. In some embodiments, the targeted enrichment panel includes probes targeting at least 100 of the genes listed in Table 2. In some embodiments, the targeted enrichment panel includes probes targeting all of the genes listed in Table 2.

In some embodiments, the probe set includes probes targeting one or more of the genes listed in Table 1. In some embodiments, the probe set includes probes targeting at least 5 of the genes listed in Table 1. In some embodiments, the probe set includes probes targeting at least 10 of the genes listed in Table 1. In some embodiments, the probe set includes probes targeting at least 25 of the genes listed in Table 1. In some embodiments, the probe set includes probes targeting at least 50 of the genes listed in Table 1. In some embodiments, the probe set includes probes targeting at least 75 of the genes listed in Table 1. In some embodiments, the probe set includes probes targeting at least 100 of the genes listed in Table 1. In some embodiments, the probe set includes probes targeting all of the genes listed in Table 1.

In some embodiments, the probe set includes probes targeting one or more of the genes listed in Table 2. In some embodiments, the probe set includes probes targeting at least 5 of the genes listed in Table 2. In some embodiments, the probe set includes probes targeting at least 10 of the genes listed in Table 2. In some embodiments, the probe set includes probes targeting at least 25 of the genes listed in Table 2. In some embodiments, the probe set includes probes targeting at least 50 of the genes listed in Table 2. In some embodiments, the probe set includes probes targeting at least 75 of the genes listed in Table 2. In some embodiments, the probe set includes probes targeting at least 100 of the genes listed in Table 2. In some embodiments, the probe set includes probes targeting all of the genes listed in Table 2.

TABLE 1
An example panel of 105 genes.

| | | | | | | |
|---|---|---|---|---|---|---|
| ALK | B2M | ERRFI1 | IDH2 | MSH6 | PIK3R1 | SPOP |
| FGFR2 | BAP1 | ESR1 | JAK1 | MTOR | PMS2 | STK11 |
| FGFR3 | BRCA1 | EZH2 | JAK2 | MYCN | PTCH1 | TERT |
| NTRK1 | BRCA2 | FBXW7 | JAK3 | NF1 | PTEN | TP53 |
| RET | BTK | FGFR1 | KDR | NF2 | PTPN11 | TSC1 |
| ROS1 | CCND1 | FGFR4 | KEAP1 | NFE2L2 | RAD51C | TSC2 |
| BRAF | CCND2 | FLT3 | KIT | NOTCH1 | RAF1 | UGT1A1 |
| AKT1 | CCND3 | FOXL2 | KRAS | NPM1 | RB1 | VHL |
| AKT2 | CDH1 | GATA3 | MAP2K1 | NRAS | RHEB | CCNE1 |
| APC | CDK4 | GNA11 | MAP2K2 | PALB2 | RHOA | CD274 |
| AR | CDK6 | GNAQ | MAPK1 | PBRM1 | RIT1 | EGFR |
| ARAF | CDKN2A | GNAS | MLH1 | PDCD1LG2 | RNF43 | ERBB2 |
| ARID1A | CTNNB1 | HNF1A | MPL | PDGFRA | SDHA | MET |
| ATM | DDR2 | HRAS | MSH2 | PDGFRB | SMAD4 | MYC |
| ATR | DPYD | IDH1 | MSH3 | PIK3CA | SMO | KMT2A |

TABLE 2
An example panel of 523 genes.

| | | | | | |
|---|---|---|---|---|---|
| BCC3 | CIC | FGFR4 | KLF4 | PBRM1 | SIRPA |
| ABL1 | CKS1B | FH | KLHL6 | PDCD1 | SLC34A2 |
| ABL2 | CREBBP | FHIT | KLLN | PDCD1LG2 | SLC9A3R1 |
| ABRAXAS1 | CRKL | FLCN | KMT2A | PDGFRA | SLFN11 |
| ACVR1 | CSF1R | FLT1 | KMT2C | PDGFRB | SLIT2 |
| ACVR1B | CSF3R | FLT3 | KMT2D | PDK1 | SMAD2 |
| AJUBA | CTC1 | FLT4 | KRAS | PHGDH | SMAD3 |
| AKT1 | CTCF | FOLH1 | LATS1 | PHLPP1 | SMAD4 |
| AKT2 | CTLA4 | FOXA1 | LCK | PHLPP2 | SMARCA2 |
| AKT3 | CTNNA1 | FOXL2 | LMO1 | PIAS4 | SMARCA4 |
| ALK | CTNNB1 | FOXO1 | LRP1B | PIK3C2B | SMARCB1 |
| ALOX12B | CUL3 | FOXO3 | LTK | PIK3C2G | SMC1A |
| AMER1 | CUL4A | FOXP1 | LYN | PIK3CA | SMC3 |
| APC | CUX1 | FRS2 | LZTR1 | PIK3CB | SMO |
| APLNR | CXCR4 | FUBP1 | MAF | PIK3CD | SNCAIP |
| AR | CYLD | GABRA6 | MALT1 | PIK3CG | SOCS1 |
| ARAF | CYP17A1 | GALNT12 | MAP2K1 | PIK3R1 | SOS1 |
| ARFRP1 | CYSLTR2 | GATA1 | MAP2K2 | PIK3R2 | SOX2 |
| ARID1A | DAXX | GATA3 | MAP2K4 | PIM1 | SOX9 |
| ARID1B | DDB2 | GATA4 | MAP3K1 | PLCG1 | SPEN |
| ARID2 | DDR1 | GATA6 | MAP3K13 | PLCG2 | SPOP |
| ASNS | DDR2 | GID4 | MAP3K21 | PMS1 | SRC |
| ASXL1 | DDX3X | GLI2 | MAP3K7 | PMS2 | SRSF2 |
| ATM | DDX41 | GNA11 | MAPK1 | POLA1 | STAG2 |
| ATR | DEPTOR | GNA13 | MAPK3 | POLD1 | STAT3 |
| ATRX | DICER1 | GNAQ | MAX | POLE | STAT5B |
| AURKA | DIS3 | GNAS | MC1R | POLQ | STAT6 |
| AURKB | DNMT1 | GPC3 | MCL1 | POT1 | STK11 |
| AURKC | DNMT3A | GPS2 | MDM2 | PPARG | SUFU |
| AXIN1 | DOT1L | GREM1 | MDM4 | PPM1D | SUZ12 |
| AXIN2 | DPYD | GRIN2A | MED12 | PPP2R1A | SYK |
| AXL | EBF1 | GRM3 | MEF2B | PPP2R2A | TBX3 |
| B2M | EED | GSK3B | MEN1 | PPP6C | TCF7L2 |
| BAP1 | EEF2 | GSTP1 | MERTK | PRDM1 | TEK |
| BARD1 | EGFR | H3F3A | MET | PREX2 | TERC |
| BAX | EGLN1 | HAVCR2 | MITF | PRKACA | TERT |
| BCL2 | EIF1AX | HDAC1 | MKNK1 | PRKAR1A | TET2 |
| BCL2L1 | ELF3 | HDAC2 | MLH1 | PRKCI | TFEB |
| BCL2L11 | EMSY | HGF | MLH3 | PRKN | TGFB1 |
| BCL2L2 | EP300 | HIF1A | MPL | PTCH1 | TGFBR1 |
| BCL6 | EPCAM | HIST1H3B | MRE11 | PTEN | TGFBR2 |
| BCLAF1 | EPHA2 | HLA-B | MS4A1 | PTK2 | TIGIT |
| BCOR | EPHA3 | HNF1A | MSH2 | PTPN11 | TIPARP |
| BCORL1 | EPHB1 | HNF1B | MSH3 | PTPN13 | TMEM127 |
| BCR | EPHB4 | HOXB13 | MSH6 | PTPRD | TMPRSS2 |
| BIRC3 | ERBB2 | HRAS | MST1R | PTPRO | TNFAIP3 |
| BLM | ERBB3 | HSD3B1 | MTAP | PTPRT | TNFRSF14 |
| BMPR1A | ERBB4 | HSP90AA1 | MTHFR | QKI | TNFRSF17 |
| BRAF | ERCC2 | HSPH1 | MTOR | RAC1 | TOP1 |
| BRCA1 | ERCC3 | ID3 | MUC16 | RAD21 | TOP2A |
| BRCA2 | ERCC4 | IDH1 | MUTYH | RAD50 | TP53 |
| BRD4 | ERCC6 | IDH2 | MYB | RAD51 | TP53BP1 |
| BRIP1 | ERG | IFNA21 | MYC | RAD51B | TP63 |
| BTG1 | ERRFI1 | IFNAR1 | MYCL | RAD51C | TRAF3 |
| BTG2 | ESR1 | IFNAR2 | MYCN | RAD51D | TRAF7 |
| BTK | ETNK1 | IFNG | MYD88 | RAD52 | TSC1 |
| CALR | ETV1 | IFNGR1 | NBN | RAD54L | TSC2 |
| CARD11 | ETV4 | IFNGR2 | NCOA2 | RAF1 | TSHR |
| CARM1 | ETV5 | IFNW1 | NCOR1 | RARA | TYMS |
| CASP8 | ETV6 | IGF1 | NF1 | RASA1 | TYRO3 |
| CBFB | EWSR1 | IGF1R | NF2 | RB1 | U2AF1 |
| CBL | EZH2 | IKBKE | NFE2L2 | RBM10 | UGT1A1 |
| CCND1 | EZR | IKZF1 | NFKBIA | RECQL4 | VEGFA |
| CCND2 | FAM46C | IL10RA | NKX2-1 | REL | VHL |
| CCND3 | FANCA | IL32 | NOTCH1 | RET | VSIR |
| CCNE1 | FANCC | IL6R | NOTCH2 | RHEB | WEE1 |
| CD22 | FANCD2 | IL7R | NOTCH3 | RHOA | WNK1 |
| CD274 | FANCE | IMPDH1 | NOTCH4 | RICTOR | WRN |
| CD70 | FANCG | ING1 | NPM1 | RIT1 | WT1 |
| CD74 | FANCI | INPP4B | NQO1 | RNF43 | XBP1 |
| CD79A | FANCL | INSR | NRAS | ROS1 | XPA |
| CD79B | FANCM | IRF1 | NRG1 | RPS6KB1 | XPC |
| CDC73 | FAS | IRF2 | NSD1 | RPTOR | XPO1 |
| CDH1 | FAT1 | IRF4 | NSD2 | RRM1 | XRCC1 |
| CDK12 | FBXW7 | IRS2 | NSD3 | RSF1 | XRCC2 |
| CDK4 | FCGR2A | JAK1 | NT5C2 | RSPO2 | YEATS4 |
| CDK6 | FCGR3A | JAK2 | NTRK1 | RUNX1 | ZFHX3 |
| CDK8 | FGF10 | JAK3 | NTRK2 | RXRA | ZMYM3 |
| CDK9 | FGF12 | JUN | NTRK3 | SDC4 | ZNF217 |
| CDKN1A | FGF14 | KAT6A | NUTM1 | SDHA | ZNF703 |
| CDKN1B | FGF19 | KDM5A | P2RY8 | SDHAF2 | ZNF750 |
| CDKN2A | FGF23 | KDM5C | PAK1 | SDHB | ZNRF3 |
| CDKN2B | FGF3 | KDM5D | PALB2 | SDHC | ZRSR2 |
| CDKN2C | FGF4 | KDM6A | PALLD | SDHD | |
| CEBPA | FGF6 | KDR | PARP1 | SETBP1 | |
| CHD4 | FGFR1 | KEAP1 | PARP2 | SETD2 | |
| CHEK1 | FGFR2 | KEL | PARP3 | SF3B1 | |
| CHEK2 | FGFR3 | KIT | PAX5 | SGK1 | |

In some embodiments, a total cfDNA fragment sequencing reaction is performed at a read depth of at least 1×. In some embodiments, a panel-enriched sequencing reaction is performed at a read depth of at least 2×, at least 3×, at least 4×, at least 5×, at least 10×, at least 25×, at least 50×, at least 100×, at least 250×, or greater. In some embodiments, a total cfDNA fragment sequencing reaction is performed at a read depth of no more than 1000×, no more than 500×, no more than 100×, no more than 50×, or less. In some embodiments, a total cfDNA fragment sequencing reaction is performed at a read depth of from 1× to 500×, from 1× to 100×, or from 1× to 50×. In some embodiments, a total cfDNA fragment sequencing reaction is performed at a read depth of from 2.5× to 500×, from 2.5× to 100×, or from 2.5× to 50×. In some embodiments, a total cfDNA fragment sequencing reaction is performed at a read depth of from 5× to 500×, from 5× to 100×, or from 5× to 50×. In some embodiments, a total cfDNA fragment sequencing reaction is performed at a read depth of from 10× to 500×, from 10× to 100×, or from 10× to 50×.

In some embodiments, the probe set includes probes targeting one or more of the genes listed in List 1, provided below. In some embodiments, the probe set includes probes targeting at least 5 of the genes listed in List 1. In some embodiments, the probe set includes probes targeting at least 10 of the genes listed in List 1. In some embodiments, the probe set includes probes targeting at least 25 of the genes listed in List 1. In some embodiments, the probe set includes probes targeting at least 50 of the genes listed in List 1. In some embodiments, the probe set includes probes targeting at least 70 of the genes listed in List 1. In some embodiments, the probe set includes probes targeting all of the genes listed in List 1.

In some embodiments, the probe set includes probes targeting one or more of the genes listed in List 2, provided below. In some embodiments, the probe set includes probes targeting at least 5 of the genes listed in List 2. In some embodiments, the probe set includes probes targeting at least 10 of the genes listed in List 2. In some embodiments, the probe set includes probes targeting at least 25 of the genes listed in List 2. In some embodiments, the probe set includes probes targeting at least 50 of the genes listed in List 2. In some embodiments, the probe set includes probes targeting at least 75 of the genes listed in List 2. In some embodiments, the probe set includes probes targeting at least 100 of the genes listed in List 2. In some embodiments, the probe set includes probes targeting all of the genes listed in List 2.

In some embodiments, panels of genes including one or more genes from the following lists are used for analyzing specimens, sequencing, and/or identification. In some embodiments, panels of genes for analyzing specimens, sequencing, and/or identification include one or more genes from List 1 or List 2. In some embodiments, panels of genes for analyzing specimens, sequencing, and/or identification include one or more genes from: List 1: AKT1 (14q32.33), ALK (2p23.2-23.1), APC (5q22.2), AR (Xq12), ARAF (Xp11.3), ARID1A (1p36.11), ATM (11q22.3), BRAF (7q34), BRCA1 (17q21.31), BRCA2 (13q13.1), CCND1 (11q13.3), CCND2 (12p13.32), CCNE1 (19q12), CDH1 (16q22.1), CDK4 (12q14.1), CDK6 (7q21.2), CDKN2A (9p21.3), CTNNB1 (3p22.1), DDR2 (1923.3), EGFR (7p11.2), ERBB2 (17q12), ESR1 (6q25.1-25.2), EZH2 (7q36.1), FBXW7 (4q31.3), FGFR1 (8p11.23), FGFR2 (10q26.13), FGFR3 (4p16.3), GATA3 (10p14), GNA11 (19p13.3), GNAQ (9q21.2), GNAS (20q13.32), HNFIA (12q24.31), HRAS (11p15.5), IDH1 (2q34), IDH2 (15q26.1), JAK2 (9p24.1), JAK3 (19p13.11), KIT (4q12), KRAS (12p12.1), MAP2K1 (15q22.31), MAP2K2 (19p13.3), MAPK1 (22q11.22), MAPK3 (16p11.2), MET (7q31.2), MLH1 (3p22.2), MPL (1p34.2), MTOR (1p36.22), MYC (8q24.21), NF1 (17q11.2), NFE2L2 (2q31.2), NOTCH1 (9q34.3), NPM1 (5q35.1), NRAS (1p13.2), NTRK1 (1q23.1), NTRK3 (15q25.3), PDGFRA (4q12), PIK3CA (3q26.32), PTEN (10q23.31), PTPN11 (12q24.13), RAF1 (3p25.2), RB1 (13q14.2), RET (10q11.21), RHEB (7q36.1), RHOA (3p21.31), RIT1 (1922), ROS1 (6922.1), SMAD4 (18q21.2), SMO (7q32.1), STK11 (19p13.3), TERT (5p15.33), TP53 (17p13.1), TSC1 (9q34.13), and VHL (3p25.3).

List 2: ABL1, ACVR1B, AKT1, AKT2, AKT3, ALK, ALOX12B, AMER1 (FAM123B), APC, AR, ARAF, ARFRP1, ARID1A, ASXL1, ATM, ATR, ATRX, AURKA, AURKB, AXIN1, AXL, BAP1, BARD1, BCL2, BCL2L1, BCL2L2, BCL6, BCOR, BCORL1, BRAF, BRCA1, BRCA2, BRD4, BRIP1, BTG1, BTG2, BTK, C11orf30 (EMSY), C17orf39 (GID4), CALR, CARD11, CASP8, CBFB, CBL, CCND1, CCND2, CCND3, CCNE1, CD22, CD274 (PD-L1), CD70, CD79A, CD79B, CDC73, CDH1, CDK12, CDK4, CDK6, CDK8, CDKN1A, CDKN1B, CDKN2A, CDKN2B, CDKN2C, CEBPA, CHEK1, CHEK2, CIC, CREBBP, CRKL, CSF1R, CSF3R, CTCF, CTNNA1, CTNNB1, CUL3, CUL4A, CXCR4, CYP17A1, DAXX, DDR1, DDR2, DIS3, DNMT3A, DOT1L, EED, EGFR, EP300, EPHA3, EPHB1, EPHB4, ERBB2, ERBB3, ERBB4, ERCC4, ERG, ERRFI1, ESR1, EZH2, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GNA11, GNA13, GNAQ, GNAS, GRM3, GSK3B, H3F3A, HDAC1, HGF, HNF1A, HRAS, HSD3B1, ID3, IDH1, IDH2, IGF1R, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A, KMT2D (MLL2), KRAS, LTK, LYN, MAF, MAP2K1 (MEK1), MAP2K2 (MEK2), MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MST1R, MTAP, MTOR, MUTYH, MYC, MYCL (MYCL1), MYCN, MYD88, NBN, NF1, NF2, NFE2L2, NFKBIA, NKX2-1, NOTCH1, NOTCH2, NOTCH3, NPM1, NRAS, NSD3 (WHSCIL1), NT5C2, NTRK1, NTRK2, NTRK3, P2RY8, PALB2, PARK2, PARP1, PARP2, PARP3, PAX5, PBRM1, PDCD1 (PD-1), PDCD1LG2 (PD-L2), PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKAR1A, PRKC1, PTCH1, PTEN, PTPN11, PTPRO, QKI, RACI, RAD21, RAD51, RAD51B, RAD51C, RAD51D, RAD52, RAD54L, RAF1, RARA, RB1, RBM10, REL, RET, RICTOR, RNF43, ROS1, RPTOR, SDHA, SDHB, SDHC, SDHD, SETD2, SF3B1, SGK1, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, ncRNA, TGFBR2, TIPARP, TNFAIP3, TNFRSF14, TP53, TSC1, TSC2, TYRO3, U2AF1, VEGFA, VHL, WHSC1, WT1, XPO1, XRCC2, ZNF217, and ZNF703.

Generally, probes for enrichment of nucleic acids (e.g., cfDNA obtained from a liquid biopsy sample) include DNA, RNA, or a modified nucleic acid structure with a base sequence that is complementary to a locus of interest. For instance, a probe designed to hybridize to a locus in a cfDNA molecule can contain a sequence that is complementary to either strand, because the cfDNA molecules are double stranded. In some embodiments, each probe in the plurality of probes includes a nucleic acid sequence that is identical or complementary to at least 10, at least 11, at least 12, at least 13, at least 14, or at least 15 consecutive bases of a locus of interest. In some embodiments, each probe in the plurality of probes includes a nucleic acid sequence that is identical or complementary to at least 20, 25, 30, 40, 50, 75, 100, 150, 200, or more consecutive bases of a locus of interest.

Targeted panels provide several benefits for nucleic acid sequencing. For example, in some embodiments, algorithms for discriminating between, e.g., a first and second cancer condition can be trained on smaller, more informative data sets (e.g., fewer genes), which leads to more computationally efficient training of classifiers that discriminate between the first and second cancer states. Such improvements in computational efficiency, owing to the reduced size of the discriminating gene set, can advantageously either be used to speed up classifier training or be used to improve the performance of such classifiers (e.g., through more extensive training of the classifier).

In some embodiments, the gene panel is a whole-exome panel that analyzes the exomes of a biological sample. In some embodiments, the gene panel is a whole-genome panel that analyzes the genome of a specimen. In some preferred embodiments, the gene panel is optimized for use with liquid biopsy samples (e.g., to provide clinical decision support for solid tumors). See, for example, Table 1 above.

In some embodiments, the probes include additional nucleic acid sequences that do not share any homology to the loci of interest. For example, in some embodiments, the probes also include nucleic acid sequences containing an identifier sequence, e.g., a unique molecular identifier (UMI), e.g., that is unique to a particular sample or subject. Examples of identifier sequences are described, for example, in Kivioja et al., 2011, Nat. Methods 9 (1), pp. 72-74 and Islam et al., 2014, Nat. Methods 11 (2), pp. 163-66, which are incorporated by reference herein. Similarly, in some embodiments, the probes also include primer nucleic acid sequences useful for amplifying the nucleic acid molecule of interest, e.g., using PCR. In some embodiments, the probes also include a capture sequence designed to hybridize to an anti-capture sequence for recovering the nucleic acid molecule of interest from the sample.

Likewise, in some embodiments, the probes each include a non-nucleic acid affinity moiety covalently attached to nucleic acid molecule that is complementary to the loci of interest, for recovering the nucleic acid molecule of interest. Non-limited examples of non-nucleic acid affinity moieties include biotin, digoxigenin, and dinitrophenol. In some embodiments, the probe is attached to a solid-state surface or particle, e.g., a dipstick or magnetic bead, for recovering the nucleic acid of interest. In some embodiments, the methods described herein include amplifying the nucleic acids that bound to the probe set prior to further analysis, e.g., sequencing. Methods for amplifying nucleic acids, e.g., by PCR, are well known in the art.

Next-generation sequencing produces millions of short reads (e.g., sequence reads) for each biological sample. Accordingly, in some embodiments, the plurality of sequence reads obtained by next-generation sequencing of cfDNA molecules are DNA sequence reads. In some embodiments, the sequence reads have an average length of at least fifty nucleotides. In other embodiments, the sequence reads have an average length of at least 50, 60, 70, 80, 90, 100, 150, 200, 250, 300, or more nucleotides.

In some embodiments, sequencing is performed after enriching for nucleic acids (e.g., cfDNA, gDNA, and/or RNA) encompassing a plurality of predetermined target sequences, e.g., human genes and/or non-coding sequences associated with cancer. Advantageously, sequencing a nucleic acid sample that has been enriched for target nucleic acids, rather than all nucleic acids isolated from a biological sample, significantly reduces the average time and cost of the sequencing reaction. Accordingly, in some preferred embodiments, the methods described herein include obtaining a plurality of sequence reads of nucleic acids that have been hybridized to a probe set for hybrid-capture enrichment (e.g., of one or more genes listed in Table 1 or of one or more genes listed in Table 2, or one or more genes listed in List 2).

In some embodiments, panel-targeting sequencing is performed to an average on-target depth of at least 500×, at least 750×, at least 1000×, at least 2500×, at least 500×, at least 10,000×, or greater depth. In some embodiments, samples are further assessed for uniformity above a sequencing depth threshold (e.g., 95% of all targeted base pairs at 300× sequencing depth). In some embodiments, the sequencing depth threshold is a minimum depth selected by a user or practitioner.

In some embodiments, the sequence reads are obtained by a whole genome or whole exome sequencing methodology. In some such embodiments, whole exome capture is performed with an automated system, using a liquid handling robot (for example, a SciClone NGSx). Whole genome sequencing, and to some extent whole exome sequencing, is typically performed at lower sequencing depth than smaller target-panel sequencing reactions, because many more loci are being sequenced. For example, in some embodiments, whole genome or whole exome sequencing is performed to an average sequencing depth of at least 3×, at least 5×, at least 10×, at least 15×, at least 20×, or greater. In some embodiments, low-pass whole genome sequencing (LPWGS) techniques are used for whole genome or whole exome sequencing. LPWGS is typically performed to an average sequencing depth of about 0.25× to about 5×, more typically to an average sequencing depth of about 0.5× to about 3×.

Because of the differences in the sequencing methodologies, data obtained from targeted-panel sequencing is better suited for certain analyses than data obtained from whole genome/whole exome sequencing, and vice versa. For instance, because of the higher sequencing depth achieved by targeted-panel sequencing, the resulting sequence data is better suited for the identification of variant alleles present at low allelic fractions in the sample, e.g., less than 20%. By contrast, data generated from whole genome/whole exome sequencing is better suited for the estimation of genome-wide metrics, such as tumor mutational burden, because the entire genome is better represented in the sequencing data. Accordingly, in some embodiments, a nucleic acid sample, e.g., a cfDNA, gDNA, or mRNA sample, is evaluated using both targeted-panel sequencing and whole genome/whole exome sequencing (e.g., LPWGS).

In some embodiments, the raw sequence reads resulting from the sequencing reaction are output from the sequencer in a native file format, e.g., a BCL file. In some embodiments, the native file is passed directly to a bioinformatics pipeline (e.g., variant analysis 206), components of which are described in detail below. In other embodiments, pre-processing is performed prior to passing the sequences to the bioinformatics platform. For instance, in some embodiments, the format of the sequence read file is converted from the native file format (e.g., BCL) to a file format compatible with one or more algorithms used in the bioinformatics pipeline (e.g., FASTQ or FASTA). In some embodiments, the raw sequence reads are filtered to remove sequences that do not meet one or more quality thresholds. In some embodiments, raw sequence reads generated from the same unique nucleic acid molecule in the sequencing read are collapsed into a single sequence read representing the molecule, e.g., using UMIs as described above. In some embodiments, one or more of these pre-processing activities is performed within the bioinformatics pipeline itself.

In one example, a sequencer may generate a BCL file. A BCL file may include raw image data of a plurality of patient specimens which are sequenced. BCL image data is an image of the flow cell across each cycle during sequencing. A cycle may be implemented by illuminating a patient specimen with a specific wavelength of electromagnetic radiation, generating a plurality of images which may be processed into base calls via BCL to FASTQ processing algorithms which identify which base pairs are present at each cycle. The resulting FASTQ file includes the entirety of reads for each patient specimen paired with a quality metric, e.g., in a range from 0 to 64 where a 64 is the best quality and a 0 is the worst quality. In embodiments where both a liquid biopsy sample and a normal tissue sample are sequenced, sequence reads in the corresponding FASTQ files may be matched, such that a liquid biopsy-normal analysis may be performed.

FASTQ format is a text-based format for storing both a biological sequence, such as a nucleotide sequence, and its corresponding quality scores. These FASTQ files are analyzed to determine what genetic variants or copy number changes are present in the sample. Each FASTQ file contains reads that may be paired-end or single reads, and may be short-reads or long-reads, where each read represents one detected sequence of nucleotides in a nucleic acid molecule that was isolated from the patient sample or a copy of the nucleic acid molecule, detected by the sequencer. Each read in the FASTQ file is also associated with a quality rating. The quality rating may reflect the likelihood that an error occurred during the sequencing procedure that affected the associated read. In some embodiments, the results of paired-end sequencing of each isolated nucleic acid sample are contained in a split pair of FASTQ files, for efficiency. Thus, in some embodiments, forward (Read 1) and reverse (Read 2) sequences of each isolated nucleic acid sample are stored separately but in the same order and under the same identifier.

In various embodiments, the bioinformatics pipeline may filter FASTQ data from the corresponding sequence data file for each respective biological sample. Such filtering may include correcting or masking sequencer errors and removing (trimming) low quality sequences or bases, adapter sequences, contaminations, chimeric reads, overrepresented sequences, biases caused by library preparation, amplification, or capture, and other errors.

While workflow 200 illustrates obtaining a biological sample, extracting nucleic acids from the biological sample, and sequencing the isolated nucleic acids, in some embodiments, sequencing data used in the improved systems and methods described herein (e.g., which include improved methods for determining accurate circulating tumor fraction estimates) is obtained by receiving previously generated sequence reads, in electronic form.

In some embodiments, sequencing of the plurality of cell-free nucleic acids in the liquid biopsy sample of the subject is performed at a central laboratory or sequencing facility. In some such embodiments, the method comprises accessing one or more sequencing datasets and/or one or more auxiliary files, in electronic form, through a cloud-based interface. For example, a dataset can be obtained by performing a bioinformatics pipeline using tumor BAM files, normal BAM files, a human reference genome file, a target region BED file, a list of mappable regions of the genome, and/or a blacklist of recurrent problematic areas of the genome.

In some embodiments, the obtaining the dataset comprises accessing the dataset, in electronic form, through a cloud-based interface. For example, a dataset can comprise one or more outputs from a bioinformatics pipeline (e.g., CNVkit outputs “.cns” and/or “.cnr”).

In some embodiments, the subject is a patient with a cancer. In some such embodiments, the cancer is a solid tumor cancer. In some embodiments, the cancer is Ovarian Cancer, Cervical Cancer, Uveal Melanoma, Colorectal Cancer, Chromophobe Renal Cell Carcinoma, Liver Cancer, Endocrine Tumor, Oropharyngeal Cancer, Retinoblastoma, Biliary Cancer, Adrenal cancer, Neural, Neuroblastoma, Basal Cell Carcinoma, Brain Cancer, Breast Cancer, Melanoma, Non-Clear Cell Renal Cell Carcinoma, Glioblastoma, Glioma, Tumor of Unknown Origin, Kidney Cancer, Gastrointestinal Stromal Tumor, Medulloblastoma, Bladder Cancer, Gastric Cancer, Bone Cancer, Non-Small Cell Lung Cancer, Thymoma, Low Grade Glioma, Prostate Cancer, Clear Cell Renal Cell Carcinoma, Skin Cancer, Thyroid Cancer, Sarcoma, Testicular cancer, Head and Neck Cancer, Head and Neck Squamous Cell Carcinoma, Meningioma, Peritoneal cancer, Endometrial Cancer, Pancreatic Cancer, Mesothelioma, Esophageal Cancer, Small Cell Lung Cancer, Her2 Negative Breast Cancer, Solid Tumor, Ovarian Serous Carcinoma, HR+ Breast Cancer, Uterine Serous Carcinoma, Endometrial Cancer, Uterine Corpus Endometrial Carcinoma, Gastroesophageal Junction Adenocarcinoma, Gallbladder Cancer, Chordoma, or Papillary Renal Cell Carcinoma. In some embodiments, the test subject is a patient in a clinical trial.

In some embodiments, the sequencing data is processed (e.g., using sequence data processing module 141) to prepare it for genomic feature identification 385. For instance, in some embodiments as described above, the sequencing data is present in a native file format provided by the sequencer. Accordingly, in some embodiments, the system (e.g., system 100) applies a pre-processing algorithm 142 to convert the file format (318) to one that is recognized by one or more upstream processing algorithms. For example, BCL file outputs from a sequencer can be converted to a FASTQ file format using the bc12fastq or bc12fastq2 conversion software (Illumina®). FASTQ format is a text-based format for storing both a biological sequence, such as nucleotide sequence, and its corresponding quality scores. These FASTQ files are analyzed to determine what genetic variants, copy number changes, etc., are present in the sample.

In some embodiments, other preprocessing functions are performed, e.g., filtering sequence reads 122 based on a desired quality, e.g., size and/or quality of the base calling. In some embodiments, quality control checks are performed to ensure the data is sufficient for variant calling. For instance, entire reads, individual nucleotides, or multiple nucleotides that are likely to have errors may be discarded based on the quality rating associated with the read in the FASTQ file, the known error rate of the sequencer, and/or a comparison between each nucleotide in the read and one or more nucleotides in other reads that has been aligned to the same location in the reference genome. Filtering may be done in part or in its entirety by various software tools, for example, a software tool such as Skewer. Sec, Jiang, H. et al., BMC Bioinformatics 15 (182): 1-12 (2014). FASTQ files may be analyzed for rapid assessment of quality control and reads, for example, by a sequencing data QC software such as AfterQC, Kraken, RNA-SeQC, FastQC, or another similar software program. For paired end reads, reads may be merged.

In some embodiments, when both a liquid biopsy sample and a normal tissue sample from the patient are sequenced, two FASTQ output files are generated, one for the liquid biopsy sample and one for the normal tissue sample. A ‘matched’ (e.g., panel-specific) workflow is run to jointly analyze the liquid biopsy-normal matched FASTQ files. When a matched normal sample is not available from the patient, FASTQ files from the liquid biopsy sample are analyzed in the ‘tumor-only’ mode. See, for example, FIG. 4B. If two or more patient samples are processed simultaneously on the same sequencer flow cell, e.g., a liquid biopsy sample and a normal tissue sample, a difference in the sequence of the adapters used for each patient sample barcodes nucleic acids extracted from both samples, to associate each read with the correct patient sample and facilitate assignment to the correct FASTQ file.

For efficiency, in some embodiments, the results of paired-end sequencing of each isolate are contained in a split pair of FASTQ files. Forward (Read 1) and reverse (Read 2) sequences of each tumor and normal isolate are stored separately but in the same order and under the same identifier. See, for example, FIG. 4C. In various embodiments, the bioinformatics pipeline may filter FASTQ data from each isolate. Such filtering may include correcting or masking sequencer errors and removing (trimming) low quality sequences or bases, adapter sequences, contaminations, chimeric reads, overrepresented sequences, biases caused by library preparation, amplification, or capture, and other errors. See, for example, FIG. 4D.

Similarly, in some embodiments, sequencing (312) is performed on a pool of nucleic acid sequencing libraries prepared from different biological samples, e.g., from the same or different patients. Accordingly, in some embodiments, the system demultiplexes (320) the data (e.g., using demultiplexing algorithm 144) to separate sequence reads into separate files for each sequencing library included in the sequencing pool, e.g., based on UMI or patient identifier sequences added to the nucleic acid fragments during sequencing library preparation, as described above. In some embodiments, the demultiplexing algorithm is part of the same software package as one or more pre-processing algorithms 142. For instance, the bc12fastq or bc12fastq2 conversion software (Illumina®) include instructions for both converting the native file format output from the sequencer and demultiplexing sequence reads 122 output from the reaction.

The sequence reads are then aligned (322), e.g., using an alignment algorithm 143, to a reference sequence construct 158, e.g., a reference genome, reference exome, or other reference construct prepared for a particular targeted-panel sequencing reaction. For example, in some embodiments, individual sequence reads 123, in electronic form (e.g., in FASTQ files), are aligned against a reference sequence construct for the species of the subject (e.g., a reference human genome) by identifying a sequence in a region of the reference sequence construct that best matches the sequence of nucleotides in the sequence read. In some embodiments, the sequence reads are aligned to a reference exome or reference genome using known methods in the art to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. Any of a variety of alignment tools can be used for this task.

For instance, local sequence alignment algorithms compare subsequences of different lengths in the query sequence (e.g., sequence read) to subsequences in the subject sequence (e.g., reference construct) to create the best alignment for each portion of the query sequence. In contrast, global sequence alignment algorithms align the entirety of the sequences, e.g., end to end. Examples of local sequence alignment algorithms include the Smith-Waterman algorithm (see, for example, Smith and Waterman, J Mol. Biol., 147 (1): 195-97 (1981), which is incorporated herein by reference), Lalign (see, for example, Huang and Miller, Adv. Appl. Math, 12:337-57 (1991), which is incorporated by reference herein), and PatternHunter (see, for example, Ma et al., Bioinformatics, 18 (3): 440-45 (2002), which is incorporated by reference herein).

In some embodiments, the read mapping process starts by building an index of either the reference genome or the reads, which is then used to retrieve the set of positions in the reference sequence where the reads are more likely to align. Once this subset of possible mapping locations has been identified, alignment is performed in these candidate regions with slower and more sensitive algorithms. See, for example, Hatem et al., 2013, “Benchmarking short sequence mapping tools,” BMC Bioinformatics 14: p. 184; and Flicek and Birney, 2009, “Sense from sequence reads: methods for alignment and assembly,” Nat Methods 6 (Suppl. 11), S6-S12, each of which is hereby incorporated by reference. In some embodiments, the mapping tools methodology makes use of a hash table or a Burrows-Wheeler transform (BWT). See, for example, Li and Homer, 2010, “A survey of sequence alignment algorithms for next-generation sequencing,” Brief Bioinformatics 11, pp. 473-483, which is hereby incorporated by reference.

Other software programs designed to align reads include, for example, Novoalign (Novocraft, Inc.), Bowtie, Burrows Wheeler Aligner (BWA), and/or programs that use a Smith-Waterman algorithm. Candidate reference genomes include, for example, hg19, GRCh38, hg38, GRCh37, and/or other reference genomes developed by the Genome Reference Consortium. In some embodiments, the alignment generates a SAM file, which stores the locations of the start and end of each read according to coordinates in the reference genome and the coverage (number of reads) for each nucleotide in the reference genome.

For example, in some embodiments, each read of a FASTQ file is aligned to a location in the human genome having a sequence that best matches the sequence of nucleotides in the read. There are many software programs designed to align reads, for example, Novoalign (Novocraft, Inc.), Bowtie, Burrows Wheeler Aligner (BWA), programs that use a Smith-Waterman algorithm, etc. Alignment may be directed using a reference genome (for example, hg19, GRCh38, hg38, GRCh37, other reference genomes developed by the Genome Reference Consortium, etc.) by comparing the nucleotide sequences in each read with portions of the nucleotide sequence in the reference genome to determine the portion of the reference genome sequence that is most likely to correspond to the sequence in the read. In some embodiments, one or more SAM files are generated for the alignment, which store the locations of the start and end of each read according to coordinates in the reference genome and the coverage (number of reads) for each nucleotide in the reference genome. The SAM files may be converted to BAM files. In some embodiments, the BAM files are sorted, and duplicate reads are marked for deletion, resulting in de-duplicated BAM files.

In some embodiments, adapter-trimmed FASTQ files are aligned to the 19th edition of the human reference genome build (HG19). Following alignment, reads are grouped by alignment position and UMI family and collapsed into consensus sequences. Bases with insufficient quality or significant disagreement among family members (for example, when it is uncertain whether the base is an adenine, cytosine, guanine, etc.) may be replaced by N's to represent a wildcard nucleotide type. PHRED scores are then scaled based on initial base calling estimates combined across all family members. Following single-strand consensus generation, duplex consensus sequences are generated by comparing the forward and reverse oriented PCR products with mirrored UMI sequences. In various embodiments, a consensus can be generated across read pairs. Otherwise, single-strand consensus calls will be used. Following consensus calling, filtering is performed to remove low-quality consensus fragments. The consensus fragments are then re-aligned to the human reference genome using BWA. A BAM output file is generated after the re-alignment, then sorted by alignment position, and indexed.

In some embodiments, where both a liquid biopsy sample and a normal tissue sample are analyzed, this process produces a liquid biopsy BAM file (e.g., Liquid BAM 124-1-i-cf) and a normal BAM file (e.g., Germline BAM 124-1-i-g), as illustrated in FIG. 4A. In various embodiments, BAM files may be analyzed to detect genetic variants and other genetic features, including single nucleotide variants (SNVs), copy number variants (CNVs), gene rearrangements, etc.

In some embodiments, the sequencing data is normalized, e.g., to account for pull-down, amplification, and/or sequencing bias (e.g., mappability, GC bias etc.).

In some embodiments, SAM files generated after alignment are converted to BAM files 124. Thus, after preprocessing sequencing data generated for a pooled sequencing reaction, BAM files are generated for each of the sequencing libraries present in the master sequencing pools. For example, as illustrated in FIG. 4A, separate BAM files are generated for each of three samples acquired from subject 1 at time i (e.g., tumor BAM 124-1-i-t corresponding to alignments of sequence reads of nucleic acids isolated from a solid tumor sample from subject 1, Liquid BAM 124-1-i-cf corresponding to alignments of sequence reads of nucleic acids isolated from a liquid biopsy sample from subject 1, and Germline BAM 124-1-i-g corresponding to alignments of sequence reads of nucleic acids isolated from a normal tissue sample from subject 1), and one or more samples acquired from one or more additional subjects at time j (e.g., Tumor BAM 124-2-j-t corresponding to alignments of sequence reads of nucleic acids isolated from a solid tumor sample from subject 2). In some embodiments, BAM files are sorted, and duplicate reads are marked for deletion, resulting in de-duplicated BAM files. For example, tools like SamBAMBA mark and filter duplicate alignments in the sorted BAM files.

Many of the embodiments described below, in conjunction with FIG. 4, relate to analyses performed using sequencing data from cfDNA of a cancer patient, e.g., obtained from a liquid biopsy sample of the patient. Generally, these embodiments are independent and, thus, not reliant upon any particular sequencing data generation methods, e.g., sample preparation, sequencing, and/or data pre-processing methodologies. However, in some embodiments, the methods described below include one or more features 204 of generating sequencing data, as illustrated in FIGS. 2A and 3.

Alignment files prepared as described above (e.g., BAM files 124) are then passed to a feature extraction module 145, where the sequences are analyzed (324) to identify genomic alterations (e.g., SNVs/MNVs, indels, genomic rearrangements, copy number variations, etc.) and/or determine various characteristics of the patient's cancer (e.g., MSI status, TMB, tumor ploidy, HRD status, tumor fraction, tumor purity, methylation patterns, etc.). Many software packages for identifying genomic alterations are known in the art. For a review of many of these variant calling packages see, for example, Cameron et al., Nat. Commun., 10 (3240): 1-11 (2019), the content of which is hereby incorporated by reference, in its entirety, for all purposes. Generally, these software packages identify variants in sorted SAM or BAM files 124, relative to one or more reference sequence constructs 158. The software packages then output a file e.g., a raw VCF (variant call format), listing the variants (e.g., genomic features 131) called and identifying their location relevant to the reference sequence construct (e.g., where the sequence of the sample nucleic acids differ from the corresponding sequence in the reference construct). In some embodiments, system 100 digests the contents of the native output file to populate feature data 125 in test patient data store 120. In other embodiments, the native output file serves as the record of these genomic features 131 in test patient data store 120.

Generally, the systems described herein can employ any combination of available variant calling software packages and internally developed variant identification algorithms. In some embodiments, the output of a particular algorithm of a variant calling software is further evaluated, e.g., to improve variant identification. Accordingly, in some embodiments, system 100 employs an available variant calling software package to perform some of all of the functionality of one or more of the algorithms shown in feature extraction module 145.

In some embodiments, as illustrated in FIG. 1A, separate algorithms (or the same algorithm implemented using different parameters) are applied to identify variants unique to the cancer genome of the patient and variants existing in the germline of the subject. In other embodiments, variants are identified indiscriminately and later classified as either germline or somatic, e.g., based on sequencing data, population data, or a combination thereof. In some embodiments, variants are classified as germline variants, and/or non-actionable variants, when they are represented in the population above a threshold level, e.g., as determined using a population database such as ExAC or gnomAD. For instance, in some embodiments, variants that are represented in at least 1% of the alleles in a population are annotated as germline and/or non-actionable. In other embodiments, variants that are represented in at least 2%, at least 3%, at least 4%, at least 5%, at least 7.5%, at least 10%, or more of the alleles in a population are annotated as germline and/or non-actionable. In some embodiments, sequencing data from a matched sample from the patient, e.g., a normal tissue sample, is used to annotate variants identified in a cancerous sample from the subject. That is, variants that are present in both the cancerous sample and the normal sample represent those variants that were in the germline prior to the patient developing cancer and can be annotated as germline variants.

In various aspects, the detected genetic variants and genetic features are analyzed as a form of quality control. For example, a pattern of detected genetic variants or features may indicate an issue related to the sample, sequencing procedure, and/or bioinformatics pipeline (e.g., example, contamination of the sample, mislabeling of the sample, a change in reagents, a change in the sequencing procedure and/or bioinformatics pipeline, etc.).

FIG. 4E illustrates an example workflow for genomic feature identification (324). This particular workflow is only an example of one possible collection and arrangement of algorithms for feature extraction from sequencing data 124. Generally, any combination of the modules and algorithms of feature extraction module 145, e.g., illustrated in FIG. 1A, can be used for a bioinformatics pipeline, and particularly for a bioinformatics pipeline for analyzing liquid biopsy samples. For instance, in some embodiments, an architecture useful for the methods and systems described herein includes at least one of the modules or variant calling algorithms shown in feature extraction module 145. In some embodiments, an architecture includes at least 2, 3, 4, 5, 6, 7, 8, 9, 10, or more of the modules or variant calling algorithms shown in feature extraction module 145. Further, in some embodiments, feature extraction modules and/or algorithms not illustrated in FIG. 1A find use in the methods and systems described herein.

Blocks 504-506. Referring to block 504, in some embodiments, the liquid biopsy sample is blood. Referring to block 506, in some embodiments, the liquid biopsy sample includes blood, whole blood, peripheral blood, plasma, serum, or lymph of the subject. In some alternative embodiments, the liquid biopsy sample is any of the embodiments described above (see, e.g., Definitions: Liquid Biopsy).

In some embodiments, one or more of the biological samples obtained from the patient are a biological liquid sample, also referred to as a liquid biopsy sample. In some embodiments, one or more of the biological samples obtained from the patient are selected from blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. In some embodiments, the liquid biopsy sample includes blood and/or saliva. In some embodiments, the liquid biopsy sample is peripheral blood. In some embodiments, blood samples are collected from patients in commercial blood collection containers, e.g., using a PAXGENE® Blood DNA Tubes. In some embodiments, saliva samples are collected from patients in commercial saliva collection containers, e.g., using an ORAGENE® DNA Saliva Kit.

In some embodiments, the liquid biopsy sample has a volume of from about 1 mL to about 50 mL For example, in some embodiments, the liquid biopsy sample has a volume of about 1 mL, about 2 mL, about 3 mL, about 4 mL, about 5 mL, about 6 mL, about 7 mL, about 8 mL, about 9 mL, about 10 mL, about 11 mL, about 12 mL, about 13 mL, about 14 mL, about 15 mL, about 16 mL, about 17 mL, about 18 mL, about 19 mL, about 20 mL, or greater.

Liquid biopsy samples include cell free nucleic acids, including cell-free DNA (cfDNA). As described above, cfDNA isolated from cancer patients includes DNA originating from cancerous cells, also referred to as circulating tumor DNA (ctDNA), cfDNA originating from germline (e.g., healthy or non-cancerous) cells, and cfDNA originating from hematopoietic cells (e.g., white blood cells). The relative proportions of cancerous and non-cancerous cfDNA present in a liquid biopsy sample varies depending on the characteristics (e.g., the type, stage, lineage, genomic profile, etc.) of the patient's cancer. As used herein, the ‘tumor burden’ of the subject refers to the percentage cfDNA that originated from cancerous cells.

As described herein, cfDNA is a particularly useful source of biological data for various implementations of the methods and systems described herein, because it is readily obtained from various body fluids. Advantageously, use of bodily fluids facilitates serial monitoring because of the ease of collection, as these fluids are collectable by non-invasive or minimally invasive methodologies. This is in contrast to methods that rely upon solid tissue samples, such as biopsies, which often times require invasive surgical procedures. Further, because bodily fluids, such as blood, circulate throughout the body, the cfDNA population represents a sampling of many different tissue types from many different locations.

In some embodiments, a liquid biopsy sample is separated into two different samples. For example, in some embodiments, a blood sample is separated into a blood plasma sample, containing cfDNA, and a buffy coat preparation, containing white blood cells.

In some embodiments, a plurality of liquid biopsy samples is obtained from a respective subject at intervals over a period of time (e.g., using serial testing). For example, in some such embodiments, the time between obtaining liquid biopsy samples from a respective subject is at least 1 day, at least 2 days, at least 1 week, at least 2 weeks, at least 1 month, at least 2 months, at least 3 months, at least 4 months, at least 6 months, or at least 1 year.

In some alternative embodiments, one or more biological samples collected from the patient are solid tissue samples, e.g., a solid tumor sample or a solid normal tissue sample. Methods for obtaining solid tissue samples, e.g., of cancerous and/or normal tissue are known in the art and are dependent upon the type of tissue being sampled. For example, bone marrow biopsies and isolation of circulating tumor cells can be used to obtain samples of blood cancers, endoscopic biopsies can be used to obtain samples of cancers of the digestive tract, bladder, and lungs, needle biopsies (e.g., fine-needle aspiration, core needle aspiration, vacuum-assisted biopsy, and image-guided biopsy, can be used to obtain samples of subdermal tumors, skin biopsies, e.g., shave biopsy, punch biopsy, incisional biopsy, and excisional biopsy, can be used to obtain samples of dermal cancers, and surgical biopsies can be used to obtain samples of cancers affecting internal organs of a patient. In some embodiments, a solid tissue sample is a formalin-fixed tissue (FFT). In some embodiments, a solid tissue sample is a macro-dissected formalin fixed paraffin embedded (FFPE) tissue. In some embodiments, a solid tissue sample is a fresh frozen tissue sample.

In some embodiments, a dedicated normal sample is collected from the patient, for co-processing with a liquid biopsy sample. Generally, the normal sample is of a non-cancerous tissue, and can be collected using any tissue collection means described above. In some embodiments, buccal cells collected from the inside of a patient's cheeks are used as a normal sample. Buccal cells can be collected by placing an absorbent material, e.g., a swab, in the subject's mouth and rubbing it against their cheek, e.g., for at least 15 second or for at least 30 seconds. The swab is then removed from the patient's mouth and inserted into a tube, such that the tip of the tube is submerged into a liquid that serves to extract the buccal cells off of the absorbent material. An example of buccal cell recovery and collection devices is provided in U.S. Pat. No. 9,138,205, the content of which is hereby incorporated by reference, in its entirety, for all purposes. In some embodiments, the buccal swab DNA is used as a source of normal DNA in circulating heme malignancies.

Referring to FIG. 2, in some embodiments the biological samples collected from the patient are, optionally, sent to various analytical environments (e.g., sequencing lab 230, pathology lab 240, and/or molecular biology lab 250) for processing (e.g., data collection) and/or analysis (e.g., feature extraction). Wet lab processing 204 may include cataloguing samples (e.g., accessioning), examining clinical features of one or more samples (e.g., pathology review), and nucleic acid sequence analysis (e.g., extraction, library prep, capture+hybridize, pooling, and sequencing). In some embodiments, the workflow includes clinical analysis of one or more biological samples collected from the subject, e.g., at a pathology lab 240 and/or a molecular and cellular biology lab 250, to generate clinical features such as pathology features 128-3, imaging data 128-3, and/or tissue culture/organoid data 128-3.

In some embodiments, the pathology data 128-1 collected during clinical evaluation includes visual features identified by a pathologist's inspection of a specimen (e.g., a solid tumor biopsy), e.g., of stained H&E or IHC slides. In some embodiments, the sample is a solid tissue biopsy sample. In some embodiments, the tissue biopsy sample is a formalin-fixed tissue (FFT), e.g., a formalin-fixed paraffin-embedded (FFPE) tissue. In some embodiments, the tissue biopsy sample is an FFPE or FFT block. In some embodiments, the tissue biopsy sample is a fresh-frozen tissue biopsy. The tissue biopsy sample can be prepared in thin sections (e.g., by cutting and/or affixing to a slide), to facilitate pathology review (e.g., by staining with immunohistochemistry stain for IHC review and/or with hematoxylin and cosin stain for H&E pathology review). For instance, analysis of slides for H&E staining or IHC staining may reveal features such as tumor infiltration, programmed death-ligand 1 (PD-L1) status, human leukocyte antigen (HLA) status, or other immunological features.

In some embodiments, a liquid sample (e.g., blood) collected from the patient (e.g., in EDTA-containing collection tubes) is prepared on a slide (e.g., by smearing) for pathology review. In some embodiments, macrodissected FFPE tissue sections, which may be mounted on a histopathology slide, from solid tissue samples (e.g., tumor or normal tissue) are analyzed by pathologists. In some embodiments, tumor samples are evaluated to determine, e.g., the tumor purity of the sample, the percent tumor cellularity as a ratio of tumor to normal nuclei, etc. For each section, background tissue may be excluded or removed such that the section meets a tumor purity threshold, e.g., where at least 20% of the nuclei in the section are tumor nuclei, or where at least 25%, 30%, 40%, 50%, 60%, 70%, 80%, 90%, or more of the nuclei in the section are tumor nuclei.

In some embodiments, pathology data 128-1 is extracted, in addition to or instead of visual inspection, using computational approaches to digital pathology, e.g., providing morphometric features extracted from digital images of stained tissue samples. In some embodiments, pathology data 128-1 includes features determined using machine learning algorithms to evaluate pathology data collected as described above.

Further details on methods, systems, and algorithms for using pathology data to classify cancer and identify targeted therapies are discussed, for example, in U.S. Pat. Nos. 10,957,041, 11,244,763, 11,848,107, and 11,145,416, the contents of which are each hereby incorporated by reference, in their entireties, for all purposes.

In some embodiments, imaging data 128-2 collected during clinical evaluation includes features identified by review of in-vitro and/or in-vivo imaging results (e.g., of a tumor site), for example a size of a tumor, tumor size differentials over time (such as during treatment or during other periods of change). In some embodiments, imaging data 128-2 includes features determined using machine learning algorithms to evaluate imaging data collected as described above.

Further details on methods, systems, and algorithms for using medical imaging to classify cancer and identify targeted therapies are discussed, for example, in U.S. Pat. Nos. 10,957,041, 11,244,763, 11,848,107, and 11,145,416, the contents of which are each hereby incorporated by reference, in their entireties, for all purposes.

In some embodiments, tissue culture/organoid data 128-3 collected during clinical evaluation includes features identified by evaluation of cultured tissue from the subject. For instance, in some embodiments, tissue samples obtained from the patients (e.g., tumor tissue, normal tissue, or both) are cultured (e.g., in liquid culture, solid-phase culture, and/or organoid culture) and various features, such as cell morphology, growth characteristics, genomic alterations, and/or drug sensitivity, are evaluated. In some embodiments, tissue culture/organoid data 128-3 includes features determined using machine learning algorithms to evaluate tissue culture/organoid data collected as described above. Examples of tissue organoid (e.g., personal tumor organoid) culturing and feature extractions thereof are described in PCT publication No. WO2021/081253 and U.S. Pat. No. 11,629,385, the contents of which are each hereby incorporated by reference, in their entireties, for all purposes.

In some embodiments, the method further comprises obtaining the liquid biopsy sample from a sample repository or database (e.g., BioIVT, TSC Biosample Repository, BioLINCC, etc.). In some embodiments, the liquid biopsy sample is obtained from the subject at least 1 hour, at least 2 hours, at least 12 hours, at least 1 day, at least 2 days, at least 1 week, at least 1 month, or at least 1 year prior to processing and/or sequencing the liquid biopsy sample. In some such embodiments, the liquid biopsy sample is fresh, frozen, dried, and/or fixed. In some embodiments, the liquid biopsy sample is processed and/or sequenced at least 1 day, at least 2 days, at least 1 week, at least 1 month, or at least 1 year prior to obtaining the first dataset. For example, in some embodiments, the sequencing data for the liquid biopsy sample are obtained from a data repository (e.g., GenBank, NCBI Assembly, DNA DataBank of Japan, European Nucleotide Archive, European Variation Archive, etc.).

Block 508. Referring to block 508, in some embodiments, method 500 includes identifying a candidate variant at a first nucleotide position based on at least a difference between a respective nucleic acid sequence for a respective cfDNA fragment in the plurality of cfDNA fragments and a corresponding nucleic acid sequence for a locus in a reference sequence to which the respective nucleic acid sequence maps.

In some embodiments the candidate variant is disqualified from analysis when the support for the candidate variant does not include both strands of at least one cell-free nucleic acid fragment bearing the mutant (alt) allele. Stated differently, in some embodiments, there is a requirement that, in order to retain a given candidate variant for analysis, both strands of at least 1 cell-free nucleic acid fragment bearing the mutant (alt) allele of the given candidate variant be identified from (supported by) one or more sequence reads of the plurality of sequence reads of the sequencing reaction. In some embodiments, there is a requirement that, in order to retain a given candidate variant in the set of given candidate variants, both strands of at least 2, 3, 4, or 5 cell-free nucleic acid fragments bearing the mutant (alt) allele of the given candidate variant be identified from (supported by) one or more sequence reads of the plurality of sequence reads of the sequencing reaction.

In some embodiments a candidate variant that (a) maps to a repeat region in the one or more reference sequences of the species and (b) is not annotated as a known somatic mutation in a database of known somatic mutations for the species of the subject is removed from consideration as a somatic variant. Repeat regions are repeating sequences of two or more base pairs that are adjacent to one another and are abundant throughout genomes, such as the human genome. See, for example, Madsen et al., 2008, “Short tandem repeats in human exons: a target for disease mutations,” BMC genomics, 9, p. 410, which is hereby incorporated by reference. Databases of known somatic mutations for humans include, but are not limited to, Clin Var and COSMIC. See, Landrum et al., 2020, “ClinVar: improvements to accessing data,” Nucleic Acids Res. 48 (D1), pp. D835-D844; and Tate et al., “COSMIC: the Catalogue of Somatic Mutations in Cancer,” Nucleic Acids Research 47 D1, pp. D941-D947, each of which is hereby incorporated by reference. Thus, in some embodiments, if a candidate variant maps to a repeat region and is not in ClinVar it is removed from the set of candidate variants that are evaluated in accordance with FIG. 5. In some embodiments, if a candidate variant maps to a repeat region and is not in COSMIC it is removed from the set of candidate variants that is evaluated in accordance with FIG. 5. In some embodiments, if a candidate variant maps to a repeat region and is not in COSMIC or in Clin Var it is removed from the set of candidate variants that is evaluated in FIG. 5. Clin Var and COSMIC are nonlimiting examples of databases of known somatic mutations and other databases, or any combination of such databases may be used to filter the candidate somatic variant.

In some embodiments, a candidate variant that maps to a region of clonal hematopoiesis of indeterminate potential (CHIP) is removed from consideration for whether it is a somatic variant derived from cell free DNA. Such embodiments are employed to mitigate the risk that a CHIP variant is passed as a candidate somatic variant.

CHIP describes an expansion of hematopoietic stem cells that harbor somatic mutations without an underlying malignancy. CHIP has been identified through genomic profiling of peripheral blood from healthy individuals. See, Busque et al., 2012, “Recurrent somatic TET2 mutations in normal elderly individuals with clonal hematopoiesis,” Nat Genet. 44 (11), pp. 1179-1181, which is hereby incorporated by reference. Its incidence increases with age and has been detected in peripheral blood of patients with solid tumors. See, Xie et al., 2014, “Age-related mutations associated with clonal hematopoietic expansion and malignancies,” Nat Med. 20 (12), pp, 1472-1478, which is hereby incorporated by reference. Hematopoietic cells permeate all tissues and are present in solid tumor specimens. See Severson et al., 2018, “Detection of clonal hematopoiesis of indeterminate potential in clinical sequencing of solid tumor specimens,” Blood 131 (22), pp. 2501-2502, which is hereby incorporated by reference. The application of comprehensive genomic profiling (CGP) to tumor samples provides an unbiased view of heterogeneous cancer cells and admixed nontumor populations.

In one approach to mitigate the risk that a CHIP variant is passed as a candidate somatic variant, an upper bound on how frequently it is expected that the CHIP variant would be passed as a candidate somatic variant is determined by using historical CHIP variant prevalence from comprehensive genomic profiling of tumor samples, such as Tempus xT (Beaubier et al., 2019, “Clinical validation of the Tempus xT next-generation targeted oncology sequencing assay,” Oncotarget 10, pp. 2384-2396, which is hereby incorporated by reference), and calculating the frequency it would, in fact, alter an MRD call on the basis that the CHIP variant was given high confidence and the logic for calling MRD characterizing an MRD+/− call. In some embodiments such historical presence is matched to an age bracket of the subject. For instance, in some embodiments, only that historical CHIP variant prevalence from comprehensive genomic profiling of tumor samples matched to the subject's age bracket is considered. In some such embodiments, a candidate somatic variant is removed from consideration in accordance with FIG. 5 when the candidate somatic variant maps to ASXL1, BCOR, BCORL1, CBL, CREBBP, CUX1, DNMT3A, GNB1, JAK2, PPM1D, PRPF8, SETDB1, SF3B1, SRSF2, TET2, or U2AF1. In some embodiments any subset of these genes is used for this CHIP filter. In some embodiments this set of genes, or any subset thereof, is used for this CHIP filter when the cancer condition is a BRCA-associated cancer. See Marshall et al., “Germline mutations and the presence of clonal hematopoiesis of indeterminate potential (CHIP) in 20,963 patients with BRCA-associated cancers.,” DOI: 10.1200/JCO.2023.41.16_suppl.10522 Journal of Clinical Oncology 41, no. 16_suppl (Jun. 1, 2023) 10522-10522, which is hereby incorporated by reference.

In some embodiments, a candidate somatic variant that maps to TET2, DNMT3A, ASXL1, or SF3B1 is removed from consideration for whether it is a somatic variant derived from cell free DNA. In some embodiments any subset of these genes is used for this CHIP filter. In some embodiments this set of genes, or any subset thereof, is used for the CHIP filter when the cancer condition is a BRCA-associated cancer.

In some embodiments, a candidate variant that maps to TET2, DNMT3A, ASXL1, SF3B1, CBL, U2AF1, IDH2,2,3, MYD88,13, EP300, CDKN2C, HNF1A is removed from consideration for whether it is a somatic variant derived from cell free DNA. In some embodiments any subset set of these genes is used for the CHIP filter.

In some embodiments, the candidate variant is removed from consideration as to whether it is a somatic variant derived from cell free DNA somatic variant when it maps to TET2, DNMT3A, ASXL1, SF3B1, CBL, U2AF1, IDH2,2,3, MYD88,13, EP300, CDKN2C, or HNF1A when the subject is 70 years of age or older. Such genes are known CHIP genes in subjects of this age. See, Severson, 2018, “Detection of clonal hematopoiesis of indeterminate potential in clinical sequencing of solid tumor specimens,” Blood 131 (22), pp. 2501-2505, which is hereby incorporated by reference.

In some embodiments, variant analysis of aligned sequence reads, e.g., in SAM or BAM format, includes identification of single nucleotide variants (SNVs), multiple nucleotide variants (MNVs), indels (e.g., nucleotide additions and deletions), and/or genomic rearrangements (e.g., inversions, translocations, and gene fusions) using variant identification module 146, e.g., which includes a SNV/MNV calling algorithm (e.g., SNV/MNV calling algorithm 147), an indel calling algorithm (e.g., indel calling algorithm 148), and/or one or more genomic rearrangement calling algorithms (e.g., genomic rearrangement calling algorithm 149). An overview of an example method for variant identification is shown in FIG. 4E. Essentially, the module first identifies a difference between the sequence of an aligned sequence read 124 and the reference sequence to which the sequence read is aligned (e.g., an SNV/MNV, an indel, or a genomic rearrangement) and makes a record of the variant, e.g., in a variant call format (VCF) file. For instance, software packages such as freebayes and pindel are used to call variants using sorted BAM files and reference BED files as the input. For a review of variant calling packages see, for example, Cameron, D. L. et al., Nat. Commun., 10 (3240): 1-11 (2019). A raw VCF file (variant call format) file is output, showing the locations where the nucleotide base in the sample is not the same as the nucleotide base in that position in the reference sequence construct.

In some embodiments, as illustrated in FIG. 4E, raw VCF data is then normalized, e.g., by parsimony and left alignment. For example, software packages such as vcfbreakmulti and vt are used to normalize multi-nucleotide polymorphic variants in the raw VCF file and a variant normalized VCF file is output. See, for example, E. Garrison, “Vcflib: A C++ library for parsing and manipulating VCF files, GitHub, available on the internet at github.com/ekg/vcflib (2012), the content of which is hereby incorporated by reference, in its entirety, for all purposes. In some embodiments, a normalization algorithm is included within the architecture of a broader variant identification software package.

An algorithm is then used to annotate the variants in the (e.g., normalized) VCF file, e.g., determines the source of the variation, e.g., whether the variant is from the germline of the subject (e.g., a germline variant), a cancerous tissue (e.g., a somatic variant), a sequencing error, or of an undeterminable source. In some embodiments, an annotation algorithm is included within the architecture of a broader variant identification software package. However, in some embodiments, an external annotation algorithm is applied to (e.g., normalized) VCF data obtained from a conventional variant identification software package. The choice to use a particular annotation algorithm is well within the purview of the skilled artisan, and in some embodiments is based upon the data being annotated.

For example, in some embodiments, where both a liquid biopsy sample and a normal tissue sample of the patient are analyzed, variants identified in the normal tissue sample inform annotation of the variants in the liquid biopsy sample. In some embodiments, where a particular variant is identified in the normal tissue sample, that variant is annotated as a germline variant in the liquid biopsy sample. Similarly, in some embodiments, where a particular variant identified in the liquid biopsy sample is not identified in the normal tissue sample, the variant is annotated as a somatic variant when the variant otherwise satisfies any additional criteria placed on somatic variant calling, e.g., a threshold variant allele fraction (VAF) in the sample.

By contrast, in some embodiments, where only a liquid biopsy sample is being analyzed, the annotation algorithm relies on other characteristics of the variant in order to annotate the origin of the variant. For instance, in some embodiments, the annotation algorithm evaluates the VAF of the variant in the sample, e.g., alone or in combination with additional characteristics of the sample, e.g., tumor fraction. Accordingly, in some embodiments, where the VAF is within a first range encompassing a value that corresponds to a 1:1 distribution of variant and reference alleles in the sample, the algorithm annotates the variant as a germline variant, because it is presumably represented in cfDNA originating from both normal and cancer tissues. Similarly, in some embodiments, where the VAF is below a baseline variant threshold, the algorithm annotates the variant as undeterminable, because there is not sufficient evidence to distinguish between the possibility that the variant arose as a result of an amplification or sequencing error and the possibility that the variant originated from a cancerous tissue. Similarly, in some embodiments, where the VAF falls between the first range and the baseline variant threshold, the algorithm annotates the variant as a somatic variant derived from cell free DNA.

In some embodiments, the baseline variant threshold is a value from 0.01% VAF to 0.5% VAF. In some embodiments, the baseline variant threshold is a value from 0.05% VAF to 0.35% VAF. In some embodiments, the baseline variant threshold is a value from 0.1% VAF to 0.25% VAF. In some embodiments, the baseline variant threshold is about 0.01% VAF, 0.015% VAF, 0.02% VAF, 0.025% VAF, 0.03% VAF, 0.035% VAF, 0.04% VAF, 0.045% VAF, 0.05% VAF, 0.06% VAF, 0.07% VAF, 0.075% VAF, 0.08% VAF, 0.09% VAF, 0.1% VAF, 0.15% VAF, 0.2% VAF, 0.25% VAF, 0.3% VAF, 0.35% VAF, 0.4% VAF, 0.45% VAF, 0.5% VAF, or greater. In some embodiments, the baseline variant threshold is different for variants located in a first region, e.g., a region identified as a mutational hotspot and/or having high genomic complexity, than for variants located in a second region, e.g., a region that is not identified as a mutational hotspot and/or having average genomic complexity. For example, in some embodiments, the baseline variant threshold is a value from 0.01% to 0.25% for variants located in the first region and is a value from 0.1% to 0.5% for variants located in the second region.

In some embodiments, the first region is a region of interest in the genome that may have been manually selected based on criteria (for example, selection may be based on a known likelihood that a region is associated with variants) and the second region is a region that did not meet the selection criteria. In some embodiments, the baseline variant threshold is a value from 0.01% to 0.5% for variants located in the first region and is a value from 1% to 5% for variants located in the second region. In some embodiments, the first region is a region of interest in the genome that may have been manually selected based on criteria (for example, selection may be based on a known likelihood that a region is associated with variants) and the second region is a region selected based on a second set of criteria.

In some embodiments, a baseline variant threshold is influenced by the sequencing depth of the reaction, e.g., a locus-specific sequencing depth and/or an average sequencing depth (e.g., across a targeted panel and/or complete reference sequence construct). In some embodiments, the baseline variant threshold is dependent upon the type of variant being detected. For example, in some embodiments, different baseline variant thresholds are set for SNPs/MNVs than for indels and/or genomic rearrangements. For instance, while an apparent SNP may be introduced by amplification and/or sequencing errors, it is much less likely that a genomic rearrangement is introduced this way and, thus, a lower baseline variant threshold may be appropriate for genomic rearrangements than for SNPs/MNVs.

In some embodiments, one or more additional criteria are required to be satisfied before a variant can be annotated as a somatic variant derived from cell free DNA. For instance, in some embodiments, a threshold number of unique sequence reads encompassing the variant must be present to annotate the variant as a somatic variant derived from cell free DNA. In some embodiments, the threshold number of unique sequence reads is 2, 3, 4, 5, 7, 10, 12, 15, or greater. In some embodiments, the threshold number of unique sequence reads is only applied when certain conditions are met, e.g., when the variant allele is located in a region of a certain genomic complexity. In some embodiments, the certain genomic complexity is a low genomic complexity. In some embodiments, the certain genomic complexity is an average genomic complexity. In some embodiments, the certain genomic complexity is a high genomic complexity.

In some embodiments, a threshold sequencing coverage, e.g., a locus-specific and/or an average sequencing depth (e.g., across a targeted panel and/or complete reference sequence construct) must be satisfied to annotate the variant as a somatic variant derived from cell free DNA. In some embodiments, the threshold sequencing coverage is 50×, 100×, 150×, 200×, 250×, 300×, 350×, 400× or greater. In some embodiments, the variant is located in a microsatellite instable (MSI) region. In some embodiments, the variant is not located in a microsatellite instable (MSI) region. In some embodiments, the variant has sufficient signal-to-noise ratio.

In some embodiments, bases contributing to the variant satisfy a threshold mapping quality to annotate the variant as a somatic variant derived from cell free DNA. In some embodiments, alignments contributing to the variant must satisfy a threshold alignment quality to annotate the variant as a somatic variant derived from cell free DNA. In some embodiments, a threshold value is determined for a variant detected in a somatic (cancer) sample by analyzing the threshold metric (for example, the baseline variant threshold is determined by analyzing VAF, or the threshold sequencing coverage is determined by analyzing coverage) associated with that variant in a group of germline (normal) samples that were each processed by the same sample processing and sequencing protocol as the somatic sample (process-matched). This may be used to ensure the variants are not caused by observed artifact generating processes.

In some embodiments, the threshold value is set above the median base fraction of the threshold metric value associated with the variant in more than a specified percentage of process-matched germline samples, e.g., 1, 2, 3, 4, 5, 6, 7, 8, 9, 10, or more standard deviations above the median base fraction of the threshold metric value associated with 25%, 30, 40, 50, 60, 70, 75, or more of the processed-matched germline samples. For example, in one embodiment, the threshold value is set to a value 5 standard deviations above the median base fraction of the threshold metric value associated with the variant in more than 50% of the process matched germline samples.

In some embodiments, variants around homopolymer and multimer regions known to generate artifacts may be specifically filtered to avoid such artifacts. For example, in some embodiments, strand specific filtering is performed in the direction of the read in order to minimize stranded artifacts. Similarly, in some embodiments, variants that do not exceed the stranded minimum deviation for their specific locus within a known artifact-generating region may be filtered to avoid artifacts.

Variants may be filtered using dynamic methods, such as through the application of Bayes' Theorem through a likelihood ratio test. The dynamic threshold may be based on, for example, factors such as sample specific error rate, the error rate from a healthy reference pool, and information from internal human solid tumors. Accordingly, in some embodiments, the dynamic filtering method employs a tri-nucleotide context-based Bayesian model. That is, in some embodiments, the threshold for filtering any particular putative variant is dynamically calibrated using a context-based Bayesian model that considers one or more of a sample-specific sequencing error rate, a process-matched control sequencing error rate, and/or a variant-specific frequency (e.g., determined from similar cancers). In this fashion, a minimum number of alternative alleles required to positively identify a true variant is determined for individual alleles and/or loci. An example of methods and systems for applying a variable threshold that consider one or more of these factors is described in U.S. Pat. No. 11,475,981, the disclosure of which is disclosed herein by reference in its entirety for all purposes.

In some embodiments, certain variants pre-identified on a whitelist may be rescued, e.g., not filtered out, when they fail to pass selective filters, e.g., MSI/SN, a Bayesian filtering method, and/or a coverage, VAF or region-based filter. The rationale for whitelisting a variant is to apply less stringent filtering criteria to such a variant so that it can be reviewed and/or reported. In some embodiments, one or more variant on the whitelist is a common pathogenic variant, e.g., with high clinical relevance. In this fashion, when a variant on the whitelist fails to pass certain filters, it will be rescued and not filtered out. As used herein, MSI/SN refers to a variant filter for filtering out potential artifactual variants based on the MSI (microsatellite instable) and SN (signal-to-noise ratio) values calculated by the variant caller VarDict. See, for example, VarDict documentation, available on the internet at github.com/AstraZeneca-NGS/VarDictJava.

In some embodiments, one or more locus and/or genomic region is blacklisted, preventing somatic variant annotation for variants identified at the locus or region. In some embodiments, the variant has a length of 120, 100, 80, 60, 40, 20, 10, 5 or less base pairs. In various embodiments, any combination of the additional criteria, as well as additional criteria not listed above, may be applied to the variant calling process. Again, in some embodiments, different criteria are applied to the annotation of different types of variants.

In some embodiments, liquid biopsy assays are used to detect variant alterations present at low circulating fractions in the patient's blood. In such circumstances, it may be warranted to lower the requirements for positively identifying a variant as a somatic variant derived from cell free DNA. That is, in some embodiments, low levels of support may be sufficient to call a variant as a somatic variant derived from cell free DNA, dependent upon the reason for using the liquid biopsy assay.

In some embodiments, SNV/INDEL detection is accomplished using VarDict (available on the internet at github.com/AstraZeneca-NGS/VarDictJava). Both SNVs and INDELs are called and then sorted, deduplicated, normalized and annotated. The annotation uses SnpEff to add transcript information, 1000 genomes minor allele frequencies, COSMIC reference names and counts, ExAC allele frequencies, and Kaviar population allele frequencies. The annotated variants are then classified as germline, somatic, or uncertain using a Bayesian model based on prior expectations informed by databases of germline and cancer variants. In some embodiments, uncertain variants are treated as somatic for filtering and reporting purposes.

In some embodiments, genomic rearrangements (e.g., inversions, translocations, and gene fusions) are detected following de-multiplexing by aligning tumor FASTQ files against a human reference genome using a local alignment algorithm, such as BWA. In some embodiments, DNA reads are sorted, and duplicates may be marked with a software, for example, SAMBlaster. Discordant and split reads may be further identified and separated. These data may be read into a software, for example, LUMPY, for structural variant detection. In some embodiments, structural alterations are grouped by type, recurrence, and presence and stored within a database and displayed through a fusion viewer software tool.

The fusion viewer software tool may reference a database, for example, Ensembl, to determine the gene and proximal exons surrounding the breakpoint for any possible transcript generated across the breakpoint. The fusion viewer tool may then place the breakpoint 5′ or 3′ to the subsequent exon in the direction of transcription. For inversions, this orientation may be reversed for the inverted gene. After positioning of the breakpoint, the translated amino acid sequences may be generated for both genes in the chimeric protein, and a plot may be generated containing the remaining functional domains for each protein, as returned from a database, for example, Uniprot.

For instance, in an example implementation, gene rearrangements are detected using the SpeedSeq analysis pipeline. Chiang et al., 2015, “SpeedSeq: ultra-fast personal genome analysis and interpretation,” Nat Methods, (12), pg. 966. Briefly, FASTQ files are aligned to hg19 using BWA. Split reads mapped to multiple positions and read pairs mapped to discordant positions are identified and separated, then utilized to detect gene rearrangements by LUMPY. Layer et al., 2014, “LUMPY: a probabilistic framework for structural variant discovery,” Genome Biol, (15), pg. 84. Fusions can then be filtered according to the number of supporting reads.

In some embodiments, putative fusion variants supported by fewer than a minimum number of unique sequence reads are filtered. In some embodiments, the minimum number of unique sequence reads is 2, 3, 4, 5, 6, 7, 8, 9, 10, 12, 15, or 20 unique sequence reads.

Blocks 510-512. Referring to block 510, in some embodiments, the candidate variant is a single nucleotide variant (SNV). Referring to block 512, in some embodiments, the candidate somatic variant is an indel. In some embodiments the indel has a size of no more than three nucleotides. In some embodiments the indel has a size of no more than 4, 5, 6, 7, 8, 9, or 10 nucleotides. In some embodiments the indel has a size of no more than 10, 20, 30, 40, 50, 60, 70, 80, 90, or 100 nucleotides. In some embodiments there is no size limit on the indel.

Block 514. Referring to block 514, in some embodiments, method 500 includes identifying a plurality of unclassified variants based on, for each respective unclassified variant in the plurality of unclassified variants, at least a difference between a respective nucleic acid sequence for a respective cfDNA fragment in the plurality of cfDNA fragments and a nucleic acid sequence for a locus in a reference sequence to which the respective nucleic acid sequence maps. In other words, in some embodiments a candidate variant at a first locus is identified based on at least a difference between a respective nucleic acid sequence for a respective cfDNA fragment in the plurality of cfDNA fragments and a nucleic acid sequence for the first locus in a reference sequence.

In some embodiments, the plurality of unclassified variants is then classified by evaluating each respective unclassified variant in the plurality of unclassified variants against one or more classification criteria, thereby forming a set of germline variants and a set of candidate somatic variants. The candidate somatic variant can then be identified (e.g., validated as actually somatic) from the set of candidate somatic variants.

Blocks 516-518. Referring to block 516, in some embodiments, method 500 includes filtering the set of candidate variants against a set of genes associated with clonal hematopoiesis of indeterminate potential (CHIP). Referring to block 518, in some embodiments, the set of genes includes at least 5 genes selected from the genes listed in Table 3. In some embodiments, the set of genes includes at least 10 genes selected from the genes listed in Table 3. In some embodiments, the set of genes includes at least 20 genes selected from the genes listed in Table 3. in some embodiments, the set of genes includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or all of the genes listed in Table 3.

Table 3. Example genes known to have variants in clinical examples of clonal hematopoiesis of indeterminate potential (CHIP).

TABLE 3
CHIP Genes

| | | | | |
|---|---|---|---|---|
| CSF3R | IDH2 | CALR | U2AF1 | JAK2 |
| MPL | CREBBP | CEBPA | MYD88 | NOTCH1 |
| NRAS | TP53 | DNMT3A | RHOA | PIGA |
| WT1 | STAT5B | CXCR4 | KIT | ZRSR2 |
| ATM | STAT3 | SF3B1 | TET2 | BCOR |
| CBL | PPM1D | IDH1 | TERT | STAG2 |
| PTPN11 | SRSF2 | ASXL1 | NPM1 | BCORL1 |
| FLT3 | SETBP1 | RUNX1 | EZH2 | |

Block 520. Referring to block 520, in some embodiments, the set of genes includes DNMT3A, TET2, PPM1D, ASXL1, and SETBP1. In some embodiments, the set of genes includes at least 10 genes selected from the genes listed in Table 3, including DNMT3A, TET2, PPM1D, ASXL1, and SETBP1. In some embodiments, the set of genes includes at least 20 genes selected from the genes listed in Table 3, including DNMT3A, TET2, PPM1D, ASXL1, and SETBP1. In some embodiments, the set of genes includes at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes listed in Table 3, including DNMT3A, TET2, PPM1D, ASXL1, and SETBP1. In some embodiments, the set of genes includes at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes listed in Table 3, without any requirement that the set of genes include DNMT3A, TET2, PPM1D, ASXL1, or SETBP1.

In some embodiments, other genes associated with CHIP can be used for training models as described herein. Similarly, in some embodiments, candidate somatic variants located within genomic regions, e.g., genic regions or intergenic regions, other than those genes listed in Table 3 can be filtered using the methods and systems described herein.

Block 522. Referring to block 522, in some embodiments, method 500 includes using an identity of a first set of cfDNA fragments in the plurality of cfDNA fragments including the candidate variant to determine one or more fragment length metrics for the candidate variant.

In some embodiments, the lengths of all cfDNA fragments containing the candidate variant are characterized to generate a metric for evaluating whether an identified variant is of hematopoietic or somatic lineage.

In some embodiments, the fragment length metric is a measure of the distribution of the lengths of all cfDNA fragments encompassing the variant, e.g., a measure of central tendency, a characterization of the shape of the distribution of the lengths of all cfDNA fragments encompassing the variant.

In some embodiments, the fragment length metric is a measure of a difference between the distribution of the lengths of all cfDNA fragments encompassing the variant and a distribution of lengths of cfDNA fragments that map to the locus of the variant but do not contain the variant.

Block 524. Referring to block 524, in some embodiments, a fragment length metric in the one or more fragment metrics is a measure of central tendency of the first set of cfDNA fragments. In some embodiments, the fragment length metric is a mean of the lengths of all cfDNA fragments containing the candidate somatic variant.

Block 526. Referring to block 526, in some embodiments, a fragment length metric in the one or more fragment metrics is a kurtosis of the first set of cfDNA fragments. Kurtosis is a measure of the “tailedness” of the probability distribution of a real-valued random variable. A description of determining kurtosis is found, for example, in Joanes and Gill, 1998, “Comparing measures of sample skewness and kurtosis,” Journal of the Royal Statistical Society, Series D, 47 (1): 1831-89, the content of which is incorporated herein by reference in its entirety for all purposes.

Block 528. Referring to block 528, in some embodiments, a fragment length metric in the one or more fragment metrics is a skew of the first set of cfDNA fragments. Skewness is a measure of the asymmetry of the probability distribution of a real-valued random variable about its mean. A description of determining skew is found, for example, in Joanes and Gill, 1998, “Comparing measures of sample skewness and kurtosis,” Journal of the Royal Statistical Society, Series D, 47 (1): 183-189, the content of which is incorporated herein by reference in its entirety for all purposes.

Block 530. Referring to block 530, in some embodiments, a fragment length metric in the one or more fragment length metrics is a p-value determined by application of a Kolmogorov-Smirnov test to a difference in (i) a distribution of fragment lengths of the first set of cfDNA fragments versus (ii) a distribution of fragment lengths of a second set of cfDNA fragments in the plurality of cfDNA fragments.

Block 532. Referring to block 532, in some embodiments, the second set of cfDNA fragments is the cfDNA fragments in the plurality of cfDNA fragments, other than the set of cfDNA fragments, that include the locus of the candidate somatic variant.

Block 534. Referring to block 534, in some embodiments, a fragment length metric in the one or more fragment length metrics is determined by application of a Kolmogorov-Smirnov test to a difference in (i) the distribution of fragment lengths of the first set of cfDNA fragments versus (ii) the distribution of fragment lengths of a third set of cfDNA fragments in the plurality of cfDNA fragments.

Block 536. Referring to block 536, in some embodiments, the third set of cfDNA fragments is the cfDNA fragments in the plurality of cfDNA fragments, other than the set of cfDNA fragments, that map to the locus of the candidate somatic variant.

Block 538. Referring to block 538 of FIG. 5D, in some embodiments, method 500 includes determining a variant allele fraction for the candidate variant in the plurality of cfDNA fragments based on (i) the number of times the candidate variant is observed across the corresponding nucleic acid sequences for each cfDNA fragment in the plurality of cfDNA fragments and (ii) the number of times the first nucleotide position is observed across the corresponding nucleic acid sequences for each cfDNA fragment in the plurality of cfDNA fragments. In other words, in some embodiments the variant allele fraction for the candidate variant is identified by (i) the number of cfDNA fragments in the plurality of cfDNA fragments having the candidate variant and (ii) the number of cfDNA fragments in the plurality of cfDNA fragments mapping to the first locus. For instance, in some embodiment the variant allele fraction is (i) the number of cfDNA fragments in the plurality of cfDNA fragments having the candidate variant divided by (ii) the number of cfDNA fragments in the plurality of cfDNA fragments mapping to the first locus.

In some embodiments, the analysis of aligned sequence reads, e.g., in SAM or BAM format, includes determination of variant allele fractions (133) for one or more of the variant alleles 132 identified as described above. In some embodiments, a variant allele fraction module 151 tallies the instances that each allele is represented by a unique sequence read encompassing the variant locus of interest (e.g., candidate somatic variation), generating a count for each allele represented at that locus. In some embodiments, these tallies are used to determine the ratio of the variant allele, e.g., an allele other than the most prevalent allele in the subject's population for a respective locus, to a reference allele. In the context of block 538, the allele in question is the candidate somatic variation).

Block 540. Referring to block 540, in some embodiments, method 500 includes determining an estimated circulating tumor fraction (ctFE) for the liquid biopsy sample based on the plurality of sequence reads. Tumor fraction or circulating tumor fraction is the fraction of cell free nucleic acid molecules in the sample that originates from a cancerous tissue of the subject, rather than from a non-cancerous tissue (e.g., a germline or hematopoietic tissue). Several open source analysis packages have modules for calculating tumor fraction from solid tumor samples. For instance, PureCN (Riester et al., Source Code Biol Med, 11:13 (2016)) is designed to estimate tumor purity from targeted short-read sequencing data of solid tumor samples. Similarly, FACETS (Shen, 2016, Nucleic Acids Res., 44 (16):e131) is designed to estimate tumor fraction from sequencing data of solid tumor samples. However, estimating tumor fraction from a liquid biopsy sample is more difficult because of the, generally, lower tumor fraction relative to a solid tumor sample and typically small size of a targeted panel used for liquid biopsy sequencing. Indeed, packages such as PureCN and FACETS perform poorly at low tumor fractions and with sequencing data generated using small targeted-panels.

In some embodiments, circulating tumor fraction is estimated from a targeted-panel sequencing reaction of a liquid biopsy sample using an off-target read methodology, e.g., as described herein with reference to FIG. 4F. Briefly, a circulating tumor fraction estimate is determined from reads in the target captured regions, as well as off-target reads uniformly distributed across the human reference genome. Segments having similar copy ratios, e.g., as assigned via circular binary segmentation (CBS) during CNV analysis, are fit to integer copy states, e.g., via an expectation-maximization algorithm using the sum of squared error of the segment log 2 ratios (normalized to genomic interval size) to expected ratios given a putative copy state and tumor fraction. For more information on expectation maximization algorithms see, for example, Sundberg, 1974, “Maximum likelihood theory for incomplete data from an exponential family,” Scandinavian Journal of Statistics. 1 (2): 49-58, the content of which is hereby incorporated by reference in its entirety. A measure of fit between corresponding segment-level coverage ratios and assigned integer copy states across the plurality of simulated circulating tumor fractions is then used to select the simulated circulating tumor fraction to be used as the circulating tumor fraction for the liquid biopsy sample. In some embodiments, error minimization is used to identify the simulated tumor fraction providing the best fit to the data.

In some embodiments, a measure of fit between corresponding segment-level coverage ratios and assigned integer copy states across the plurality of simulated circulating tumor fractions (e.g., using an error minimization algorithm) provides a number of local optima (e.g., local minima for an error minimization model or local maxima for a fix maximization model) for the best fit between the segment-level coverage ratios and assigned integer copy states. In some such embodiments, a second estimate of circulating tumor fraction is used to select the local optima (e.g., the local minima in best agreement with the second estimate of circulating tumor fraction) to be used as the circulating tumor fraction for the liquid biopsy sample.

For example, in some embodiments, multiple local optima (e.g., minima) can be disambiguated based on a difference between somatic and germline variant allele fractions. The assumption is that the variant allele fraction (VAF) of germline variants that exhibit loss of heterozygosity (LOH) will increase or decrease by the amount approximately equal to half of the tumor purity (e.g., the circulating tumor fraction for a liquid biopsy sample). With a matched normal sample (e.g., where sequencing data for both a liquid biopsy sample and a non-cancerous sample from the subject is available, or where sequencing data for both a solid tumor sample and a non-cancerous sample from the subject is available), for a given heterozygous germline variant, the VAF delta can be calculated as delta=abs (VAFₜᵤₘₒᵣ−VAFₙₒᵣₘₐₗ). However, for tumor only sequencing (e.g., where sequencing data is only available for a liquid biopsy sample or a solid tumor sample), the VAFₙₒᵣₘₐₗ is unknown. In some embodiments, the VAFₙₒᵣₘₐₗ is assumed to be 50%. To increase statistical power and account for the imprecision in the VAF by sequencing, the delta for all such variants are calculated and the circulating tumor fraction estimate (ctFE) for this method is calculated as ctFE=max(2×delta) for all variant delta values. While this can be used as a method for ctFE alone, its precision is limited by the number of detected LOH variants. For a small panel, there are few expected LOH variants and thus the ctFE may not be precise on its own. However, it can be used to disambiguate multiple local optima (e.g., minima), especially for high tumor fraction values estimated by the off-target read methodology described herein. For that, the off-target read methodology ctFE peaks corresponding to all the local optima (e.g., minima) are identified and the one closest to the ctFE estimated by LOH delta is chosen as the most likely global optima (e.g., minima).

Several other methods may also be used to estimate circulating tumor fractions. In some embodiments, these methods are used in combination with the off-target tumor estimate method described herein. For example, in some embodiments, one or more of these methodologies is used to generate an estimate of tumor fraction, which is then used to identify the nearest local optima (e.g., minima) obtained from the tumor fraction estimation methods described above, and further herein.

For example, the ichorCNA package applies a probabilistic model to normalized read coverages from ultra-low pass whole genome sequencing data of cell-free DNA to estimate tumor fraction in the liquid biopsy sample. For more information, see, Adalsteinsson et al., Nat Commun 8:1324 (2017), the content of which is disclosed herein for its description of a probabilistic tumor fraction estimation model in the “methods” section. Similarly, Tiancheng et al., describe a Maximum Likelihood model based on the copy number of an allele in the sample and variant allele frequency in paired-control samples. For more information, see, Tiancheng et al., 2019, Journal of Clinical Oncology 37:15 suppl, e13053-e13053, the content of which is disclosed herein for its description of a maximum likelihood tumor fraction estimation model.

In some embodiments, a statistic for somatic variant allele fractions determined for the liquid biopsy sample is used as an estimate for the circulating tumor fraction of the liquid biopsy sample. For example, in some embodiments, a measure of central tendency (e.g., a mean or median) for a plurality of variant allele fractions determined for the liquid biopsy sample is used as an estimate of circulating tumor fraction. In some embodiments, a lowest (minimum) variant allele fraction determined for the liquid biopsy sample is used as an estimate of circulating tumor fraction. In some embodiments, a highest (maximum) variant allele fraction determined for the liquid biopsy sample is used as an estimate of circulating tumor fraction. In some embodiments, a range defined by two or more of these statistics is used to limit the range of simulated tumor fraction analysis via the off-target read methodology described herein. For instance, in some embodiments, lower and upper bounds of the simulated tumor fraction analysis are defined by the minimum variant allele fraction and the maximum variant allele fraction determined for a liquid biopsy sample, respectively. In some embodiments, the range is further expanded, e.g., on either or both the lower and upper bounds. For example, in some embodiments, the lower bound of a simulated tumor fraction analysis is defined as 0.5-times the minimum variant allele fraction, 0.75-times the minimum variant allele fraction, 0.9-times the minimum variant allele fraction, 1.1-times the minimum variant allele fraction, 1.25-times the minimum variant allele fraction, 1.5-times the minimum variant allele fraction, or a similar multiple of the minimum variant allele fraction determined for the liquid biopsy sample. Similarly, in some embodiments, the upper bound of a simulated tumor fraction analysis is defined as 2.5-times the maximum variant allele fraction, 2-times the maximum variant allele fraction, 1.75-times the maximum variant allele fraction, 1.5-times the maximum variant allele fraction, 1.25-times the maximum variant allele fraction, 1.1-times the maximum variant allele fraction, 0.9-times the maximum variant allele fraction, or a similar multiple of the maximum variant allele fraction determined for the liquid biopsy sample.

In some embodiments, circulating tumor fraction is estimated based on a distribution of the lengths of cfDNA in the liquid biopsy sample. In some embodiments, sequence reads are binned according to their position within the genome, e.g., as described elsewhere herein. For each bin, the length of each fragment is determined. Each fragment is then classified as belonging to one of a plurality of classes, e.g., one of two classes corresponding to a population of short fragments and a population of long fragments. In some embodiments, the classification is performed using a static length threshold, e.g., that is the same across all the bins. In some embodiments, the classification is performed using a dynamic length threshold. In some embodiments, a dynamic length threshold is determined by comparing the distribution of fragment lengths in liquid biopsy samples from reference subjects that do not have cancer to the distribution of fragment lengths in liquid biopsy samples from reference subjects that have cancer, in a positional fashion.

For example, in some embodiments, the comparison is done over windows spanning entire chromosomes, e.g., each chromosome defines a comparison window over which a dynamic length threshold is determined. In some embodiments, the comparison is done over a window spanning a single bin, e.g., each bin defines a comparison window over which a dynamic length threshold is determined. In certain embodiments, the bin determination may be made according to various genomic features. For example, the comparison window may be based on a chromosome by chromosome basis, or a chromosomal arm by chromosomal arm basis. In some embodiments, the comparison window is based on a gene level basis. In some embodiments, the comparison window is a fixed size, such as 1 KB, 5 KB, 10 KB, 25 KB, 50 KB, 100 KB, 25 KB, 500 KB, 1 MB, 2 MB, 3 MB, or more. In some embodiments, the reference subjects having cancer used to determine the dynamic fragment length is matched to the cancer type of the subject whose liquid biopsy sample is being evaluated.

Once each fragment is classified as belonging to either the population of short fragments or the population of long fragments, a model trained to estimate circulating tumor fraction based on fragment length distribution data across the genome is applied to the binned data to generate an estimate of the circulating tumor fraction for the liquid biopsy sample. In some embodiments, a comparison of (i) the population of short fractions and (ii) the population of long fragments is made for each bin, e.g., a fraction of the number of short fragments to the number of long fragments in each bin is determined and used as an input for the model. In some embodiments, the model is a probabilistic model (e.g., an application of Bayes theorem), a deep learning model (e.g., a neural network, such as a convolutional neural network), or an admixture model.

In some embodiments, two or more of the circulating tumor estimation models described herein are used to generate respective tumor fraction estimates, which are combined to form a final tumor fraction estimate. For example, in some embodiments, a measure of central tendency (e.g., a mean) for several tumor fraction estimates is determined and used as the final tumor fraction estimate. In some embodiments, a tumor fraction estimate derived from a plurality of estimation models, e.g., a measure of central tendency for several tumor fraction estimates is used to identify the nearest local optima (e.g., minima) obtained from the tumor fraction estimation methods described above, and further herein.

Blocks 542-544. Referring to block 542, in some embodiments, method 500 includes obtaining one or more clonal hematopoiesis prevalence metrics for the candidate variant. Referring to block 544, in some embodiments, a clonal hematopoiesis prevalence metric in the one or more clonal hematopoiesis prevalence metrics is a frequency, in a cohort of solid tumors, of a variant of hematopoietic lineage at a locus encompassing the first nucleotide position.

In some embodiments, a clonal hematopoiesis prevalence metric (e.g., CHIP likelihood of Example 3) is determined for a particular variant based on sequencing results for a cohort of solid tumor samples with matched liquid biopsy samples. The metric is calculated as the total number of occurrences of the variant in the solid tumor samples that were classified as somatic divided by the total number of occurrences of the variant in the solid tumor samples that were classified as either somatic or non-somatic (e.g., either of germline or CHIP lineage).

Block 546. Referring to block 546, in some embodiments, a clonal hematopoiesis prevalence metric in the one or more clonal hematopoiesis prevalence metrics is a comparison of (i) instances of the candidate variant, in a cohort of solid tumors, that are of hematopoietic or germline lineage and (ii) total instances of the candidate variant in the cohort of solid tumors, or a comparison of (i) instances of the candidate variant, in the cohort of solid tumors, that are of somatic lineage and (ii) total instances of the candidate variant in the cohort of solid tumors.

Block 548. Referring to block 548, method 500 includes inputting information into a first model comprising a plurality of parameters thereby obtaining as output from the first model, through application of the plurality of parameters to the information, whether the candidate variant is (a) a somatic variant derived from cell free DNA (b) other than a somatic variant derived from cell free DNA.

Block 550. Referring to block 550, in some embodiments, the information includes (i) the one or more fragment length metrics, (ii) the variant allele fraction for the candidate somatic variant or one or more features determined from the variant allele fraction for the candidate somatic variant for the liquid biopsy sample, and/or (iii) the one or more learned clonal hematopoiesis metrics for the first locus, and/or an arithmetic combination of (i), (ii), and (iii).

Block 552. Referring to block 552, in some embodiments, an estimated circulating tumor fraction (ctFE) for the liquid biopsy sample is determined. In some embodiments the ctFE is determined based on the plurality of sequence reads. In some such embodiments, the information further comprises the ctFE for the liquid biopsy sample or one or more features determined from the ctFE for the liquid biopsy sample. In some embodiments the ctFE is determined using and of the methods for ctFE determination that are disclosed in PCT Application Number PCT/US24/51101, entitled “SYSTEMS AND METHODS FOR MOLECULAR RESIDUAL DISEASE LIQUID BIOPSY ASSAY,” filed Oct. 11, 2024, which is hereby incorporated by reference.

Block 554. Referring to block 554, in some embodiments, the information includes the variant allele fraction and the ctFE for the liquid biopsy sample.

Block 556. Referring to block 556, in some embodiments, the information includes the one or more features determined from the variant allele fraction for the candidate variant and the ctFE for the liquid biopsy sample. In some such embodiments a feature in the one or more features (determined from the variant allele fraction) is a residual value calculated by inputting the variant allele fraction into a second model to obtain as output from the second model an expected circulating tumor fraction and comparing (i) the expected circulating tumor fraction with (ii) the ctFE, In alternative embodiments a feature in the one or more features (determined from the variant allele fraction) is a residual value calculated by inputting the ctFE into a third model to obtain as output from the third model an expected variant allele fraction and comparing (i) the expected variant allele fraction with (ii) the variant allele fraction for the candidate somatic variant. In some embodiments the second model is trained on the relationship between circulating tumor fractions of liquid biopsies and variant allele fractions of somatic variants in the liquid biopsies. In some embodiments the second model is a regression model. In some alternative embodiments the second model has any of the architectures described in the present disclosure. In some embodiments the third model is trained on the relationship between circulating tumor fractions of liquid biopsies and variant allele fractions of somatic variants in the liquid biopsies. In some embodiments the third model is a regression model. In some alternative embodiments the third model has any of the architectures described in the present disclosure.

In some embodiments, the first model uses any 1, 2, 3, 4, 5, 6, 7, 8, 9, or 10 features associated with a variant that is being evaluated (to validate whether it is (a) a somatic variant derived from cell free DNA (b) other than a somatic variant derived from cell free DNA) from the group consisting of gene frequency, alternate allele fragment length median, CHIP likelihood, Kolmogorov-Smirnov test metric for fragment length, p-value for Kolmogorov-Smirnov test metric for fragment length, alternate allele fragment length kurtosis, estimated circulating tumor fraction, ensemble variant allele fraction residual, alternate allele fragment length skew, and variant allele fraction, where such features are further described in Examples 3 and 4 below.

In some embodiments the first model uses the following features of a variant to determine whether the variant is a CHIP variant: VAF, polymorphism length, reference and alternate allele fragment length distribution statistics, and historical variant chip likelihood. In some embodiments the first model is an ensemble model. It will be appreciated that in those embodiments where the first model does not use a feature that is acquired in accordance with FIG. 5, acquisition of such feature is optional and need not be obtained.

In some embodiments the information inputted into the first model includes a fragment length feature representing the frequency that cfDNA fragments encompassing the candidate variant have fragment lengths more frequently associated with non-somatic sequence variants than somatic sequence variants. For example, Marass et al., 2020, Clin Chem., 66 (4): 616-18, the disclosure of which is incorporated herein by reference, reports that cfDNA fragments containing a CHIP variant are more likely to have a length between 173-191 bp or between 346-361 bp than other lengths, while cfDNA fragments containing a somatic variant are more likely to have a length between 127-141 bp or between 272-292 bp than other lengths. In some embodiments, this feature is used in addition to any combination of features disclosed herein in the information inputted into the first model. In some embodiments, this feature is used in place of an existing feature in any combination of features disclosed herein, e.g., it is swapped in to replace a different feature. In some embodiments, this feature is used in combination with any other feature or features described herein in the information inputted into the first model.

Accordingly, in some embodiments, such a feature is a comparison of (i) the number of cfDNA fragments encompassing a respective sequence variant with fragment lengths falling within a range of fragment lengths enriched for CHIP variants and (ii) the total number of cfDNA fragments encompassing the respective sequence variant. However, the comparison can also be determined against other related measurements. For example, in some embodiments, such a feature is a comparison of (i) the number of cfDNA fragments encompassing a respective candidate variant with fragment lengths falling within a range of fragments lengths enriched for CHIP variants and (ii) the total number of cfDNA fragments encompassing the respective sequence variant having fragment lengths that either (a) fall within a range of fragment lengths enriched for CHIP variants or (b) fall within a range of fragment lengths enriched for somatic variants. In yet other embodiments, such a feature is a comparison of (i) the number of cfDNA fragments encompassing a respective sequence variant with fragment lengths falling within a range of fragment lengths enriched for CHIP variants and (ii) the number of cfDNA fragments encompassing a respective sequence variant with fragment lengths falling within a range of fragment lengths enriched for somatic variants. In some embodiments, any of the comparisons described above can be made with respect to a number of cfDNA fragments encompassing a respective sequence variant with fragment lengths falling within a range of fragment lengths enriched for non-somatic variants, rather than just for CHIP variants.

In some embodiments, the first model as described herein for distinguishing whether a variant is (a) a somatic variant derived from cell free DNA (b) other than a somatic variant derived from cell free DNA uses, as part of the information inputted into the first model, a historical prevalence feature representing the comparison of the frequency at which the sequence variant is identified in cfDNA of cancer patients relative to the frequency at which the sequence variant is identified in solid tumor samples. This feature is based on the observation that some CHIP variants are identified more frequently in cfDNA than in DNA from solid tumors. Without intending to be limited to any particular theory, this observation may be based on the rational that cfDNA should contain a higher frequency of nucleic acids originating from cells of hematopoietic lineage than DNA extracted from solid tumors. Accordingly, a sequence variant that is more frequently identified in cfDNA than in DNA from solid tumors should have a higher likelihood of having a hematopoietic lineage than a sequence variant that is not more frequently identified in cfDNA than in DNA from solid tumors. In some embodiments, this feature is used in addition to any combination of features disclosed herein as information that is inputted into the first model. In some embodiments, this feature is used in place of an existing feature in any combination of features disclosed herein, e.g., it is swapped in to replace a different feature in the information that is inputted into the first model. In some embodiments, this feature is used in combination with any other feature or features described herein in the information that is inputted into the first model.

In some embodiments, the first model as described herein for determining whether a variant is (a) a somatic variant derived from cell free DNA (b) other than a somatic variant derived from cell free DNA, uses a historical prevalence feature representing how uniformly a sequence variant is detected across a range of cancer types. Such a feature is based on the observation that some sequence variants occur more frequently in one or more cancer types than in other cancer types and thus, without being limited to any particular theory, may be drivers of those specific cancer types and, therefore, more likely to be a somatic variant. In contrast, variants that occur at a consistent frequency across many types of cancers are more likely to be passenger mutations without a somatic lineage. In some embodiments, this feature is used in addition to any combination of features disclosed herein in the information that is inputted into the first model. In some embodiments, this feature is used in place of an existing feature in any combination of features disclosed herein, e.g., it is swapped in to replace a different feature in the information that is inputted into the first model. In some embodiments, this feature is used in combination with any other feature or features described herein in the information that is inputted into the first model.

In some embodiments, the first model as described herein for distinguishing whether a variant is (a) a somatic variant derived from cell free DNA (b) other than a somatic variant derived from cell free DNA, uses a feature representing whether a frequency of identifying the candidate allele in samples, e.g., liquid biopsy samples, from the subject is stable over time. For example, in some such embodiments, the feature is a representation of the amount of change in the variant allele frequency of the variant in biological samples from the test subject over time. In some embodiments, the change in frequency is normalized against the measure of the amount of tumor DNA in the sample over time, e.g., against a circulating tumor fraction estimate for the sample(s). In some embodiments, the change in frequency is alternatively, or additionally, normalized against relative changes in the frequency of other sequence variants over time. For example, it has been observed that the prevalence of some somatic variants in cfDNA should increase over time when the circulating tumor fraction is increasing and that the prevalence of non-somatic variants should decrease over time when the circulating tumor fraction is decreasing over time. In some embodiments, this feature is used in addition to any combination of features disclosed herein as information that is inputted into the first model. In some embodiments, this feature is used in place of an existing feature in any combination of features disclosed herein, e.g., it is swapped in to replace a different feature in the information that is inputted into the first model. In some embodiments, this feature is used in combination with any other feature or features described herein in the information that is inputted into the first model.

In some embodiments, the first model as described herein for distinguishing whether a variant is (a) a somatic variant derived from cell free DNA (b) other than a somatic variant derived from cell free DNA uses, as part of the information inputted into the first model, any 2, 3, 4, 5, 6, 7, 8, 9, 10, 11, 12, 13, 14, 15, 16, 17, 18, 19, 20, 21, 22, 23, 24, 25 or more features from Table 6.

TABLE 6
Feature

Variant allele frequency
Third quartile cfDNA allele frequency
Median cfDNA allele frequency
COSMIC solid tumor frequency
GnomAD allele frequency
Gene name: DNMT3A
COSMIC heme/lymph frequency
SBS55
ExAC frequency
SBS2
SBS35
SBS7a
SBS54
SBS8
SBS6
SBS44
SBS10a
SBS59
SBS25
SBS21
SBS19
SBS30
Gene name: TET2
SBS10b
SBS7b
Gene name: EGFR
SBS11
Gene name: KMT2C
SBS32
SBS17b
SBS26
CH rule class: CHIP
SBS4
SBS33

In Table 6, the “variant allele fraction” feature is calculated as the fraction of all cfDNA fragments in a liquid biopsy sample encompassing the locus for the candidate variant that contain the candidate variant in the biopsy sample from the test subject. Thus, if the “variant allele fraction” feature of Table 6 is used, the observed variant allele fraction of the candidate variant in the biopsy sample from the test subject is inputted into the first model.

In Table 6, the “third quartile cfDNA allele frequency” feature is a binary indication of whether or not a particular variant is in the third quartile of allele fractions of variants called using the biopsy sample from the test subject. Thus, if the “third quartile cfDNA allele frequency” feature of Table 6 is used, a binary indication as to whether or not the observed variant allele fraction for the candidate variant that is observed using the biopsy sample from the test subject is in the third quartile or not is inputted into the first model. For instance, in one nonlimiting example, if the observed variant allele fraction for the candidate variant that is observed using the biopsy sample from the test subject is in the third quartile, a “1” is entered for this feature into the first model, and otherwise a “0” is entered into the first model for this feature.

In Table 6, the “median cfDNA allele frequency” feature is calculated as the median allele frequency of all variants called using the biopsy sample from the test subject. For instance, if there are three variants called with respective fractions of 5 percent, 10 percent, and 15 percent, the median cfDNA is 10. Thus, if the “median cfDNA allele frequency” feature of Table 6 is used, the median cfDNA allele frequency across the plurality of candidate variants observed in the biopsy sample from the test subject is inputted into the first model.

In Table 6, the “COSMIC solid tumor frequency” feature is the variant allele frequency of the variant in those subjects in the Catalog of Somatic Mutations in Cancer (COSMIC) database that have solid tumors. See Tate et al, 2019, “COSMIC: The catalogue of somatic mutations in cancer,” Nucleic Acids Res 47, D941-D947, which is hereby incorporated by reference. Thus, in such embodiments that make use of the feature “COSMIC solid tumor frequency,” the information inputted into the first model includes the variant allele frequency of the variant subjects having solid tumors that are in the COSMIC database. The frequency of the variant in solid tumors was calculated by summing all instances of the candidate somatic variant found in non-other and nonhematopoietic and lymphoid tissues in the COSMIC database and dividing by the total number of instances of the candidate somatic variant found in non-other tissues.

In Table 6, the feature “GnomAD allele frequency” is calculated as the variant allele frequency of the candidate somatic variant in the Genome Aggregation Database (gnomAD). See Karczewski et al, 2020, “The mutational constraint spectrum quantified from variation in 141,456 humans,” Nature 581, pp. 434-443, which is hereby incorporated by reference. Thus, in such embodiments that make use of the feature “GnomAD allele frequency,” the information inputted into the model includes the variant allele frequency of the variant in the GnomAD database.

In Table 6, if gene name DNMT3A, TET2, EGFR, and/or KMT2C, feature is used as a feature, what is inputted into the first model is a binary indication as to whether or not the variant occurs in the specified gene. Thus, for example, if the DNMT3A feature is inputted into (used by) the first model, the DNMT3A feature is a binary indication as to whether or not the variant occurs in the DNMT3A gene.

In Table 6, the “COSMIC heme/lymph frequency” feature is calculated as the observed frequency of the candidate somatic variant in those subjects in the COSMIC database that have the hematologic disorders (hematopoietic or lymphoid). It is obtained by dividing the number of instances the candidate somatic variant is observed in COSMIC subjects that have hematopoietic or lymphoid disorders by the number of instances the candidate somatic variant is observed in all other tissues of COSMIC subjects.

In Table 6, SBS55, SBS2, SBS35, SBS7a, SBS54, SBS8, SBS6, SBS44, SBS10a, SBS59, SBS25, SBS21, SBS19, SBS30, SBS10b, SBS7b, SBS11, SBS32, SBS17b, SBS26, SBS4, and SBS33 are each single-base substitution signatures. They each represent specific patterns of single-base substitutions that can be linked to different biological processes, mutational causes, or environmental factors (e.g., UV light, smoking, or DNA repair deficiencies). While there are 96 possible SBS mutation types based on the nucleotide change and the two surrounding bases for a candidate somatic variation that is a single nucleotide polymorphism, they are grouped into 60 distinct mutational patterns for analysis purposes. See Alexandrov et al., 2020, “The repertoire of mutational signatures in human cancer,” Nature 578, 94-101, which is hereby incorporated by reference. If one of these features is used, the score for the candidate variant for this feature is included in the information inputted into the first model. For instance, if the “SBS55” feature is used, the score for the candidate variant against the SBS55 single-base substitution signature is inputted into the first model. Sec Fairchild et al., 2023, “Clonal hematopoiesis detection in patients with cancer using cell-free DNA sequencing,” Science Translational Medicine 15, eabm8729 for methods for calculating such a score.

In Table 6, the “ExAC frequency” feature is calculated as the variant allele frequency of the candidate somatic variant in the Exome Aggregation Consortium Database (ExAC). See Karczewski et al, 2017, “The ExAC browser: Displaying reference data information form over 60,000 exomes,” Nucleic Acids Res 45, D840-D845, which is hereby incorporated by reference. Thus, in such embodiments that make use of the feature “ExAC frequency,” the information inputted into the model includes the variant allele frequency of the variant in the ExAC database.

In Table 6, the “CH rule class: CHIP” feature is a binary indication as to whether the candidate somatic variant is within a CHIP driver gene or not. In some embodiments the CHIP driver genes are DNMT3A, TET2, ASXL1, JAK2, SF3B1, and PPM1D. In some embodiments the CHIP driver genes are DNMT3A, TET2, ASXL1, JAK2, SF3B1, PPM1D, CBL, and KMT2C. In some embodiments the CHIP driver genes are DNMT3A and TET2. In some embodiments the CHIP driver genes are DNMT3A, TET2, and ASXL1.

In some embodiments, any of the features of Table 6 is used in addition to any combination of features disclosed herein as information that is inputted into the first model. In some embodiments, any feature of Table 6 is used in place of an existing feature in any combination of features disclosed herein, e.g., it is swapped in to replace a different feature in the information that is inputted into the first model. In some embodiments, any combination of feature of Table 6 is used in combination with any other feature or features described herein in the information that is inputted into the first model.

In some embodiments, a feature used in the information inputted into the first model in accordance with block 548 is a score for the candidate somatic variant against any one of the 60 single-base substitution signatures SB1-SB60 as defined in Alexandrov et al., 2020, “The repertoire of mutational signatures in human cancer,” Nature 578, 94-101, which is hereby incorporated by reference

Blocks 558-560. Referring to blocks 558-560, in some embodiments, the first model is a regression model, a neural network, a support vector machine, a Naive Bayes model, a nearest neighbor model, a boosted trees model, a random forest model, a decision tree, a gradient boosted tree, an elastic net, or a clustering model. Referring to block 552, in some embodiments, the first model is a random forest model. In some embodiments the first model is an XBboost model.

As used herein, the term “model” refers to a machine learning model, algorithm, or task. In some embodiments, a model is an unsupervised learning algorithm. One example of an unsupervised learning algorithm is cluster analysis. In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level classifier). In some embodiments, a model is utilized to normalize a value or data set, such as by transforming the value or a set of values to a common frame of reference for comparison purposes.

In some embodiments, an untrained model (e.g., “untrained classifier” and/or “untrained neural network”) includes a machine learning model or algorithm, such as a classifier or a neural network, that has not been trained on a target dataset. In some embodiments, training a model (e.g., training a neural network) refers to the process of training an untrained or partially trained model (e.g., an untrained or partially trained neural network). For instance, consider the case of a plurality of training samples comprising a corresponding plurality of medical images (e.g., of a medical dataset). The plurality of medical images is applied as collective input to an untrained or partially trained model, in conjunction with a corresponding measured indication of one or more features for each respective medical image (hereinafter training dataset) to train the untrained or partially trained model on indications that identify features related to morphological classes, thereby obtaining a trained model. Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8th Iberian Conference Proceedings, 243-250, which is hereby incorporated by reference in its entirety for all purposes, provides non-limiting examples of such transfer learning. In instances where transfer learning is used, the untrained model described above is provided with additional data over and beyond that of the primary training dataset. That is, in non-limiting examples of transfer learning embodiments, the untrained model receives (i) the plurality of images and the measured indications for each respective image (“primary training dataset”) and (ii) additional data. In some embodiments, this additional data is in the form of parameters (e.g., coefficients, weights, and/or hyperparameters) that were from another, auxiliary training dataset. Moreover, while a description of a single auxiliary training dataset has been disclosed, it will be appreciated that there is no limit on the number of auxiliary training datasets that may be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The parameters learned from the first auxiliary training dataset (by application of a first model to the first auxiliary training dataset) may be applied to the second auxiliary training dataset using transfer learning techniques (e.g., a second model that is the same or different from the first model), which in turn may result in a trained intermediate model whose parameters are then applied to the primary training dataset and this, in conjunction with the primary training dataset itself, is applied to the untrained model. Alternatively, a first set of parameters learned from the first auxiliary training dataset (by application of a first model to the first auxiliary training dataset) and a second set of parameters learned from the second auxiliary training dataset (by application of a second model that is the same or different from the first model to the second auxiliary training dataset) may each individually be applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) may then be applied to the untrained model in order to train the untrained model. In some instances, additionally or alternatively, knowledge regarding objects related to morphological classes derived from an auxiliary training dataset is used, in conjunction with the object and/or class-labeled images in the primary training dataset, to train the untrained model.

Support vector machines. In some embodiments, the model is a support vector machine (SVM). SVM algorithms suitable for use as models are described in, for example, Cristianini and Shawe-Taylor, 2000, An Introduction to Support Vector Machines, Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furcy et al., 2000, Bioinformatics 16, 906-914, each of which is hereby incorporated by reference in its entirety for all purposes. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes algorithms. In some embodiments, the model is a Naive Bayes algorithm. Naïve Bayes classifiers suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference in its entirety for all purposes. A Naive Bayes classifier is any classifier in a family of “probabilistic classifiers” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference in its entirety for all purposes.

Nearest neighbor algorithms. In some embodiments, a model is a nearest neighbor algorithm. Nearest neighbor models can be memory-based and include no model to be fit. For nearest neighbors, given a query point x₀ (a first image), the k training points x₍ᵣ₎, r, . . . , k (here the training images) closest in distance to x₀ are identified and then the point x₀ is classified using the k nearest neighbors. In some embodiments, the distance to these neighbors is a function of the values of a discriminating set. In some embodiments, Euclidean distance in feature space is used to determine distance as d₍ᵢ₎=∥x₍ᵢ₎−x₍₀₎∥. In some embodiments, when the nearest neighbor algorithm is used, the value data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastic, 2001, The Elements of Statistical Learning, Springer, New York, each of which is hereby incorporated by reference in its entirety for all purposes.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference in its entirety for all purposes. In some embodiments, the number of distance calculations needed to solve the k-nearest neighbor model is such that a computer is used to solve the model for a given input because it cannot be mentally performed.

Random forest, decision tree, and boosted tree algorithms. In some embodiments, the model is a decision tree. Decision trees suitable for use as models are described generally by Duda, 2001, Pattern Classification, John Wiley & Sons, Inc., New York, pp. 395-396, which is hereby incorporated by reference in its entirety for all purposes. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference in its entirety for all purposes. CART, MART, and C4.5 are described in Hastie et al., 2001, The Elements of Statistical Learning, Springer-Verlag, New York, Chapter 9, which is hereby incorporated by reference in its entirety for all purposes. Random Forests are described in Breiman, 1999, “Random Forests—Random Features,” Technical Report 567, Statistics Department, U.C. Berkeley, September 1999, which is hereby incorporated by reference in its entirety for all purposes. In some embodiments, the decision tree model includes at least 10, at least 20, at least 50, or at least 100 parameters (e.g., weights and/or decisions) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the model (e.g., a linear classifier) in some embodiments of the present disclosure.

Mixture model and Hidden Markov model. In some embodiments, the model is a mixture model, such as that described in McLachlan et al., Bioinformatics 18 (3): 413-422, 2002. In some embodiments, in particular, those embodiments including a temporal component, the model is a hidden Markov model such as described by Schliep et al., 2003, Bioinformatics 19 (1): 1255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter, “Duda 1973”) which is hereby incorporated by reference in its entirety for all purposes. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in a training dataset. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering includes unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning algorithms to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

The term “classification” can refer to any number(s) or other characters(s) that are associated with a particular property of a sample. For example, a “+” symbol (or the word “positive”) can signify that a sample is classified as having a desired outcome or characteristic, whereas a “−” symbol (or the word “negative”) can signify that a sample is classified as having an undesired outcome or characteristic. In another example, the term “classification” refers to a respective outcome or characteristic (e.g., high risk, medium risk, low risk). In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff value refers to a value above which results are excluded. In some embodiments, a threshold value is a value above or below which a particular classification applies. Either of these terms can be used in either of these contexts.

One of skill in the art will readily appreciate other models that are applicable to the systems and methods of the present disclosure. In some embodiments, the systems, methods, and devices of the present disclosure utilize more than one model to provide an evaluation (e.g., arrive at an evaluation given one or more inputs) with an increased accuracy. For instance, in some embodiments, each respective model arrives at a corresponding evaluation when provided a respective data set. Accordingly, each respective model can independently arrive at a result and then the result of each respective model is collectively verified through a comparison or amalgamation of the models. From this, a cumulative result is provided by the models. However, the present disclosure is not limited thereto.

Block 562. In some embodiments, the first model applies the plurality of parameters to the information through a plurality of computations and the plurality of computations is at least 10,000 computations. In some embodiments, the plurality of parameters is at least 1000, at least 10,000, at least 15,000, at least 50,000, at least 100,000, at least 250,000, at least 500,000, or at least 1,000,000 parameters, at least 2,500,000 parameters, at least 5,000,000 parameters, at least 10,000,000 parameters, or more.

In some embodiments, the first model applies the plurality of parameters to the information through at least 1000 computation, at least 5000 computations, at least 10,000 computations, at least 25,000 computations, at least 50,000 computations, at least 100,000 computations, at least 250,000 computations, at least 500,000 computations, at least 1,000,000 computations, at least 2,500,000 computations, at least 5,000,000 computations, at least 10,000,000 computations, or more.

Block 564. Referring to block 564, in some embodiments, the first model was trained against a training dataset including, for each respective matched pair of samples in a plurality of matched pairs of samples, where each respective matched pair of samples includes a corresponding liquid biopsy sample and a corresponding solid tumor sample acquired from the same respective training subject in a plurality of training samples, (i) corresponding information for a corresponding variant identified in the corresponding liquid biopsy sample as independent variables for the training and (ii) a corresponding determination of whether the corresponding variant is of somatic lineage based on evaluation of the corresponding liquid biopsy sample and the corresponding solid tumor sample.

Block 566. Referring to block 566, in some embodiments, method 500 includes providing a validation of the somatic sequence variant when the indication of whether the candidate somatic variant has a somatic lineage satisfies a criterion, or a rejection of the somatic sequence variant when the indication of whether the candidate somatic variant has a somatic lineage fails to satisfy the criterion. In some embodiments the criterion is a threshold as described in Example 3. In some embodiments the first model directly validates whether or not a candidate somatic variant is, in fact, a somatic variant derived from cell free DNA and block 566 and its associated criterion are not used.

Block 568. Referring to block 568, in some embodiments, when the first model indicates that the candidate variant is other than a somatic variant derived from cell free DNA, the method includes classifying the candidate somatic variant as a variant of hematopoietic lineage or germline lineage. In some embodiments, the classification is a heuristic, labeling the identified variants based on base fraction and CHIP likelihood for each specific variant.

Blocks 570-574. In some embodiments, identified somatic variants derived from cell free DNA and/or identified variants of hematopoietic lineage are used for further downstream analysis and biomarker detection. In some embodiments, classified variants are used as a metric for disease detection, diagnosis, and/or treatment. In some embodiments, classified variants are included in a clinical report made available to the patient or a clinician. In some embodiments, classified variants are used to select appropriate therapies and/or clinical trials.

As described herein, in some embodiments, one or more validated somatic variant statuses 132-v (validated as being somatic variants derived from cell free DNA) are used to match the subject with a targeted therapy and/or a clinical trial. In some embodiments, as described in further detail herein, one or more validated somatic variant statuses 132-v for one or more actionable variants 139-1-1, one or more matched therapies 139-1-2, and/or one or more matched clinical trials are used to generate a patient report 139-1-3. In some embodiments, the patient report is transmitted to a medical professional treating the subject. In some embodiments, the patient is then administered a personalized course of therapy, e.g., based on a matched therapy and/or clinical trial.

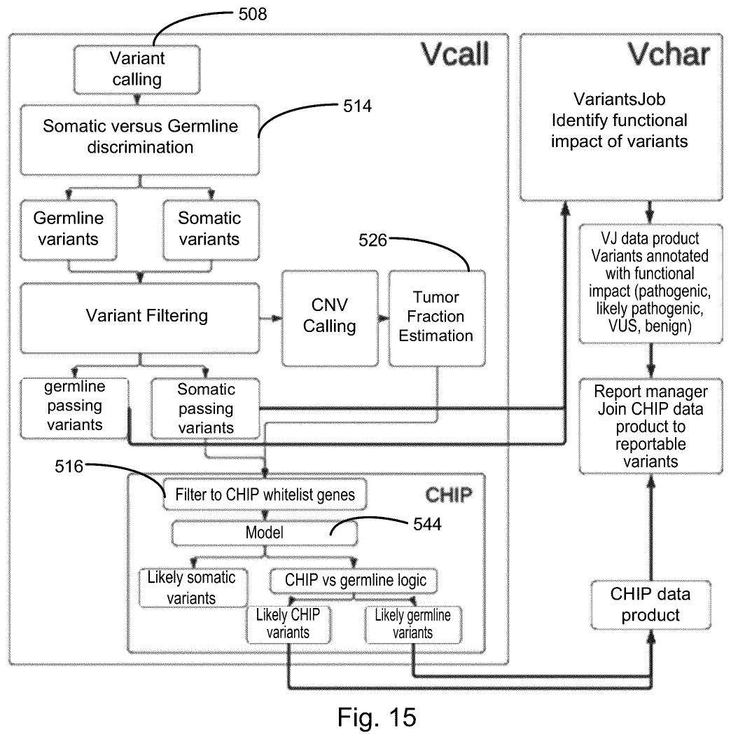

In some embodiments, the methods of validating a somatic variant in a liquid biopsy assay described herein fall within the context of a larger variant detection method such as illustrated in FIG. 15. For example, in some embodiments, the method includes obtaining cfDNA sequence reads, as described herein, and aligning those reads to a reference construct (e.g., a reference genome or mapped representation of several reference genomes), to generate aligned sequences 124 (e.g., a plurality of unique sequence reads). In some embodiments, putative somatic sequence variants are identified, e.g., those sequence variants having a variant allele fraction that is lower than expected for a germline sequence variant (which should be around 50% after accounting for an estimated circulating tumor fraction for the liquid biopsy sample), e.g., less than 30%, less than 20%, less than 10% etc. In some embodiments, one or more candidate somatic sequence variants are further validated by applying one or more filters. For instance, in some embodiments as described in U.S. Pat. No. 11,475,981, a dynamic variant count threshold is used to apply a dynamic probabilistic variant count filter to sequencing data for the candidate somatic sequence variant. In some embodiments, the method also includes applying a variant loci coverage filter. In some embodiments, the method also includes applying a variant allele fraction filter. In some embodiments, the method also includes applying a variant support mapping filter. In some embodiments, the method also includes applying a variant support sequencing quality filter. In some embodiments, the method also includes applying a low complexity region filter. In some embodiments, one or more candidate somatic variants is then further validated using the methods and systems described herein for filtering clonal hematopoiesis variants in a liquid biopsy assay. When all selected candidate somatic sequence variants have been validated or rejected according to these filters, the process proceeds with a reporting function.

In some embodiments, the method also includes validating one or more germline mutations. In some embodiments, candidate germline sequence variants are identified, e.g., those sequence variants having a variant allele fraction that is higher than expected for a somatic sequence variant. In some embodiments, the validation includes applying a germline-specific variant allele fraction filter. In some embodiments, the validation includes applying a variant support mapping filter. In some embodiments, the validation includes applying a variant support sequencing quality filter. When all selected candidate germline sequence variants have been validated or rejected according to these filters, the process proceeds with a reporting function.

As described herein, in some embodiments, one or more validated variant statuses 132-v are used to match the subject with a targeted therapy and/or a clinical trial. In some embodiments, as described herein, one or more validated variant statuses 132-v for one or more actionable variants 139-1-1, one or more matched therapies 139-1-2, and/or one or more matched clinical trials are used to generate a patient report 139-1-3. In some embodiments, the patient report is transmitted to a medical professional treating the subject. In some embodiments, the patient is then administered a personalized course of therapy, e.g., based on a matched therapy and/or clinical trial.

In some embodiments, different sets of sequence variants are evaluated depending on the type of cancer being evaluated. That is, when the subject has a first type of cancer, candidate sequence variants in a first set of genomic loci are evaluated, typically associated with the etiology of the first type cancer and/or a particular course of actionable therapy for the first type cancer, and when the subject has a second type of cancer, candidate sequence variants in a second set of genomic loci are evaluated, typically associated with the etiology of the second type cancer and/or a particular course of actionable therapy for the second type of cancer. These selections may be applied at the level of initial sequence read evaluation (e.g., only sequence reads corresponding to a defined set of loci are evaluated to identify a candidate sequence variant) or the validation level (e.g., sequence reads corresponding to a larger set of loci are evaluated to identify candidate sequence variants, but only those candidates corresponding to a defined set are further validated).

Similarly, in some embodiments, for one or more target loci falling within a gene exon, only candidate sequence variants that would result in an amino acid change in the amino acid sequence encoded by the gene are evaluated. In some embodiments, any candidate sequence variant resulting in an amino acid change are evaluated. In some embodiments, candidate sequence variants resulting in a defined amino acid change, e.g., an amino acid change associated with cancer etiology and/or a particular actionable cancer therapy, are evaluated. In some embodiments, only a subset of validated sequence variants is included on a clinical report for the sample. That is, in some embodiments, aligned sequence reads corresponding to all or a subset of genomic loci are evaluated to identify candidate sequence variants, all or a subset of identified candidate sequence variants are evaluated for validation, and only a subset of all possibly validated sequence variants are included on a clinical report generated for the sample.

For example, lists of example candidate sequence variants for evaluation in breast cancer, non-small cell lung cancer, prostate cancer, pan cancer, and cancer of unknown origin are provided below. Standard nomenclature is used to describe chromosomal location and specific amino acid variants, as described further by the Human Genome Variation Society, e.g., at the URL varnomen.hgvs.org/recommendations/protein/variant/substitution/.

For example, in some embodiments, the subject has breast cancer and candidate variants associated with at least one of the following genes and/or genetic loci are evaluated: ERBB2 (or a genetic locus including a chromosomal position of 17:37880220 and/or 17:37881064), EGFR (or a genetic locus including a chromosomal position of 7:55227926, 7:55242511, and/or 7:55249022), ESR1 (or a genetic locus including a chromosomal position of 6:152419922, 6:152419923 and/or 6:152419926), KRAS (or a genetic locus including a chromosomal position of 12:25380275, 12:25380276, 12:25380277, and/or 12:25380279), MAP2K1 (or a genetic locus including a chromosomal position of 15:66729162 and/or 15:66729163), MET (or a genetic locus including a chromosomal position of 7:116422117 and/or 7:116423413); MTOR (or a genetic locus including a chromosomal position of 1:11187094, 1:11187096, and/or 1:11187796), NTRK1 (or a genetic locus including a chromosomal position of 1:156846342, 1:156849044 and/or 1:156849144), and PIK3CA (or a genetic locus including a chromosomal position of 3:178936082, 3:178936091, 3:178936092, 3:178936093, 3:178952084, and/or 3:178952085). In some embodiments, the subject has breast cancer and candidate variants associated with at least 2, at least 3, at least 4, at least 5, at least 6, at least 7, or at least 8 of the genes listed above (or loci including the enumerated corresponding chromosomal positions) are evaluated. In some embodiments, the subject has breast cancer and candidate variants associated with any of the genes listed above (or loci including the enumerated corresponding chromosomal positions) are evaluated.

Similarly, in some embodiments, the subject has non-small cell lung cancer and candidate variants associated with at least one of the following genes and/or genetic loci are evaluated: ALK (or a genetic locus including a chromosomal position of 2:29443613, 2:29443631, 2:29443695, 2:29443697, 2:29445213, and/or 2:29445258), B2M (or a genetic locus including a chromosomal position of 15:45003745), BRAF (or a genetic locus including a chromosomal position of 7:140453135, 7:140453136, and/or 7:140453137), EGFR (or a genetic locus including a chromosomal position of 7:55227926, 7:55241704, 7:55241705, 7:55241706, 7:55242469, 7:55242511, 7:55249022, 7:55249071, 7:55249091, 7:55249092, 7:55249093, 7:55249094, and/or 7:55259515), ERBB2 (or a genetic locus including a chromosomal position of 17:37880220), KRAS (or a genetic locus including a chromosomal position of 12:25378562, 12:25378643, 12:25380275, 12:25380276, 12:25380277, 12:25380279, 12:25398255, 12:25398280, 12:25398281, 12:25398282, 12:25398283, 12:25398284, and/or 12:25398285), MAP2K1 (or a genetic locus including a chromosomal position of 15:66729162 and/or 15:66729163), MET (or a genetic locus including a chromosomal position of 7:116422117 and/or 7:116423413), NTRK1 (or a genetic locus including a chromosomal position of 1:156846342, 1:156849044, and/or 1:156849144), PIK3CA (or a genetic locus including a chromosomal position of 3:178936091, 3:178936092, 3:178936093, 3:178952072, 3:178952084, and/or 3:178952085), and STK11 (or a genetic locus including a chromosomal position of 19:1218483, 19:1220370, 19:1220487, 19:1220629, and/or 19:1220649). In some embodiments, the subject has non-small cell lung cancer and candidate variants associated with at least 2, at least 3, at least 4, at least 5, at least 6, at least 7, at least 8, at least 9, or at least 10 of the genes listed above (or loci including the enumerated corresponding chromosomal positions) are evaluated. In some embodiments, the subject has non-small cell lung cancer and candidate variants associated with any of the genes listed above (or loci including the enumerated corresponding chromosomal positions) are evaluated.

Similarly, in some embodiments, the subject has prostate cancer and candidate variants associated with at least one of the following genes and/or genetic loci are evaluated: AR (or a genetic locus including a chromosomal position of X:66766292, X:66931463, X:66931504, X:66937370, X:66937371, X:66937372, X:66943543, X:66943549, and/or X:66943552), EGFR (or a genetic locus including a chromosomal position of 7:55227926, 7:55242511, and/or 7:55249022), ERBB2 (or a genetic locus including a chromosomal position of 17:37880220), KRAS (or a genetic locus including a chromosomal position of 12:25380275, 12:25380276, and/or 12:25380277), MAP2K1 (or a genetic locus including a chromosomal position of 15:66729162 and/or 15:66729163), MET (or a genetic locus including a chromosomal position of 7:116422117 and/or 7:116423413), NTRK1 (or a genetic locus including a chromosomal position of 1:156846342, 1:156849044, and/or 1:156849144), and PIK3CA (or a genetic locus including a chromosomal position of 3:178952084 and/or 3:178952085). In some embodiments, the subject has prostate cancer and candidate variants associated with at least 2, at least 3, at least 4, at least 5, at least 6, or at least 7 of the genes listed above (or loci including the enumerated corresponding chromosomal positions) are evaluated. In some embodiments, the subject has prostate cancer and candidate variants associated with any of the genes listed above (or loci including the enumerated corresponding chromosomal positions) are evaluated.

In one example, the cancer condition is any type of cancer (for example, pan-cancer) and the somatic variants validated by this method include variants associated with any of the following genes: EGFR (or a genetic locus including a chromosomal position of 7:55227926, 7:55242511, and/or 7:55249022), ERBB2 (or a genetic locus including a chromosomal position of 17:37880220), KRAS (or a genetic locus including a chromosomal position of 12:25380275, 12:25380276, and/or 12:25380277), MAP2K1 (or a genetic locus including a chromosomal position of 15:66729162 and/or 15:66729163), MET (or a genetic locus including a chromosomal position of 7:116422117 and/or 7:116423413), NTRK1 (or a genetic locus including a chromosomal position of 1:156846342, 1:156849044, and/or 1:156849144), PIK3CA (or a genetic locus including a chromosomal position of 3:178952084 and/or 3:178952085), and TP53. In some embodiments, the subject has any cancer (e.g., pan cancer) and candidate variants associated with at least 2, at least 3, at least 4, at least 5, at least 6, or at least 7 of the genes listed above (or loci including the enumerated corresponding chromosomal positions) are evaluated. In some embodiments, the subject has any cancer (e.g., pan cancer) and candidate variants associated with any of the genes listed above (or loci including the enumerated corresponding chromosomal positions) are evaluated.

Similarly, in some embodiments, the subject has a tumor of unknown origin or a cancer of unknown primary and candidate variants associated with at least one of the following genes and/or genetic loci are evaluated: EGFR (or a genetic locus including a chromosomal position of 7:55227926, 7:55242511, and/or 7:55249022), ERBB2 (or a genetic locus including a chromosomal position of 17:37880220), KRAS (or a genetic locus including a chromosomal position of 12:25380275, 12:25380276, 12:25380277, and/or 12:25398255), MAP2K1 (or a genetic locus including a chromosomal position of 15:66729162 and/or 15:66729163), MET (or a genetic locus including a chromosomal position of 7:116422117 and/or 7:116423413), NRAS (or a genetic locus including a chromosomal position of 1:115258748), NTRK1 (or a genetic locus including a chromosomal position of 1:156846342, 1:156849044, and/or 1:156849144), PIK3CA (or a genetic locus including a chromosomal position of 3:178927980, 3:178952084 and/or 3:178952085), and TP53. In some embodiments, the subject has any cancer (e.g., pan cancer) and candidate variants associated with at least 2, at least 3, at least 4, at least 5, at least 6, at least 7, or at least 8 of the genes listed above (or loci including the enumerated corresponding chromosomal positions) are evaluated. In some embodiments, the subject has any cancer (e.g., pan cancer) and candidate variants associated with any of the genes listed above (or loci including the enumerated corresponding chromosomal positions) are evaluated.

In some embodiments, the method also includes generating a report for the test subject including somatic variants identified from the liquid biopsy sample. Referring to block 570, in some embodiments, when the indication of whether the candidate somatic variant has a somatic lineage satisfies the criterion, the method includes generating a report for the subject including the identity of the candidate somatic variant. Referring to block 572, in some embodiments, the report further includes a therapeutic recommendation for the subject based on the identity of the candidate somatic variant. Referring to block 574, in some embodiments, the report further includes an identification of a candidate somatic variant as having a hematopoietic lineage.

In some embodiments, the methods described herein include generating a clinical report 139-3 (e.g., a patient report), providing clinical support for personalized cancer therapy, and/or using the information curated from sequencing of a liquid biopsy sample, as described above. In some embodiments, the report is provided to a patient, physician, medical personnel, or researcher in a digital copy (for example, a JSON object, a pdf file, or an image on a website or portal), a hard copy (for example, printed on paper or another tangible medium). A report object, such as a JSON object, can be used for further processing and/or display. For example, information from the report object can be used to prepare a clinical laboratory report for return to an ordering physician. In some embodiments, the report is presented as text, as audio (for example, recorded or streaming), as images, or in another format and/or any combination thereof.

In some embodiments, the report includes information related to the specific characteristics of the patient's cancer, e.g., detected genetic variants, epigenetic abnormalities, associated oncogenic pathogenic infections, and/or pathology abnormalities. In some embodiments, other characteristics of a patient's sample and/or clinical records are also included in the report. For example, in some embodiments, the clinical report includes information on clinical variants, e.g., one or more of copy number variants (e.g., for actionable genes CCNE1, CD274 (PD-L1), EGFR, ERBB2 (HER2), MET, MYC, BRCA1, and/or BRCA2), fusions, translocations, and/or rearrangements (e.g., in actionable genes ALK, ROS1, RET, NTRK1, FGFR2, FGFR3, NTRK2 and/or NTRK3), pathogenic single nucleotide polymorphisms, insertion-deletions (e.g., somatic/tumor and/or germline/normal), therapy biomarkers, microsatellite instability status, and/or tumor mutational burden.

### Variant Characterization

In some embodiments, a predicted functional effect and/or clinical interpretation for one or more identified variants is curated by using information from variant databases. In some embodiments, a weighted-heuristic model is used to characterize each variant.

In some embodiments, identified clinical variants are labeled as “potentially actionable”, “biologically relevant”, “variants of unknown significance (VUSs)”, or “benign”. Potentially actionable alterations are protein-altering variants with an associated therapy based on evidence from the medical literature. Biologically relevant alterations are protein-altering variants that may have functional significance or have been observed in the medical literature but are not associated with a specific therapy. Variants of unknown significance (VUSs) are protein-altering variants exhibiting an unclear effect on function and/or without sufficient evidence to determine their pathogenicity. In some embodiments, benign variants are not reported. In some embodiments, variants are identified through aligning the patient's DNA sequence to the human genome reference sequence version hg19 (GRCh37). In some embodiments, actionable and biologically relevant somatic variants are provided in a clinical summary during report generation.

For instance, in some embodiments, variant classification and reporting is performed, where detected variants are investigated following criteria from known evolutionary models, functional data, clinical data, literature, and other research endeavors, including tumor organoid experiments. In some embodiments, variants are prioritized and classified based on known gene-disease relationships, hotspot regions within genes, internal and external somatic databases, primary literature, and other features of somatic drivers. Variants can be added to a patient (or sample, for example, organoid sample) report based on recommendations from the AMP/ASCO/CAP guidelines. Additional guidelines may be followed. Briefly, pathogenic variants with therapeutic, diagnostic, or prognostic significance may be prioritized in the report. Non-actionable pathogenic variants may be included as biologically relevant, followed by variants of uncertain significance. Translocations may be reported based on features of known gene fusions, relevant breakpoints, and biological relevance. Evidence may be curated from public and private databases or research and presented as 1) consensus guidelines 2) clinical research, or 3) case studies, with a link to the supporting literature. Germline alterations may be reported as secondary findings in a subset of genes for consenting patients. These may include genes recommended by the ACMG and additional genes associated with cancer predisposition or drug resistance.

In some embodiments, a clinical report 139-3 includes information about clinical trials for which the patient is eligible, therapies that are specific to the patient's cancer, and/or possible therapeutic adverse effects associated with the specific characteristics of the patient's cancer, e.g., the patient's genetic variations, epigenetic abnormalities, associated oncogenic pathogenic infections, and/or pathology abnormalities, or other characteristics of the patient's sample and/or clinical records. For example, in some embodiments, the clinical report includes such patient information and analysis metrics, including cancer type and/or diagnosis, variant allele fraction, patient demographic and/or institution, matched therapies (e.g., FDA approved and/or investigational), matched clinical trials, variants of unknown significance (VUS), genes with low coverage, panel information, specimen information, details on reported variants, patient clinical history, status and/or availability of previous test results, and/or version of bioinformatics pipeline.

In some embodiments, the results included in the report, and/or any additional results (for example, from the bioinformatics pipeline), are used to query a database of clinical data, for example, to determine whether there is a trend showing that a particular therapy was effective or ineffective in treating (e.g., slowing or halting cancer progression), and/or adverse effects of such treatments in other patients having the same or similar characteristics.

In some embodiments, the results are used to design cell-based studies of the patient's biology, e.g., tumor organoid experiments. For example, an organoid may be genetically engineered to have the same characteristics as the specimen and may be observed after exposure to a therapy to determine whether the therapy can reduce the growth rate of the organoid, and thus may be likely to reduce the growth rate of cancer in the patient associated with the specimen. Similarly, in some embodiments, the results are used to direct studies on tumor organoids derived directly from the patient. An example of such experimentation is described in U.S. Pat. No. 11,415,571, the content of which is hereby incorporated by reference, in its entirety, for all purposes.

As illustrated in FIG. 2A, in some embodiments, a clinical report is checked for final validation, review, and sign-off by a medical practitioner (e.g., a pathologist). The clinical report is then sent for action (e.g., for precision oncology applications).

### Digital and Laboratory Health Care Platform:

In some embodiments, the methods and systems described herein are utilized in combination with, or as part of, a digital and laboratory health care platform that is generally targeted to medical care and research. It should be understood that many uses of the methods and systems described above, in combination with such a platform, are possible. One example of such a platform is described in U.S. patent application Ser. No. 16/657,804, filed Oct. 18, 2019, which is hereby incorporated herein by reference in its entirety for all purposes.

For example, an implementation of one or more embodiments of the methods and systems as described above may include microservices constituting a digital and laboratory health care platform supporting analysis of liquid biopsy samples to provide clinical support for personalized cancer therapy. Embodiments may include a single microservice for executing and delivering analysis of liquid biopsy samples to clinical support for personalized cancer therapy or may include a plurality of microservices each having a particular role, which together implement one or more of the embodiments above. In one example, a first microservice may execute sequence analysis in order to deliver genomic features to a second microservice for curating clinical support for personalized cancer therapy based on the identified features. Similarly, the second microservice may execute therapeutic analysis of the curated clinical support to deliver recommended therapeutic modalities, according to various embodiments described herein.

Where embodiments above are executed in one or more micro-services with or as part of a digital and laboratory health care platform, one or more of such micro-services may be part of an order management system that orchestrates the sequence of events as needed at the appropriate time and in the appropriate order necessary to instantiate embodiments above. A microservices-based order management system is disclosed, for example, in U.S. Prov. Patent Application No. 62/873,693, filed Jul. 12, 2019, which is hereby incorporated herein by reference in its entirety for all purposes.

For example, continuing with the above first and second microservices, an order management system may notify the first microservice that an order for curating clinical support for personalized cancer therapy has been received and is ready for processing. The first microservice may execute and notify the order management system once the delivery of genomic features for the patient is ready for the second microservice. Furthermore, the order management system may identify that execution parameters (prerequisites) for the second microservice are satisfied, including that the first microservice has completed, and notify the second microservice that it may continue processing the order to curate clinical support for personalized cancer therapy, according to various embodiments described herein.

Where the digital and laboratory health care platform further includes a genetic analyzer system, the genetic analyzer system may include targeted panels and/or sequencing probes. An example of a targeted panel is disclosed, for example, in U.S. Prov. Patent Application No. 62/902,950, filed Sep. 19, 2019, which is incorporated herein by reference and in its entirety for all purposes. In one example, targeted panels may enable the delivery of next generation sequencing results for providing clinical support for personalized cancer therapy according to various embodiments described herein. An example of the design of next-generation sequencing probes is disclosed, for example, in U.S. Prov. Patent Application No. 62/924,073, filed Oct. 21, 2019, which is incorporated herein by reference and in its entirety for all purposes.

Where the digital and laboratory health care platform further includes a bioinformatics pipeline, the methods and systems described above may be utilized after completion or substantial completion of the systems and methods utilized in the bioinformatics pipeline. As one example, the bioinformatics pipeline may receive next-generation genetic sequencing results and return a set of binary files, such as one or more BAM files, reflecting nucleic acid (e.g., cfDNA, DNA and/or RNA) read counts aligned to a reference genome. The methods and systems described above may be utilized, for example, to ingest the cfDNA, DNA and/or RNA read counts and produce genomic features as a result.

When the digital and laboratory health care platform further includes an RNA data normalizer, any RNA read counts may be normalized before processing embodiments as described above. An example of an RNA data normalizer is disclosed, for example, in U.S. patent application Ser. No. 16/581,706, filed Sep. 24, 2019, which is incorporated herein by reference and in its entirety for all purposes.

When the digital and laboratory health care platform further includes a genetic data deconvoluter, any system and method for deconvoluting may be utilized for analyzing genetic data associated with a specimen having two or more biological components to determine the contribution of each component to the genetic data and/or determine what genetic data would be associated with any component of the specimen if it were purified. An example of a genetic data deconvoluter is disclosed, for example, in U.S. Patent Publication No. US2020-0210852, PCT Publication Number WO2020/142563, US19/69161, filed Dec. 31, 2019, and U.S. Patent Publication No. 2021/0118526, each of which is hereby incorporated herein by reference and in its entirety for all purposes.

When the digital and laboratory health care platform further includes an automated RNA expression caller, RNA expression levels may be adjusted to be expressed as a value relative to a reference expression level, which is often done in order to prepare multiple RNA expression data sets for analysis to avoid artifacts caused when the data sets have differences because they have not been generated by using the same methods, equipment, and/or reagents. An example of an automated RNA expression caller is disclosed, for example, in U.S. Pat. No. 11,043,283, which is incorporated herein by reference and in its entirety for all purposes.

The digital and laboratory health care platform may further include one or more insight engines to deliver information, characteristics, or determinations related to a disease state that may be based on genetic and/or clinical data associated with a patient and/or specimen. Exemplary insight engines may include a tumor of unknown origin engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, and so forth. An example tumor of unknown origin engine is disclosed, for example, in U.S. Prov. U.S. Pat. No. 11,527,323, which is incorporated herein by reference and in its entirety for all purposes. An example of an HLA LOH engine is disclosed, for example, in U.S. Pat. Nos. 11,081,210, and 11,475,978, each of which is incorporated herein by reference and in its entirety for all purposes. An example of a tumor mutational burden (TMB) engine is disclosed, for example, in U.S. Prov. Patent Publication No. 2020/0258601 which is incorporated herein by reference and in its entirety for all purposes. An example of a PD-L1 status engine is disclosed, for example, in U.S. Prov. Patent Publication No. 2020-0395097 which is incorporated herein by reference and in its entirety for all purposes. An additional example of a PD-L1 status engine is disclosed, for example, in U.S. Prov. Patent Application No. 62/824,039, filed Mar. 26, 2019, which is incorporated herein by reference and in its entirety for all purposes. An example of a homologous recombination deficiency engine is disclosed, for example, in U.S. Prov. Patent Application No. 62/804,730, filed Feb. 12, 2019, which is incorporated herein by reference and in its entirety for all purposes. An example of a cellular pathway activation report engine is disclosed, for example, in U.S. Prov. Patent Application No. 62/888,163, filed Aug. 16, 2019, which is incorporated herein by reference and in its entirety for all purposes. An example of an immune infiltration engine is disclosed, for example, in U.S. patent application Ser. No. 16/533,676, filed Aug. 6, 2019, which is incorporated herein by reference and in its entirety for all purposes. An additional example of an immune infiltration engine is disclosed, for example, in U.S. Patent Application No. 62/804,509, filed Feb. 12, 2019, which is incorporated herein by reference and in its entirety for all purposes. An example of an MSI engine is disclosed, for example, in U.S. patent application Ser. No. 16/653,868, filed Oct. 15, 2019, which is incorporated herein by reference and in its entirety for all purposes. An additional example of an MSI engine is disclosed, for example, in U.S. Prov. Patent Application No. 62/931,600, filed Nov. 6, 2019, which is incorporated herein by reference and in its entirety for all purposes.

When the digital and laboratory health care platform further includes a report generation engine, the methods and systems described above may be utilized to create a summary report of a patient's genetic profile and the results of one or more insight engines for presentation to a physician. For instance, the report may provide to the physician information about the extent to which the specimen that was sequenced contained tumor or normal tissue from a first organ, a second organ, a third organ, and so forth. For example, the report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ. The report may include therapies and/or clinical trials matched based on a portion or all of the genetic profile or insight engine findings and summaries. For example, the therapies may be matched according to the systems and methods disclosed in U.S. Prov. Patent Application No. 62/804,724, filed Feb. 12, 2019, which is incorporated herein by reference and in its entirety for all purposes. For example, the clinical trials may be matched according to the systems and methods disclosed in U.S. Patent Publication No. 2020/0381087, which is incorporated herein by reference and in its entirety for all purposes.

The report may include a comparison of the results to a database of results from many specimens. An example of methods and systems for comparing results to a database of results are disclosed in U.S. Prov. Patent Application No. 62/786,739, filed Dec. 31, 2018, which is incorporated herein by reference and in its entirety for all purposes. The information may be used, sometimes in conjunction with similar information from additional specimens and/or clinical response information, to discover biomarkers or design a clinical trial.

When the digital and laboratory health care platform further includes application of one or more of the embodiments herein to organoids developed in connection with the platform, the methods and systems may be used to further evaluate genetic sequencing data derived from an organoid to provide information about the extent to which the organoid that was sequenced contained a first cell type, a second cell type, a third cell type, and so forth. For example, the report may provide a genetic profile for each of the cell types in the specimen. The genetic profile may represent genetic sequences present in a given cell type and may include variants, expression levels, information about gene products, or other information that could be derived from genetic analysis of a cell. The report may include therapies matched based on a portion or all of the deconvoluted information. These therapies may be tested on the organoid, derivatives of that organoid, and/or similar organoids to determine an organoid's sensitivity to those therapies. For example, organoids may be cultured and tested according to the systems and methods disclosed in U.S. patent application Ser. No. 16/693,117, filed Nov. 22, 2019; U.S. Prov. Patent Application No. 62/924,621, filed Oct. 22, 2019; and U.S. Prov. Patent Application No. 62/944,292, filed Dec. 5, 2019, each of which is incorporated herein by reference and in its entirety for all purposes.

When the digital and laboratory health care platform further includes application of one or more of the above in combination with or as part of a medical device or a laboratory developed test that is generally targeted to medical care and research, such laboratory developed test or medical device results may be enhanced and personalized through the use of artificial intelligence. An example of laboratory developed tests, especially those that may be enhanced by artificial intelligence, is disclosed, for example, in U.S. Provisional Patent Application No. 62/924,515, filed Oct. 22, 2019, which is incorporated herein by reference and in its entirety for all purposes.

It should be understood that the examples given above are illustrative and do not limit the uses of the systems and methods described herein in combination with a digital and laboratory health care platform.

The results of the bioinformatics pipeline may be provided for report generation 208. Report generation may comprise variant science analysis, including the interpretation of variants (including somatic and germline variants as applicable) for pathogenic and biological significance. The variant science analysis may also estimate microsatellite instability (MSI) or tumor mutational burden. Targeted treatments may be identified based on gene, variant, and cancer type, for further consideration and review by the ordering physician. In some aspects, clinical trials may be identified for which the patient may be eligible, based on mutations, cancer type, and/or clinical history. Subsequent validation may occur, after which the report may be finalized for sign-out and delivery. In some embodiments, a first or second report may include additional data provided through a clinical dataflow 202, such as patient progress notes, pathology reports, imaging reports, and other relevant documents. Such clinical data is ingested, reviewed, and abstracted based on a predefined set of curation rules. The clinical data is then populated into the patient's clinical history timeline for report generation.

Further details on clinical report generation are disclosed in U.S. patent application Ser. No. 16/789,363 (PCT/US20/180002), filed Feb. 12, 2020, which is hereby incorporated herein by reference in its entirety.

### Specific Embodiments of the Disclosure.

In some aspects, the systems and methods disclosed herein may be used to support clinical decisions for personalized treatment of cancer. For example, in some embodiments, the methods described herein identify actionable genomic variants and/or genomic states with associated recommended cancer therapies. In some embodiments, the recommended treatment is dependent upon whether or not the subject has a particular actionable variant and/or genomic status. Recommended treatment modalities can be therapeutic drugs and/or assignment to one or more clinical trials. Generally, current treatment guidelines for various cancers are maintained by various organizations, including the National Cancer Institute and Merck & Co., in the Merck Manual.

In some embodiments, the methods described herein further includes assigning therapy and/or administering therapy to the subject based on the identification of an actionable genomic variant and/or genomic state, e.g., based on whether or not the subject's cancer will be responsive to a particular personalized cancer therapy regimen. For example, in some embodiments, when the subject's cancer is classified as having a first actionable variant and/or genomic state, the subject is assigned or administered a first personalized cancer therapy that is associated with the first actionable variant and/or genomic state, and when the subject's cancer is classified as having a second actionable variant and/or genomic state, the subject is assigned or administered a second personalized cancer therapy that is associated with the second actionable variant. Assignment or administration of a therapy or a clinical trial to a subject is thus tailored for treatment of the actionable variants and/or genomic states of the cancer patient.

### EXAMPLES

### Example 1 the Cancer Genome Atlas (TCGA)

The Cancer Genome Atlas (TCGA) is a publicly available dataset comprising more than two petabytes of genomic data for over 11,000 cancer patients, including clinical information about the cancer patients, metadata about the samples (e.g., the weight of a sample portion, etc.) collected from such patients, histopathology slide images from sample portions, and molecular information derived from the samples (e.g., mRNA/miRNA expression, protein expression, copy number, etc.). The TCGA dataset includes data on 33 different cancers: breast (breast ductal carcinoma, bread lobular carcinoma) central nervous system (glioblastoma multiforme, lower grade glioma), endocrine (adrenocortical carcinoma, papillary thyroid carcinoma, paraganglioma & phcochromocytoma), gastrointestinal (cholangiocarcinoma, colorectal adenocarcinoma, esophageal cancer, liver hepatocellular carcinoma, pancreatic ductal adenocarcinoma, and stomach cancer), gynecologic (cervical cancer, ovarian serous cystadenocarcinoma, uterine carcinosarcoma, and uterine corpus endometrial carcinoma), head and neck (head and neck squamous cell carcinoma, uveal melanoma), hematologic (acute myeloid leukemia, Thymoma), skin (cutaneous melanoma), soft tissue (sarcoma), thoracic (lung adenocarcinoma, lung squamous cell carcinoma, and mesothelioma), and urologic (chromophobe renal cell carcinoma, clear cell kidney carcinoma, papillary kidney carcinoma, prostate adenocarcinoma, testicular germ cell cancer, and urothelial bladder carcinoma).

### Example 2 Method of Validating a Liquid Biopsy Assay

Conducting sample collection, storage, nucleic acid isolation, and library preparation.

To validate a liquid biopsy assay in accordance with some embodiments of the present disclosure, 188 unique specimens were sequenced. These unique specimens included 10 blood specimens purchased from BioIVT, 56 residual plasma samples, 39 whole-blood samples, 4 cfDNA reference standards set in synthetic plasma (Horizon Discovery's Multiplex I cfDNA Reference Standards HD812, HD813, HD814, HD815), and 2 cfDNA reference standard isolates (Horizon Discovery's Structural Multiplex cfDNA reference standard HD786, and 100% Multiplex I Wild Type Reference Standard HD776). Furthermore, an additional 55 blood samples with matched tumor samples were utilized to compare the liquid biopsy and solid tumor tests, and 375 blood samples were sequenced for low-pass whole-genome sequencing (LPWGS) analysis. Sequence data from an additional 1,000 patient samples that were previously sequenced were utilized for retrospective and clinical analyses. All blood was received in Cell-free DNA BCT® blood collection tubes (Streck). Plasma was prepared immediately after accessioning and stored at −80° C. until later nucleic acid extraction and library preparation. At this time, cfDNA was isolated from plasma using the Qiagen QIAamp MinElute ccfDNA Midi Kit (QIAGEN), conducted according to instructions provided by the manufacturer. Automated library preparation was performed on a SciClone NGSx (Perkin Elmer). All cfDNA samples were normalized with molecular grade water to a maximum of 50 microliters (μL).

### Conducting the Liquid Biopsy Sequencing Assay.

The liquid biopsy assay utilized New England BioLab's NEBNext® Ultra™ II DNA Library Prep Kit for Illumina®, IDT's xGen CS Adapters, unique molecular indices (UMI), and 96 pairs of barcodes to prepare cfDNA sequencing libraries with unique sample identifiers (IDs). Each sample was ligated to a dual unique index. The dual unique index enables multiplexed sequencing of up to 7 patients and 1 positive control per SP NovaSeq flow cell, 16 patients and 1 positive control per S1 NovaSeq flow cell, 34 patients and 1 positive control per S2 NovaSeq flow cell, and 84 patients and 1 positive control per S4 NovaSeq flow cell. The library preparation protocol is optimized for greater than or equal to 20 nanograms (ng) cfDNA input to maximize mutation detection sensitivity. The final library was sequenced on an Illumina NovaSeq sequencer. Furthermore, analysis was performed using a bioinformatics pipeline and analysis server.

### The Bioinformatics Pipeline.

Adapter-trimmed FASTQ files are aligned to the nineteenth edition of the human reference genome build (hg19) using Burrows-Wheeler Aligner (BWA). Li et al., 2009, “Fast and accurate short read alignment with Burrows-Wheeler transform,” Bioinformatics, (25), pg. 1754. Following alignment, reads were grouped by alignment position and UMI family, and collapsed into consensus sequences using fgbio tools (available online at fulcrumgenomics.github.io/fgbio/). Bases with insufficient quality or significant disagreement among family members were reverted to N's. Phred scores were scaled based on initial base calling estimates combined across all family members. Following single-strand consensus sequence generation, duplex consensus sequences were generated by comparing the forward and reverse oriented PCR products with mirrored UMI sequences. Consensus sequences were re-aligned to the human reference genome using BWA. BAM files are generated and indexed after the re-alignment.

SNV and indel variants were detected using VarDict. Lai et al., 2016, “VarDict: a novel and versatile variant caller for next-generation sequencing in cancer research,” Nucleic Acids Res, (44), pg. 108. SNVs were called down to 0.1% VAF for specified hotspot target regions and 0.25% VAF at all other base positions across the panel. Indels were called down to 0.5% VAF for variants within specific regions of interest. Any indels outside of these regions were called down to 5% VAF. All SNVs and indels were then sorted, deduplicated, normalized, and annotated accordingly. Following annotation, variants were classified as germline, somatic, or uncertain using a Bayesian model based on prior expectations informed by various internal and external databases of germline and cancer variants. Uncertain variants are treated as somatic for filtering and reporting purposes. Following classification, variants were filtered based on a plurality of quality metrics including coverage, VAF, strand bias, and genomic complexity. Additionally, variants were filtered with a Bayesian tri-nucleotide context-based model with position level background error rates estimated from a pool of process matched healthy controls. Furthermore, known artifactual variants were removed.

Copy number variants (CNVs) were analyzed utilizing CNVkit and a CNV annotation and filtering algorithm provided by the present disclosure. Talevich et al., 2016, “CNVkit: Genome-Wide Copy Number Detection and Visualization from Targeted DNA Sequencing,” PLOS Comput Biol, (12), pg. 1004873. This CNVkit provides genomic region binning, coverage calculation, bias correction, normalization to a reference pool, segmentation, and visualization. The log 2 ratios between the tumor sample and a pool of process matched healthy samples from the CNVkit output were annotated and filtered using statistical models, such that the amplification status (e.g., amplified or not-amplified) of each gene is predicted and non-focal amplifications are removed.

Rearrangements were detected using the SpeedSeq analysis pipeline. Chiang et al., 2015, “SpeedSeq: ultra-fast personal genome analysis and interpretation,” Nat Methods, (12), pg. 966. Briefly, FASTQ files were aligned to hg19 using BWA. Split reads mapped to multiple positions and read pairs mapped to discordant positions were identified and separated, then utilized to detect gene rearrangements by LUMPY. Layer et al., 2014, “LUMPY: a probabilistic framework for structural variant discovery,” Genome Biol, (15), pg. 84. Fusions were then filtered according to the number of supporting reads.

Predicted functional effect and clinical interpretation for each variant was curated by automated software using information from both internal and external databases. A weighted-heuristic model was used, which has logic-based recommendations from the AMP/ASCO/CAP/ClinGen Somatic working group and ACMG guidelines. Li et al., 2017, “Standards and Guidelines for the Interpretation and Reporting of Sequence Variants in Cancer: A Joint Consensus Recommendation of the Association for Molecular Pathology, American Society of Clinical Oncology, and College of American Pathologists,” The Journal of molecular diagnostics, (19), pg. 4; Kalia et al., 2017, “Recommendations for reporting of secondary findings in clinical exome and genome sequencing, 2016 update (ACMG SF v2.0): a policy statement of the American College of Medical Genetics and Genomics,” Genetics in Medicine, (19), pg. 249.

The relative frequency and distribution are determined for any read containing repetitive sequences to detect microsatellite instability. To predict the probability of an unstable locus, a k-nearest neighbors model (with k=100) was utilized along with normalized percent lower, mean lower, and mean log-likelihood metrics. The percentage of unstable loci was calculated from the probabilities of each sample, with greater than 50% unstable loci considered microsatellite instability-high (MSI-H).

### The Validation Approach.

The present disclosure conducted extensive validation studies to establish robust technical perform of the liquid biopsy assay. Limit of detection (LOD) was determined by assessing analytical sensitivity in reference standards with 5%, 1%, 0.5%, 0.25%, and 0.1% VAF generated from the Horizon Discovery reference set. The Horizon Discovery set includes 160 bp cfDNA fragments from human cell lines in an artificial plasma matrix to closely resemble cfDNA extracted from human plasma. VAFs of SNVs and indels, including EGFR (ΔE746-A750), EGFR (V769-D770insASV), EGFR A767_V769dup, EGFR (L858R), EGFR (T790M), KRAS (G12D), NRAS (A59T), NRAS (Q61K), AKT1 E17K, PIK3CA (E545K), and GNA11 Q209L, and CNVs and rearrangements, including CCDC6/RET, SLC34A2/ROS1, MET, MYC, and MYCN, were measured in reference samples by the liquid biopsy assay of the present disclosure. Each measurement was conducted with a minimum of three replicates at 10 ng, 30 ng, and 50 ng of DNA. Sensitivity was determined by the number of detected variants divided by the total number of variants present in the reference samples. Samples with an on-target rate of less than 30% were excluded from the instant analysis, and MET (4.5 copies) was included in CNV sensitivity determinations. Sensitivity of greater than 90% was considered reliable detection.

Analytical specificity was determined using 44 normal samples titrated at 1%, 2.5%, or 5% from a wild-type cfDNA reference standard with a list of confirmed true-negative SNVs, indels, CNVs and rearrangements. Specificity was determined by the number of known true-negative variants divided by the number of true-negative variants plus false-positive variants identified by the liquid biopsy assay.

To assess inter-instrument concordance between the sequencing instruments, 10 patient libraries were sequenced on each instrument (3 NovaSeqs). Variants seen below the lower limit of detection (LLOD) (0.25% for SNVs and 0.50% for indels) were excluded from concordance analysis.

To establish analytical accuracy, the results of 40 validation samples were compared to the results of an orthogonal reference method (Roche's AVENIO ctDNA assay). Analytical accuracy was determined by the number of detected variants divided by the total number of variants present in the sample. Variants that were off-target or below LLOD (0.25% for SNVs and 0.5% for indels) were excluded from the instant analysis.

Conducting Digital Droplet Polymerase Chain Reaction (ddPCR).

Five variants were validated on the ddPCR platform: KRAS G12D (Integrated DNA Technologies, IDT, published sequences); TERT promoter mutations c.-124C>T (C228T) & c.-146C>T (C250T) (Thermo Fisher Scientific); and TP53 p.R273H and TP53 p.R175H (Thermo Fisher Scientific). Each amplification reaction was performed in 25 μL and contained 1× Genotyping Master Mix (Thermo Fisher Scientific), 1× droplet stabilizer (RainDance), 1× of primer/probe mixture for TERT and TP53 (for KRAS: 800 nM of each primer and 500 nM of each probe) plus template. To improve the lower limit of detection, 4-cycle amplification was conducted prior to droplet generation. Amplification for KRAS was conducted using the cycling conditions of: 1 cycle of 95° C. (0.6° C./s ramp) for 10 minutes, 4 cycles of 95° C. (0.6° C./s ramp) for 15 seconds and 60° C. for 2 minutes, followed by 1 cycle of 98° C. (0.6° C./s ramp) for 10 minutes. Cycling conditions for the TP53 variants were the same as those for KRAS with the exception of the annealing and extension temperature, which was set at 55° C. for 2 minutes. Amplification for TERT followed Thermo Fisher's recommendation as follows: 1 cycle of 96° C. (1.6° C./s ramp) for 10 minutes, 4 cycles of 98° C. (1.6° C./s ramp) for 30 seconds and 55° C. for 2 minutes, followed by 1 cycle of 55° C. (1.6° C./s ramp) for 2 minutes. Accordingly, droplets generated on the RainDance Source, and amplification performed following the above cycling conditions with cycle numbers of 45 for both KRAS and TP53, and 54 for TERT. Furthermore, droplets were analyzed on a RainDance Sense droplet reader. Additionally, RainDrop Analyst II v1.1.0 analysis software was utilized to acquire and analyze data.

### The Concordance Between Liquid Biopsy and Solid Tumor Assays.

Matched liquid biopsy and solid tumor sample pairs (n=55) were used to determine analytical sensitivity and specificity. Solid tumor and matched normal samples obtained from peripheral blood buffy coat were analyzed with the solid tumor assay, and corresponding blood plasma samples were analyzed with the liquid biopsy assay of the present disclosure. Only variants in the reportable range of both the solid tumor and liquid biopsy panels were included in these analyses (e.g., genes in the liquid biopsy gene panel is a subset of genes in the solid tumor gene panel). Germline, intronic, and synonymous variants identified in the solid tumor assay and the liquid biopsy assay were excluded from analysis with the exception of intronic splice variants. To determine analytical sensitivity, the number of variants called in both the liquid biopsy assay and the solid tumor assay (e.g., true positives) was divided by the sum of true positives and those called only in the solid tumor assay. To determine analytical specificity the number of positions reported in neither the liquid biopsy assay nor the solid tumor assay (e.g., true negatives) was divided by the sum of true negatives and variants only called in the liquid biopsy assay.

To improve variant calling in the liquid biopsy assay, a strategy that dynamically determines local sequence errors using Bayes Theorem and the likelihood ratio test was developed. The dynamic threshold was determined using a sample-specific error rate, the error rate from healthy control samples, and from a reference cohort of solid tumor samples. Accordingly, the method of the present disclosure was conducted on 55 matched liquid biopsy/solid tumor tissue samples, with variants detected in the solid tumor assay as the source of truth. Using sensitivity thresholds defined by the LOD analysis, fixed post-test-odds (e.g., equal to the P (post-test)/[1−P (post-test)]), as well as pre-test-odds. The Pre-test-odds were determined using historical data from the solid tumor assay with an equation identical to the post-test-odds calculation). Accordingly, the following formula was determined based on the above: specificity=1−pre-test-odds*sensitivity/post-test-odds

The specificity was input to a beta-binomial function and yielded the minimum number of alternate alleles to call a variant at a particular depth. The pre-test-odds metric was specific to individual cancer cohorts and individual genes, allowing for cancer-specific pre-test-odds to be applied to individual exons.

### Conducting Low-Pass Whole Genome Sequencing and Analysis.

Blood samples from 375 patients were sequenced using low-pass whole-genome sequencing (LPWGS) across four flow cells. Sequencing coverage metrics for these samples were determined using Picard CollectWgsMetrics. The tumor fraction and ploidy values for each sample were estimated using ichorCNA with a specific reference panel of 47 normal samples. Adalsteinsson et al., (2017), “Scalable whole-exome sequencing of cell-free DNA reveals high concordance with metastatic tumors” Nat Commun, (8), pg. 1324. Reported variants from the corresponding liquid biopsy analysis of each sample were utilized to assess the accuracy of the tumor fraction estimates.

### Determining Estimation of Circulating Tumor Fraction.

Circulating tumor fraction estimate (ctFE) was determined using a novel method, Off-Target Tumor Estimation Routine (OTTER), from off-target reads uniformly distributed across the human reference genome. As described above, the CNVkit was conducted on each sample, and segments were assigned via circular binary segmentation (CBS). Olshen et al., 2004, “Circular binary segmentation for the analysis of array-based DNA copy number data,” Biostatistics, (5), pg. 557. Segments were then fit to integer copy states via an expectation-maximization algorithm using the sum of squared error of the segment log 2 ratios (e.g., normalized to genomic interval size) to expected ratios given a putative copy state and tumor purity. Estimates were confirmed by comparing results against LPWGS of the original patient isolate. As such, results are shown using randomly selected, de-identified samples.

### Clinical Profiling of Liquid Biopsy Patients.

De-identified molecular and abstracted clinical data were evaluated in a cohort of 1,000 patients randomly selected from a specific reference clinicogenomic database. All data were de-identified in accordance with the Health Insurance Portability and Accountability Act (HIPAA). Dates used for analyses were relative to the first liquid biopsy sequencing date of each patient, and year of the first sequencing date was randomly off-set. Variants included in the analyses were those classified as pathogenic or likely pathogenic, and further divided into actionable if matched to diagnostic, prognostic or therapeutic evidence or biologically relevant. Outcomes were determined according to the most recent clinical response noted in patient records. The study protocol was submitted to the Advarra Institutional Review Board (IRB), which determined the research was exempt from IRB oversight and approved a waiver of HIPAA authorization for this study.

### Example 3—Development of a Random Forest Model for Detecting Clonal Hematopoiesis Variants and/or Solid Variants in a Liquid Biopsy Assay

Clonal Hematopoiesis of Indeterminate Potential (CHIP, or CH) is a well-established confounder in next-generation sequencing (NGS)-based liquid biopsy cancer diagnostics. Misclassification of CH as tumor variants can lead to false positive actionable variant detection, potentially resulting in incorrect interpretation of results and therapy selection. Moreover, CH variants may also interfere with quantitative variant monitoring leading to inaccurate assessment of treatment response. While filtering of CH is possible via matched sequencing of white blood cell and plasma DNA, emerging algorithmic approaches may enable a more resource-effective, time-sensitive approach with high precision.

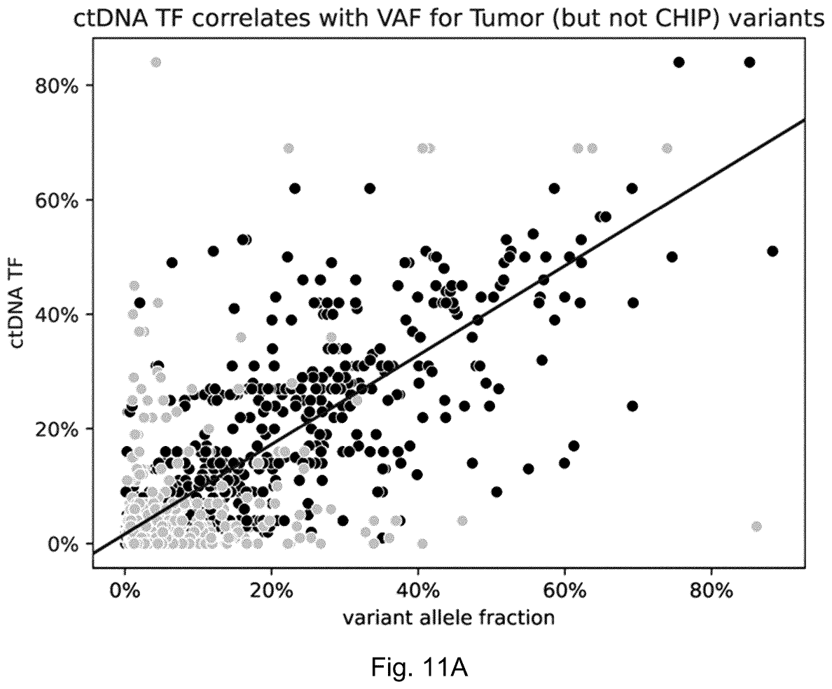

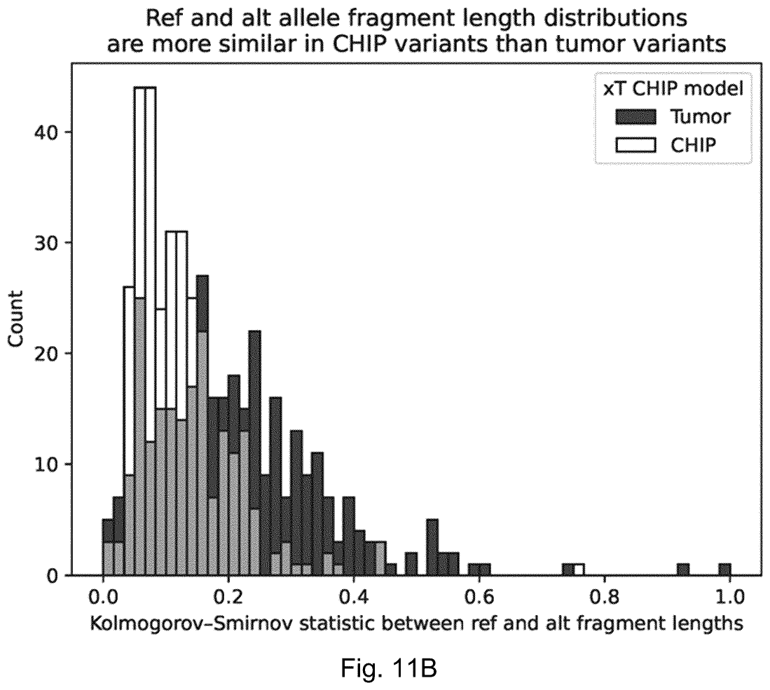

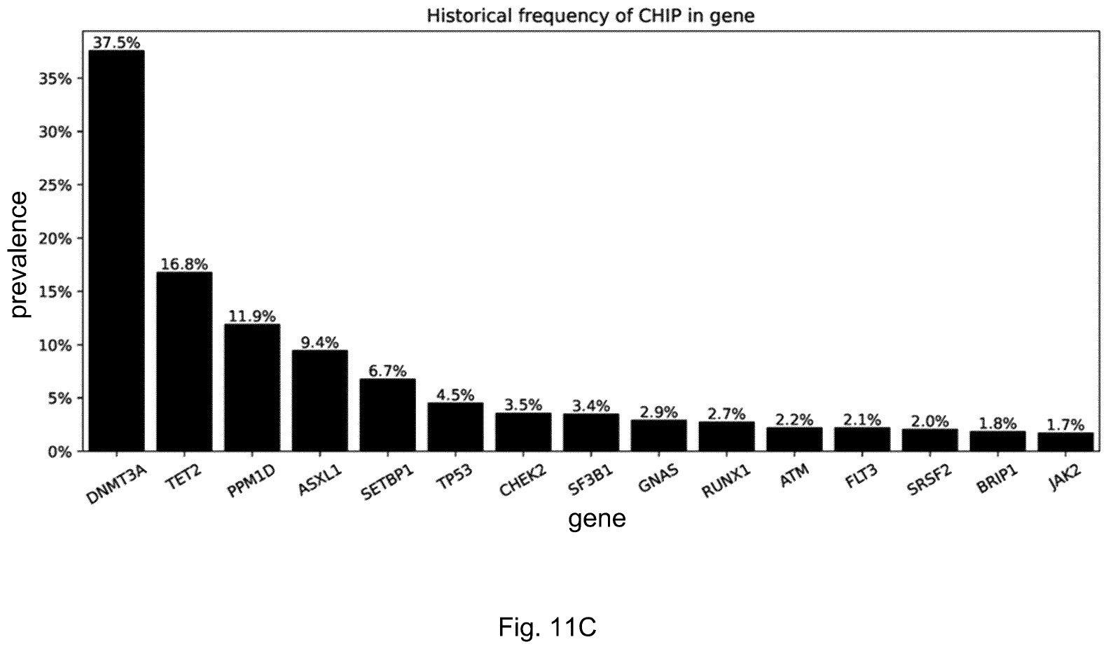

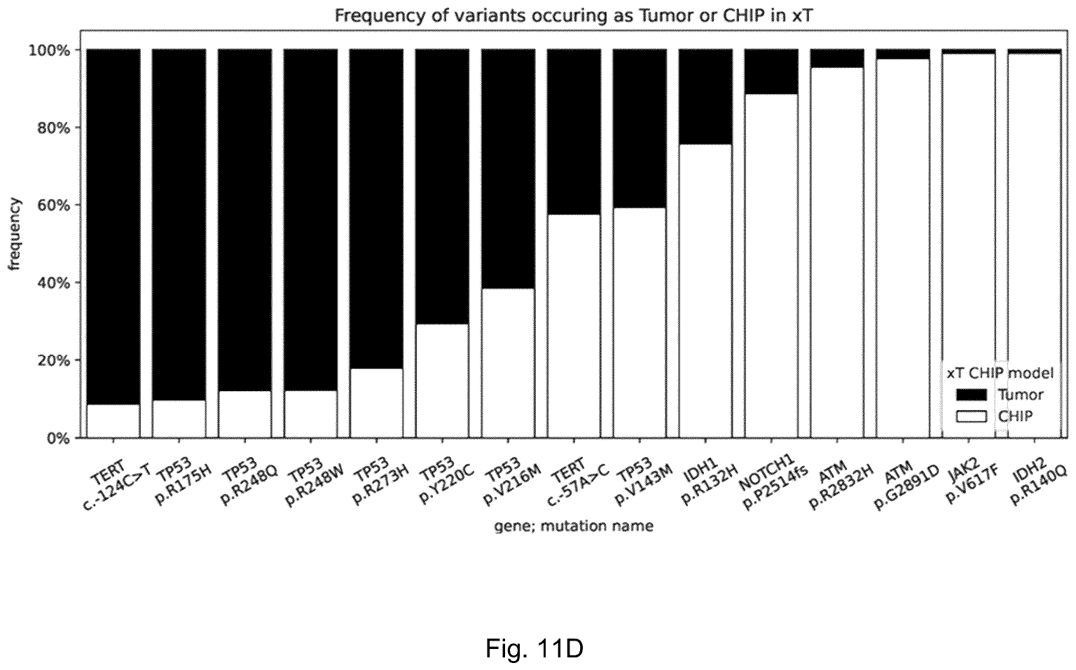

Multiple features available within conventional liquid biopsy assays strongly associate with CH or tumor origin, but no feature alone conclusively identifies CH variants. For example, as illustrated in FIG. 11A, ctDNA tumor fraction estimate has a strong correlation with the variant allele fraction of variants derived from ctDNA, while CH variants do not display this correlation. Likewise, as illustrated in FIG. 11B, DNA fragments containing circulating tumor variants have a distinct distribution of fragment lengths relative to cfDNA fragments originating from non-tumor cells. This has been previously used for tumor/CH discrimination in Marass et al., 2020, Fragment Size Analysis May Distinguish Clonal Hematopoiesis from Tumor-Derived Mutations in Cell-Free DNA, Clin. Chem. April 1; 66 (4): 616-18.

Moreover, it was noticed in historical sequencing data from matched solid tumor-buffy coat sequencing assays, that certain genes had a higher prevalence of accumulating CHIP variants than other genes. The historical CH prevalence in such genes is illustrated in FIG. 11C. The historical frequency of CH variants, relative to somatic variants, in such genes is illustrated in FIG. 11D.

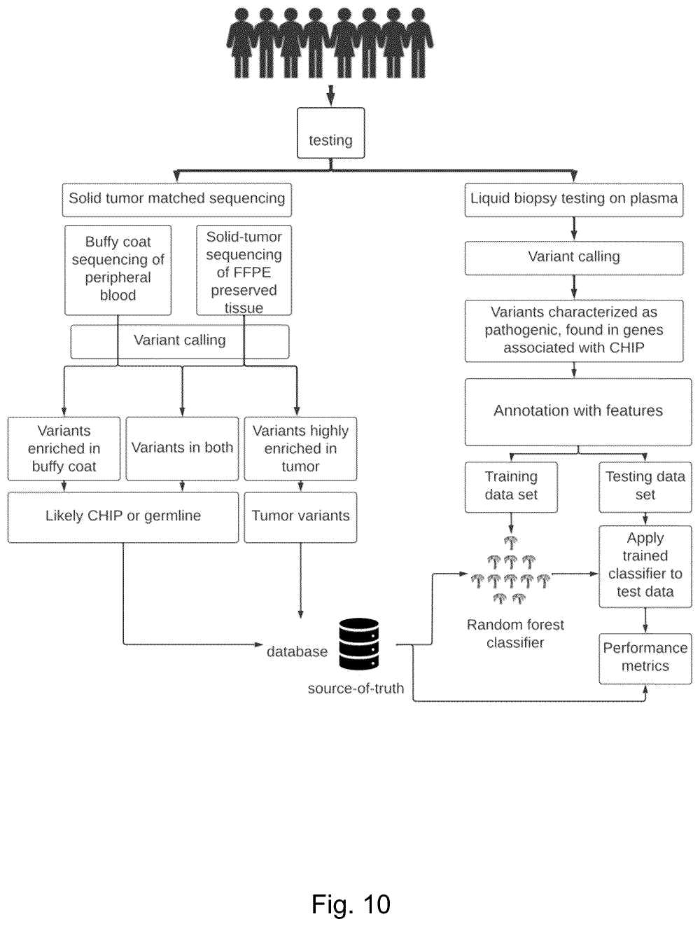

This example describes validation of the random forest algorithm trained in Example 3 for classifying CHIP sequence variants, on multiple orthogonal bioinformatics features. The results show that the model can reliably distinguish CH from tumor-derived variants using only liquid biopsy data. An ensemble approach using multiple independent features enables high performance. The classifier achieves high accuracy, including high sensitivity and high specificity. Data flow for the model training and validation is illustrated in FIG. 10.

A random forest classifier was trained and validated on 1321 advanced, pan-solid tumor cancer samples (training n=660, validation n=661) sequenced using both a liquid biopsy assay and a solid tumor with matched buffy coat assay. Variants were labeled as CH or tumor-derived based on solid-tissue results in 39 genes that are known to be associated with CH (e.g., DNMT3A, TET2, TP53). The classifier was trained to classify SNV and indel variants detected via liquid biopsy as circulating-tumor or non-tumor (CH+germline) in origin. Features used by the classifier include the fragment size of reads overlapping each variant, prevalence in solid-tumor samples from a proprietary multimodal database, and variant allele fraction relative to estimated tumor fraction. Model classifications were validated against an established commercial solid tumor biopsy assay.

This model was developed for algorithmic improvements for plasma-only CHIP calling, e.g., without using a paired plasma/buffy-coat preparation. A random forest classifier was trained using features obtained from fragment size of reads overlapping with variants, historical CHIP prevalence in data acquired from matching liquid biopsy, solid tumor, and germline tissue samples, and variant allele fraction in relation to estimated tumor fraction. It was tested based on classifications made using data from the matched tumor and germline tissue.

Examples of matched solid tumor and germline tissue genomic profiling assays are described in Beaubier et al., 2019, “Integrated genomic profiling expands clinical options for patients with cancer,” Nat Biotechnol., 37 (11): 1351-1360 and Beaubier et al., 2019, “Clinical validation of the Tempus xT next-generation targeted oncology sequencing assay,” Oncotarget., 10 (24): 2384-2396, the disclosure of which are incorporated herein by reference, in their entireties, for all purposes. Examples of liquid biopsy genomic profiling assays are described in Finkle et al., 2021, “Validation of a liquid biopsy assay with molecular and clinical profiling of circulating tumor DNA,” NPJ Precis Oncol., 5 (1): 63, the disclosure of which is incorporated herein by reference, in its entirety, for all purposes.

### Feature Details

The random forest model described in this example was trained using 10 features: gene frequency, alternate allele fragment length median, CHIP likelihood, Kolmogorov-Smirnov test metric for fragment length, p-value for Kolmogorov-Smirnov test metric for fragment length, alternate allele fragment length kurtosis, estimated circulating tumor fraction, ensemble variant allele fraction residual, alternate allele fragment length skew, and variant allele fraction.

Gene Frequency: An analysis of CHIP prevalence in sequencing reactions from solid tumor samples was performed. All variants identified by the solid tumor CHIP algorithm were collated, and used to estimate the prevalence of each individual variant and gene. To encode gene as a single feature in the random forest, this data was used to provide a quantitative value for each gene based on absolute presence in the solid tumor samples. The solid tumor prevalence assessment found that 37% of patients with CHIP called in a solid tumor fraction had a variant in DNMT3A, such that DNMT3A was encoded as 0.37. Genes that were not identified as sources of CHIP in solid tumor samples were encoded as 0. This was the most informative feature in the model.

Alternate Allele Fragment Length Median: This metric was determined as the median value for the fragment length of all cfDNA fragments sequenced in the liquid biopsy assay that include the candidate somatic variant.

CHIP Likelihood: This metric was determined for a particular variant based on sequencing results for a cohort of solid tumor samples with matched liquid biopsy samples. The metric is calculated as the total number of occurrences of the variant in the solid tumor samples that were classified as somatic divided by the total number of occurrences of the variant in the solid tumor samples that were classified as either somatic or non-somatic (e.g., either of germline or CHIP lineage).

Kolmogorov-Smirnov test metric for fragment length: This metric was determined as the Kolmogorov-Smirnov test statistic for the comparison of (i) the distribution of fragments lengths for cfDNA containing the candidate somatic variant in the liquid biopsy sample to (ii) the distribution of fragments lengths for cfDNA that don't contain the candidate somatic variant in the liquid biopsy sample.

p-value for Kolmogorov-Smirnov test metric for fragment length: This metric was determined as the p-value for the Kolmogorov-Smirnov test statistic for the comparison of (i) the distribution of fragments lengths for cfDNA containing the candidate somatic variant in the liquid biopsy sample to (ii) the distribution of fragments lengths for cfDNA that don't contain the candidate somatic variant in the liquid biopsy sample.

Alternate allele fragment length kurtosis: This metric is a kurtosis value for the distribution of fragments lengths for cfDNA containing the candidate somatic variant in the liquid biopsy sample. Further details of kurtosis are found in conjunction with block 532.

Estimated circulating tumor fraction (ctFE): This metric represents the fraction of all cfDNA fragments in a liquid biopsy sample that are derived from a cancerous cell (of somatic lineage). Example methods for estimating circulating tumor fraction as described above in the section titled “Circulating Tumor Fraction.”

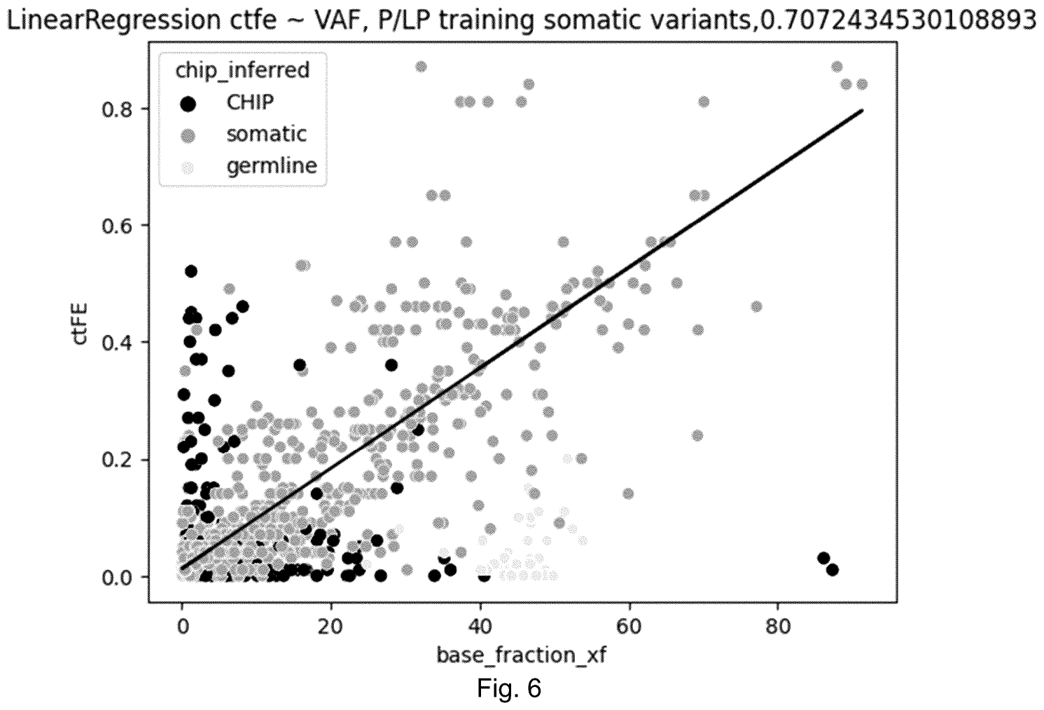

Ensemble variant allele fraction residual: This metric is determined by inputting the ctFE into a trained linear regression model of the relationship between circulating tumor fractions of liquid biopsies and variant allele fractions of somatic variants in the liquid biopsies to obtain as output from the trained linear regression model an expected variant allele fraction and comparing (i) the expected variant allele fraction with (ii) the variant allele fraction for the candidate somatic variant. CHIP can occur in patients with any level of circulating tumor, and at a wide range of base-fractions. However, it is not expected to correlate with tumor fraction. True somatic variants (classified based on the respective tumor and normal VAFs in solid tumor matched data for liquid biopsy samples) have a strong correlation with tumor fraction, as shown in FIG. 6. To capture this as a model feature, a linear regression (ensemble ctFE˜liquid biopsy variant allele frequency) was performed on somatic pathogenic variants in the training set (FIG. 6). This regression model was saved and applied to the full training and testing datasets. The absolute value of the residuals on this regression were saved as a feature.

Alternate allele fragment length skew: This metric is a skew value for the distribution of fragments lengths for cfDNA containing the candidate somatic variant in the liquid biopsy sample. Further details of skew are found in conjunction with block 534.

Variant Allele Fraction (VAF): This metric represents the fraction of all cfDNA fragments in a liquid biopsy sample encompassing the locus for the candidate somatic variant that contain the candidate somatic variant in the liquid biopsy sample.

Age was not included as a feature in this model. However, age was highly correlated with CH. CH+ patients in training had a median age of 72, while CH-patients had a median age of 65 (p-value 1.1e-10 on t-test).

### Ground Truth Determination

Ground truths, e.g., variant calls as somatic, CHIP, or germline, were established for model training and testing using sequencing data for matched liquid biopsy, solid tumor, and germline samples. Tumor variants were detected in the liquid biopsy sample and detected in the matched solid tumor sample at a VAF of 6× or greater than in the matched germline tissue sample.

Non-tumor variants were defined as variants that were detected in the liquid biopsy sample, and detected in the matched germline tissue sample at a similar or higher VAF than in the matched solid tumor sample. These were further subdivided into likely germline or likely CHIP. Likely CHIP variants had a base fraction in the germline tissue sample lower than 25% or were identified as CHIP by a matched solid tumor and germline tissue CHIP algorithm, as described in Sonnenschein et al., 2022, “Novel Associations between Clonal Hematopoiesis and Therapeutic Exposures Revealed in Patients with Solid Tumors Using Real World Evidence,” Blood 140 (Supplement 1): 2879-2880, the content of which is disclosed herein by reference in its entirety for all purposes. Likely germline variants had a base fraction of equal to or greater than 25% in the germline tissue sample and were not identified as CHIP by the matched solid tumor and germline tissue CHIP algorithm.

### Training and Testing Characteristics

The training and testing sets consist of 660 and 661 unique liquid biopsy analyses. These were reduced to pathogenic (P) and likely pathogenic (LP) variants. Table 4 shows a breakdown of somatic filtered variants and their CHIP inferred status based on sequencing of matched solid tumor and germline tissue. CHIP and germline variants were binned together as ‘positive’ calls.

TABLE 4
CHIP, germline and somatic breakdown for
variants in training and testing data.

| | | | | | # somatic |
|---|---|---|---|---|---|
| | Variant type | | | # | P/LP |
| | (based on | | # | somatic | variants in |
| | matched solid | | somatic | P/LP | CHIP |
| | tumor and | | variants | variants | genes |
| | germline | Training | (liquid | (liquid | (liquid |
| Group | analysis) | label | biopsy) | biopsy) | biopsy) |
| Train | CHIP | Positive | 1548 | 398 | 317 |
| Train | germline | | 2837 | 71 | 21 |
| Train | somatic | Negative | 3313 | 1024 | 342 |
| Test | CHIP | Positive | 1520 | 349 | 273 |
| Test | germline | | 2835 | 54 | 18 |
| Test | somatic | Negative | 2923 | 956 | 309 |

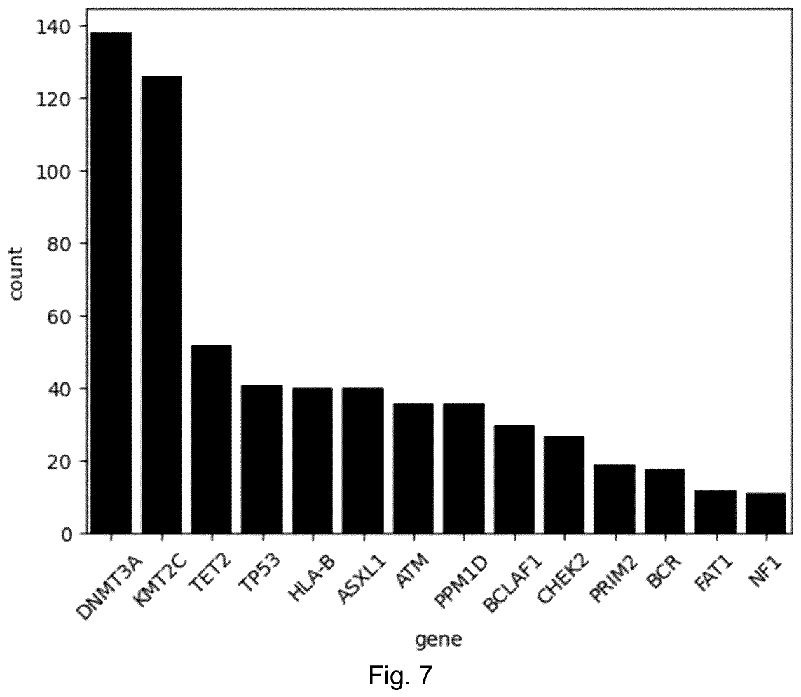

The frequency of likely CHIP variants called in this dataset was surprisingly high. Looking at the gene distribution for CHIP inferred variants in the training set, variants in KMT2C and HLA-B jump out as outliers relative to expected CHIP genes (FIG. 7). To avoid unknown phenomena impacting the model training, the dataset was further reduced to variants in canonical CHIP genes. In canonical CHIP genes, germline variants make up a very small percentage of P/LP variants. CHIP is clearly the main confounder for somatic variant calling in these genes.

In canonical CHIP genes, likely germline variants make up a small percentage of the pathogenic variants. However, as CHIP variants have very similar base fractions in plasma and buffy coat, and high VAF CHIP is not infrequent, especially in a few specific variants, in a plasma vs buffy coat validation there may be no way to distinguish high VAF and germline variants.

In matched solid tumor and buffy-coat samples, 10% of the somatic variant identified in the solid tumor sample are found in the buffy coat at trace quantities (below 1% base fraction). A ratio of 6× is applied for somatic variant calling in this case. Variants found in both tumor and buffy coat, but at much higher (e.g., at least 6×) fractions in tumor, are presumed tumor in origin. Around 1% of somatic variants identified in a solid tumor sample show more significant contamination in the normal (above 1% base fraction). This trace-to-moderate contamination is relatively low risk in a solid tumor assay, as the variants are typically enriched in the solid tumor at base fractions far greater than 6%. Liquid biopsy variant calling occurs at much lower base fractions. A 1% ct-dna contamination of buffy coat will lead to a far higher percentage of somatic variants being incorrectly subtracted in a plasma:buffy coat comparison.

### Model Performance

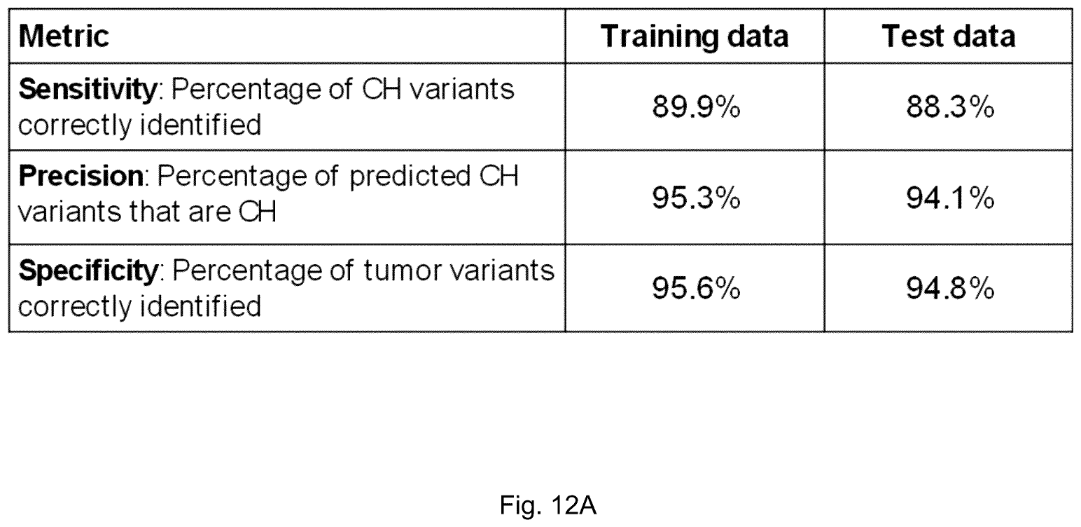

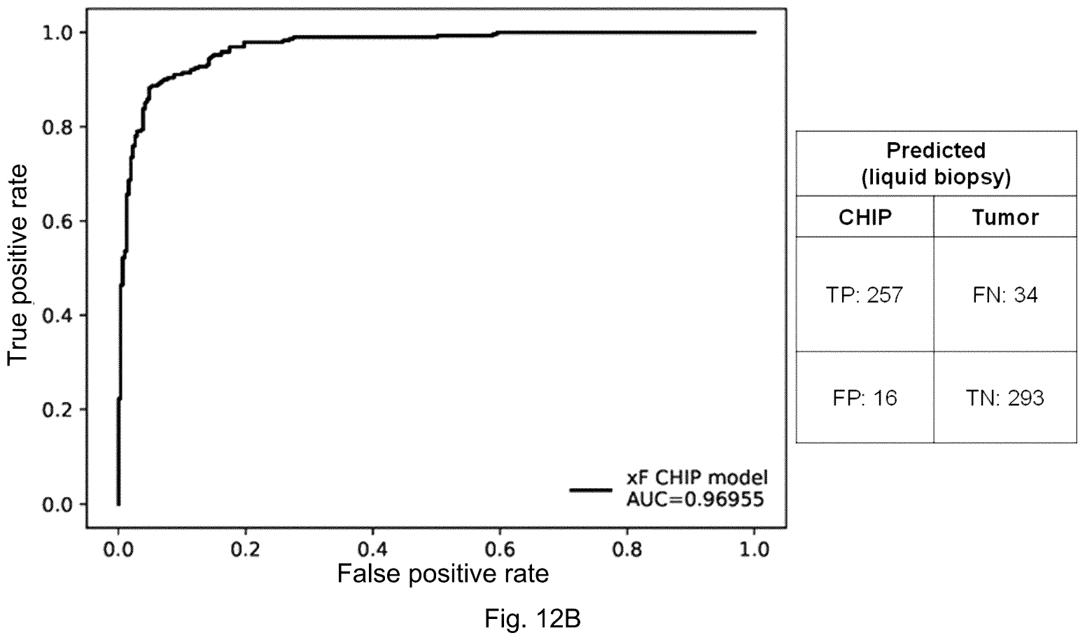

The training set (n=660 liquid biopsy samples) included 680 pathogenic variants. 50% (n=342/680) were determined to be tumor-derived, while 50% (338/680) are likely due to CH. The independent validation (n=661 samples) included 600 pathogenic variants. Model prediction accuracy on these validation set variants was 91.7%, with an ROC-AUC of 0.97. Sensitivity was 88.3% (n=257/291 true positives correctly labeled as non-tumor variants), specificity was 94.8% (n=293/309 true negatives correctly labeled as tumor variants), and precision was 94.1%. Performance data for the model, against both the training data and the independent validation (test) set, is shown in FIG. 12A. An ROC curve for the performance is illustrated in FIG. 12B.

Performance statistics for the trained random forest model, using a cutoff of 0.5 (below 0.5 probability is classified as tumor, above 0.5 as non-tumor), are shown in Table 5, below. In the table, the performance of the model is compared to a conventional assay control in which buffy coat sequencing is used to filter out CHIP variants.

TABLE 5
Variant level performance metrics for the random forest model
as compared to a convention matched buffy-coat assay.

| Metric | Random Forest Model | Conventional Control |
|---|---|---|
| ROC AUC | 0.968 | 0.92 |
| Accuracy | 91.7% | 88.1% |
| Sensitivity/PPA | 88.3% | 93.4% |
| Precision/PPV | 94.1% | 81.9% |
| Specificity | 94.8% | 81.5% |
| TP | 257 | 310 |
| FN | 34 | 15 |
| FP | 16 | 70 |
| TN | 293 | 318 |

Positives were defined as ‘non-tumor’ pathogenic or likely pathogenic variants in CHIP genes. These were determined to be non-tumor based on matched solid tumor and germline sample analysis for the same patient. See source of truth section. Negatives were tumor-originating pathogenic or likely pathogenic variants in CHIP genes. These were determined to be ‘tumor’ based on matched solid tumor and germline sample analysis for the same patient. If the base fraction in the solid tumor sample was >6× the base fraction in the matched normal sample, the variant was classified as tumor.

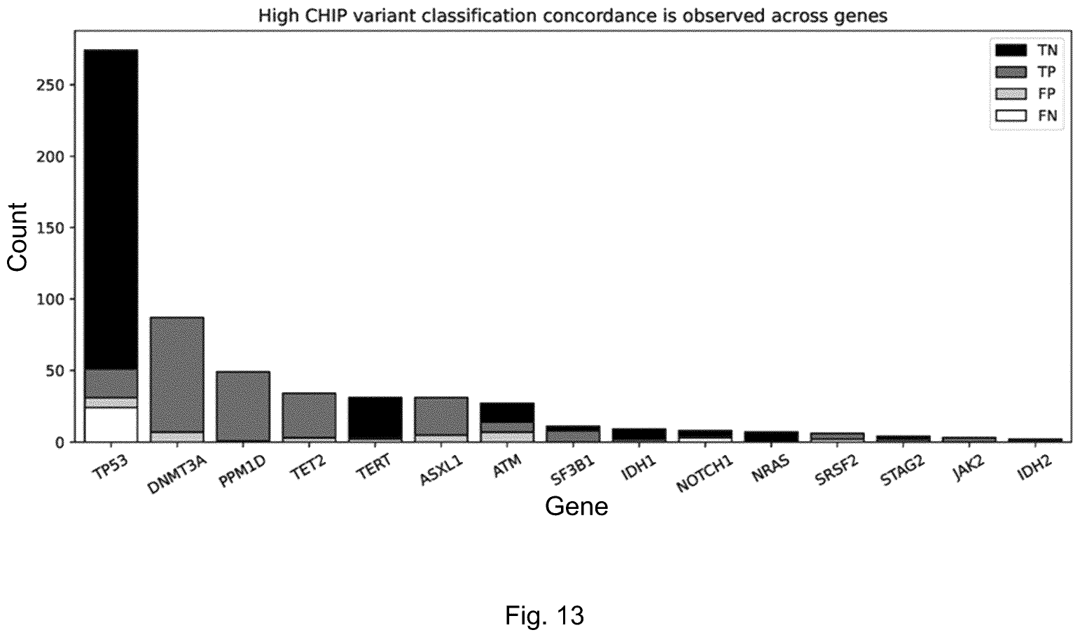

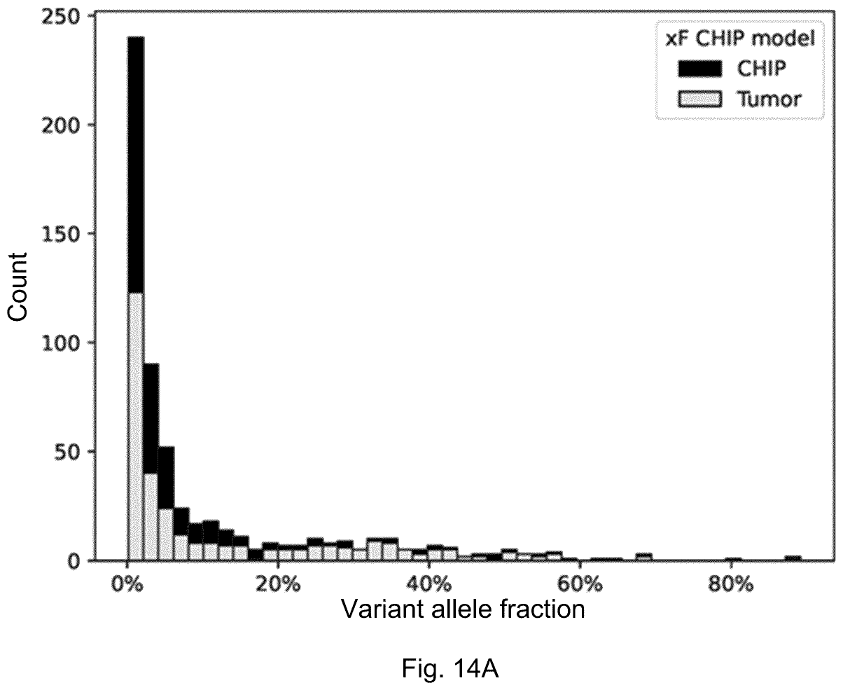

Performance of the classifier on the test set by gene is illustrated in FIG. 13. Both CH and tumor variants were seen and identified with high accuracy in many genes.

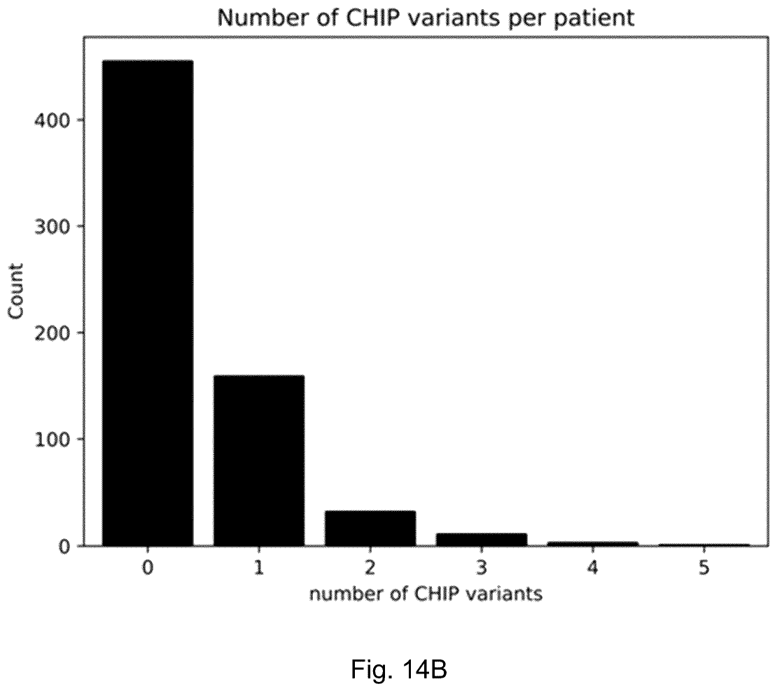

Allele fractions for CH and tumor variants were similar, as called by the classifier. Specifically, showing a median VAF of 2.8% for CH and 4.4% for tumor, as illustrated in FIG. 14A. Although no steps were taken to exclude germline variants, their prevalence in this cohort (pathogenic variants in known CH genes) appears to be small relative to true CH.

FIG. 14B illustrates the number of CH variants identified per patient. A total of 30% of the patients used in training and testing had an identifiable CH variant. Most patients with an identified CH variant had only one variant, although a minority had multiple CH variants.

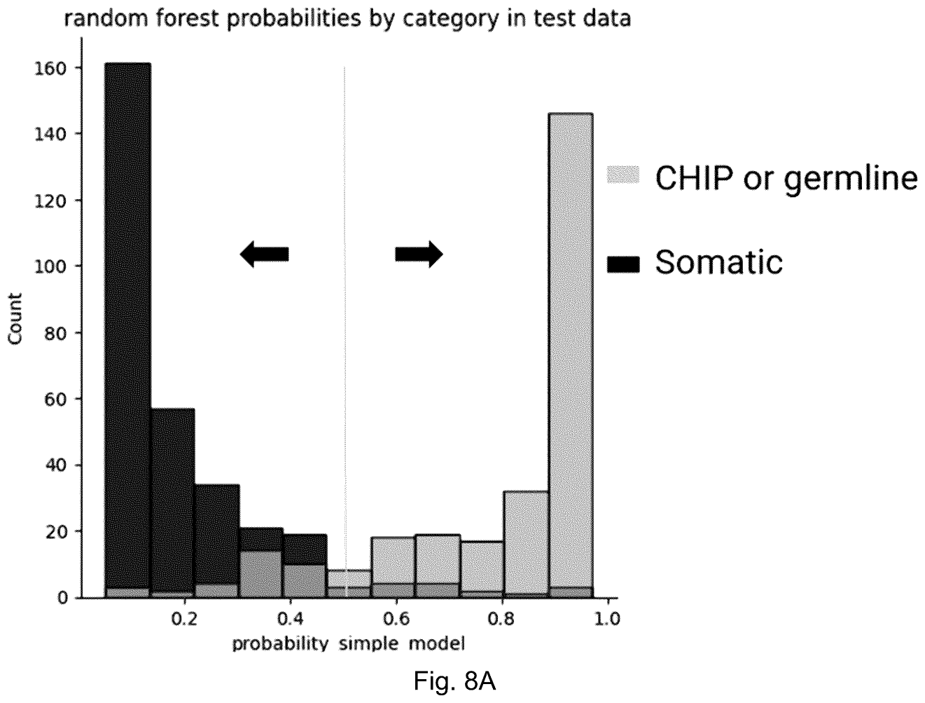

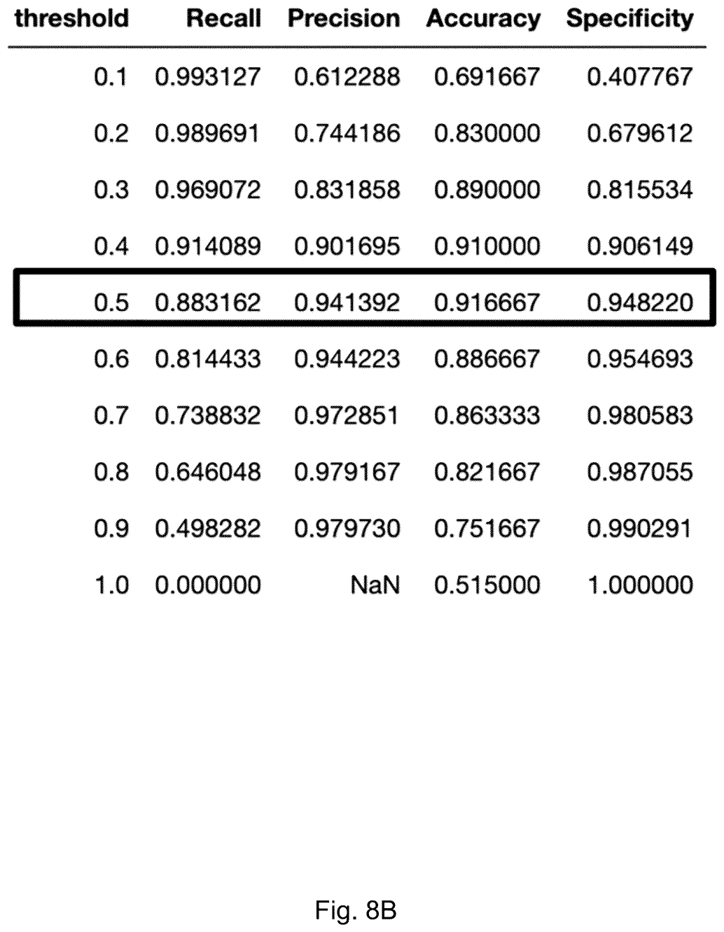

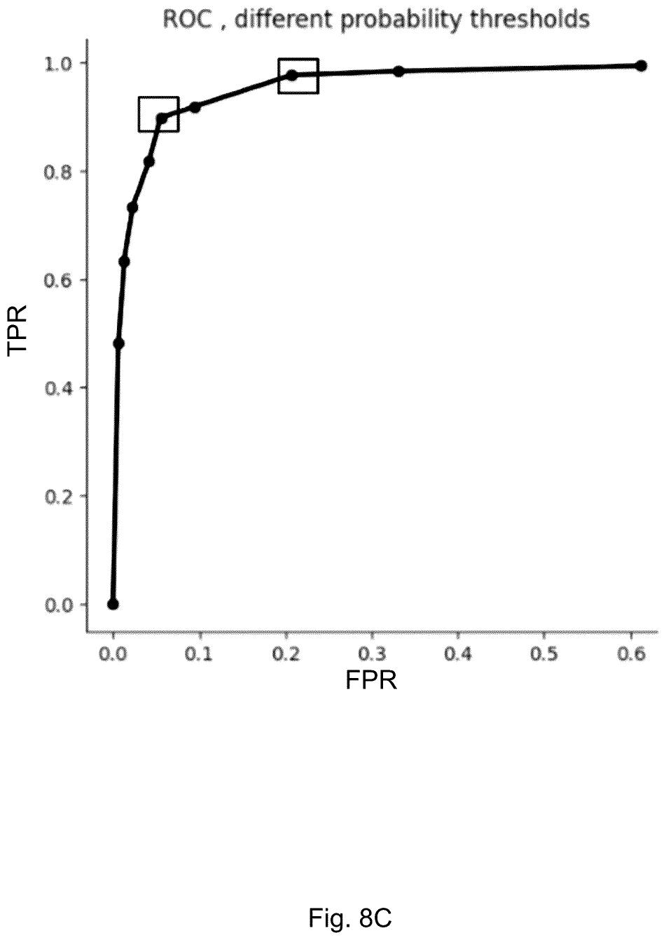

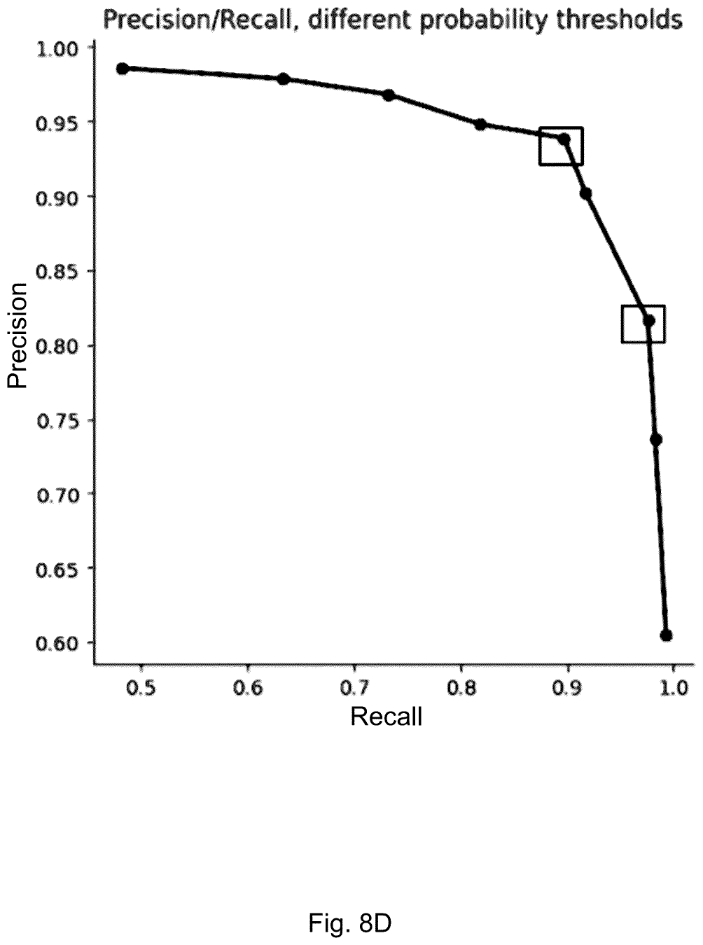

Random forest classification outputs probabilities for each variant classified by the model. FP/FN classifications were heavily enriched at mid-range probabilities (0.4-0.6). SN/SP were extremely high for classifications with high confidence (0.1 or 0.9), as shown in FIGS. 8A-8D. Using a cutoff of 0.4 brings all performance metrics above 90% in this test data, as shown in FIG. 8B.

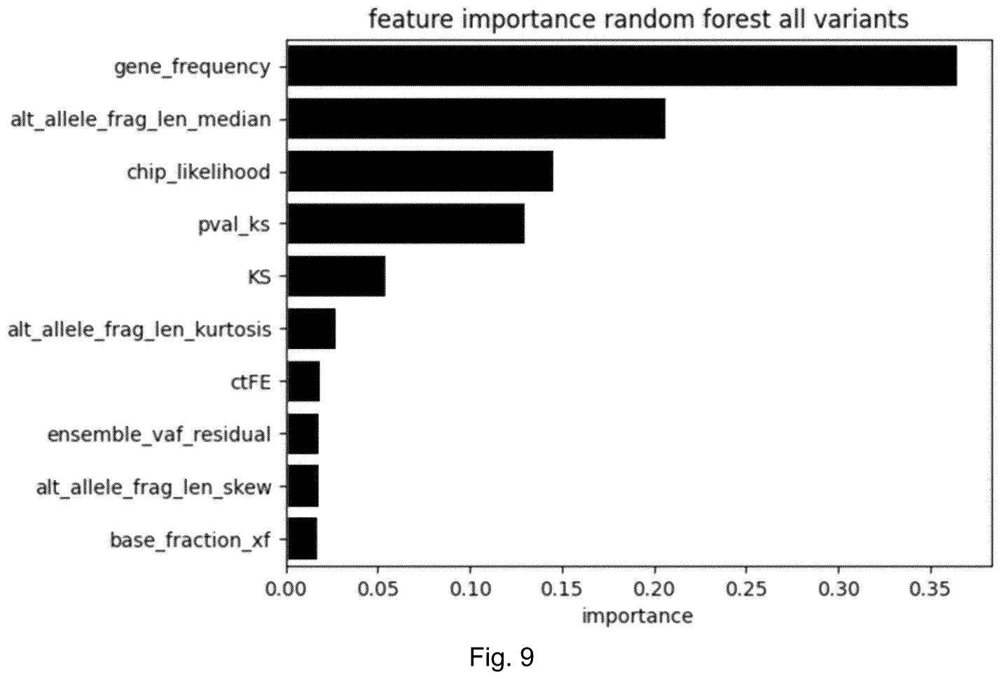

Feature analysis was performed based on decrease in Gini coefficient for each feature in the model. As shown in FIG. 9, the gene frequency, alternate allele fragment length median, CHIP likelihood, and p-value for Kolmogorov-Smirnov test metric for fragment length features were the most informative in the model.

The ensemble model described in this example is highly performant at distinguishing variants derived from CH versus ctDNA, approaching accuracy previously only seen in matched or tumor-informed assays. Notably, this cohort (pathogenic variants in genes known to be associated with CH) excludes most germline variants and is naturally close to balanced between CH and tumor categories. A model for broadly distinguishing tumor and non-tumor across all genes may favor different design. Although CH has many strongly characteristic features (association with DNMT3A, fragment size consistent with germline), it can present in diverse ways. Thus, accurate identification requires a multimodal approach.

### Example 4—Development of a Model for Validating a Somatic Sequence Variant of a Test Subject Using a Liquid Biopsy Assay Sample from the Test Subject

This example describes a method of training a model that can validate a somatic sequence variant in a tissue of a test subject. A corresponding nucleic acid sequence of each cell-free DNA (cfDNA) fragment in a plurality of cfDNA fragments is obtained from a plurality of sequence reads of a sequencing reaction of a plurality of cfDNA fragments from a liquid biopsy sample of each training subject in a plurality of training subjects.

A candidate somatic variant at a first nucleotide position is identified. In some embodiments this variant is identified based on at least a difference between a respective nucleic acid sequence for a respective cfDNA fragment in the plurality of cfDNA fragments and a corresponding nucleic acid sequence for a locus in a reference sequence to which the respective nucleic acid sequence maps.

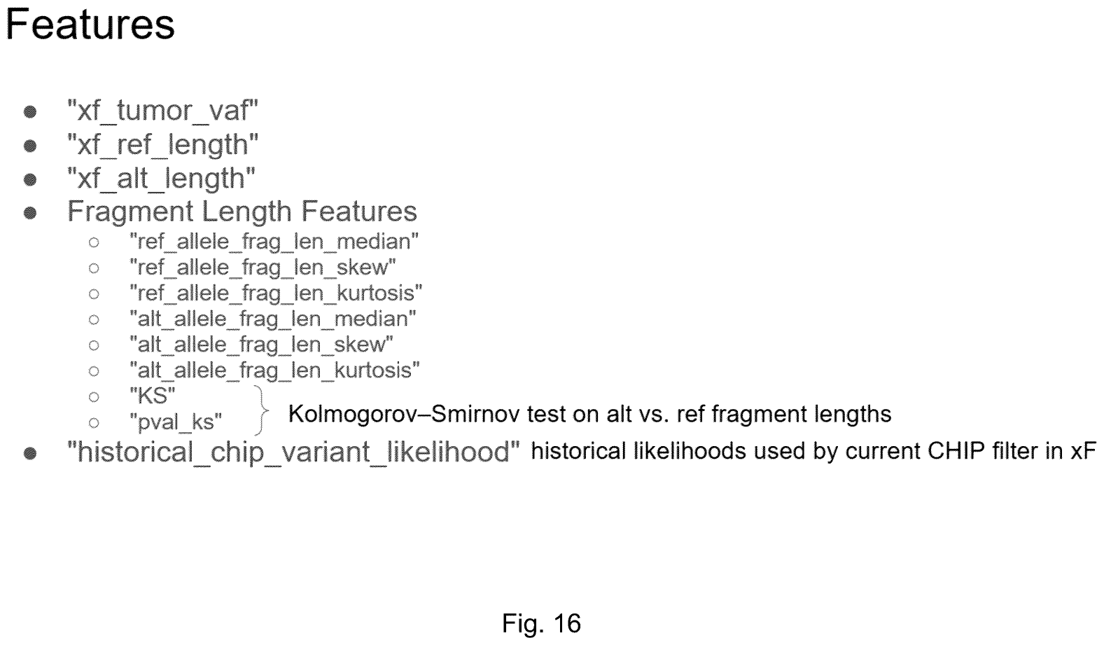

An identity of a first set of cfDNA fragments in the plurality of cfDNA fragments comprising the candidate somatic variant is used to determine one or more fragment length metrics for the candidate somatic variant. FIG. 16 illustrates eight fragment length metrics that are used in this example. These eight fragment length metrics are further described below.

(ref_allele_frag_len_median) reference allele fragment length median: This metric was determined as the median value for the fragment length of all cfDNA fragments sequenced in the liquid biopsy assay for a particular subject (test subject or training subject) that map to the position of the candidate somatic variant but have the wild-type sequence rather than the variant sequence.

(ref_allele_frag_len_skew) reference allele fragment length skew: This metric was a skew value for the distribution of fragments lengths for all cfDNA fragments sequenced in the liquid biopsy sample for a particular subject (test subject or training subject) that map to the position of the candidate somatic variant but have the wild-type sequence rather than the variant sequence. Further details of skew are found in conjunction with block 534 above.

(ref_allele_frag_len_kurtosis) reference allele fragment length kurtosis: This metric was a kurtosis value for the distribution of fragments lengths for all cfDNA fragments sequenced in the liquid biopsy sample for a particular subject (test subject or training subject) that map to the position of the candidate somatic variant but have the wild-type sequence rather than the variant sequence. Further details of kurtosis are found in conjunction with block 532.

(alt_allele_frag_len_median) alternate allele fragment length median: This metric was determined as the median value for the fragment length of all cfDNA fragments sequenced in the liquid biopsy assay for a particular subject that include the candidate somatic variant.

(alt_allele_frag_len_skew) alternate allele fragment length skew: This metric is a skew value for the distribution of fragments lengths for all cfDNA containing the candidate somatic variant in the liquid biopsy sample of a particular subject. Further details of skew are found in conjunction with block 534 above.

(alt_allele_frag_len_kurtosis) alternate allele fragment length kurtosis: This metric is a kurtosis value for the distribution of fragments lengths for all cfDNA containing the candidate somatic variant in the liquid biopsy sample of a particular subject. Further details of kurtosis are found in conjunction with block 532.

(KS) Kolmogorov-Smirnov test metric for fragment length: This metric was determined as the Kolmogorov-Smirnov test statistic for the comparison of (i) the distribution of fragments lengths for cfDNA containing the candidate somatic variant in the liquid biopsy sample of a particular subject to (ii) the distribution of fragments lengths for cfDNA that map to the position of the candidate somatic variant but don't contain the candidate somatic variant in the liquid biopsy sample.

(pval_ks) p-value for Kolmogorov-Smirnov test metric for fragment length: This metric was determined as the p-value for the Kolmogorov-Smirnov test statistic for the comparison of (i) the distribution of fragments lengths for cfDNA containing the candidate somatic variant in the liquid biopsy sample for a particular subject to (ii) the distribution of fragments lengths for cfDNA that map to the position of the candidate variant but that don't contain the candidate somatic variant in the liquid biopsy sample for the particular subject.

A variant allele fraction for the candidate somatic variant in the plurality of cfDNA fragments was determined based on (i) the number of times the candidate somatic variant is observed across the corresponding nucleic acid sequences for each cfDNA fragment in the plurality of cfDNA fragments for a particular subject and (ii) the number of times the first nucleotide position is observed across the corresponding nucleic acid sequences for each cfDNA fragment in the plurality of cfDNA fragments for the particular subject. This feature is referred to as “xf_tumor_vaf” in FIG. 16.

A clonal hematopoiesis prevalence metrics was obtained for the first nucleotide position. This metric is referred to as “historical_chip_variant_likelihood” in FIG. 16. This metric was determined for a particular variant based on sequencing results for a cohort of solid tumor samples with matched liquid biopsy samples. The metric was calculated as the total number of occurrences of the variant in the solid tumor samples that were classified as somatic divided by the total number of occurrences of the variant in the solid tumor samples that were classified as either somatic or non-somatic (e.g., either of germline or CHIP lineage).

In some embodiments, the variant was an indel. In such instances, two additional features are used, “xf_ref_length” and “xf_alt_length.” In some embodiments the indel is limited to a maximum of three nucleotides. In some embodiments no size restrictions are imposed on the indel. Here “xf_alt_length” is the length of the indel while “xf_ref_length” is length of a reference sequence at a position of the indel in a reference genome. For instance, if the indel is a deletion of three residues, xf_alt_length is zero and xf_ref_length is three. Conversely, if the indel is an insertion of three residues, xf_alt_length is three and xf_ref_length is zero.

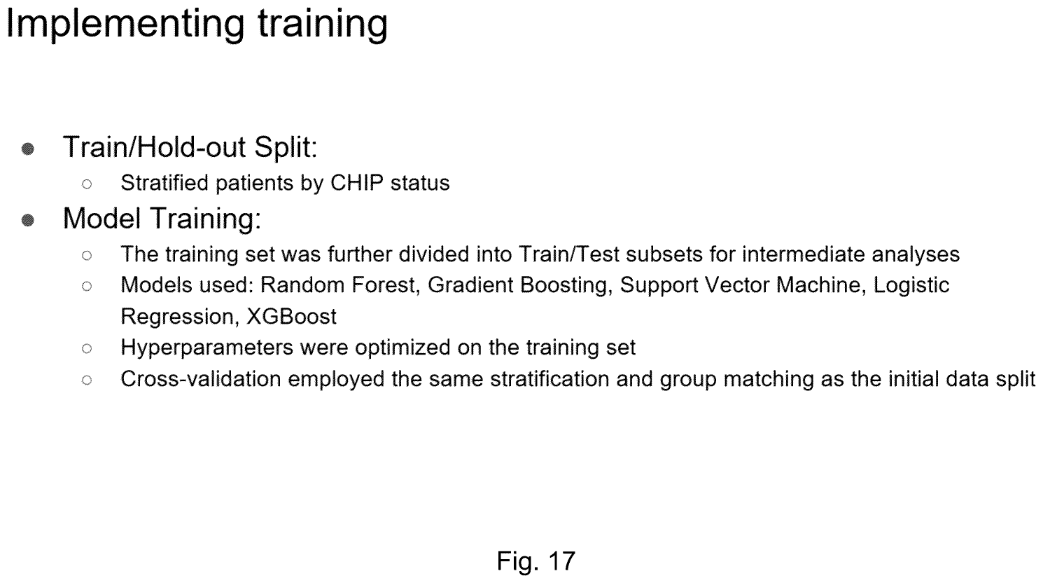

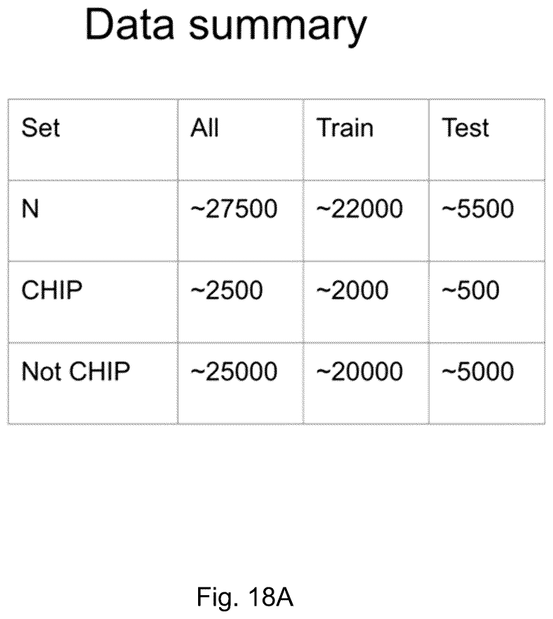

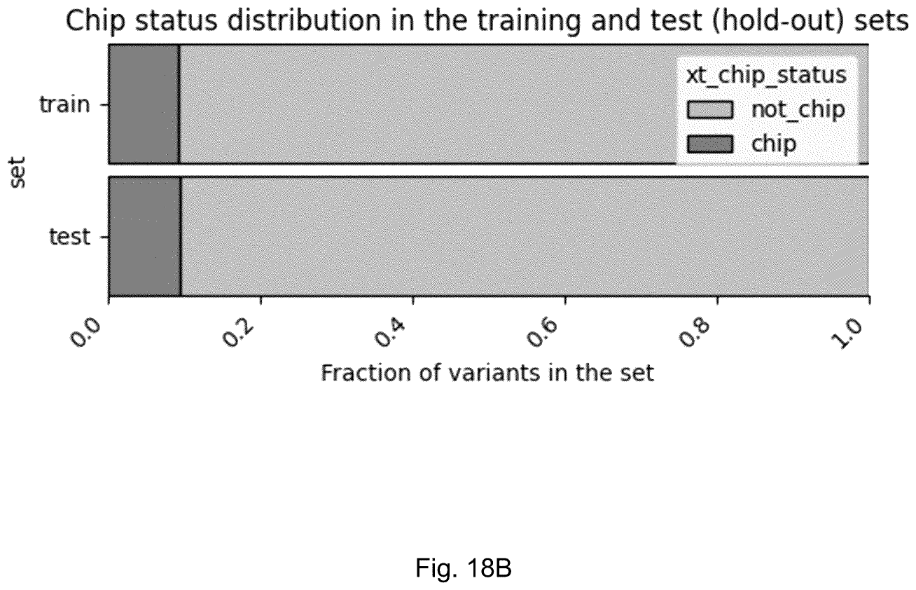

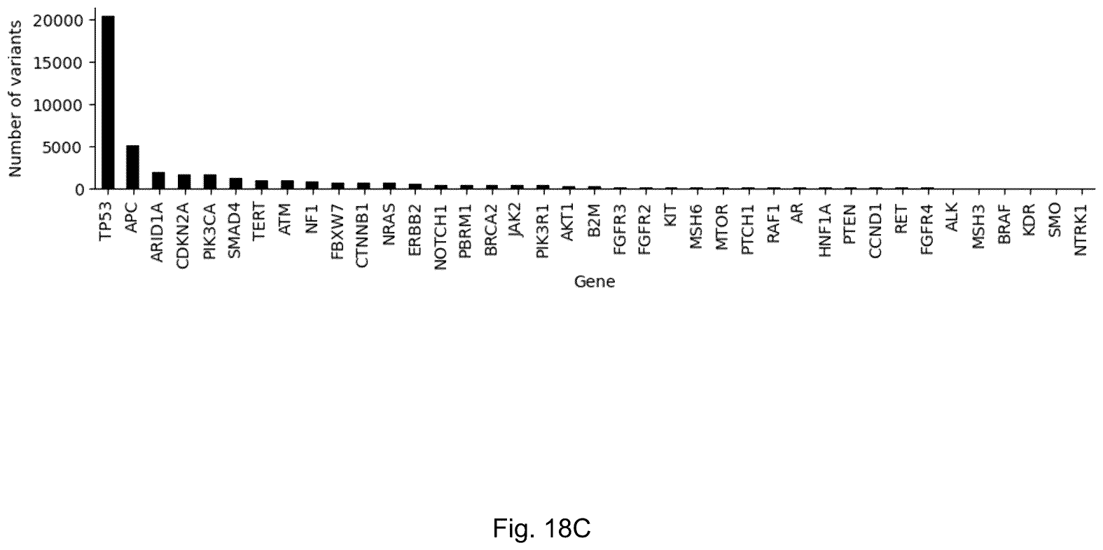

FIG. 17 illustrated details of how the model of Example 4 was trained. The training cohort was defined in expert DEID. An expert DEID ensures that the data used for training, analysis, or research is safely de-identified, allowing for the use of real-world datasets without compromising privacy. The subjects in the training cohort were stratified by CHIP status. In other words, subjects in the training cohort that have blood cells derived from a single hematopoietic (blood-forming) stem cell that has acquired specific mutations associated with Clonal Hematopoiesis of Indeterminate Potential (CHIP) were stratified between the training and test datasets as illustrated in FIG. 18B. The training set was used to train model parameters while the test set was used to evaluate model performance.

As illustrated in FIG. 17, the training set was divided into the train and test subsets referenced in FIG. 18B. As further illustrated in FIG. 17, several models were evaluated in this example for their ability to correctly validate a somatic sequence variant of a test subject including random forest, gradient boosting, support vector machine, logistic regression, and XGBoost. FIG. 18A illustrates that there were ˜ 27500 variants in the training set, divided into ˜22,000 variants for model training and ˜5,500 for model testing. Of the ˜27500 training variants ˜2500 had CHIP detected whereas ˜25000 were free of any CHIP mutations. FIG. 18B further illustrates how the CHIP subjects were distributed in the training and test subsets. FIG. 18C illustrates how many variants in each gene listed on the X-axis of the Figure were identified in the ˜27500 training variants.

Information was inputted into a model comprising a plurality of parameters thereby obtaining as output from the model an indication of whether the candidate somatic variant has a somatic lineage. The information comprised (i) the eight fragment length metrics described above in conjunction with FIG. 16 (“ref_allele_frag_len_median,” “ref_allele_frag_len_skew,” “ref_allele_frag_len_kurtosis,” “alt_allele_frag_len_median,” “alt_allele_frag_len_skew,” “alt_allele_frag_len_kurtosis,” “KS,” and “pval_ks,” (ii) the variant allele fraction for the candidate somatic variant (“xf_tumor_vaf”) described above in conjunction with FIG. 16, and (iii) the clonal hematopoiesis metric for the first nucleotide position described above in conjunction with FIG. 16 (“historical_chip_variant_likelihood”). Additionally, the “xf_ref_length” and “xf_alt_length” features were used in instance where the mutation was an indel.

In this example, a somatic sequence variant was validated when the indication of whether the candidate somatic variant has a somatic lineage satisfies a criterion. The somatic sequence variant was rejected (as being somatic) when the indication of whether the candidate somatic variant has a somatic lineage fails to satisfy the criterion.

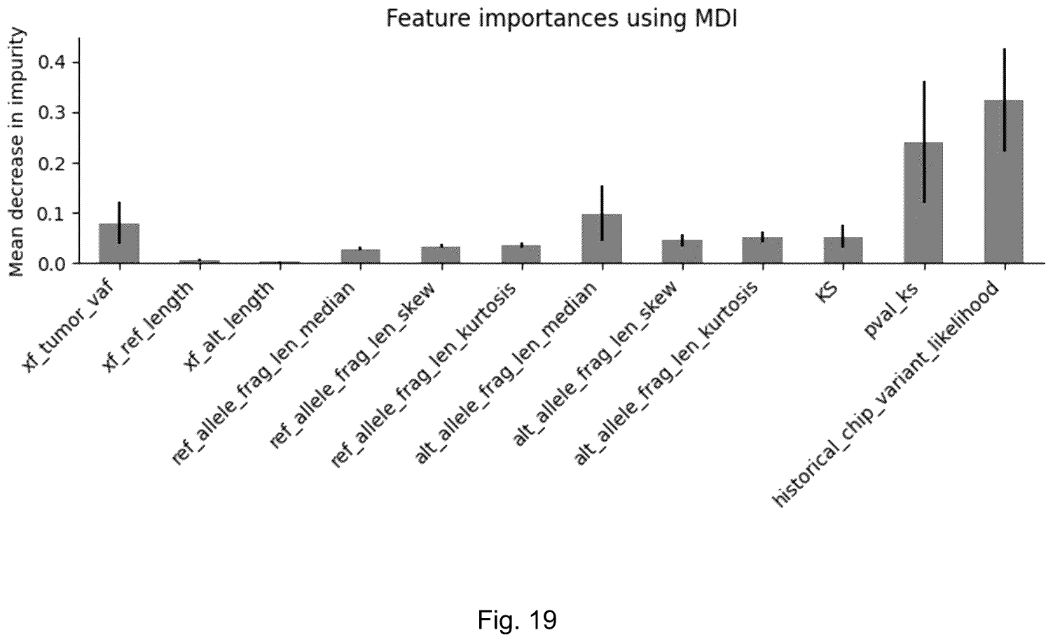

Feature analysis was performed based on decrease in Gini coefficient for each feature in the model. FIG. 19 illustrates the relative importance of each of the features in the XBBoost model. The Figure indicates that “historical_chip_variant_likelihood,” “pval_ks,” and xf_tumor_vaf” were the top 3 features in the model.

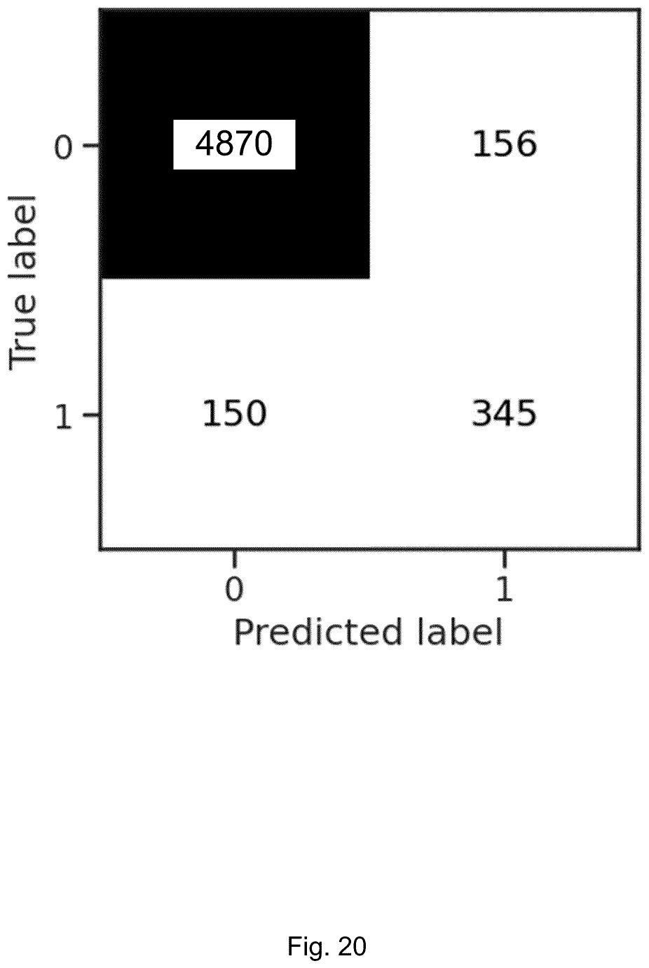

FIG. 20 illustrates the performance of the trained XGBoost model with respect to the ˜5,500 variant test dataset described in FIG. 18A.

### Additional Embodiments

Another aspect of the present disclosure provides a computer system comprising one or more processors and a non-transitory computer-readable medium including computer-executable instructions that, when executed by the one or more processors, cause the processors to perform any of the methods and/or embodiments disclosed herein.

Yet another aspect of the present disclosure provides a non-transitory computer-readable storage medium having stored thereon program code instructions that, when executed by a processor, cause the processor to perform any of the methods and/or embodiments disclosed herein.

Although inventions have been particularly shown and described with reference to a preferred embodiment and various alternate embodiments, it will be understood by persons skilled in the relevant art that various changes in form and details can be made therein without departing from the spirit and scope of the invention.

### Equivalents and Incorporation by Reference.

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57 (b) (2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### Additional Consideration

The foregoing description of the embodiments has been presented for the purpose of illustration; it is not intended to be exhaustive or to limit the patent rights to the precise forms disclosed. Persons skilled in the relevant art will appreciate that many modifications and variations are possible in light of the above disclosure.

Any feature mentioned in one claim category, e.g., method, can be claimed in another claim category, e.g., computer program product, system, storage medium, as well. The dependencies or references back in the attached claims are chosen for formal reasons only. However, any subject matter resulting from a deliberate reference back to any previous claims (in particular multiple dependencies) can be claimed as well, so that any combination of claims and the features thereof is disclosed and can be claimed regardless of the dependencies chosen in the attached claims. The subject matter, in some embodiments, includes not only the combinations of features as set out in the disclosed embodiments but also any other combination of features from different embodiments. Various features mentioned in the different embodiments can be combined with explicit mentioning of such combination or arrangement in an example embodiment or without any explicit mentioning. Furthermore, any of the embodiments and features described or depicted herein, in some embodiments, are claimed in a separate claim and/or in any combination with any embodiment or feature described or depicted herein or with any of the features.

Some portions of this description describe the embodiments in terms of algorithms and symbolic representations of operations on information. These operations and algorithmic descriptions, while described functionally, computationally, or logically, are understood to be implemented by computer programs or equivalent electrical circuits, microcode, or the like. Furthermore, it has also proven convenient at times, to refer to these arrangements of operations as engines, without loss of generality. The described operations and their associated engines are, in some embodiments, embodied in software, firmware, hardware, or any combinations thereof.

Any of the steps, operations, or processes described herein, in some embodiments, are performed or implemented with one or more hardware or software engines, alone or in combination with other devices. In one embodiment, a software engine is implemented with a computer program product comprising a computer-readable medium containing computer program code, which can be executed by a computer processor for performing any or all of the steps, operations, or processes described. The term “steps” does not mandate or imply a particular order. For example, while this disclosure describes, in some embodiments, a process that includes multiple steps sequentially with arrows present in a flowchart, the steps in the process do not need to be performed by the specific order claimed or described in the disclosure. In some implementations, some steps are performed before others even though the other steps are claimed or described first in this disclosure. Likewise, any use of (i), (ii), (iii), etc., or (a), (b), (c), etc. in the specification or in the claims, unless specified, is used to better enumerate items or steps and also does not mandate a particular order.

What is claimed is:

1. A method of validating a sequence variant of a test subject as a somatic variant derived from cell free DNA, the method comprising:
at a computer system having one or more processors, and memory storing one or more programs for execution by the one or more processors:
A) obtaining a nucleic acid sequence of each cell-free DNA (cfDNA) fragment in a plurality of cfDNA fragments from a plurality of sequence reads of a sequencing reaction of the plurality of cfDNA fragments from a liquid biopsy sample of the test subject;
B) identifying a candidate variant at a first locus based on at least a difference between a respective nucleic acid sequence for a respective cfDNA fragment in the plurality of cfDNA fragments and a nucleic acid sequence for the first locus in a reference sequence;
C) using an identity of a first set of cfDNA fragments in the plurality of cfDNA fragments comprising the candidate variant to determine one or more fragment length metrics for the candidate variant;
D) determining a variant allele fraction for the candidate variant in the plurality of cfDNA fragments based on (i) the number of cfDNA fragments in the plurality of cfDNA fragments having the candidate variant and (ii) the number of cfDNA fragments in the plurality of cfDNA fragments mapping to the first locus;
E) obtaining one or more clonal hematopoiesis prevalence metrics for the candidate variant;
F) inputting information into a first model comprising a plurality of parameters thereby obtaining as output from the first model, through application of the plurality of parameters to the information, whether the candidate variant is (a) a somatic variant derived from cell free DNA (b) other than a somatic variant derived from cell free DNA,
wherein the information comprises (i) the one or more fragment length metrics, (ii) the variant allele fraction for the candidate variant or one or more features determined from the variant allele fraction for the candidate variant, and (iii) the one or more clonal hematopoiesis metrics for the candidate variant.

2. The method of claim 1, further comprising determining an estimated circulating tumor fraction (ctFE) for the test subject, and wherein the information further comprises the ctFE for the test subject.

3. The method of claim 2, wherein the information comprises the variant allele fraction and the ctFE.

4. The method of claim 2, wherein the information comprises the one or more features determined from (i) the variant allele fraction for the candidate variant and (ii) the ctFE for the liquid biopsy sample and a feature in the one or more features is a residual value calculated by:
inputting the variant allele fraction into a second model to obtain as output from the second model an expected circulating tumor fraction and comparing (i) the expected circulating tumor fraction with (ii) the ctFE; or
inputting the ctFE into a third model to obtain as output from the third model an expected variant allele fraction and comparing (i) the expected variant allele fraction with (ii) the variant allele fraction for the candidate variant.

5. The method of claim 1, wherein a clonal hematopoiesis prevalence metric in the one or more clonal hematopoiesis prevalence metrics is a frequency, in a cohort of solid tumors, of a variant of hematopoietic lineage at the first locus.

6. The method of claim 1, wherein a clonal hematopoiesis prevalence metric in the one or more clonal hematopoiesis prevalence metrics is:
a comparison of (i) instances of the candidate variant, in a cohort of solid tumors, that are of hematopoietic or germline lineage and (ii) total instances of the candidate variant in the cohort of solid tumors; or
a comparison of (i) instances of the candidate variant, in the cohort of solid tumors, that are of solid tumor lineage and (ii) total instances of the candidate variant in the cohort of solid tumors.

7. The method of claim 1, wherein a fragment length metric in the one or more fragment metrics is a measure of central tendency of the first set of cfDNA fragments, a kurtosis of the first set of cfDNA fragments, a skew of the first set of cfDNA fragments, a measure of central tendency of a second set of cfDNA fragments, a kurtosis of the second set of cfDNA fragments, or a skew of the second set of cfDNA fragments, wherein the second set of cfDNA fragments is the cfDNA fragments in the plurality of cfDNA fragments, other than the first set of cfDNA fragments, comprising the first locus.

8. The method of claim 1, wherein a fragment length metric in the one or more fragment length metrics is a p-value determined by application of a Kolmogorov-Smirnov test to a difference in (i) a distribution of fragment lengths of the first set of cfDNA fragments versus (ii) a distribution of fragment lengths of a second set of cfDNA fragments in the plurality of cfDNA fragments, wherein the second set of cfDNA fragments is the cfDNA fragments in the plurality of cfDNA fragments, other than the first set of cfDNA fragments, comprising the first locus.

9. The method of claim 1, wherein a fragment length metric in the one or more fragment length metrics is determined by application of a Kolmogorov-Smirnov test to a difference in (i) a distribution of fragment lengths of the first set of cfDNA fragments versus (ii) a distribution of fragment lengths of a second set of cfDNA fragments in the plurality of cfDNA fragments, wherein the second set of cfDNA fragments is the cfDNA fragments in the plurality of cfDNA fragments, other than the first set of cfDNA fragments, comprising the first locus.

10. The method of claim 1, wherein the first model is selected from the group consisting of a regression model, a neural network, a support vector machine, a Naive Bayes model, a nearest neighbor model, a boosted trees model, a random forest model, a decision tree, a gradient boosted tree, an elastic net, a logistic regression model, a clustering model, and an XGboost model.

11. The method of claim 1, wherein the first model applies the plurality of parameters to the information through a plurality of computations and the plurality of computations is at least 10,000 computations.

12. The method of claim 1, wherein the liquid biopsy sample comprises blood, whole blood, peripheral blood, plasma, serum, or lymph of the test subject.

13. The method of claim 1, wherein the candidate variant is a single nucleotide variant (SNV) or an indel.

14. The method of claim 13, wherein the information further comprises a length of the indel or a length of a reference sequence at the first locus in a reference genome.

15. The method of claim 1, wherein the sequencing reaction is a panel-based sequencing reaction of a plurality of loci, wherein the plurality of loci comprises at least 5 genes in Table 1, at least 25 genes in Table 1, at least 50 genes in Table 1, at least 5 genes in Table 2, at least 25 genes in Table 2, or at least 50 genes in Table 2.

16. The method of claim 1, wherein the test subject is afflicted with a cancer condition.

17. The method of claim 16, wherein the cancer condition is lung cancer, breast cancer, ovarian cancer, cervical cancer, a uveal melanoma, colorectal cancer, chromophobe renal cell carcinoma, liver cancer, an endocrine tumor, oropharyngeal cancer, retinoblastoma, biliary cancer, adrenal cancer, neural cancer, neuroblastoma, basal cell carcinoma, brain cancer, a non-clear cell renal cell carcinoma, a glioblastoma, a glioma, kidney cancer, gastrointestinal stromal tumor, a medulloblastoma, bladder cancer, gastric cancer, bone cancer, thymoma, prostate cancer, a clear cell renal cell carcinoma, skin cancer, thyroid cancer, a sarcoma, testicular cancer, head and neck cancer, a meningioma, peritoneal cancer, endometrial cancer, pancreatic cancer, mesothelioma, esophageal cancer, small cell lung cancer, HER2 negative breast cancer, ovarian serous carcinoma, HR+ breast cancer, uterine serous carcinoma, a uterine corpus endometrial carcinoma, a gastroesophageal junction adenocarcinoma, gallbladder cancer, chordoma, or a papillary renal cell carcinoma.

18. The method of claim 1, further comprising, when the first model indicates the candidate variant is a somatic variant derived from cell free DNA, generating a report for the test subject comprising the identity of the candidate variant, wherein the report further comprises a therapeutic recommendation for the test subject based on the identity of the candidate variant.

19. A computer system comprising:
one or more processors; and
a non-transitory computer-readable medium including computer-executable instructions that, when executed by the one or more processors, cause the processors to perform a method comprising:
A) obtaining a nucleic acid sequence of each cell-free DNA (cfDNA) fragment in a plurality of cfDNA fragments from a plurality of sequence reads of a sequencing reaction of the plurality of cfDNA fragments from a liquid biopsy sample of the test subject;
B) identifying a candidate variant at a first locus based on at least a difference between a respective nucleic acid sequence for a respective cfDNA fragment in the plurality of cfDNA fragments and a nucleic acid sequence for the first locus in a reference sequence;
C) using an identity of a first set of cfDNA fragments in the plurality of cfDNA fragments comprising the candidate variant to determine one or more fragment length metrics for the candidate variant;
D) determining a variant allele fraction for the candidate variant in the plurality of cfDNA fragments based on (i) the number of cfDNA fragments in the plurality of cfDNA fragments having the candidate variant and (ii) the number of cfDNA fragments in the plurality of cfDNA fragments mapping to the first locus;
E) obtaining one or more clonal hematopoiesis prevalence metrics for the candidate variant;
F) inputting information into a first model comprising a plurality of parameters thereby obtaining as output from the first model, through application of the plurality of parameters to the information, whether the candidate variant is (a) a somatic variant derived from cell free DNA (b) other than a somatic variant derived from cell free DNA,
wherein the information comprises (i) the one or more fragment length metrics, (ii) the variant allele fraction for the candidate variant or one or more features determined from the variant allele fraction for the candidate variant, and (iii) the one or more clonal hematopoiesis metrics for the candidate variant.

20. A non-transitory computer-readable storage medium having stored thereon program code instructions that, when executed by a processor, cause the processor to perform a method comprising:
A) obtaining a nucleic acid sequence of each cell-free DNA (cfDNA) fragment in a plurality of cfDNA fragments from a plurality of sequence reads of a sequencing reaction of the plurality of cfDNA fragments from a liquid biopsy sample of the test subject;
B) identifying a candidate variant at a first locus based on at least a difference between a respective nucleic acid sequence for a respective cfDNA fragment in the plurality of cfDNA fragments and a nucleic acid sequence for the first locus in a reference sequence;
C) using an identity of a first set of cfDNA fragments in the plurality of cfDNA fragments comprising the candidate variant to determine one or more fragment length metrics for the candidate variant;
D) determining a variant allele fraction for the candidate variant in the plurality of cfDNA fragments based on (i) the number of cfDNA fragments in the plurality of cfDNA fragments having the candidate variant and (ii) the number of cfDNA fragments in the plurality of cfDNA fragments mapping to the first locus;
E) obtaining one or more clonal hematopoiesis prevalence metrics for the candidate variant;
F) inputting information into a first model comprising a plurality of parameters thereby obtaining as output from the first model, through application of the plurality of parameters to the information, whether the candidate variant is (a) a somatic variant derived from cell free DNA (b) other than a somatic variant derived from cell free DNA,
wherein the information comprises (i) the one or more fragment length metrics, (ii) the variant allele fraction for the candidate variant or one or more features determined from the variant allele fraction for the candidate variant, and (iii) the one or more clonal hematopoiesis metrics for the candidate variant.
